(19) United States
(12) Patent Application Publication
(10) Pub. No.: US 2022/0216424 A1
(43) Pub. Date: Jul. 7, 2022

(54) MATERIALS FOR ORGANIC ELECTROLUMINESCENT DEVICES

(71) Applicant: Merck Patent GmbH, Darmstadt (DE)

(72) Inventors: Amir Hossain PARHAM, Frankfurt am Main (DE); Jonas Valentin KROEBER, Frankfurt am Main (DE); Jens ENGELHART, Darmstadt (DE); Anja JATSCH, Frankfurt am Main (DE); Christian EICKHOFF, Mannheim (DE); Christian EHRENREICH, Darmstadt (DE)

(21) Appl. No.: 17/442,788

(22) PCT Filed: Mar. 23, 2020
(86) PCT No.: PCT/EP2020/057907

§ 371 Date: Sep. 24, 2021

(30) Foreign Application Priority Data
Mar. 25, 2019 (EP) 19164857.5

(51) Int. Cl. H01L 51/00; C07D 471/04; C09K 11/06; C07D 471/14; C07F 5/02; C07D 513/04

(52) U.S. Cl. CPC H01L 51/0072; H01L 51/5016; C09K 11/06; H01L 51/0073; H01L 51/006; H01L 51/0061; H01L 51/0067; C07D 471/14; C07F 5/027; H01L 51/008; C07D 513/04; H01L 51/0071; H01L 51/0054; H01L 51/0052; C07D 471/04

(57) ABSTRACT

The invention relates to compounds which are suitable for use in electronic devices, and to electronic devices, in particular organic electroluminescent devices, containing said compounds.

The present invention relates to materials for use in electronic devices, especially in organic electroluminescent devices, and to electronic devices, especially organic electroluminescent devices comprising these materials.

Emitting materials used in organic electroluminescent devices (OLEDs) are frequently phosphorescent organometallic complexes. In general terms, there is still a need for improvement in OLEDs, especially also in OLEDs which exhibit triplet emission (phosphorescence), for example with regard to efficiency, operating voltage and lifetime. The properties of phosphorescent OLEDs are not just determined by the triplet emitters used. More particularly, the other materials used, such as matrix materials, are also of particular significance here. Improvements to these materials can thus also lead to improvements in the OLED properties. Suitable matrix materials for OLEDs are, for example, aromatic lactams as disclosed, for example, in WO 2011/116865, WO 2011/137951 or WO 2013/064206.

The problem addressed by the present invention is that of providing compounds which are suitable for use in an OLED, especially as matrix material for phosphorescent emitters or as electron transport material, and which lead to improved properties therein. It is a further object of the present invention to provide further organic semiconductors for organic electroluminescent devices, in order thus to enable the person skilled in the art to have a greater possible choice of materials for the production of OLEDs.

It has been found that, surprisingly, this object is achieved by particular compounds described in detail hereinafter that are of good suitability for use in OLEDs. These OLEDs especially have a long lifetime, improved efficiency and relatively low operating voltage. The present invention therefore provides these compounds and electronic devices, especially organic electroluminescent devices, comprising these compounds.

The present invention provides a compound of formula (1)

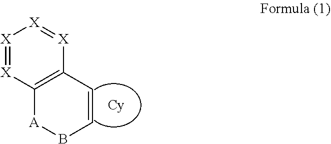

where the symbols used are as follows:
A, B are each selected from the group consisting of NAr¹, C═O, C═S, C═NR, BR, PR, P(═O)R, SO and SO₂, with the proviso that one of the symbols A and B is NAr¹ and the other of the symbols A and B is
C═O, C═S, C═NR, BR, PR, P(═O)R, SO or SO₂; Cy together with the two carbon atoms shown explicitly is a group of the following formula (2):

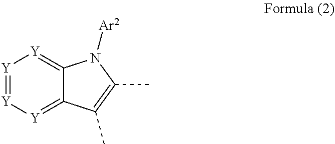

where the dotted bonds indicate the linkage of this group in the formula (1);
X is the same or different at each instance and is CR or N; or two adjacent X groups are a group of the formula (3) below, and the two other symbols X are the same or different at each instance and are CR or N,

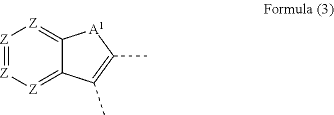

where the dotted bonds indicate the linkage of this group in the formula (1);
Y is the same or different at each instance and is CR or N; or two adjacent Y groups are a group of the formula (3) below, and the two other symbols Y are the same or different at each instance and are CR or N,

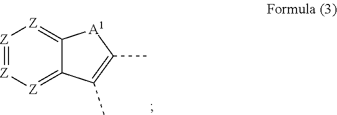

where the dotted bonds indicate the linkage of this group in the formula (1);
A¹ is the same or different at each instance and is NAr³, O, S or C(R)₂;
Z is the same or different at each instance and is CR or N;
Ar¹, Ar², Ar³ is the same or different at each instance and is an aromatic or heteroaromatic ring system which has 5 to 40 aromatic ring atoms and may be substituted by one or more R radicals;
R is the same or different at each instance and is H, D, F, Cl, Br, I, N(Ar′)₂, N(R¹)₂, OAr′, SAr, CN, NO₂, OR¹, SR¹, COOR¹, C(═O)N(R¹)₂, Si(R¹)₃, B(OR¹)₂, C(═O)R¹, P(═O)(R¹)₂, S(═O)R¹, S(═O)₂R¹, OSO₂R¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R¹ radicals, where one or more nonadjacent CH₂ groups may be replaced by Si(R¹)₂, C═O, NR¹, O, S or CONR¹, or an aromatic or heteroaromatic ring system which has 5 to 60 aromatic ring atoms, preferably 5 to 40 aromatic ring atoms, and may be substituted in each case by one or more R¹ radicals; at the same time, two R radicals together may also form an aliphatic, heteroaliphatic, aromatic or heteroaromatic ring system;
Ar′ is the same or different at each instance and is an aromatic or heteroaromatic ring system which has 5 to 40 aromatic ring atoms and may be substituted by one or more R¹ radicals;
R¹ is the same or different at each instance and is H, D, F, Cl, Br, I, N(R²)₂, CN, NO₂, OR², SR², Si(R²)₃, B(OR²)₂, C(═O)R², P(═O)(R²)₂, S(═O)R², S(═O)₂R², OSO₂R², a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may each be substituted by one or more R² radicals, where one or more nonadjacent CH₂ groups may be replaced by Si(R²)₂, C═O, NR², O, S or CONR² and where one or more hydrogen atoms in the alkyl, alkenyl or alkynyl group may be replaced by D, F, Cl, Br, I or CN, or an aromatic or heteroaromatic ring system which has 5 to 40 aromatic ring atoms and may be substituted in each case by one or more R² radicals; at the same time, two or more R¹ radicals together may form an aliphatic ring system;
R² is the same or different at each instance and is H, D, F, CN or an aliphatic, aromatic or heteroaromatic organic radical, especially a hydrocarbyl radical, having 1 to 20 carbon atoms, in which one or more hydrogen atoms may also be replaced by F;

with the proviso that at least one R group is a heteroaromatic ring system and/or that at least one Ar¹ or Ar² group is a heteroaromatic ring system and/or that the compound has at least one group of formula (3).

An aryl group in the context of this invention contains 6 to 40 carbon atoms; a heteroaryl group in the context of this invention contains 2 to 40 carbon atoms and at least one heteroatom, with the proviso that the sum total of carbon atoms and heteroatoms is at least 5. The heteroatoms are preferably selected from N, O and/or S. Here, an aryl group or heteroaryl group is understood to mean either a simple aromatic ring, i.e. benzene, or a simple heteroaromatic ring, for example pyridine, pyrimidine, thiophene, etc., or a condensed (fused) aryl or heteroaryl group, for example naphthalene, anthracene, phenanthrene, quinoline, isoquinoline, etc. Aromatic systems joined to one another by a single bond, for example biphenyl, by contrast, are not referred to as an aryl or heteroaryl group but as an aromatic ring system.

An aromatic ring system in the context of this invention contains 6 to 60 carbon atoms, preferably 6 to 40 carbon atoms, in the ring system. A heteroaromatic ring system in the context of this invention contains 2 to 60 carbon atoms, preferably 2 to 40 carbon atoms, and at least one heteroatom in the ring system, with the proviso that the sum total of carbon atoms and heteroatoms is at least 5. The heteroatoms are preferably selected from N, O and/or S. An aromatic or heteroaromatic ring system in the context of this invention shall be understood to mean a system which does not necessarily contain only aryl or heteroaryl groups, but in which it is also possible for two or more aryl or heteroaryl groups to be joined by a nonaromatic unit, for example a carbon, nitrogen or oxygen atom. These shall likewise be understood to mean systems in which two or more aryl or heteroaryl groups are joined directly to one another, for example biphenyl, terphenyl, bipyridine or phenylpyridine. For example, systems such as fluorene, 9,9′-spirobifluorene, 9,9-diarylfluorene, triarylamine, diaryl ethers, stilbene, etc. shall also be regarded as aromatic ring systems in the context of this invention, and likewise systems in which two or more aryl groups are joined, for example, by a short alkyl group. Preferred aromatic or heteroaromatic ring systems are simple aryl or heteroaryl groups and groups in which two or more aryl or heteroaryl groups are joined directly to one another, for example biphenyl or bipyridine, and also fluorene or spirobifluorene.

In the context of the present invention, an aliphatic hydrocarbyl radical or an alkyl group or an alkenyl or alkynyl group which may contain 1 to 40 carbon atoms and in which individual hydrogen atoms or CH₂ groups may also be substituted by the abovementioned groups is preferably understood to mean the methyl, ethyl, n-propyl, i-propyl, n-butyl, i-butyl, s-butyl, t-butyl, 2-methylbutyl, n-pentyl, s-pentyl, neopentyl, cyclopentyl, n-hexyl, neohexyl, cyclohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl, heptynyl or octynyl radicals. An alkoxy group OR¹ having 1 to 40 carbon atoms is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy and 2,2,2-trifluoroethoxy. A thioalkyl group SR¹ having 1 to 40 carbon atoms is understood to mean especially methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio. In general, alkyl, alkoxy or thioalkyl groups according to the present invention may be straight-chain, branched or cyclic, where one or more nonadjacent CH₂ groups may be replaced by the abovementioned groups; in addition, it is also possible for one or more hydrogen atoms to be replaced by D, F, Cl, Br, I, CN or NO₂, preferably F, Cl or CN, more preferably F or CN.

An aromatic or heteroaromatic ring system which has 5-60 aromatic ring atoms and may also be substituted in each case by the abovementioned R² radicals or a hydrocarbyl radical and which may be joined to the aromatic or heteroaromatic system via any desired positions is understood to mean especially groups derived from benzene, naphthalene, anthracene, benzanthracene, phenanthrene, pyrene, chrysene, perylene, fluoranthene, naphthacene, pentacene, benzopyrene, biphenyl, biphenylene, terphenyl, triphenylene, fluorene, spirobifluorene, dihydrophenanthrene, dihydropyrene, tetrahydropyrene, cis- or trans-indenofluorene, cis- or trans-indenocarbazole, cis- or trans-indolocarbazole, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, hexaazatriphenylene, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole, or groups derived from a combination of these systems.

The wording that two or more radicals together may form an aliphatic ring, in the context of the present description, should be understood to mean, inter alia, that the two radicals are joined to one another by a chemical bond with formal elimination of two hydrogen atoms. This is illustrated by the following scheme:

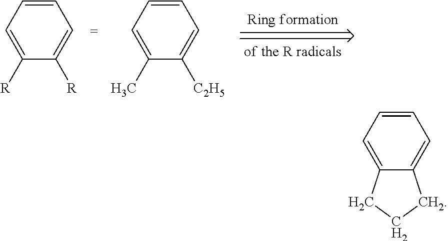

In addition, however, the abovementioned wording shall also be understood to mean that, if one of the two radicals is hydrogen, the second radical binds to the position to which the hydrogen atom was bonded, forming a ring. This shall be illustrated by the following scheme:

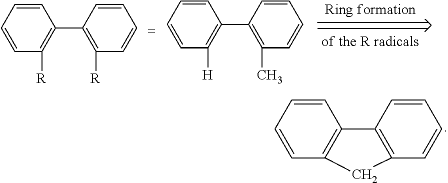

Different isomers arise according to the orientation of the group of the formula (2), as shown below by the formulae (4) and (5),

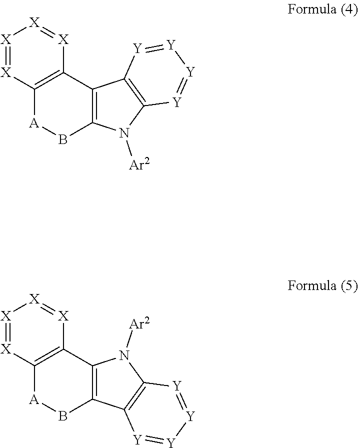

where the symbols used have the definitions given above.

In a preferred embodiment of the invention, one of the A and B groups is NAr¹ and the other of the A and B groups is C═O, P(═O)R, BR or SO₂, especially C═O.

Preferred embodiments of the compounds of the formula (4) are thus the compounds of the following formulae (4a) and (4b), and preferred embodiments of the compounds of the formula (5) are the compounds of the following formulae (5a) and (5b):

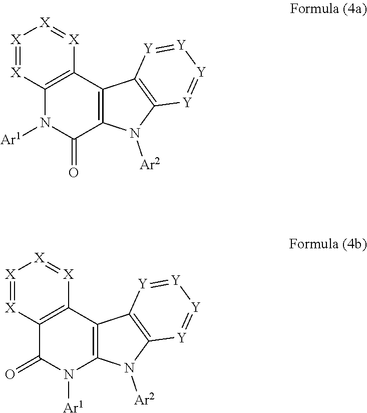

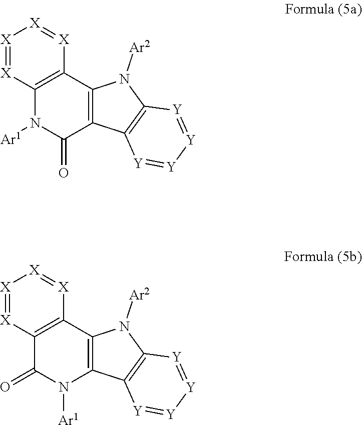

where the symbols used have the definitions given above. Particular preference is given to the compounds of the formula (5a).

In a preferred embodiment of the invention, not more than one symbol X is N and the other symbols X are the same or different and are CR. In a particularly preferred embodiment of the invention, all symbols X are the same or different at each instance and are CR. Particular preference is thus given to the compounds of the following formulae (4a-1), (4b-1), (5a-1) and (5b-1)

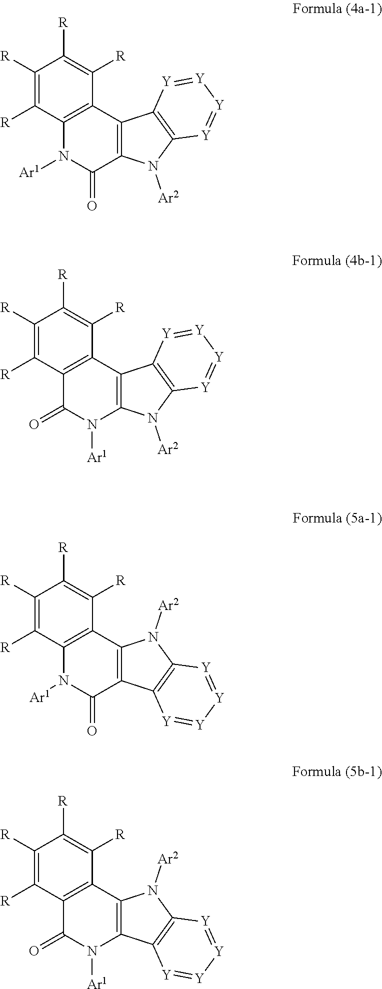

where the symbols used have the definitions given above.

In a preferred embodiment of the invention, not more than one symbol Y is N and the other symbols Y are CR. In a particularly preferred embodiment of the invention, all symbols Y are CR. Particular preference is thus given to the compounds of the following formulae (4a-2), (4b-2), (5a-2) and (5b-2)

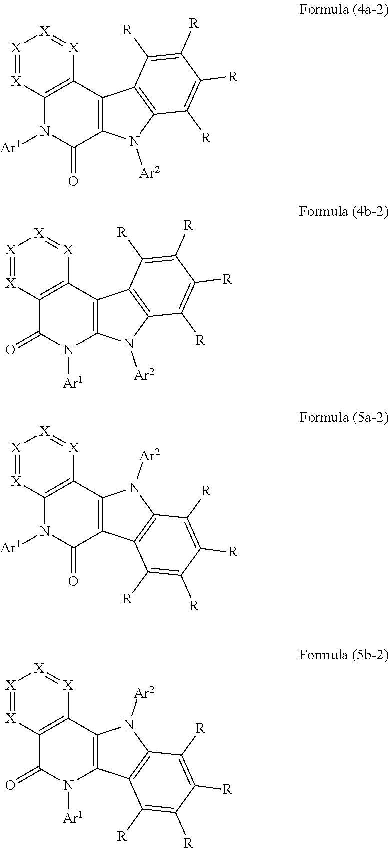

where the symbols used have the definitions given above.

More preferably, the abovementioned preferences for X and Y occur simultaneously, and so particular preference is given to structures of the following formulae: (4a-3), (4b-3), (5a-3) and (5b-3).

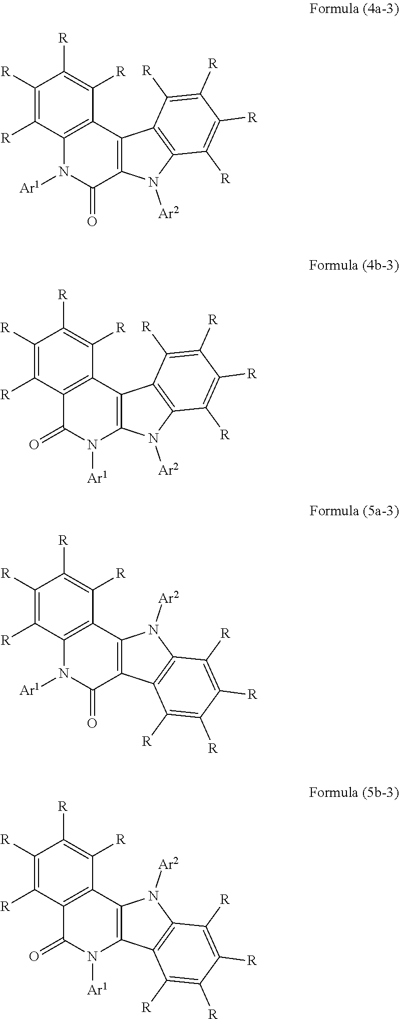

where the symbols used have the definitions given above.

In a preferred embodiment of the invention, not more than three R radicals in total, more preferably not more than two R radicals and most preferably not more than one R radical in the compound of the formula (1) or in the preferred structures detailed above are/is a group other than hydrogen.

Very particular preference is given to the compounds of the following formulae: (4a-4), (4b-4), (5a-4) and (5b-4),

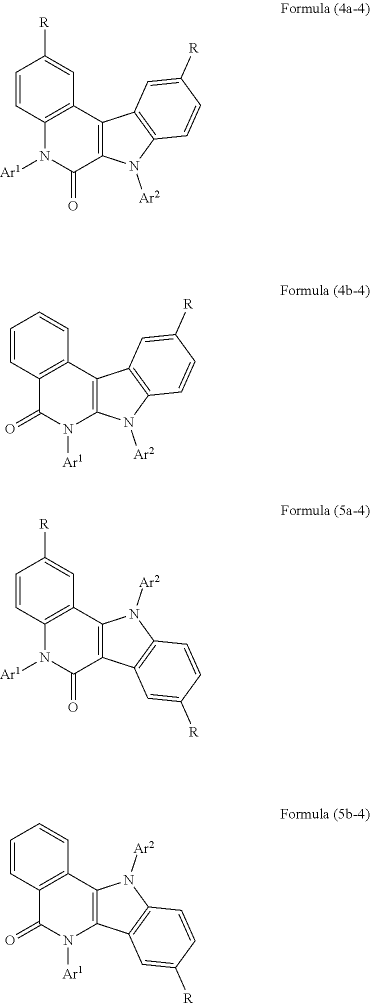

where the symbols used have the definitions given above.

In a further embodiment of the invention, two adjacent Y groups are a group of the formula (3), and the two other symbols Y are the same or different and are CR. In the group of the formula (3), the symbol A¹ is preferably NAr³. If the two Y groups are a group of the formula (3), preferred embodiments of the formula (4) are the compounds of the following formulae (6) to (11), and preferred embodiments of the formula (5) are the compounds of the following formulae (12) to (17):

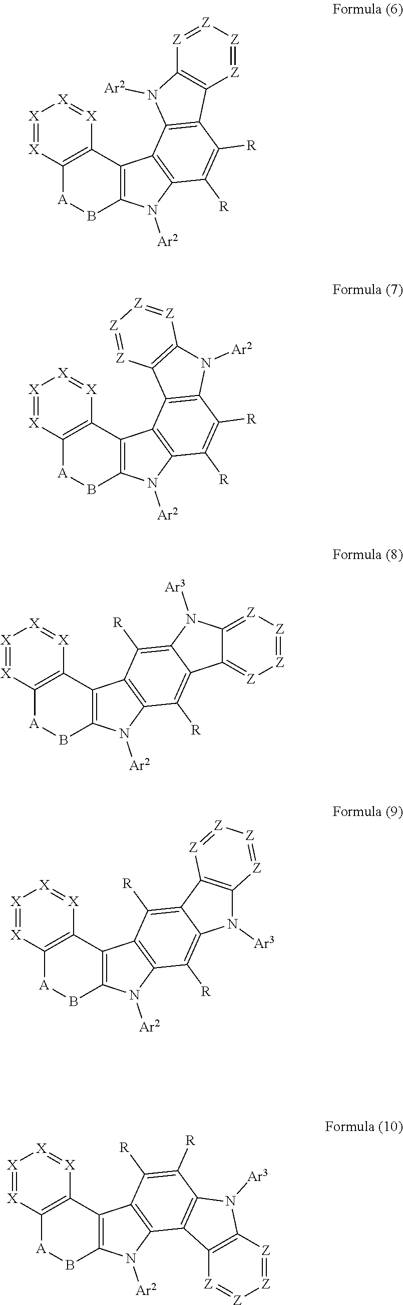

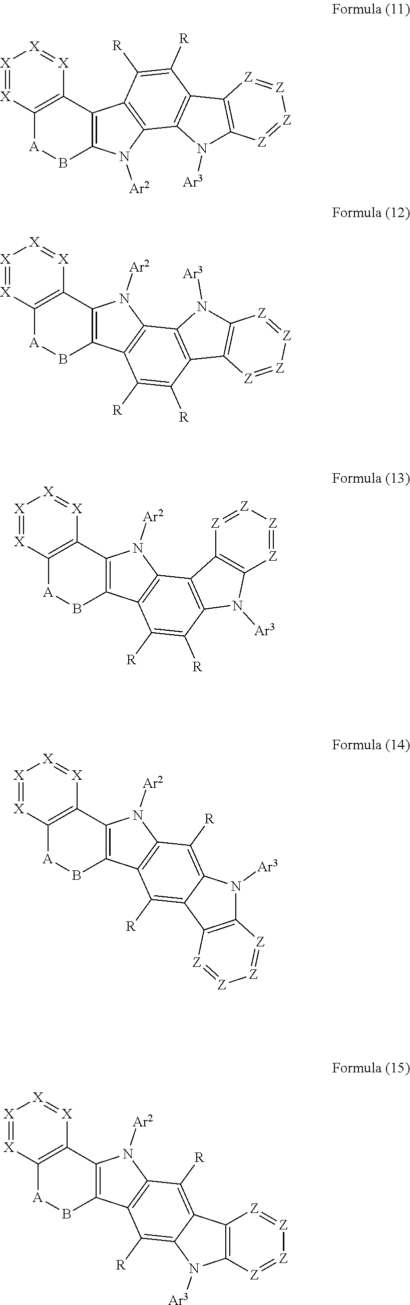

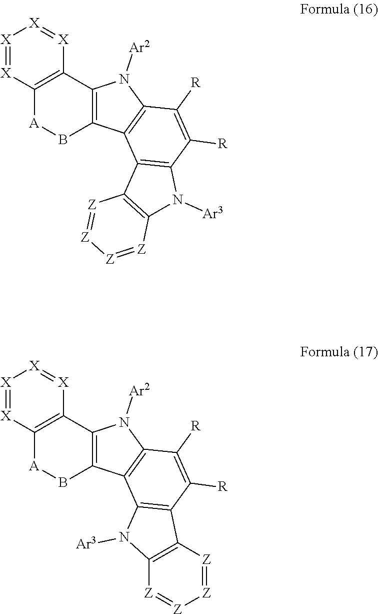

where the symbols used have the definitions given above. It is particularly preferable here when one of the A and B groups is NAr¹ and the other of the A and B groups is C═O.

In formulae (6) to (17), preferably not more than one X group is N and the other X groups are the same or different and are CR. More preferably, all X groups are the same or different and are CR.

In a further preferred embodiment of the invention, not more than one Z group is N, and the other Z groups are the same or different and are CR. More preferably, all Z groups are the same or different and are CR.

Most preferably, in the formulae (6) to (17), all symbols X and Z are the same or different and are CR, and so particular preference is given to the compounds of the following formulae (6-1) to (17-1):

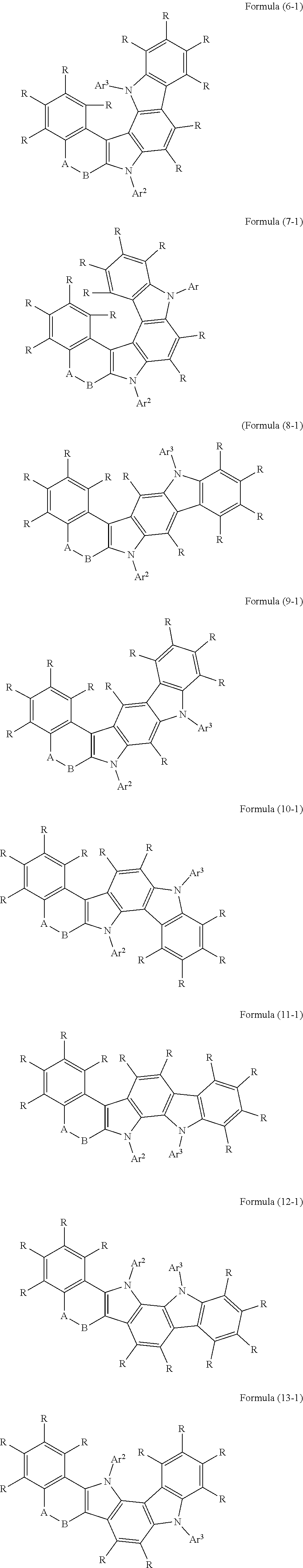

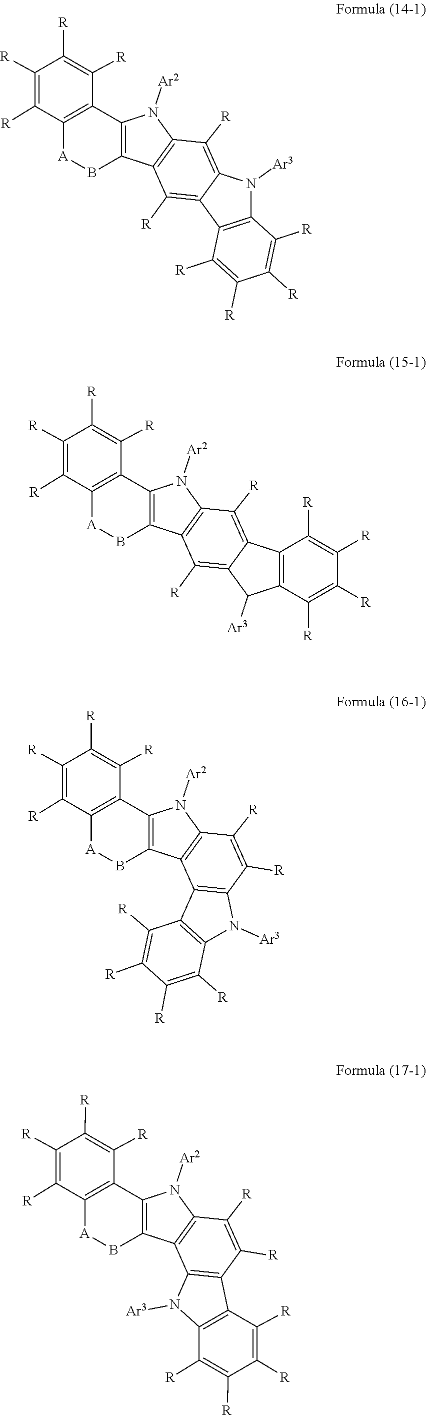

where the symbols used have the definitions given above.

For the formulae (6) to (17) and (6-1) to (17-1), it is preferable that one of the A and B groups is NAr¹ and the other of the A and B groups is C═O. Particular preference is therefore given to the structures of the following formulae (6a-1) to (17b-1):

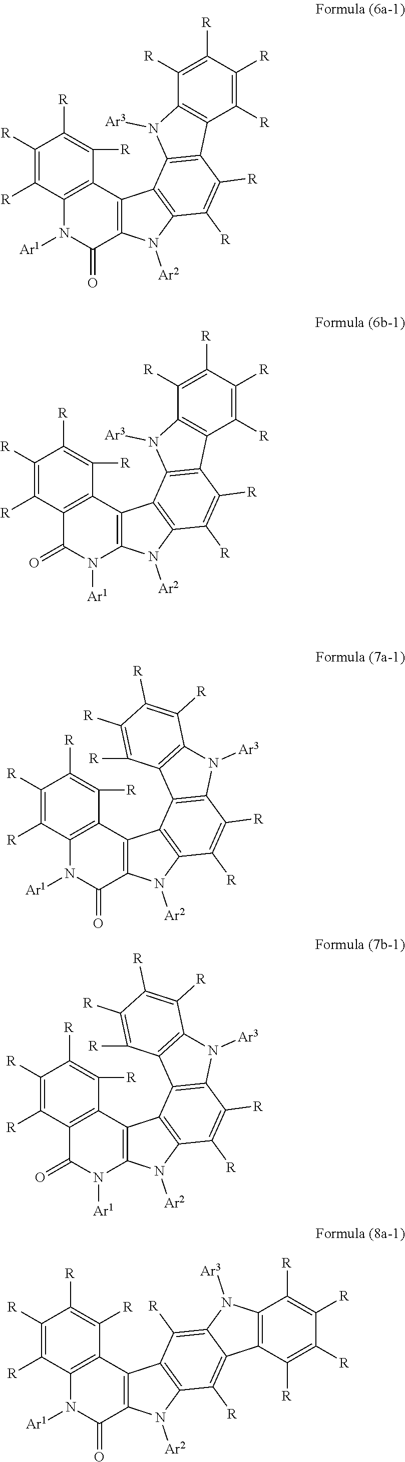

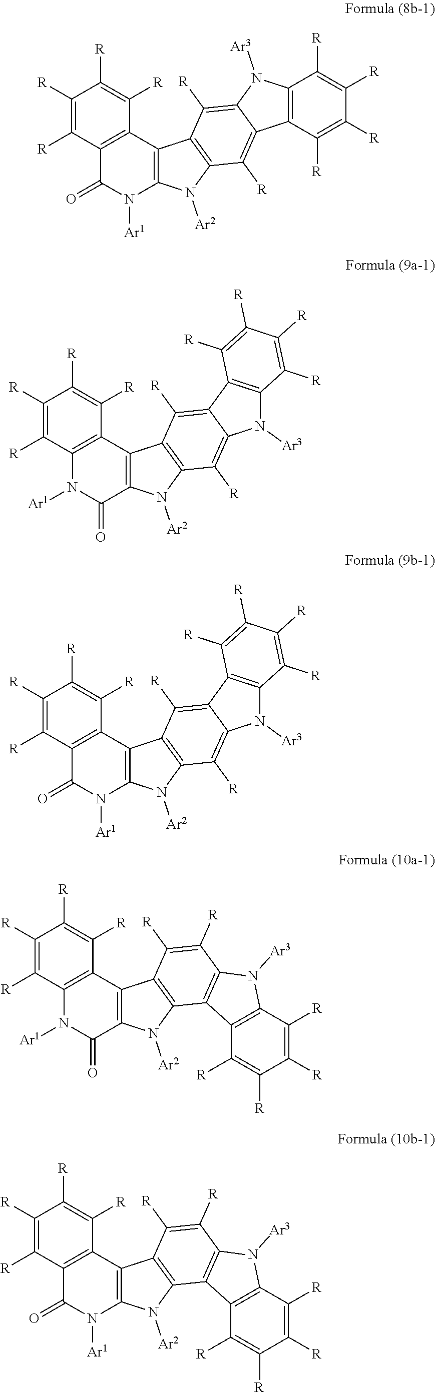

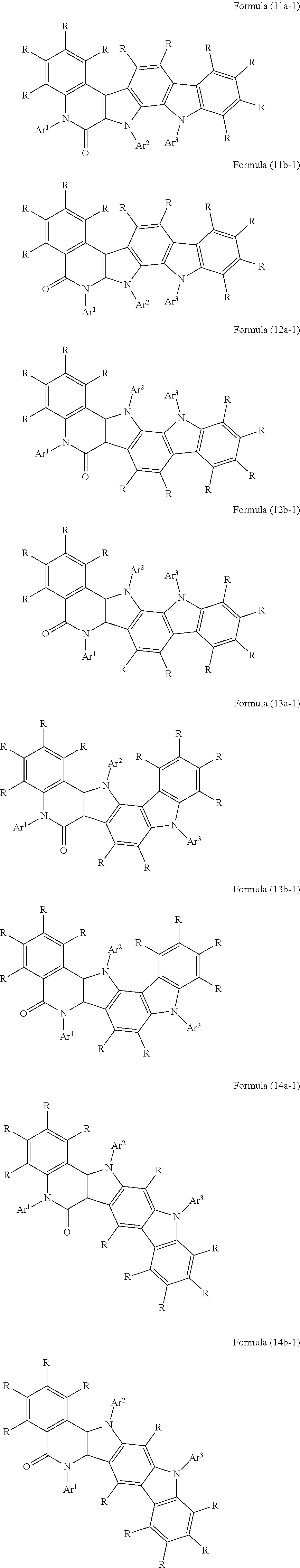

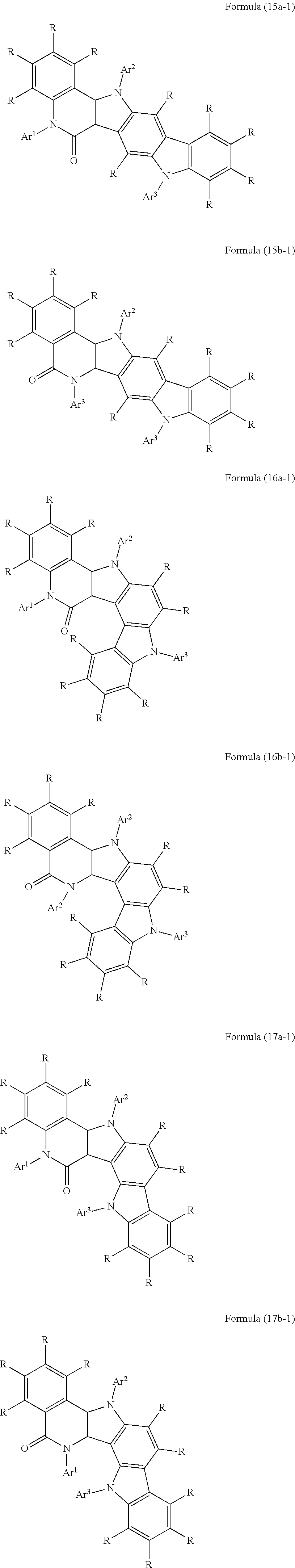

where the symbols used have the definitions given above.

In a preferred embodiment of the invention, not more than three R radicals in total, more preferably not more than two R radicals and most preferably not more than one R radical in these compounds are/is a group other than hydrogen.

Very particular preference is given to the compounds of the following formulae (6a-2) to (17b-2):

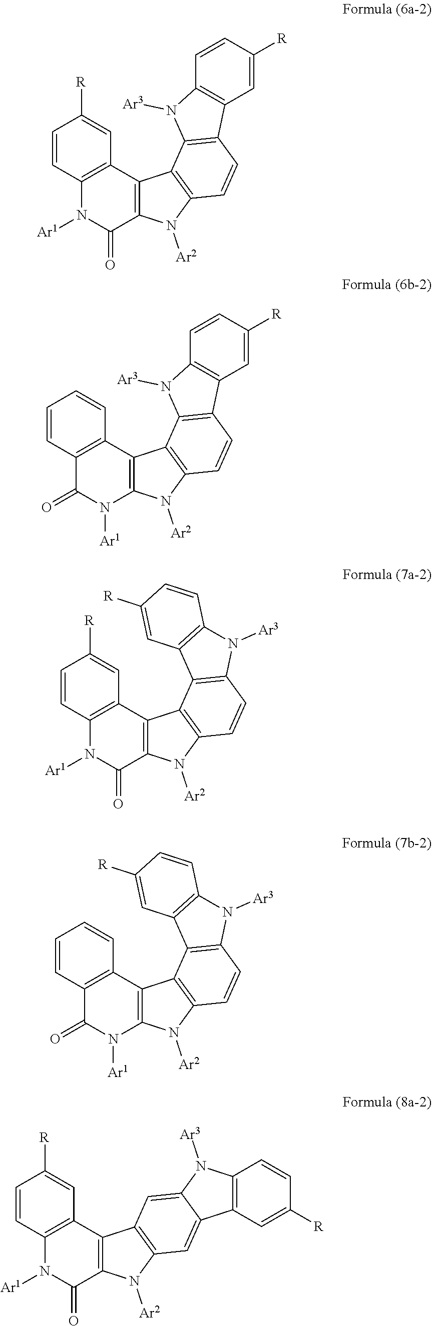

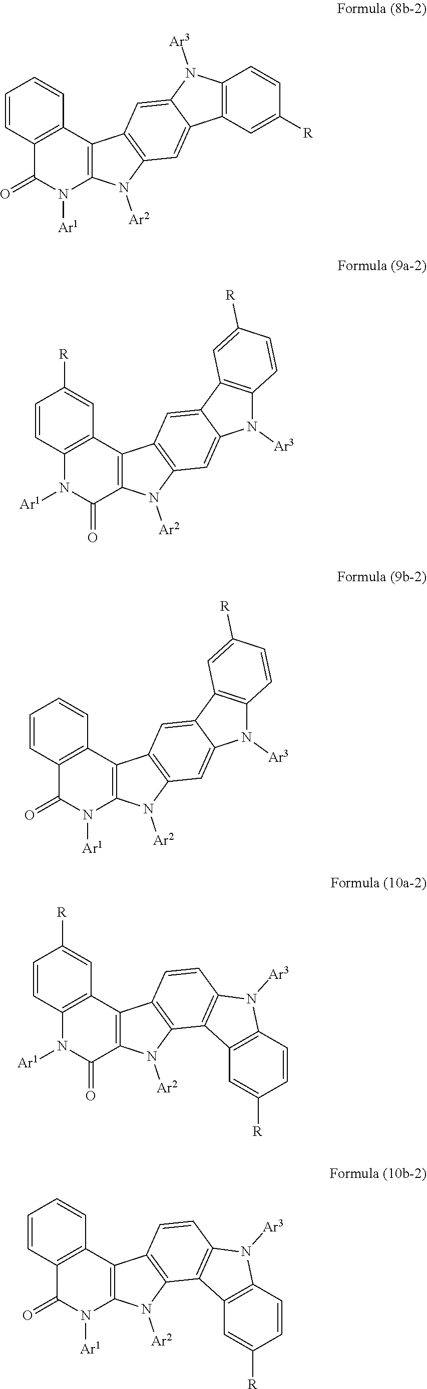

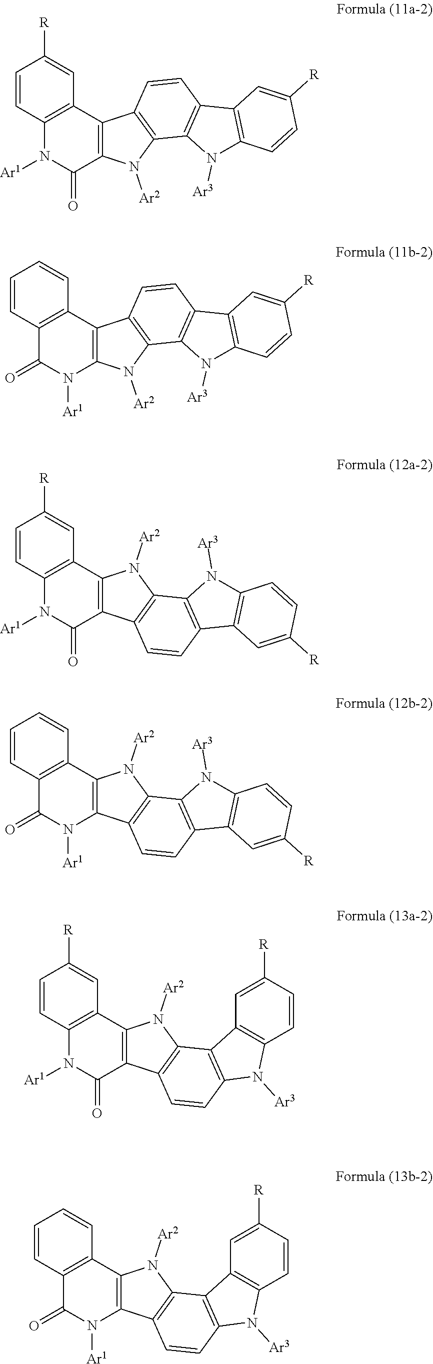

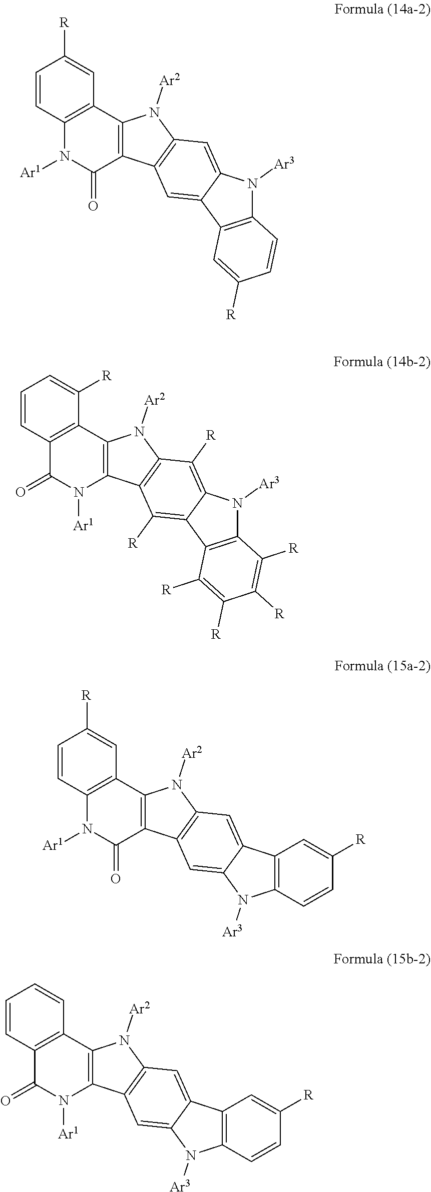

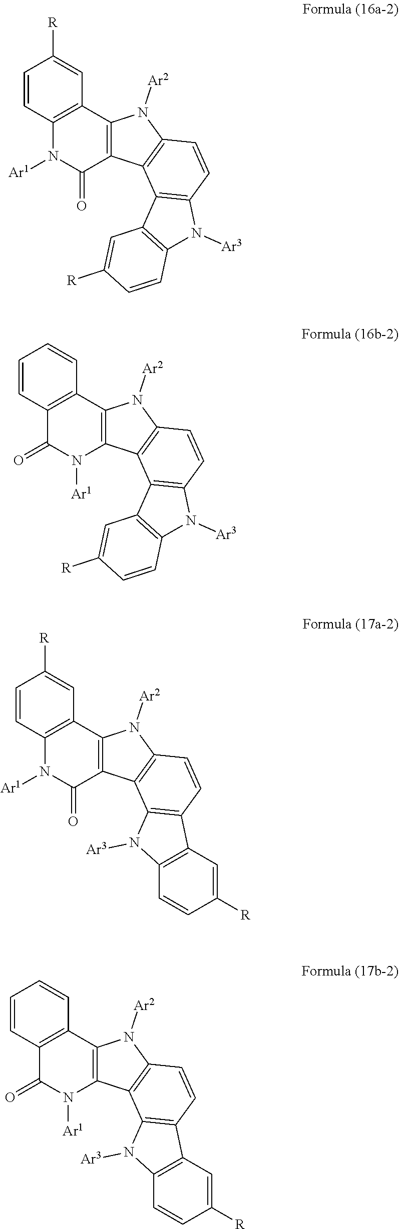

where the symbols used have the definitions given above.

In a further embodiment of the invention, two adjacent X groups are a group of the formula (3), and the two other symbols X are the same or different and are CR. In the group of the formula (3), the symbol A is preferably NAr³. If the two X groups are a group of the formula (3), preferred embodiments of the formula (4) are the compounds of the following formulae (18) to (23), and preferred embodiments of the formula (5) are the compounds of the following formulae (24) to (29):

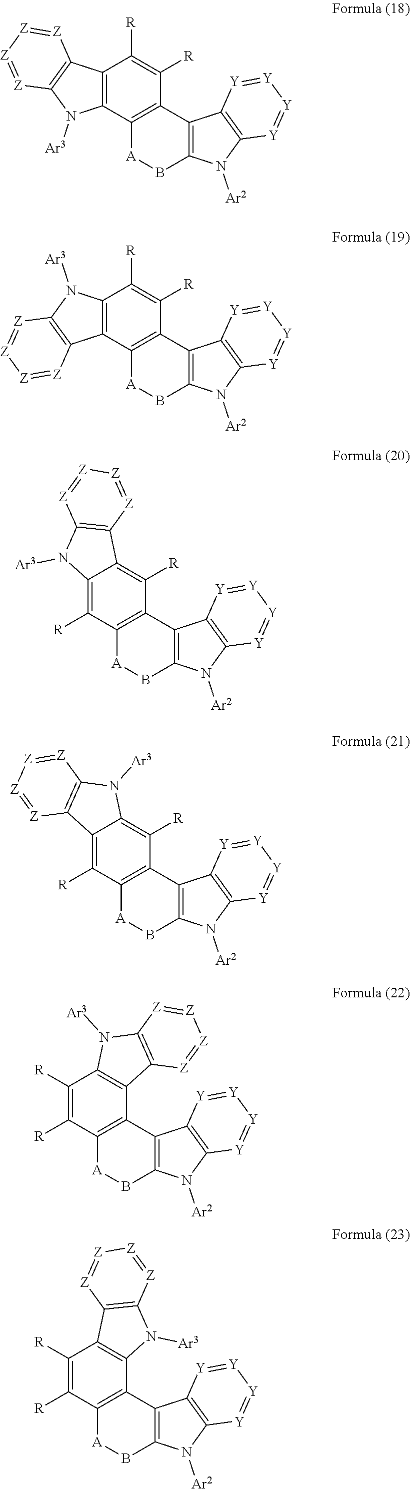

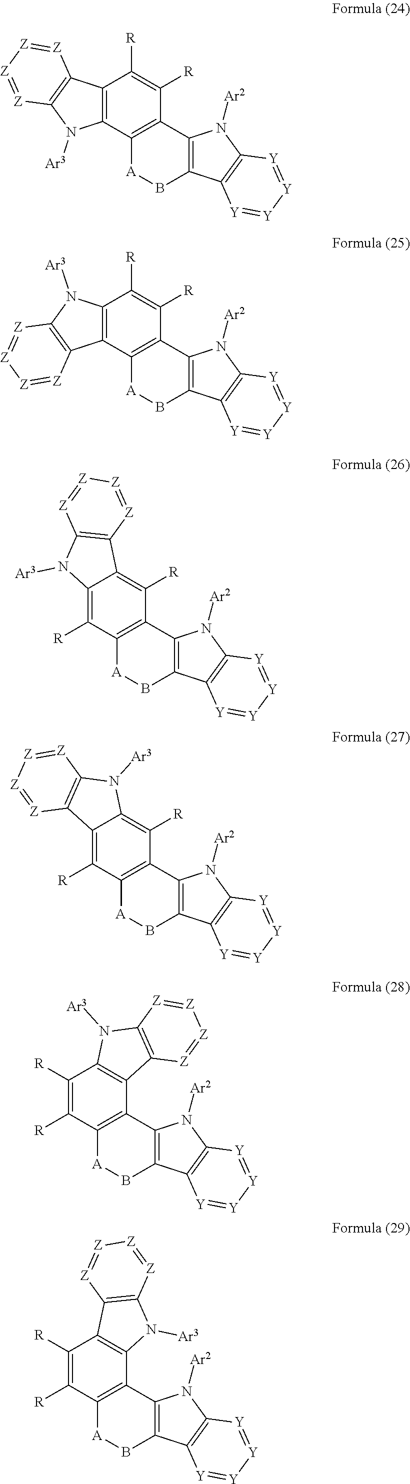

where the symbols used have the definitions given above.

In formulae (18) to (29), preferably not more than one Y group is N, and the other Y groups are the same or different and are CR. More preferably, all Y groups are the same or different and are CR.

In a further preferred embodiment of the invention, not more than one Z group is N, and the other Z groups are the same or different and are CR. More preferably, all Z groups are the same or different and are CR.

Most preferably, in the formulae (18) to (29), all symbols Y and Z are the same or different and are CR, and so particular preference is given to the compounds of the following formulae (18-1) to (29-1):

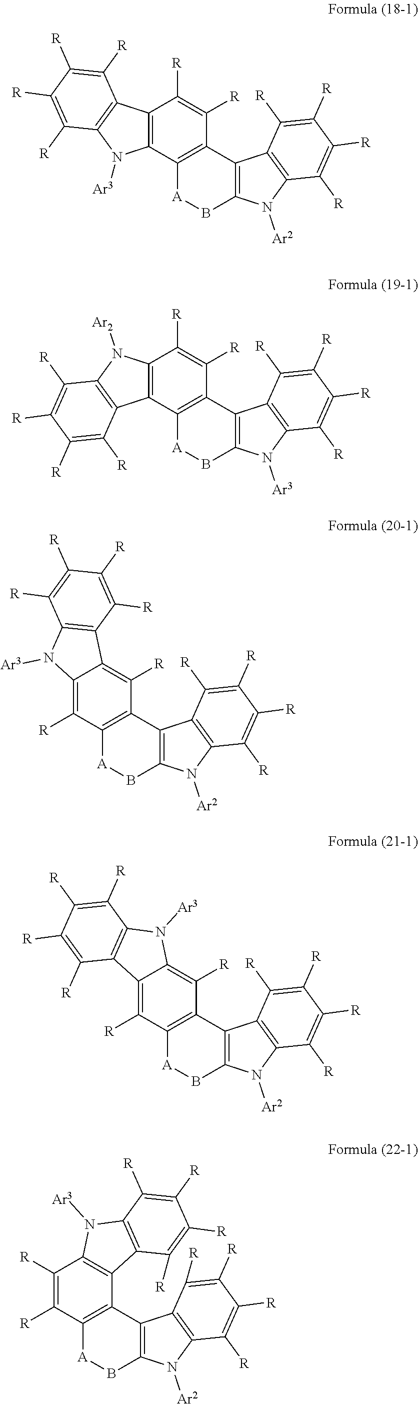

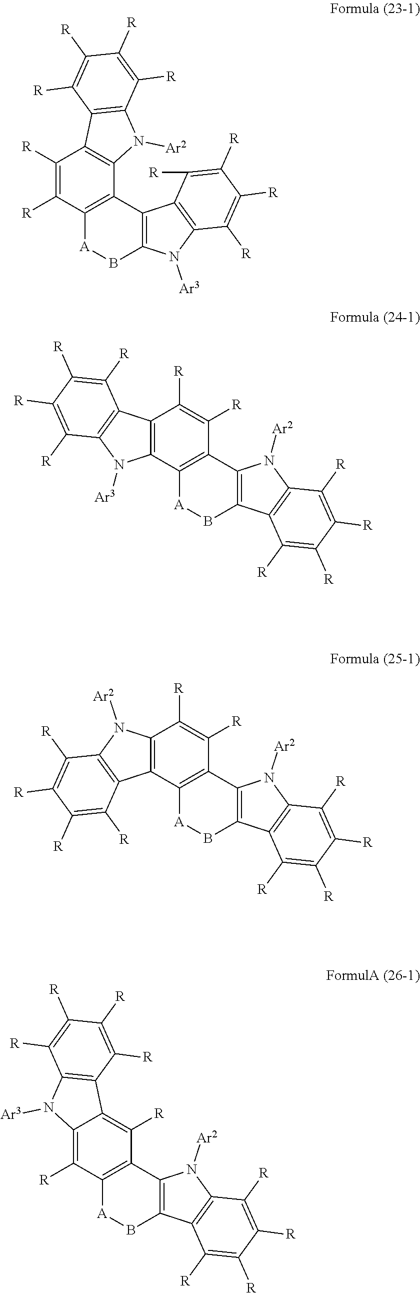

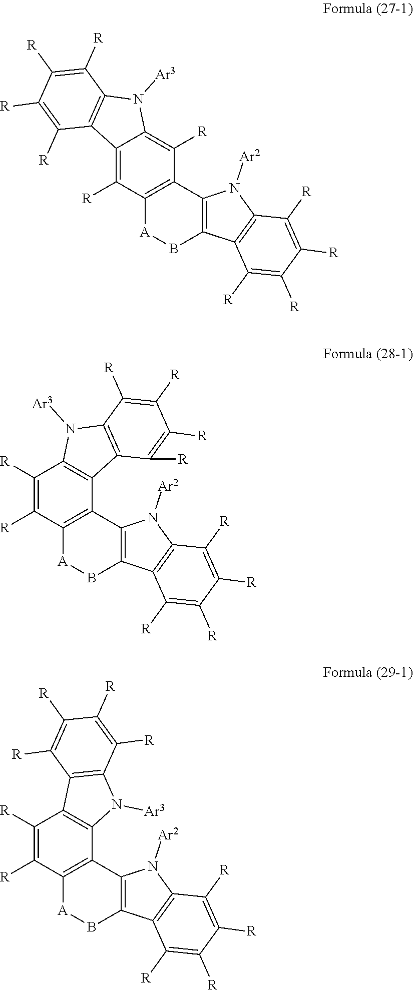

where the symbols used have the definitions given above.

For the formulae (18) to (29) and (18-1) to (29-1), it is preferable that one of the A and B groups is NAr¹ and the other of the A and B groups is C═O. Particular preference is therefore given to the structures of the following formulae (18a-1) to (29b-1):

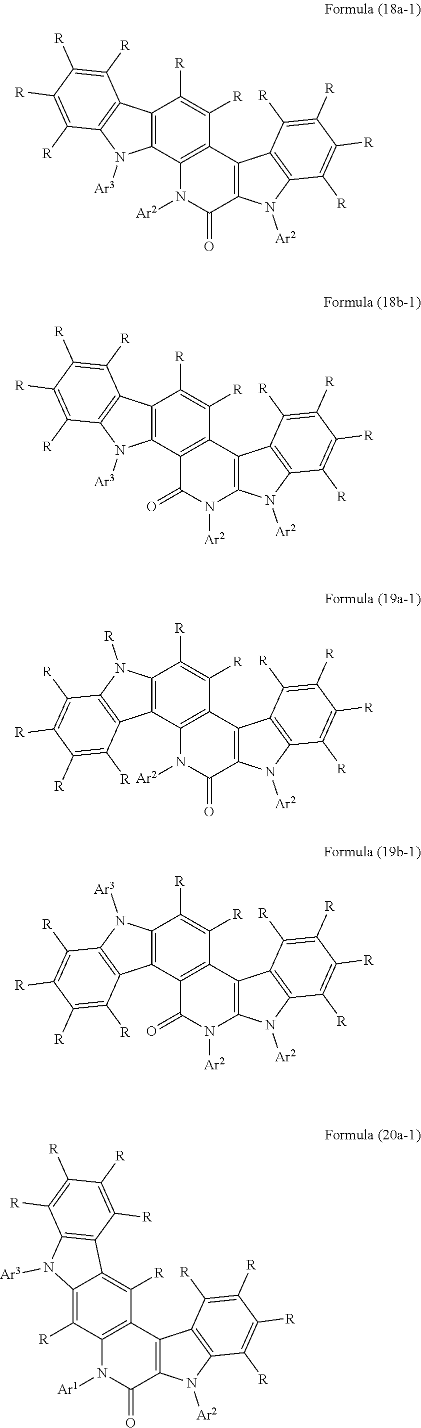

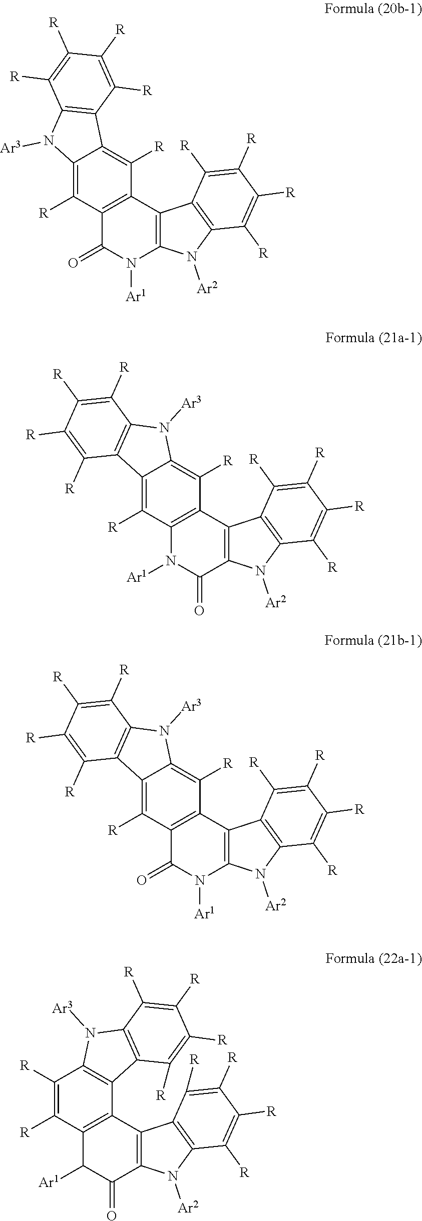

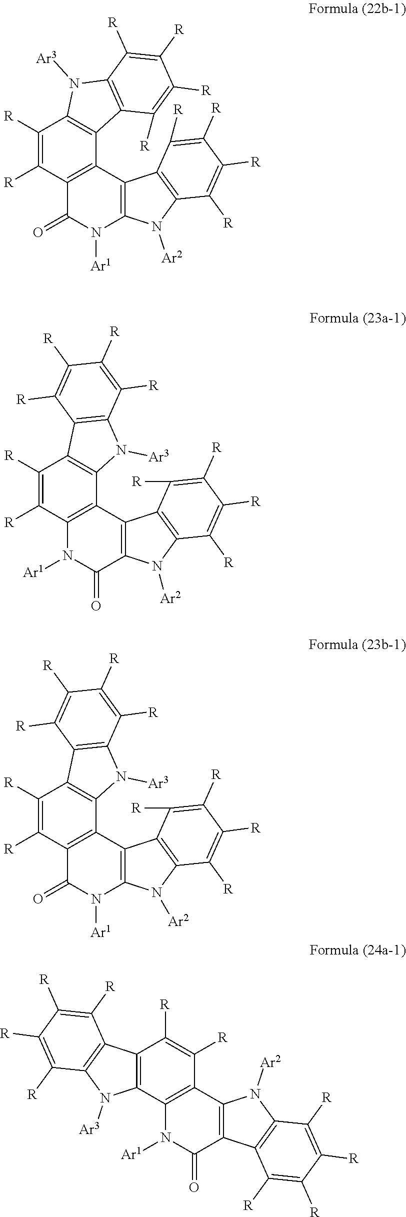

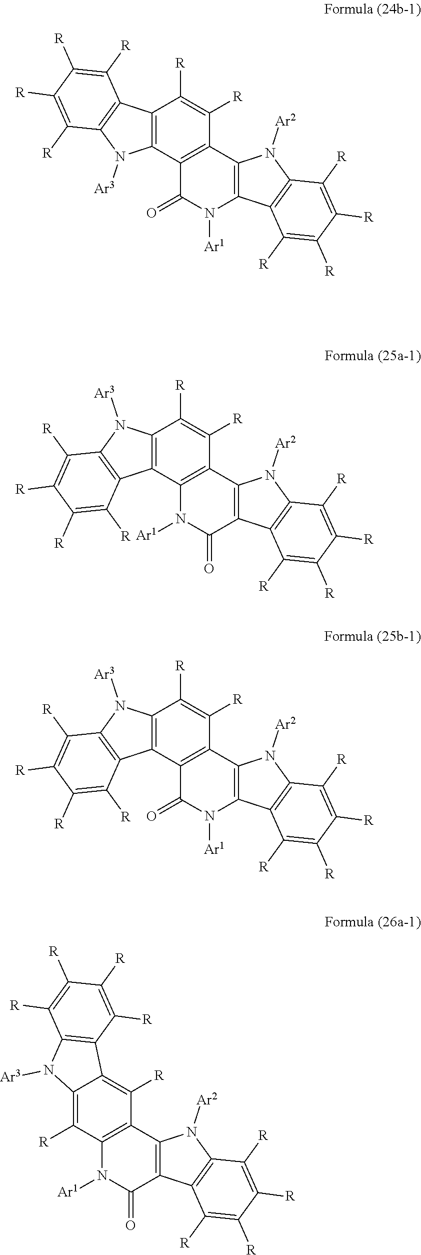

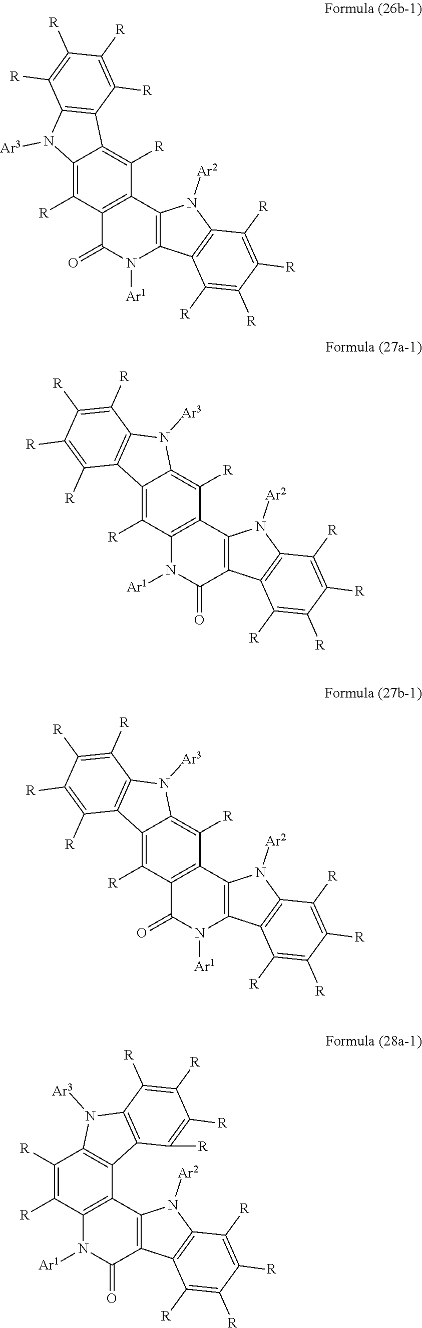

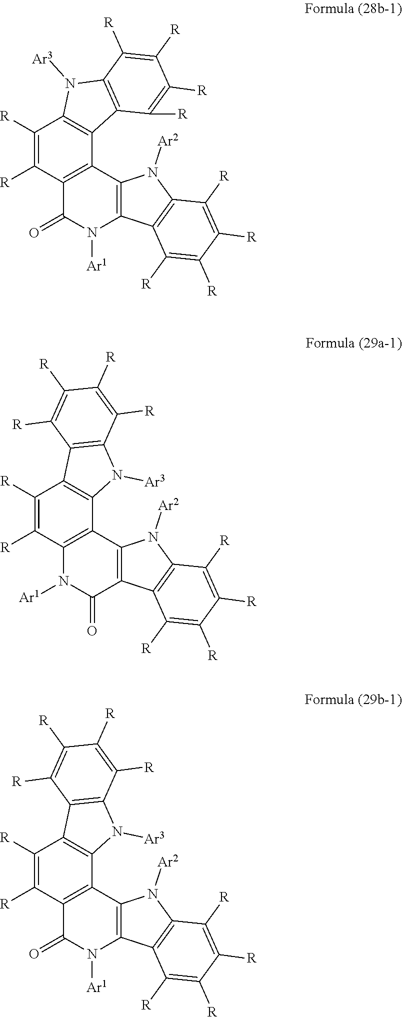

where the symbols used have the definitions given above.

In a preferred embodiment of the invention, not more than three R radicals in total, more preferably not more than two R radicals and most preferably not more than one R radical in these compounds are/is a group other than hydrogen.

Very particular preference is given to the compounds of the following formulae (18a-2) to (29b-2):

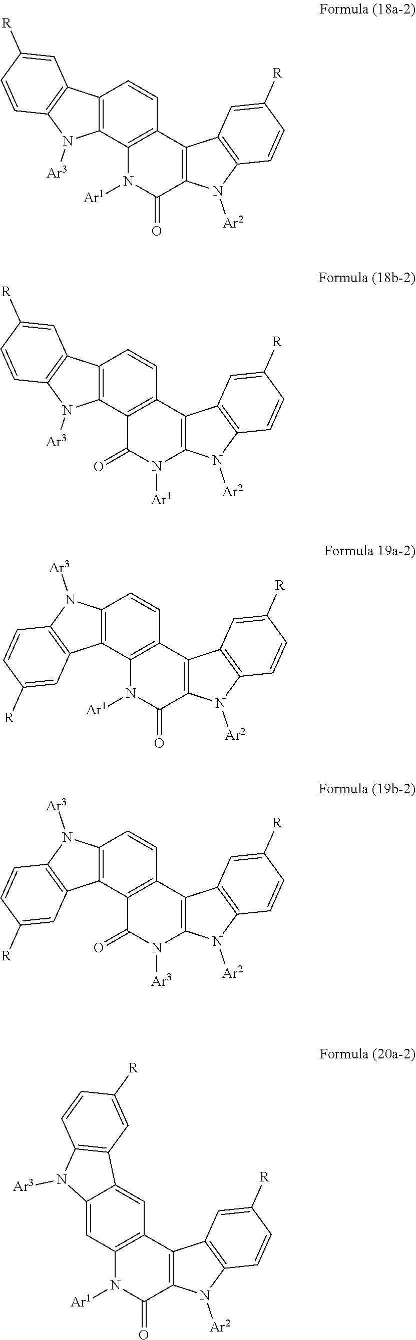

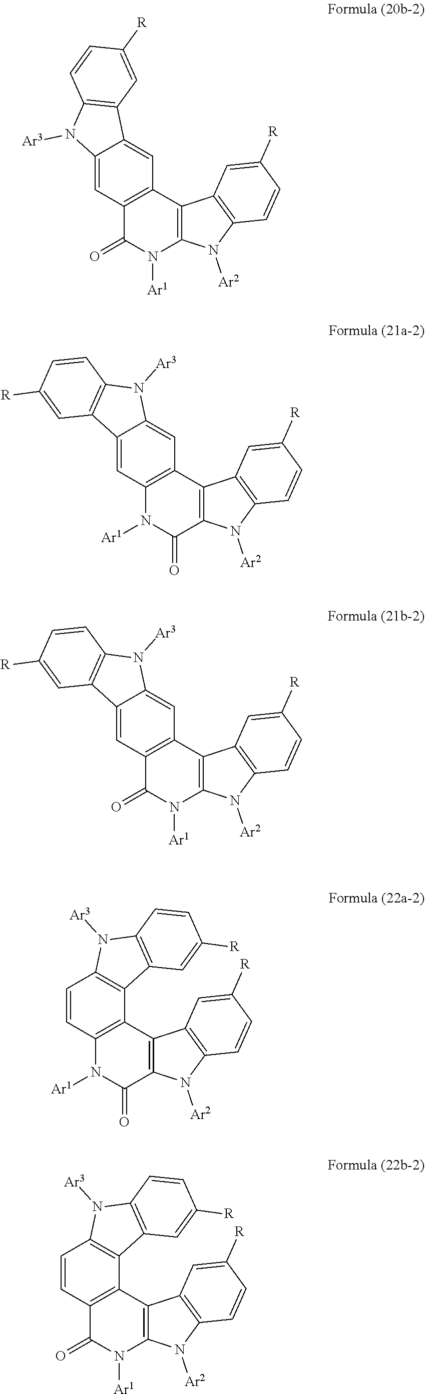

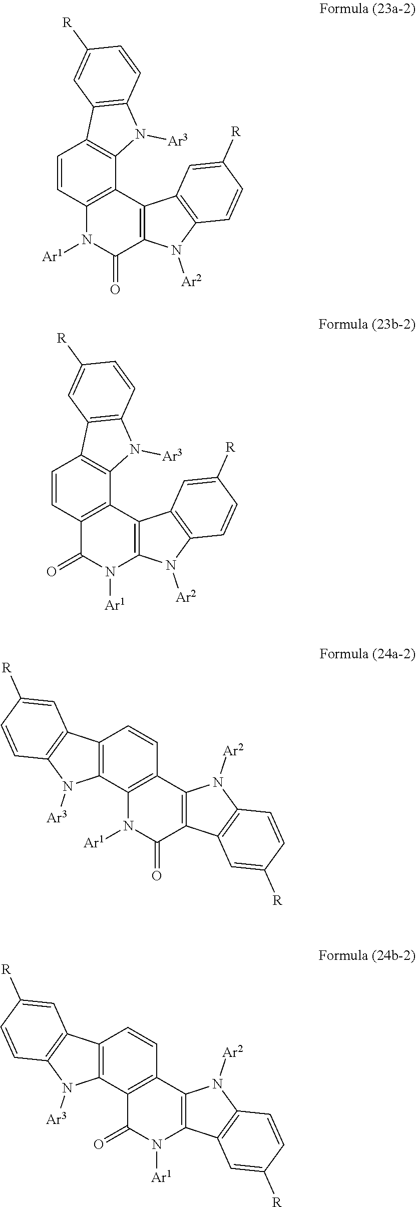

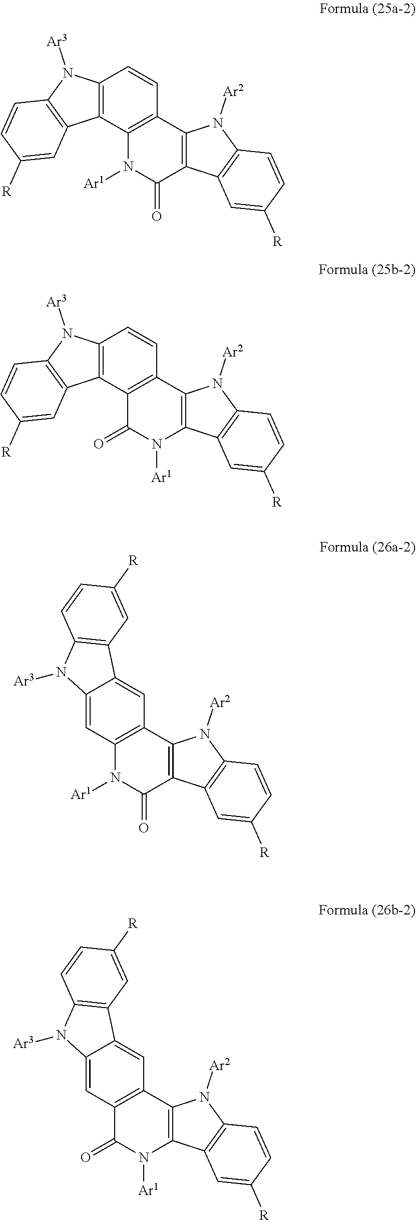

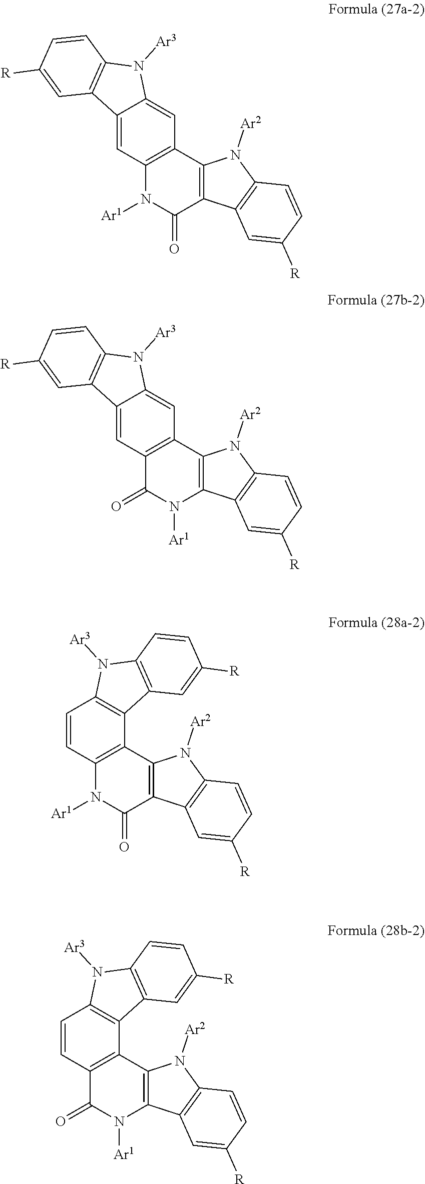

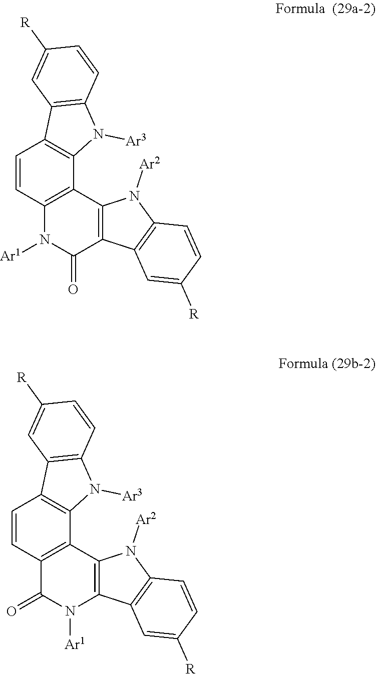

where the symbols used have the definitions given above.

There follows a description of preferred substituents Ar¹, Ar², Ar³, R, Ar′, R¹ and R² in the compounds of the invention. In a particularly preferred embodiment of the invention, the preferences specified hereinafter for Ar¹, Ar², Ar³, R, Ar′, R¹ and R² occur simultaneously and are applicable to the structures of the formula (1) and to all preferred embodiments detailed above.

In a preferred embodiment of the invention, Ar¹, Ar² and Ar³ are the same or different at each instance and are an aromatic or heteroaromatic ring system which has 6 to 30 aromatic ring atoms and may be substituted by one or more R radicals. More preferably, Ar¹, Ar² and Ar³ are the same or different at each instance and is an aromatic or heteroaromatic ring system which has 6 to 24 aromatic ring atoms, especially 6 to 13 aromatic ring atoms, and may be substituted by one or more, preferably nonaromatic, R radicals. When Ar¹, Ar² or Ar³ is a heteroaryl group, especially triazine, pyrimidine, quinazoline or carbazole, preference may also be given to aromatic or heteroaromatic substituents R on this heteroaryl group. It may further be preferable when Ar¹, Ar² or Ar³ is substituted by an N(Ar′)₂ group, such that the substituent Ar¹, Ar² or Ar³ constitutes a triarylamine or triheteroarylamine group overall.

Suitable aromatic or heteroaromatic ring systems Ar¹, Ar² and Ar³ are the same or different at each instance and are selected from the group consisting of phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene which may be joined via the 1 or 2 position, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, quinazoline, benzimidazole, phenanthrene, triphenylene or a combination of two or three of these groups, each of which may be substituted by one or more R radicals, preferably nonaromatic R radicals. When Ar¹, Ar² or Ar³ is a heteroaryl group, especially triazine, pyrimidine, quinazoline or carbazole, preference may also be given to aromatic or heteroaromatic R radicals on this heteroaryl group.

Ar¹, Ar² and Ar³ here are preferably the same or different at each instance and are selected from the groups of the following formulae Ar-1 to Ar-83:

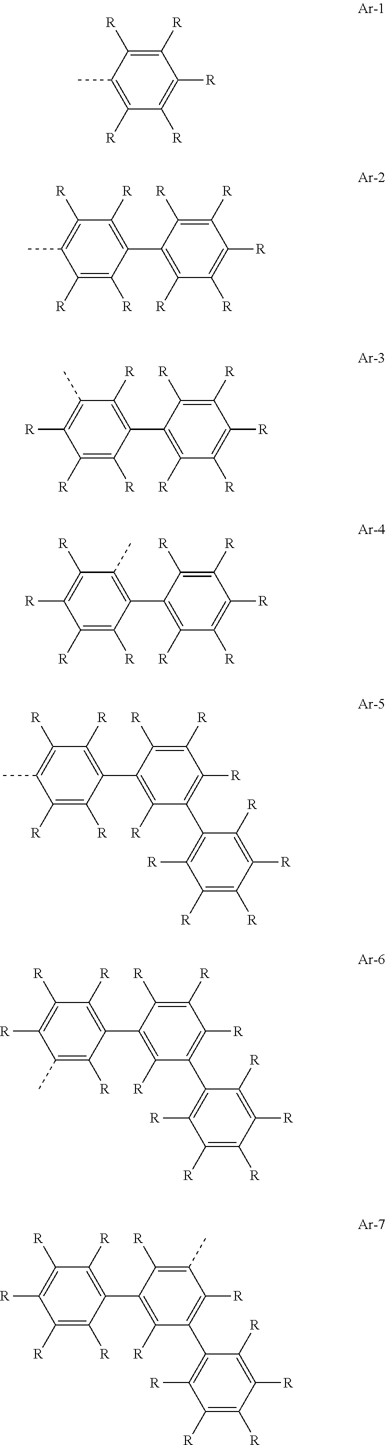

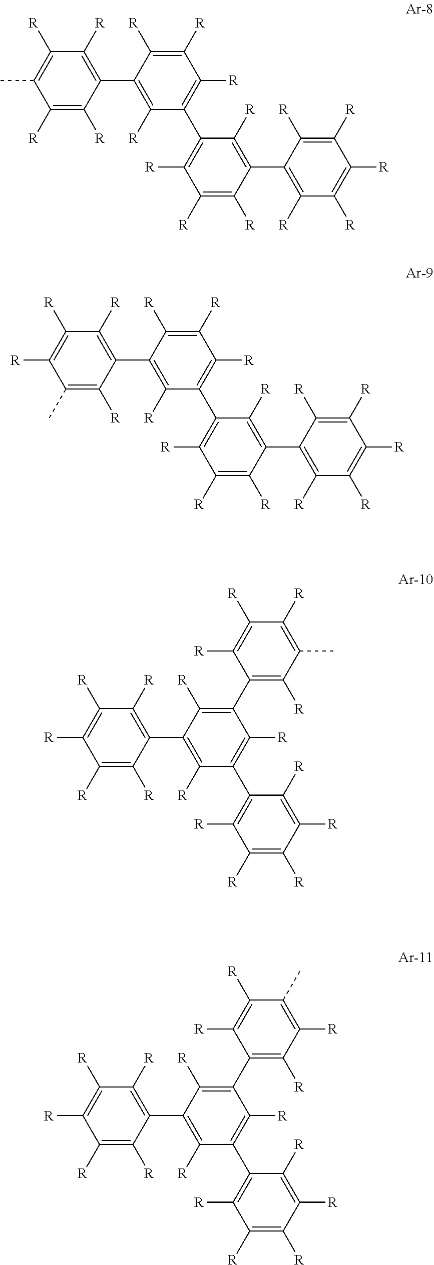

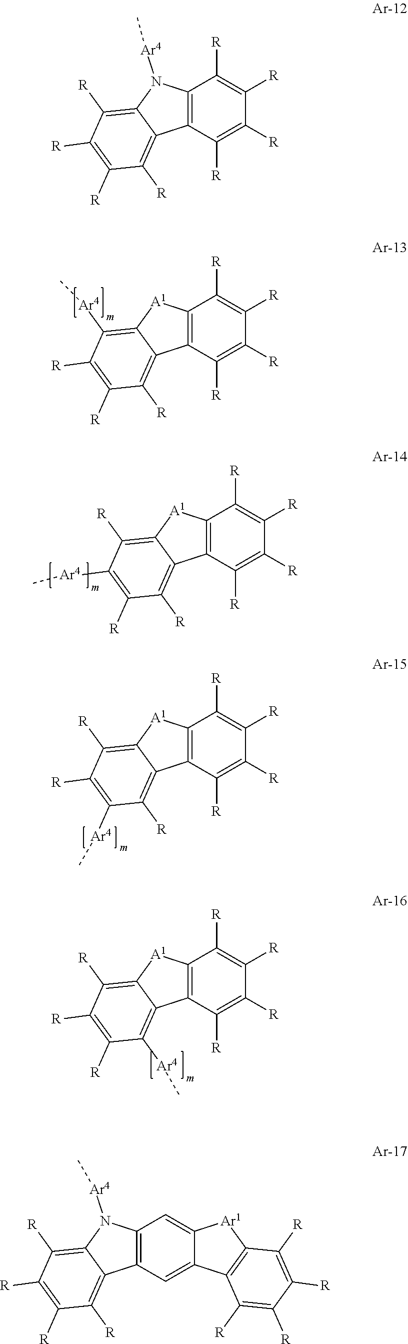

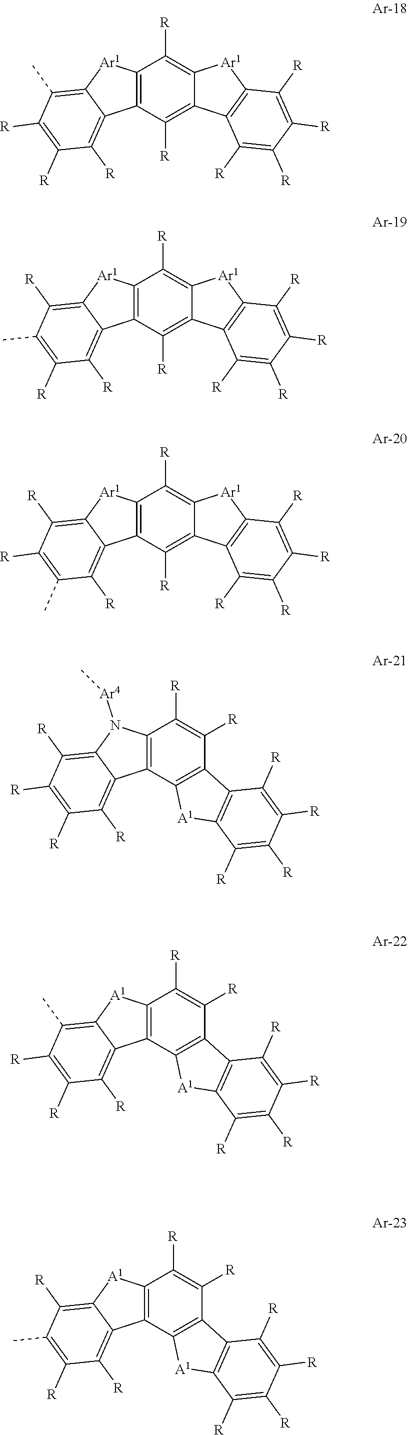

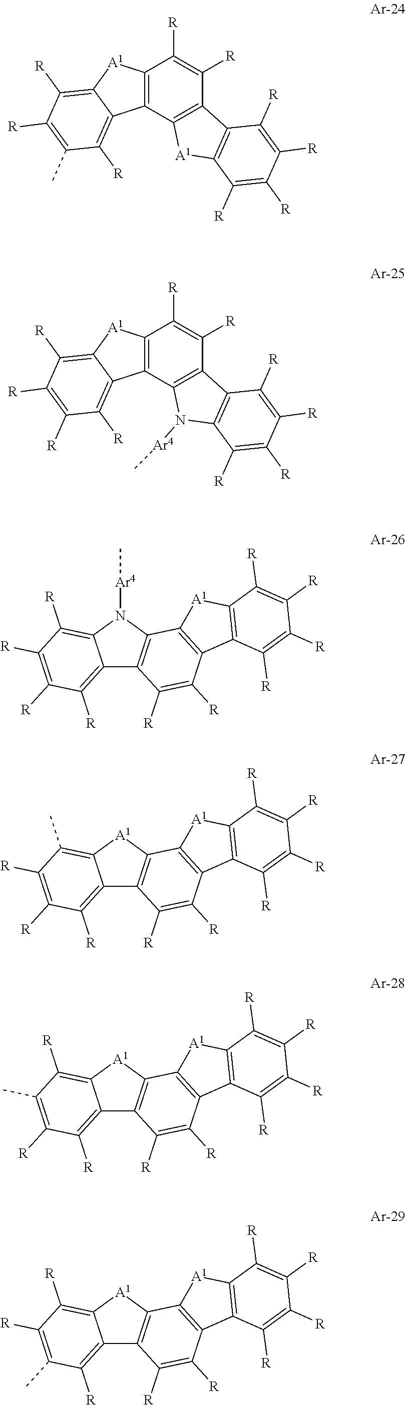

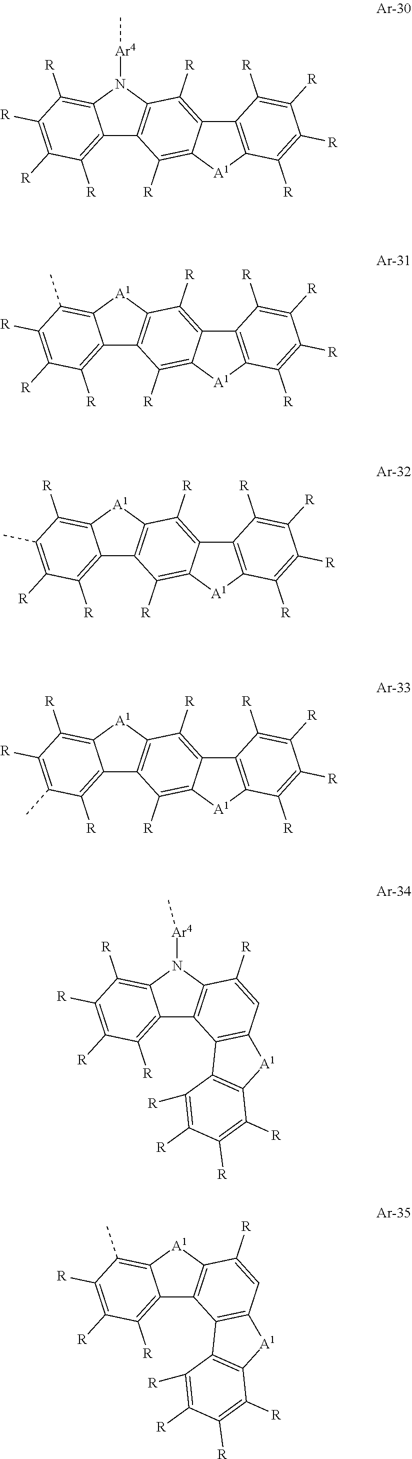

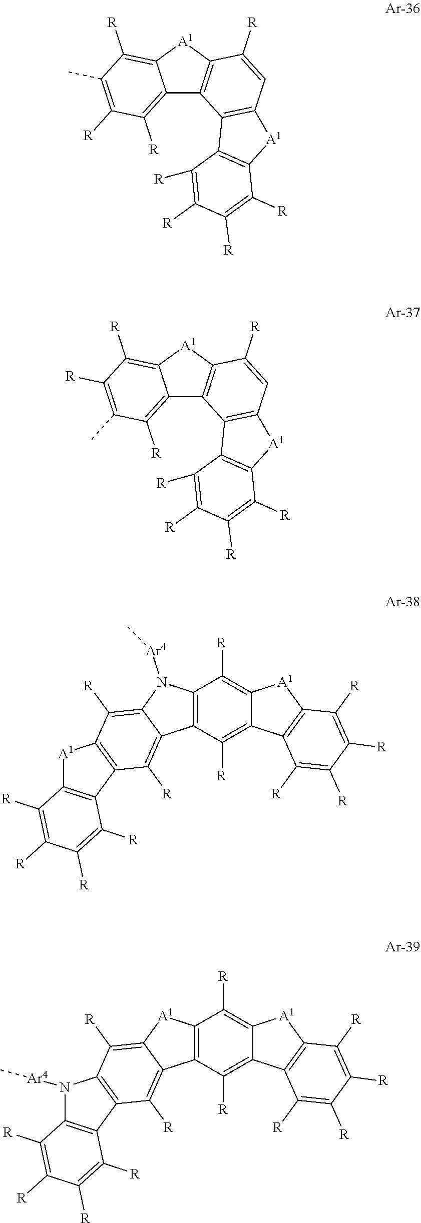

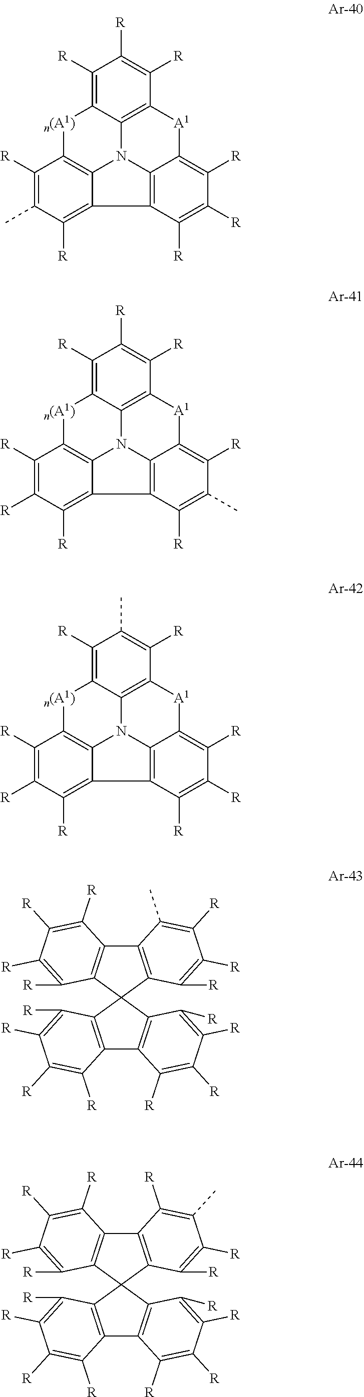

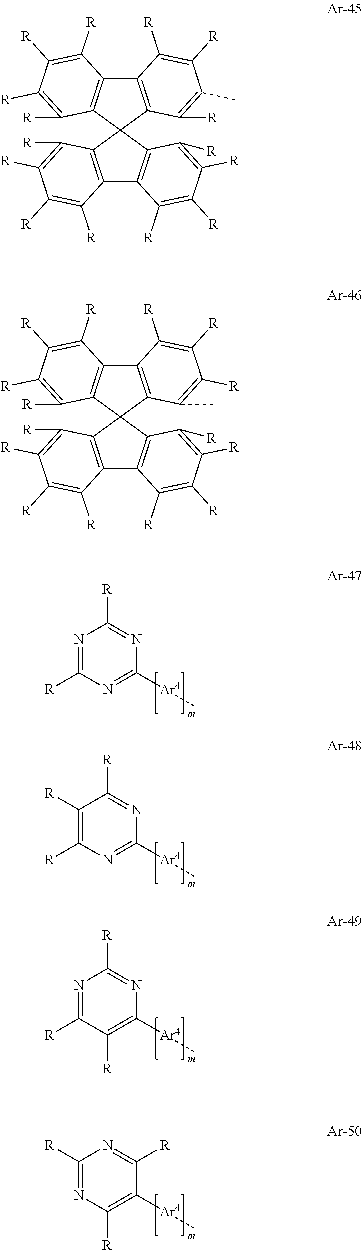

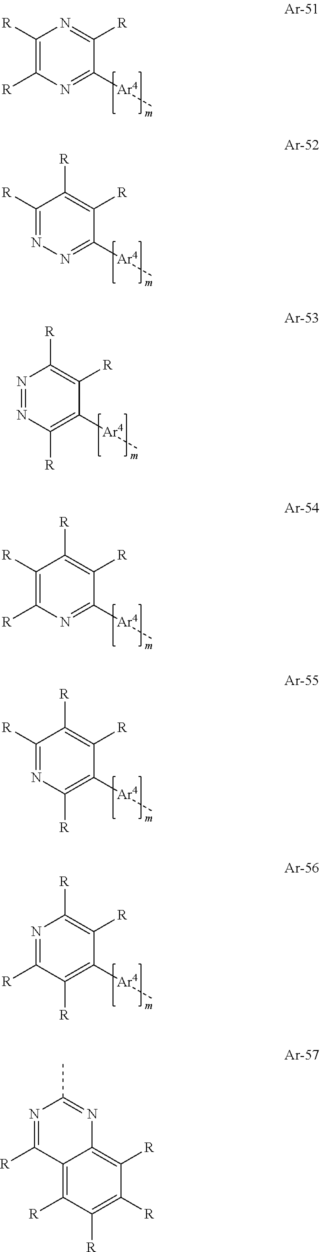

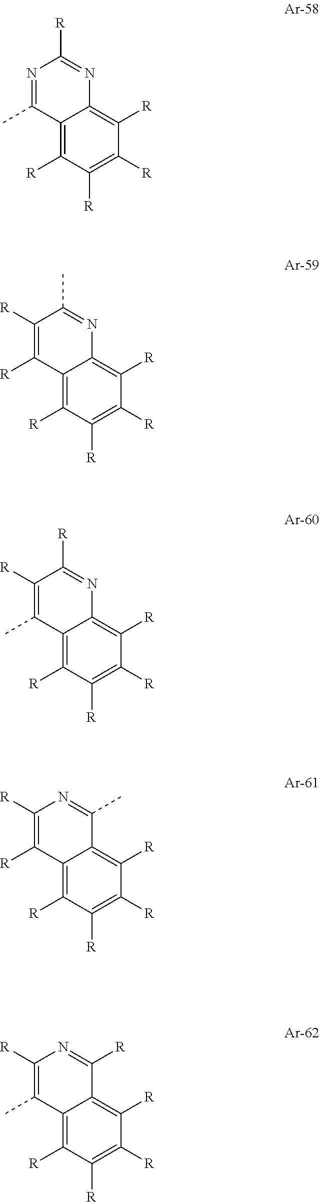

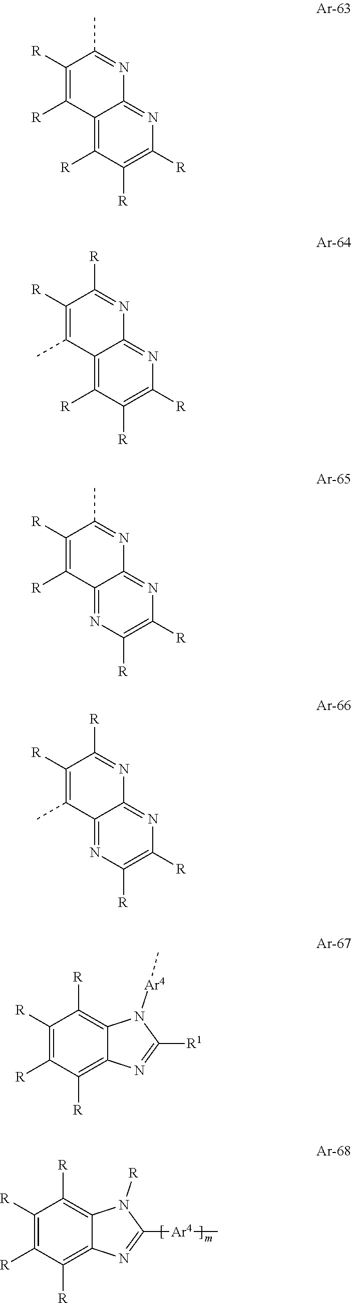

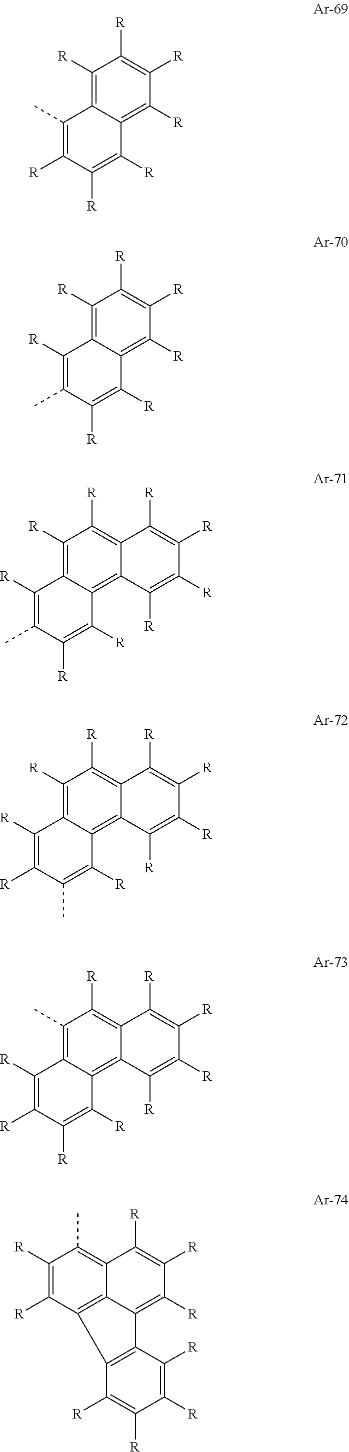

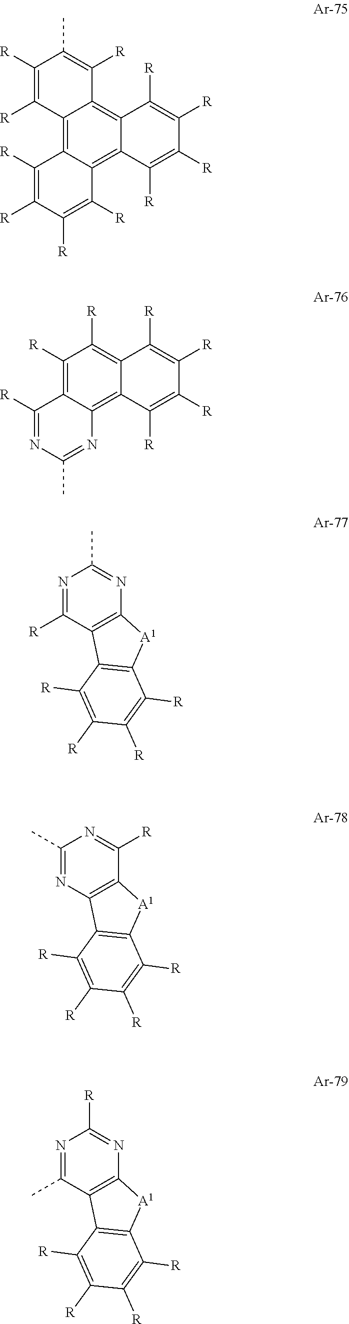

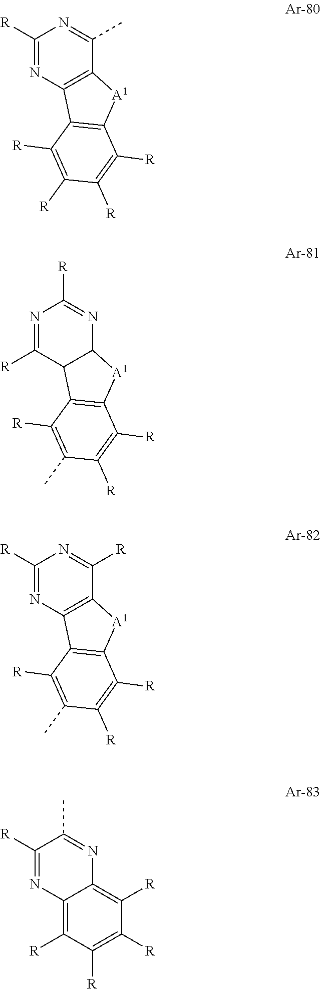

where R and A¹ have the definitions given above, the dotted bond represents the bond to the nitrogen atom, and in addition:
Ar⁴ is the same or different at each instance and is a bivalent aromatic or heteroaromatic ring system which has 6 to 18 aromatic ring atoms and may be substituted in each case by one or more R radicals;
n is 0 or 1, where n=0 means that no A¹ group is bonded at this position and R radicals are bonded to the corresponding carbon atoms instead;
m is 0 or 1, where m=0 means that the Ar⁴ group is absent and that the corresponding aromatic or heteroaromatic group is bonded directly to the nitrogen atom.

In a preferred embodiment of the invention, R is the same or different at each instance and is selected from the group consisting of H, D, F, N(Ar′)₂, CN, OR¹, a straight-chain alkyl group having 1 to 10 carbon atoms or an alkenyl group having 2 to 10 carbon atoms or a branched or cyclic alkyl group having 3 to 10 carbon atoms, where the alkyl or alkenyl group may each be substituted by one or more R¹ radicals, but is preferably unsubstituted, and where one or more nonadjacent CH₂ groups may be replaced by O, or an aromatic or heteroaromatic ring system which has 6 to 30 aromatic ring atoms and may be substituted in each case by one or more R¹ radicals; at the same time, two R radicals together may also form an aliphatic, aromatic or heteroaromatic ring system. More preferably, R is the same or different at each instance and is selected from the group consisting of H, N(Ar′)₂, a straight-chain alkyl group having 1 to 6 carbon atoms, especially having 1, 2, 3 or 4 carbon atoms, or a branched or cyclic alkyl group having 3 to 6 carbon atoms, where the alkyl group in each case may be substituted by one or more R¹ radicals, but is preferably unsubstituted, or an aromatic or heteroaromatic ring system which has 6 to 24 aromatic ring atoms and may be substituted in each case by one or more R¹ radicals, preferably nonaromatic R¹ radicals. Most preferably, R is the same or different at each instance and is selected from the group consisting of H or an aromatic or heteroaromatic ring system which has 6 to 24 aromatic ring atoms and may be substituted in each case by one or more R¹ radicals, preferably nonaromatic R¹ radicals. It may additionally be preferable when R is a triaryl- or -heteroarylamine group which may be substituted by one or more R¹ radicals. This group is one embodiment of an aromatic or heteroaromatic ring system, in which case two or more aryl or heteroaryl groups are joined to one another by a nitrogen atom. When R is a triaryl- or -heteroarylamine group, this group preferably has 18 to 30 aromatic ring atoms and may be substituted by one or more R¹ radicals, preferably nonaromatic R¹ radicals.

In a further preferred embodiment of the invention, Ar′ is an aromatic or heteroaromatic ring system which has 6 to 30 aromatic ring atoms and may be substituted by one or more R¹ radicals. In a particularly preferred embodiment of the invention, Ar′ is an aromatic or heteroaromatic ring system which has 6 to 24 aromatic ring atoms, especially 6 to 13 aromatic ring atoms, and may be substituted by one or more, preferably nonaromatic, R¹ radicals.

Suitable aromatic or heteroaromatic ring systems R or Ar′ are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene which may be joined via the 1 or 2 position, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, quinazoline, benzimidazole, phenanthrene, triphenylene or a combination of two or three of these groups, each of which may be substituted by one or more R¹ radicals. When R or Ar′ is a heteroaryl group, especially triazine, pyrimidine, quinazoline or carbazole, preference may also be given to aromatic or heteroaromatic R¹ radicals on this heteroaryl group.

The R groups here, when they are an aromatic or heteroaromatic ring system, or Ar′ are preferably selected from the groups of the following formulae R-1 to R-83:

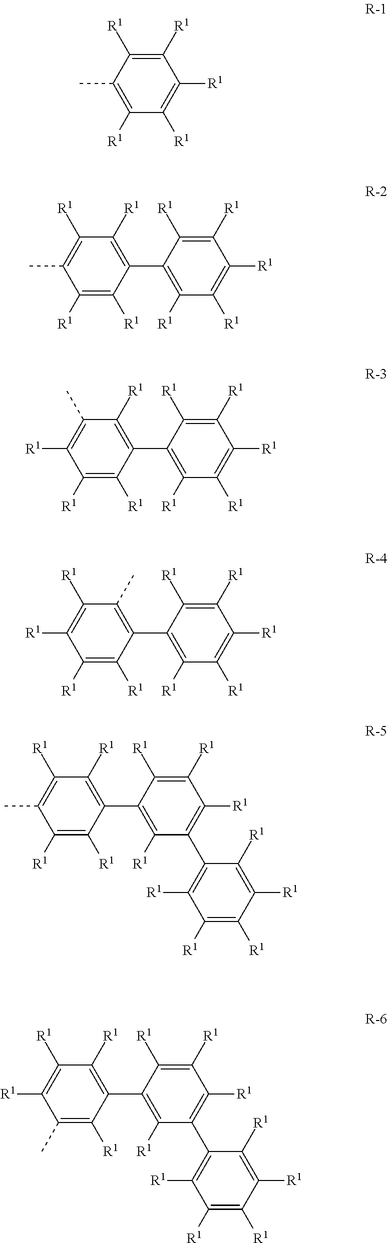

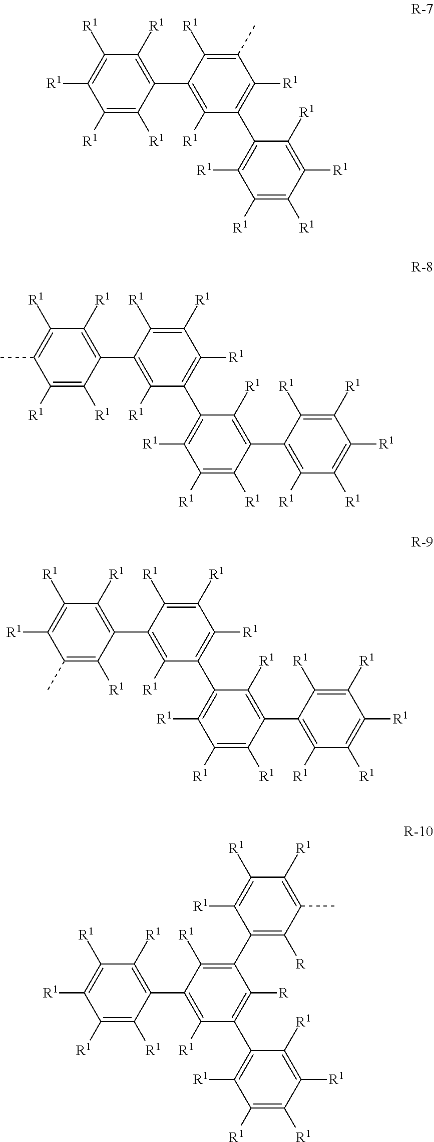

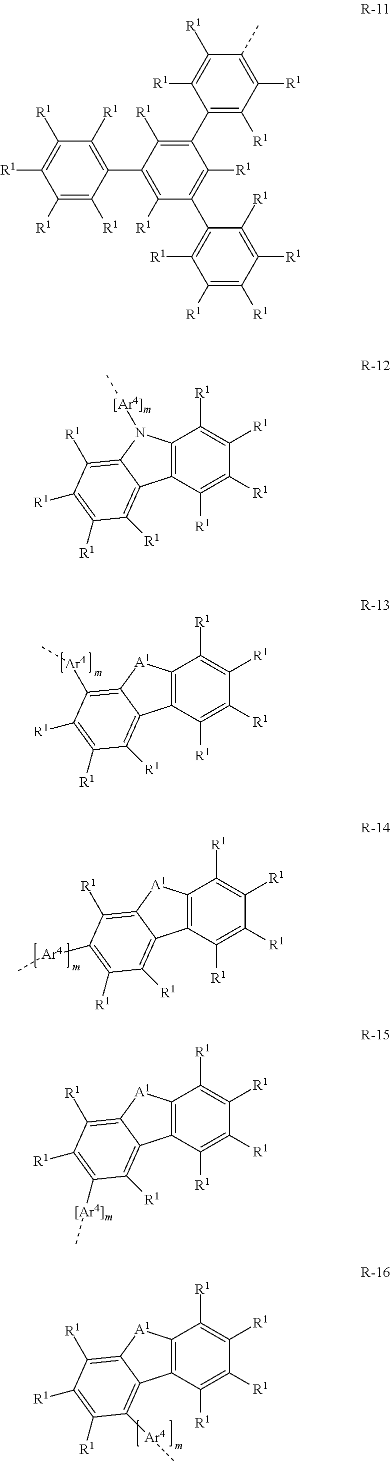

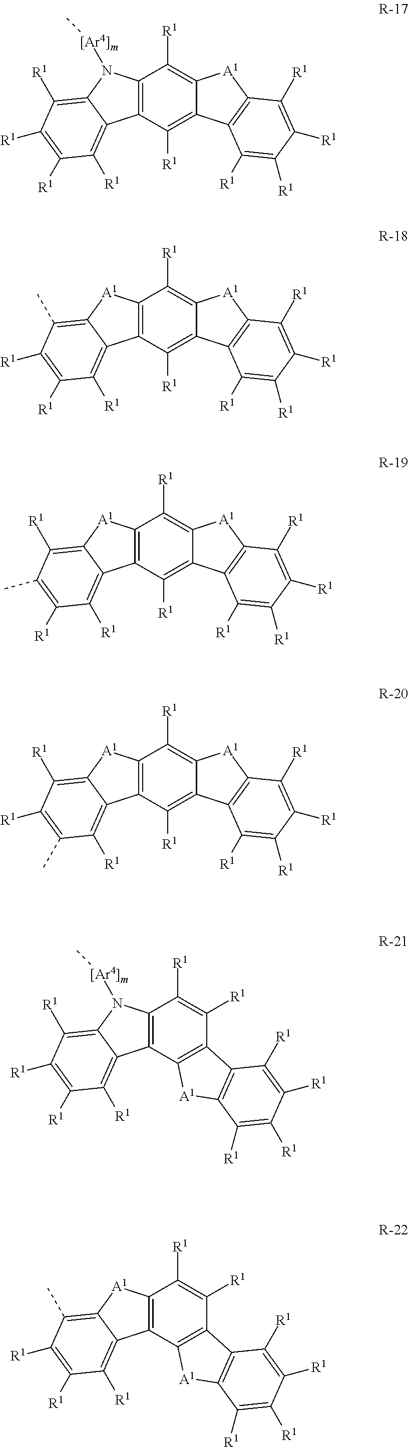

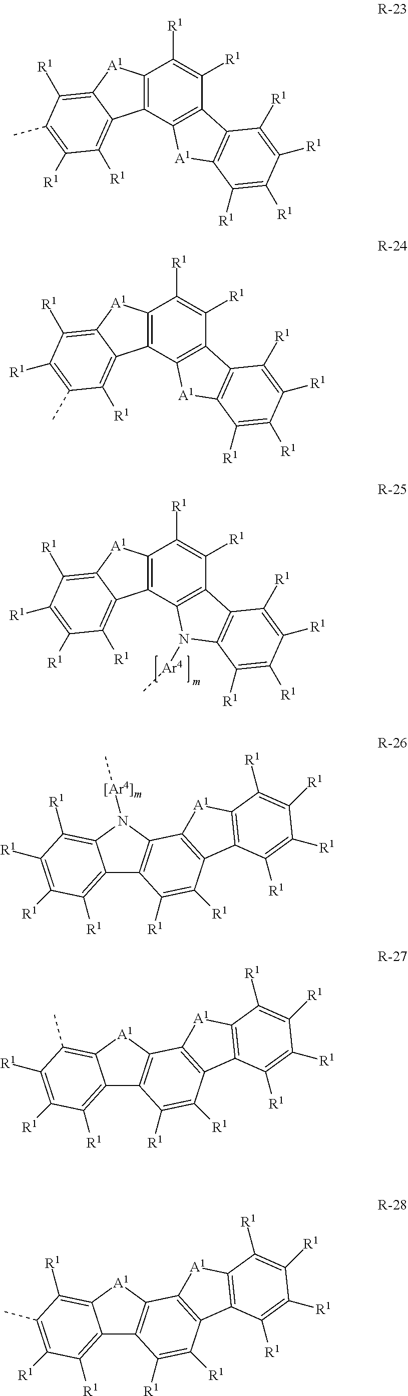

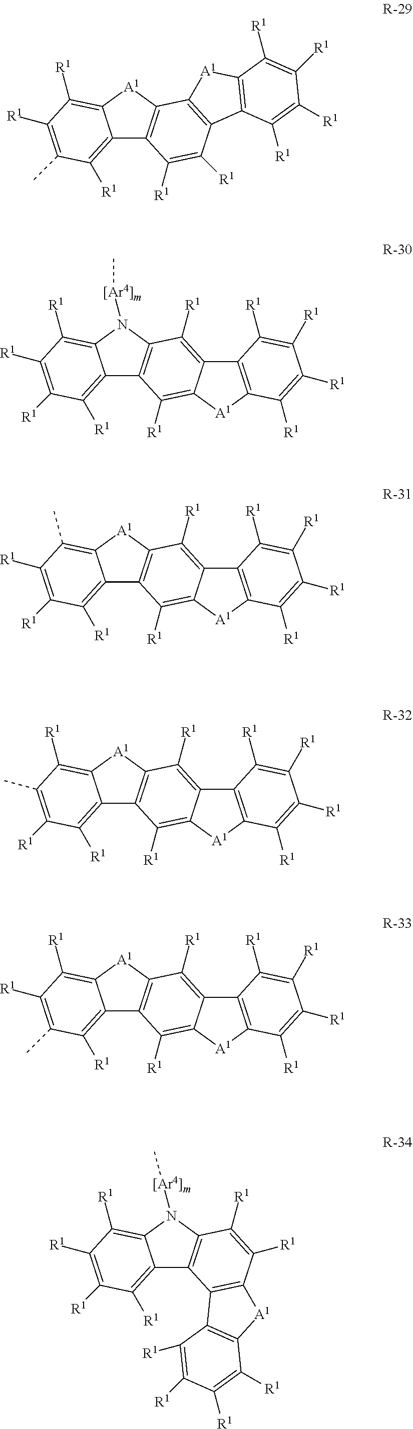

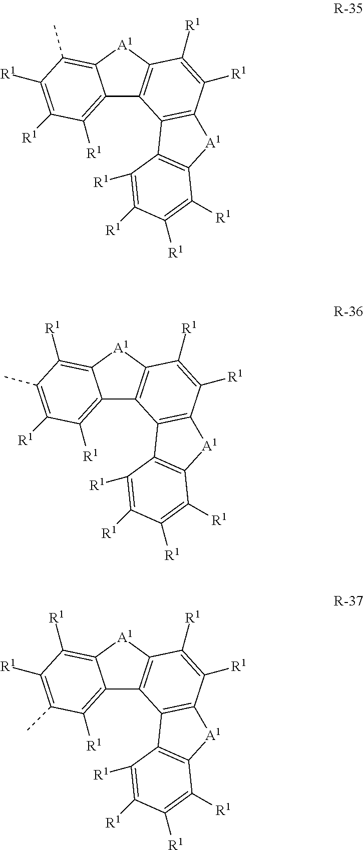

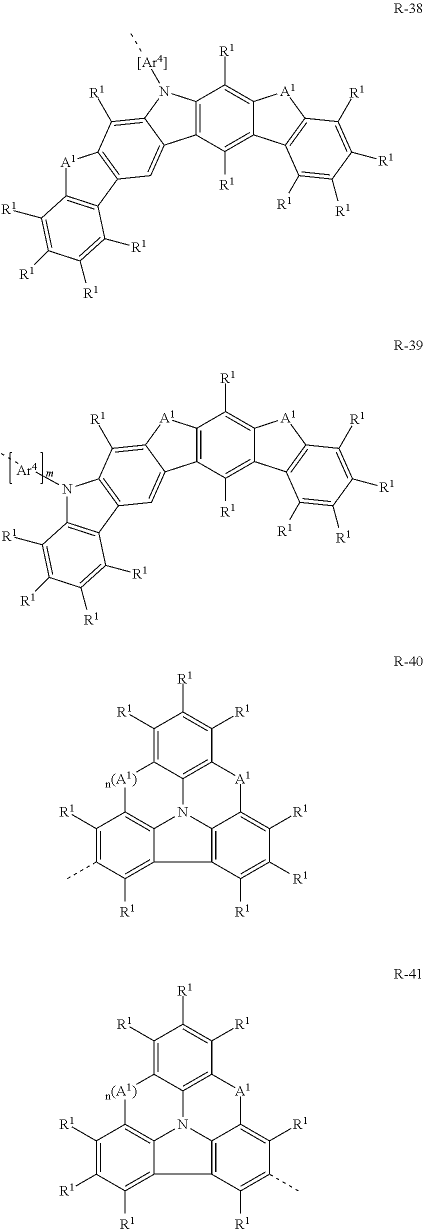

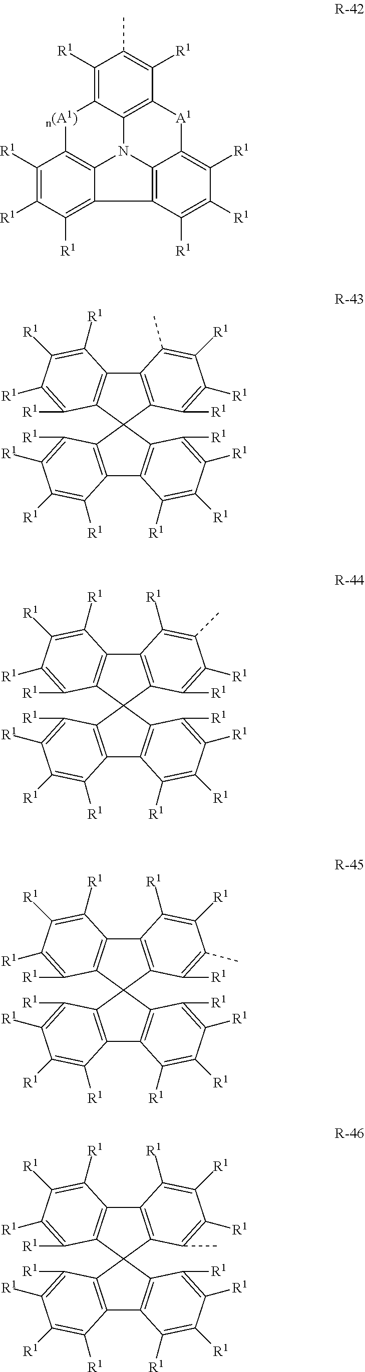

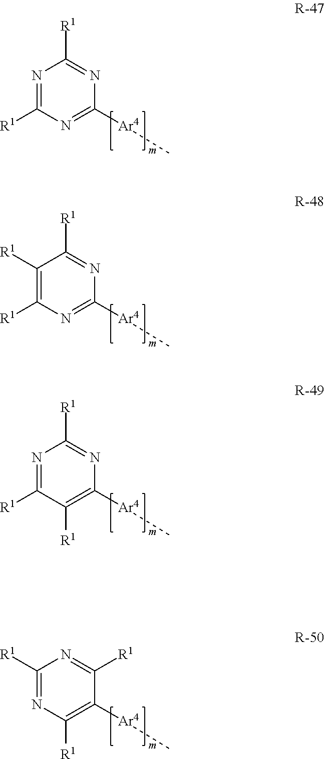

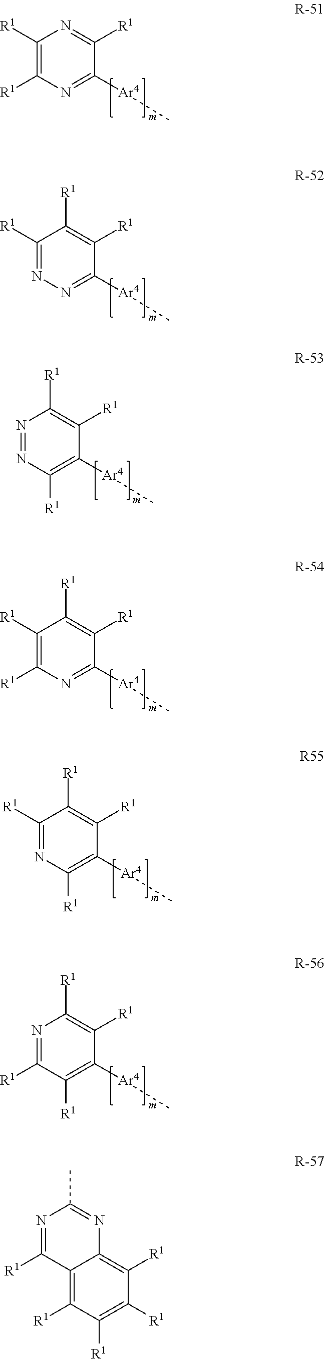

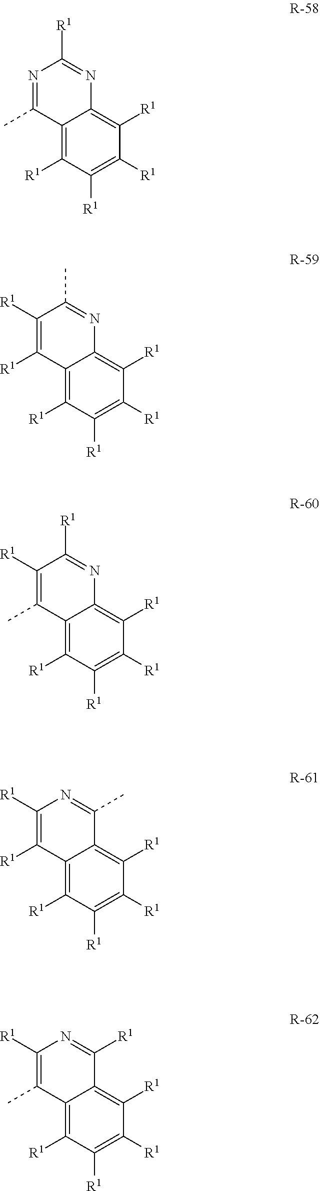

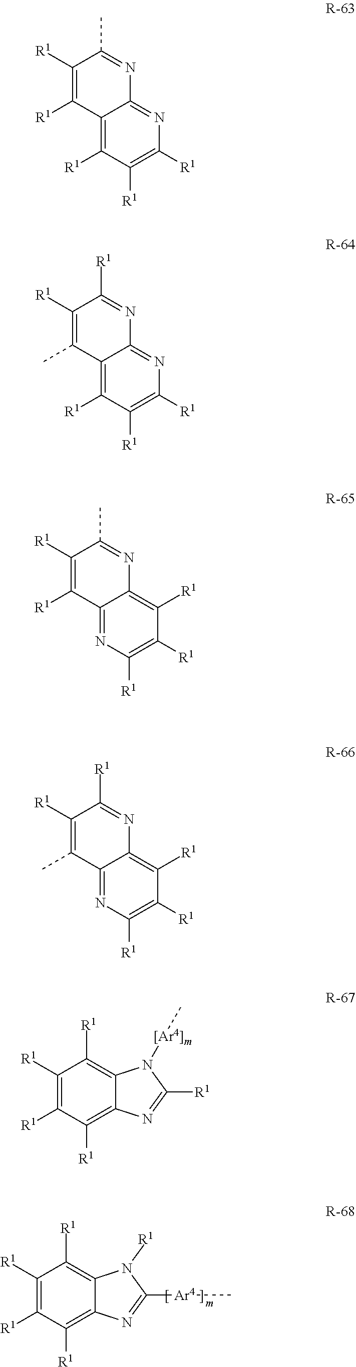

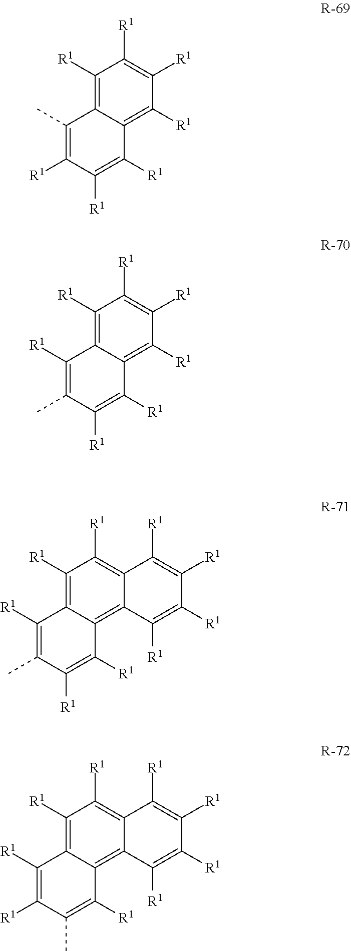

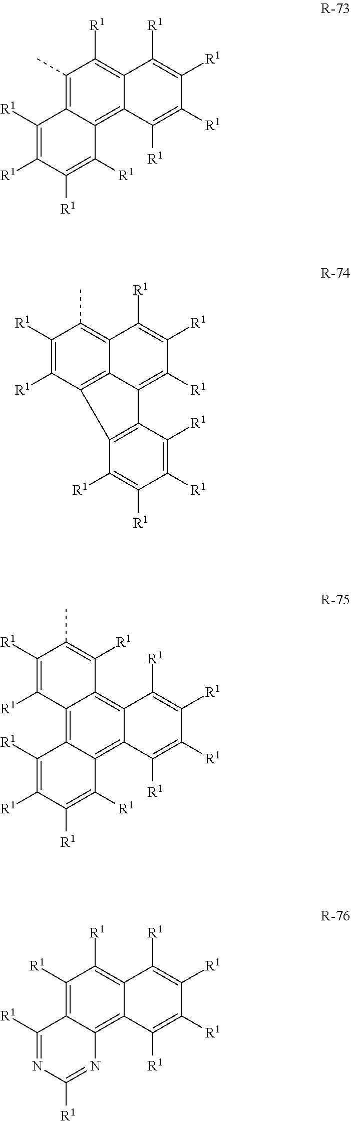

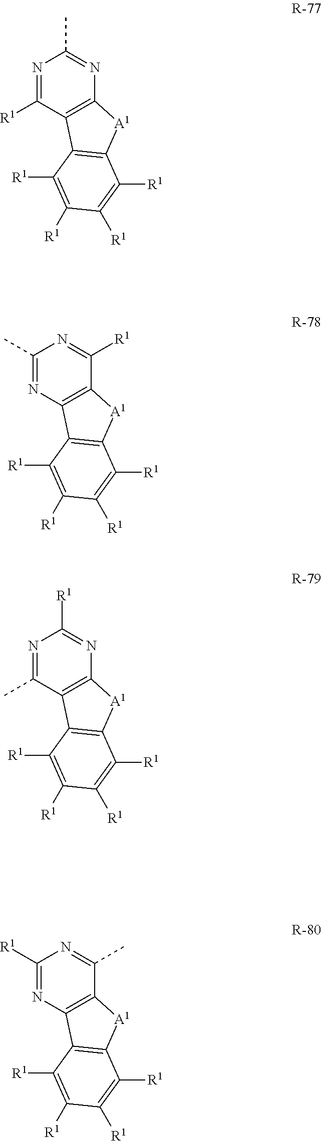

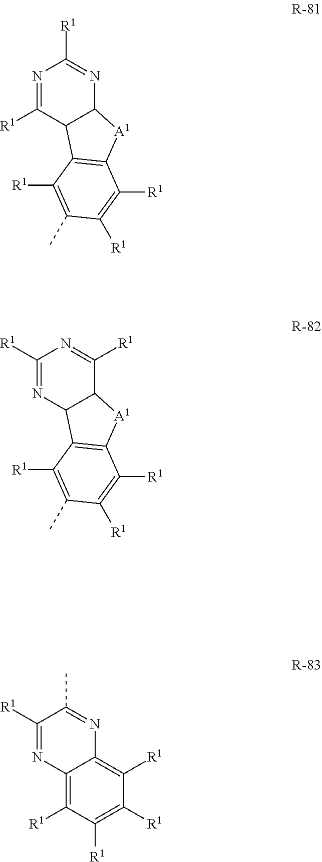

where R¹ has the definitions given above, the dotted bond represents the bond to a carbon atom of the base skeleton in formulae (1), (2) and (3) or in the preferred embodiments or to the nitrogen atom in the N(Ar′)₂ group and, in addition:
Ar⁴ is the same or different at each instance and is a bivalent aromatic or heteroaromatic ring system which has 6 to 18 aromatic ring atoms and may be substituted in each case by one or more R¹ radicals;
A¹ is the same or different at each instance and is C(R¹)₂, NR¹, O or S;
n is 0 or 1, where n=0 means that no A¹ group is bonded at this position and R¹ radicals are bonded to the corresponding carbon atoms instead;
m is 0 or 1, where m=0 means that the Ar⁴ group is absent and that the corresponding aromatic or heteroaromatic group is bonded directly to a carbon atom of the base skeleton in formula (1) or in the preferred embodiments, or to the nitrogen atom in the N(Ar′)₂ group; with the proviso that m=1 for the structures (R-12), (R-17), (R-21), (R-25), (R-26), (R-30), (R-34), (R-38) and (R-39) when these groups are embodiments of Ar′.

When the abovementioned Ar-1 to Ar-83 groups for Ar¹, Ar² or Ar³ or R-1 to R-83 groups for R or Ar′ have two or more A¹ groups, possible options for these include all combinations from the definition of A¹. Preferred embodiments in that case are those in which one A¹ group is NR or NR¹ and the other A¹ group is C(R)₂ or C(R¹)₂ or in which both A¹ groups are NR or NR¹ or in which both A¹ groups are O. In a particularly preferred embodiment of the invention, in Ar¹, Ar², Ar³, R or Ar′ groups having two or more A¹ groups, at least one A¹ group is C(R)₂ or C(R¹)₂ or is NR or NR¹.

When A¹ is NR or NR¹, the substituent R or R¹ bonded to the nitrogen atom is preferably an aromatic or heteroaromatic ring system which has 5 to 24 aromatic ring atoms and may also be substituted by one or more R¹ or R² radicals. In a particularly preferred embodiment, this R or R¹ substituent is the same or different at each instance and is an aromatic or heteroaromatic ring system which has 6 to 24 aromatic ring atoms, preferably 6 to 12 aromatic ring atoms, and which does not have any fused aryl groups or heteroaryl groups in which two or more aromatic or heteroaromatic 6-membered ring groups are fused directly to one another, and which may also be substituted in each case by one or more R¹ or R² radicals. Particular preference is given to phenyl, biphenyl, terphenyl and quaterphenyl having bonding patterns as listed above for Ar-1 to Ar-11 or R-1 to R-11, where these structures may be substituted by one or more R¹ or R² radicals, but are preferably unsubstituted.

When A¹ is C(R)₂ or C(R¹)₂, the substituents R or R¹ bonded to this carbon atom are preferably the same or different at each instance and are a linear alkyl group having 1 to 10 carbon atoms or a branched or cyclic alkyl group having 3 to 10 carbon atoms or an aromatic or heteroaromatic ring system which has 5 to 24 aromatic ring atoms and may also be substituted by one or more R¹ or R² radicals. Most preferably, R or R¹ is a methyl group or a phenyl group. In this case, the R or R¹ radicals together may also form a ring system, which leads to a spiro system.

As described above, it is essential to the invention that the compound has at least one R radical which is a heteroaromatic ring system and/or that at least one Ar¹ or Ar² group is a heteroaromatic ring system and/or that the compound has a group of the formula (3).

In one embodiment of the invention, at least one R radical is an electron-rich heteroaromatic ring system. This electron-rich heteroaromatic ring system is preferably selected from the above-depicted R-13 to R-42 groups, where, in the R-13 to R-16, R-18 to R-20, R-22 to R-24, R-27 to R-29, R-31 to R-33 and R-35 to R-37 groups, at least one A¹ group is NR¹ where R¹ is preferably an aromatic or heteroaromatic ring system, especially an aromatic ring system. Particular preference is given to the R-15 group with m=0 and A¹=NR¹.

In a further embodiment of the invention, at least one R radical is an electron-deficient heteroaromatic ring system. This electron-deficient heteroaromatic ring system is preferably selected from the above-depicted R-47 to R-50, R-57, R-58 and R-76 to R-83 groups.

In a further embodiment of the invention, Ar¹ and/or Ar² is an electron-deficient heteroaromatic ring system. This electron-deficient heteroaromatic ring system is preferably selected from the above-depicted Ar-47 to Ar-50, Ar-57, Ar-58 and Ar-76 to Ar-83 groups.

In a further preferred embodiment of the invention, R¹ is the same or different at each instance and is selected from the group consisting of H, D, F, CN, OR², a straight-chain alkyl group having 1 to 10 carbon atoms or an alkenyl group having 2 to 10 carbon atoms or a branched or cyclic alkyl group having 3 to 10 carbon atoms, where the alkyl or alkenyl group may in each case be substituted by one or more R² radicals, and where one or more nonadjacent CH₂ groups may be replaced by O, or an aromatic or heteroaromatic ring system which has 6 to 30 aromatic ring atoms and may be substituted in each case by one or more R² radicals; at the same time, two or more R¹ radicals together may form an aliphatic ring system. In a particularly preferred embodiment of the invention, R¹ is the same or different at each instance and is selected from the group consisting of H, a straight-chain alkyl group having 1 to 6 carbon atoms, especially having 1, 2, 3 or 4 carbon atoms, or a branched or cyclic alkyl group having 3 to 6 carbon atoms, where the alkyl group may be substituted by one or more R² radicals, but is preferably unsubstituted, or an aromatic or heteroaromatic ring system which has 6 to 24 aromatic ring atoms and may be substituted in each case by one or more R² radicals, but is preferably unsubstituted.

In a further preferred embodiment of the invention, R² is the same or different at each instance and is H, F, an alkyl group having 1 to 4 carbon atoms or an aryl group having 6 to 10 carbon atoms, which may be substituted by an alkyl group having 1 to 4 carbon atoms, but is preferably unsubstituted.

Further suitable Ar¹, Ar², Ar³, R or Ar′ groups are groups of the formula —Ar⁷—N(Ar⁵)(Ar⁶) where Ar⁵, Ar⁶ and Ar⁷ are the same or different at each instance and are an aromatic or heteroaromatic ring system which has 5 to 24 aromatic ring atoms and may be substituted in each case by one or more R¹ radicals. Ar¹, Ar² or Ar³ gives rise to such a group when the Ar¹, Ar² or Ar³ group is substituted by an N(Ar′)₂ group. The total number of aromatic ring atoms in Ar⁵, Ar⁶ and Ar^{F} here is not more than 60 and preferably not more than 40.

In this case, Ar⁷ and Ar⁵ may also be bonded to one another and/or Ar⁵ and Ar⁶ to one another via a group selected from C(R¹)₂, NR¹, O or S. Preferably, Ar⁷ and Ar⁵ are joined to one another and Ar⁵ and Ar⁶ to one another in the respective ortho position to the bond to the nitrogen atom. In a further embodiment of the invention, none of the Ar⁵, Ar⁶ and Ar⁷ groups are bonded to one another.

Preferably, Ar⁷ is an aromatic or heteroaromatic ring system which has 6 to 24 aromatic ring atoms, especially 6 to 12 aromatic ring atoms, and may be substituted in each case by one or more R¹ radicals. More preferably, Ar⁷ is selected from the group consisting of ortho-, meta- or para-phenylene or ortho-, meta- or para-biphenyl, each of which may be substituted by one or more R¹ radicals, but are preferably unsubstituted. Most preferably, Ar⁷ is an unsubstituted phenylene group. This is especially true when Ar⁷ is bonded to Ar⁵ via a single bond.

Preferably, Ar⁵ and Ar⁶ are the same or different at each instance and are an aromatic or heteroaromatic ring system which has 6 to 24 aromatic ring atoms and may be substituted in each case by one or more R¹ radicals. Particularly preferred Ar⁵ and Ar⁶ groups are the same or different at each instance and are selected from the group consisting of benzene, ortho-, meta- or para-biphenyl, ortho-, meta- or para-terphenyl or branched terphenyl, ortho-, meta- or para-quaterphenyl or branched quaterphenyl, 1-, 2-, 3- or 4-fluorenyl, 1-, 2-, 3- or 4-spirobifluorenyl, 1- or 2-naphthyl, indole, benzofuran, benzothiophene, 1-, 2-, 3- or 4-carbazole, 1-, 2-, 3- or 4-dibenzofuran, 1-, 2-, 3- or 4-dibenzothiophene, indenocarbazole, indolocarbazole, 2-, 3- or 4-pyridine, 2-, 4- or 5-pyrimidine, pyrazine, pyridazine, triazine, phenanthrene, triphenylene or combinations of two, three or four of these groups, each of which may be substituted by one or more R¹ radicals. More preferably, Ar⁵ and Ar⁶ are the same or different at each instance and are an aromatic ring system which has 6 to 24 aromatic ring atoms and may be substituted by one or more R¹ radicals, especially selected from the groups consisting of benzene, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene, especially 1-, 2-, 3- or 4-fluorene, or spirobifluorene, especially 1-, 2-, 3- or 4-spirobifluorene.

At the same time, the alkyl groups in compounds of the invention which are processed by vacuum evaporation preferably have not more than five carbon atoms, more preferably not more than 4 carbon atoms, most preferably not more than 1 carbon atom. For compounds that are processed from solution, suitable compounds are also those substituted by alkyl groups, especially branched alkyl groups, having up to 10 carbon atoms or those substituted by oligoarylene groups, for example ortho-, meta- or para-terphenyl or branched terphenyl or quaterphenyl groups.

When the compounds of the formula (1) or the preferred embodiments are used as matrix material for a phosphorescent emitter or in a layer directly adjoining a phosphorescent layer, it is further preferable when the compound does not contain any fused aryl or heteroaryl groups in which more than two six-membered rings are fused directly to one another. It is especially preferable that the Ar¹, Ar², Ar³, R, Ar′, R¹ and R² groups do not contain any fused aryl or heteroaryl groups in which two or more six-membered rings are fused directly to one another. An exception to this is formed by phenanthrene and triphenylene which, because of their high triplet energy, may be preferable in spite of the presence of fused aromatic six-membered rings.

The abovementioned preferred embodiments may be combined with one another as desired within the restrictions defined in claim 1. In a particularly preferred embodiment of the invention, the abovementioned preferences occur simultaneously.

Examples of suitable compounds according to the above-detailed embodiments are the compounds detailed in the following table:

| | |
|---|---|
| 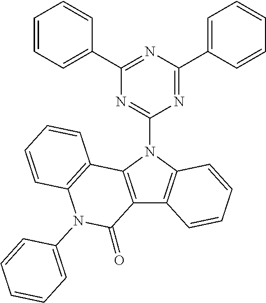 [SMILES: O=c1c2c3ccccc3n(-c3nc(-c4ccccc4)nc(-c4ccccc4)n3)c2c2ccccc2n1-c1ccccc1] | 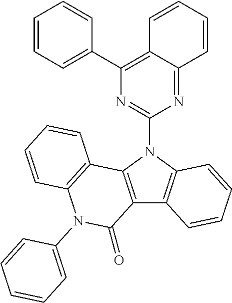 [SMILES: O=c1c2c3ccccc3n(-c3nc(-c4ccccc4)c4ccccc4n3)c2c2ccccc2n1-c1ccccc1] |
| 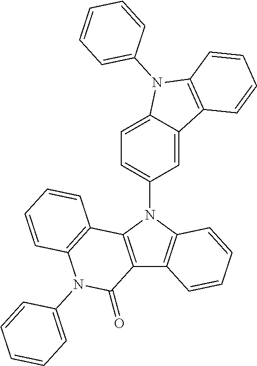 [SMILES: O=c1c2c3ccccc3n(-c3ccc4c(c3)c3ccccc3n4-c3ccccc3)c2c2ccccc2n1-c1ccccc1] | 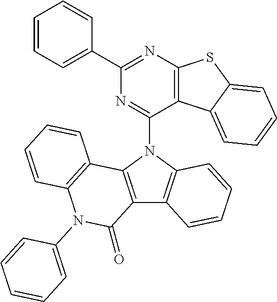 [SMILES: O=c1c2c3ccccc3n(-c3nc(-c4ccccc4)nc4sc5ccccc5c34)c2c2ccccc2n1-c1ccccc1] |
| 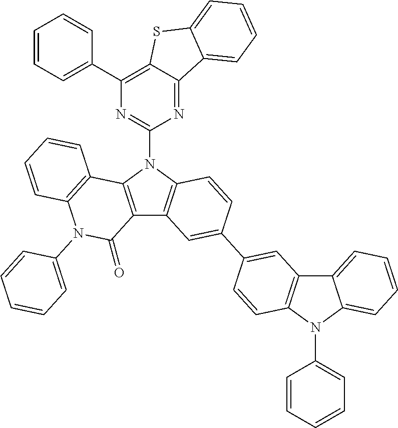 [SMILES: O=c1c2c3cc(-c4ccc5c(c4)c4ccccc4n5-c4ccccc4)ccc3n(-c3nc(-c4ccccc4)c4sc5ccccc5c4n3)c2c2ccccc2n1-c1ccccc1] | 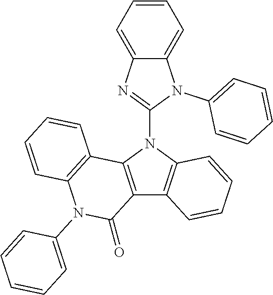 [SMILES: O=c1c2c3ccccc3n(-c3nc4ccccc4n3-c3ccccc3)c2c2ccccc2n1-c1ccccc1] |
| 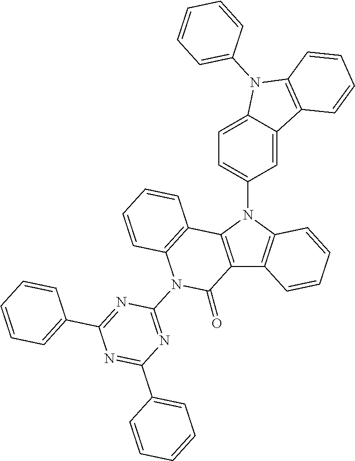 [SMILES: O=c1c2c3ccccc3n(-c3ccc4c(c3)c3ccccc3n4-c3ccccc3)c2c2ccccc2n1-c1nc(-c2ccccc2)nc(-c2ccccc2)n1] | 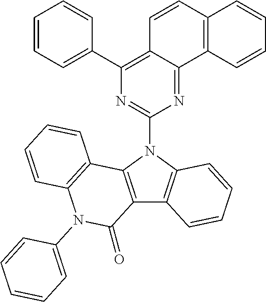 [SMILES: O=c1c2c3ccccc3n(-c3nc(-c4ccccc4)c4ccc5ccccc5c4n3)c2c2ccccc2n1-c1ccccc1] |
| 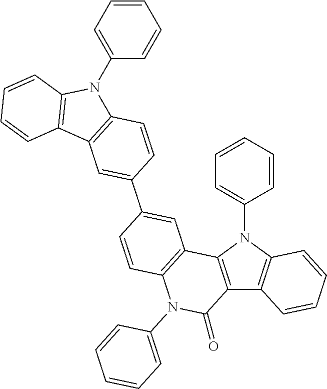 [SMILES: O=c1c2c3ccccc3n(-c3ccccc3)c2c2cc(-c3ccc4c(c3)c3ccccc3n4-c3ccccc3)ccc2n1-c1ccccc1] | 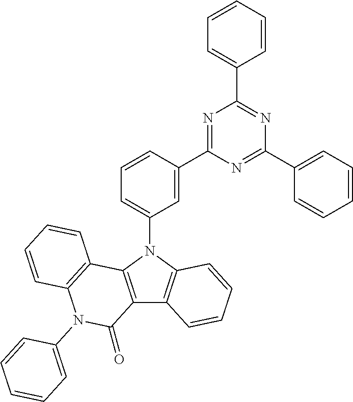 [SMILES: O=c1c2c3ccccc3n(-c3cccc(-c4nc(-c5ccccc5)nc(-c5ccccc5)n4)c3)c2c2ccccc2n1-c1ccccc1] |
| 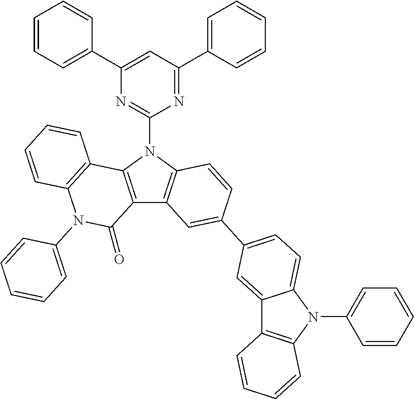 [SMILES: O=c1c2c3cc(-c4ccc5c(c4)c4ccccc4n5-c4ccccc4)ccc3n(-c3nc(-c4ccccc4)cc(-c4ccccc4)n3)c2c2ccccc2n1-c1ccccc1] | 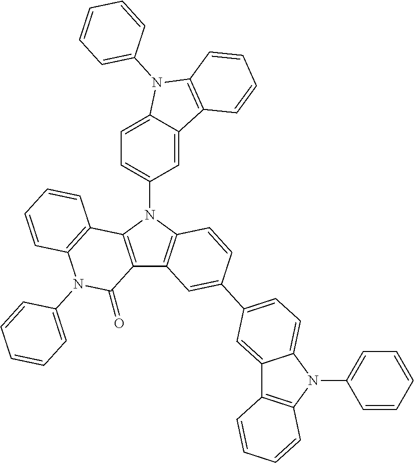 [SMILES: O=c1c2c3cc(-c4ccc5c(c4)c4ccccc4n5-c4ccccc4)ccc3n(-c3ccc4c(c3)c3ccccc3n4-c3ccccc3)c2c2ccccc2n1-c1ccccc1] |

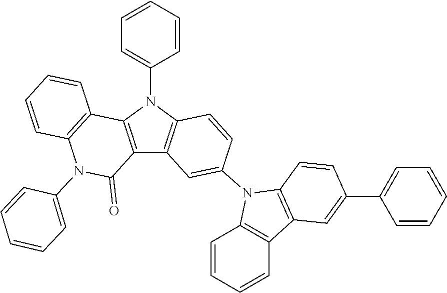 [SMILES: O=c1c2c3cc(-n4c5ccccc5c5cc(-c6ccccc6)ccc54)ccc3n(-c3ccccc3)c2c2ccccc2n1-c1ccccc1]

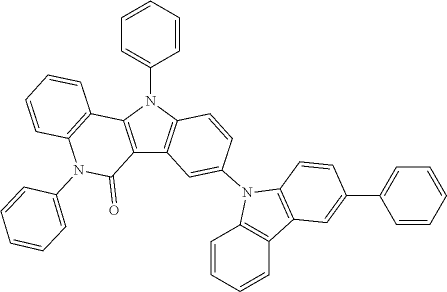 [SMILES: O=c1c2c3cc(-n4c5ccccc5c5cc(-c6ccccc6)ccc54)ccc3n(-c3ccccc3)c2c2ccccc2n1-c1ccccc1]

| | |
|---|---|
| 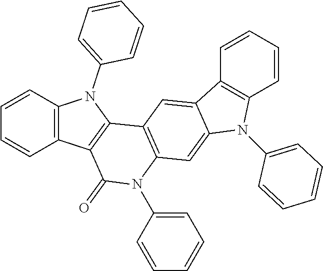 [SMILES: O=c1c2c3ccccc3n(-c3ccccc3)c2c2cc3c4ccccc4n(-c4ccccc4)c3cc2n1-c1ccccc1] | 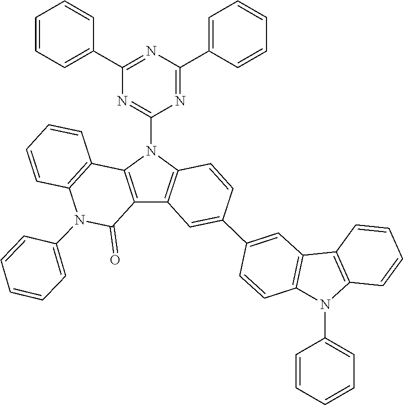 [SMILES: O=c1c2c3cc(-c4ccc5c(c4)c4ccccc4n5-c4ccccc4)ccc3n(-c3nc(-c4ccccc4)nc(-c4ccccc4)n3)c2c2ccccc2n1-c1ccccc1] |
| 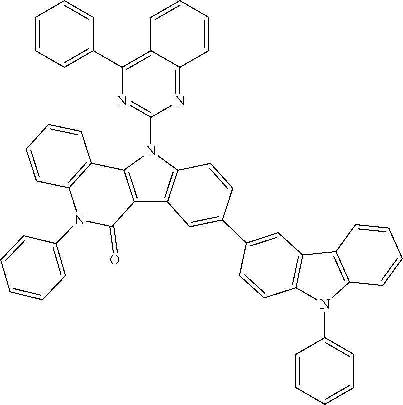 [SMILES: O=c1c2c3cc(-c4ccc5c(c4)c4ccccc4n5-c4ccccc4)ccc3n(-c3nc(-c4ccccc4)c4ccccc4n3)c2c2ccccc2n1-c1ccccc1] | 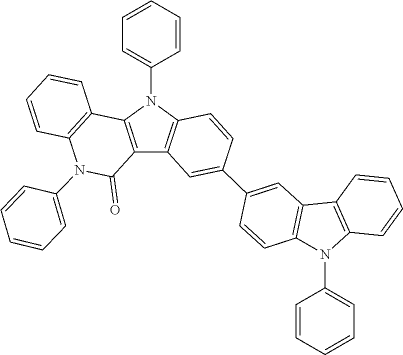 [SMILES: O=c1c2c3cc(-c4ccc5c(c4)c4ccccc4n5-c4ccccc4)ccc3n(-c3ccccc3)c2c2ccccc2n1-c1ccccc1] |
| 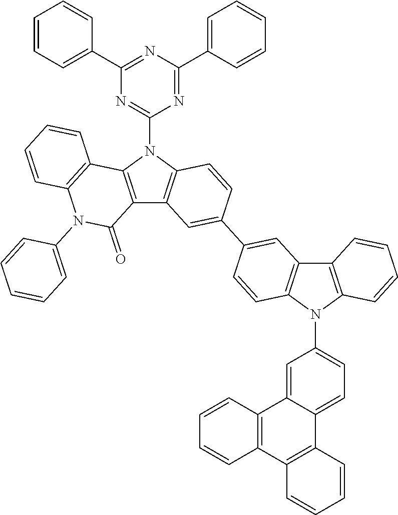 [SMILES: O=c1c2c3cc(-c4ccc5c(c4)c4ccccc4n5-c4ccc5c6ccccc6c6ccccc6c5c4)ccc3n(-c3nc(-c4ccccc4)nc(-c4ccccc4)n3)c2c2ccccc2n1-c1ccccc1] | 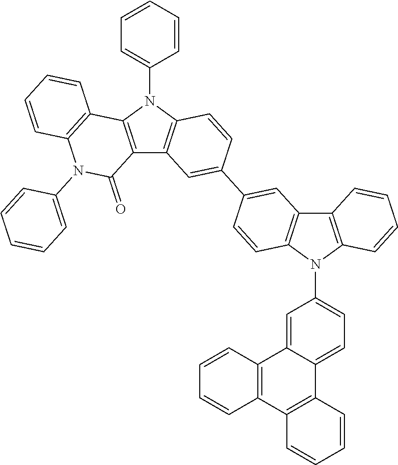 [SMILES: O=c1c2c3cc(-c4ccc5c(c4)c4ccccc4n5-c4ccc5c6ccccc6c6ccccc6c5c4)ccc3n(-c3ccccc3)c2c2ccccc2n1-c1ccccc1] |
| 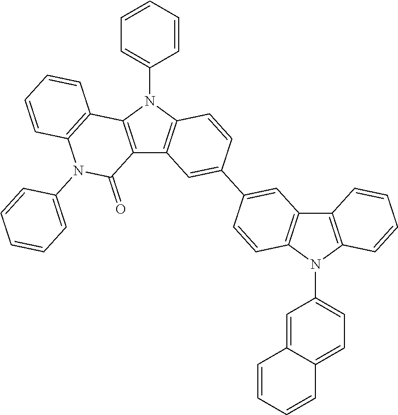 [SMILES: O=c1c2c3cc(-c4ccc5c(c4)c4ccccc4n5-c4ccc5ccccc5c4)ccc3n(-c3ccccc3)c2c2ccccc2n1-c1ccccc1] | 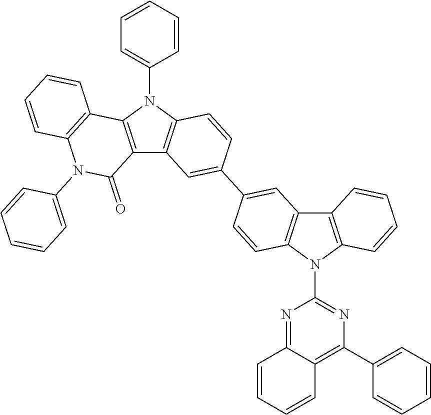 [SMILES: O=c1c2c3cc(-c4ccc5c(c4)c4ccccc4n5-c4nc(-c5ccccc5)c5ccccc5n4)ccc3n(-c3ccccc3)c2c2ccccc2n1-c1ccccc1] |
| 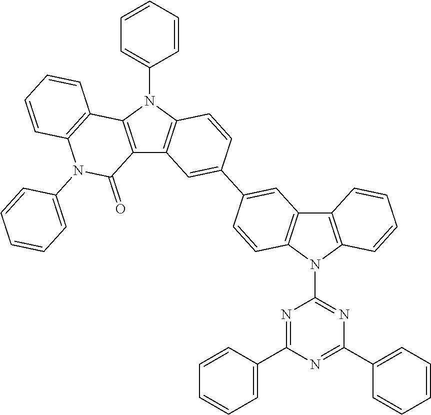 [SMILES: O=c1c2c3cc(-c4ccc5c(c4)c4ccccc4n5-c4nc(-c5ccccc5)nc(-c5ccccc5)n4)ccc3n(-c3ccccc3)c2c2ccccc2n1-c1ccccc1] | 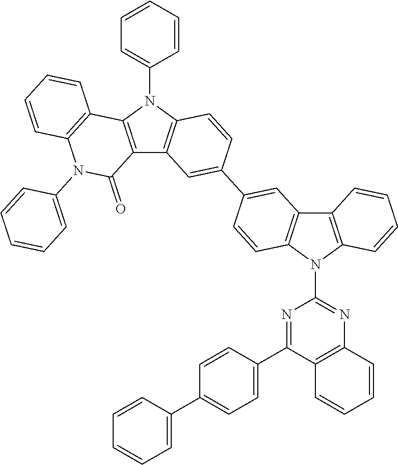 [SMILES: O=c1c2c3cc(-c4ccc5c(c4)c4ccccc4n5-c4nc(-c5ccc(-c6ccccc6)cc5)c5ccccc5n4)ccc3n(-c3ccccc3)c2c2ccccc2n1-c1ccccc1] |
| 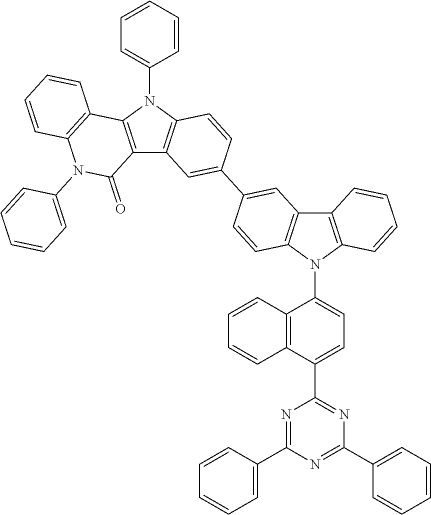 [SMILES: O=c1c2c3cc(-c4ccc5c(c4)c4ccccc4n5-c4ccc(-c5nc(-c6ccccc6)nc(-c6ccccc6)n5)c5ccccc45)ccc3n(-c3ccccc3)c2c2ccccc2n1-c1ccccc1] | 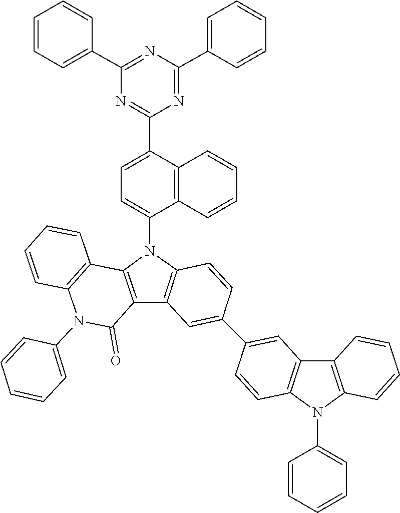 [SMILES: O=c1c2c3cc(-c4ccc5c(c4)c4ccccc4n5-c4ccccc4)ccc3n(-c3ccc(-c4nc(-c5ccccc5)nc(-c5ccccc5)n4)c4ccccc34)c2c2ccccc2n1-c1ccccc1] |
| 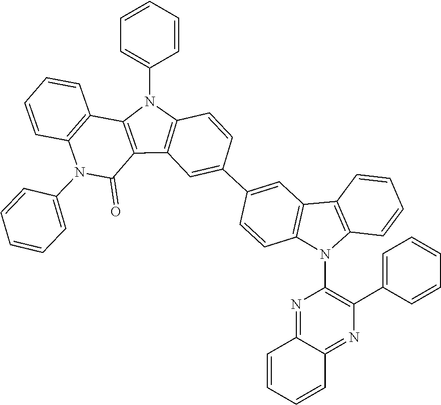 [SMILES: O=c1c2c3cc(-c4ccc5c(c4)c4ccccc4n5-c4nc5ccccc5nc4-c4ccccc4)ccc3n(-c3ccccc3)c2c2ccccc2n1-c1ccccc1] | 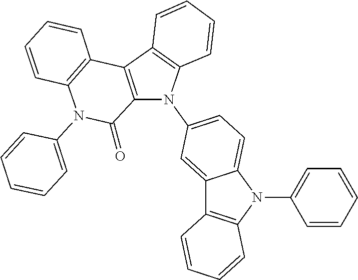 [SMILES: O=c1c2c(c3ccccc3n1-c1ccccc1)c1ccccc1n2-c1ccc2c(c1)c1ccccc1n2-c1ccccc1] |
| 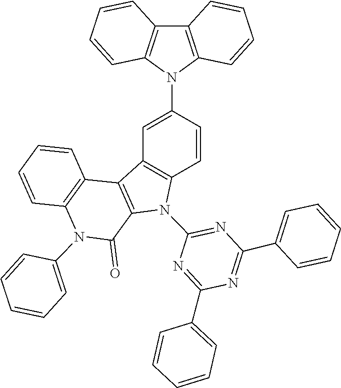 [SMILES: O=c1c2c(c3ccccc3n1-c1ccccc1)c1cc(-n3c4ccccc4c4ccccc43)ccc1n2-c1nc(-c2ccccc2)nc(-c2ccccc2)n1] | 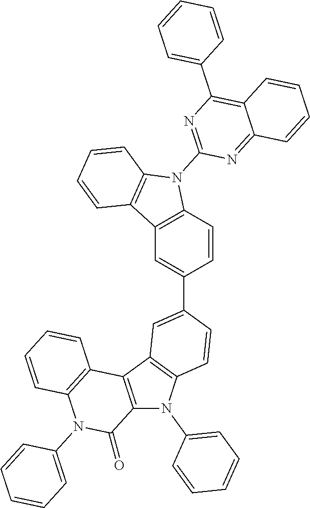 [SMILES: O=c1c2c(c3ccccc3n1-c1ccccc1)c1cc(-c3ccc4c(c3)c3ccccc3n4-c3nc(-c4ccccc4)c4ccccc4n3)ccc1n2-c1ccccc1] |
| 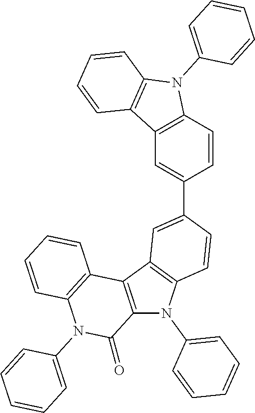 [SMILES: O=c1c2c(c3ccccc3n1-c1ccccc1)c1cc(-c3ccc4c(c3)c3ccccc3n4-c3ccccc3)ccc1n2-c1ccccc1] | 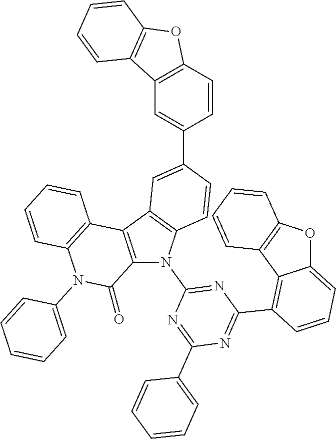 [SMILES: O=c1c2c(c3ccccc3n1-c1ccccc1)c1cc(-c3ccc4oc5ccccc5c4c3)ccc1n2-c1nc(-c2ccccc2)nc(-c2cccc3oc4ccccc4c23)n1] |
| 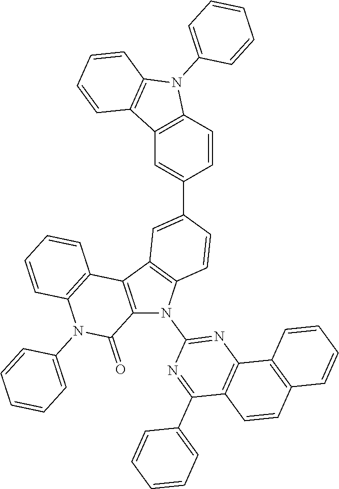 [SMILES: O=c1c2c(c3ccccc3n1-c1ccccc1)c1cc(-c3ccc4c(c3)c3ccccc3n4-c3ccccc3)ccc1n2-c1nc(-c2ccccc2)c2ccc3ccccc3c2n1] | 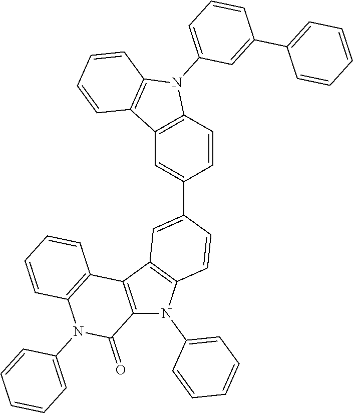 [SMILES: O=c1c2c(c3ccccc3n1-c1ccccc1)c1cc(-c3ccc4c(c3)c3ccccc3n4-c3cccc(-c4ccccc4)c3)ccc1n2-c1ccccc1] |
| 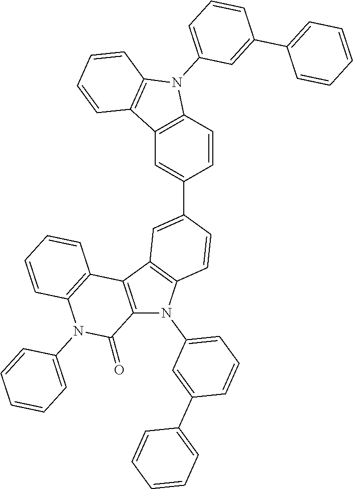 [SMILES: O=c1c2c(c3ccccc3n1-c1ccccc1)c1cc(-c3ccc4c(c3)c3ccccc3n4-c3cccc(-c4ccccc4)c3)ccc1n2-c1cccc(-c2ccccc2)c1] | 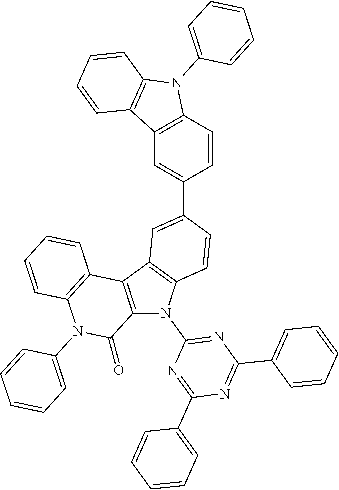 [SMILES: O=c1c2c(c3ccccc3n1-c1ccccc1)c1cc(-c3ccc4c(c3)c3ccccc3n4-c3ccccc3)ccc1n2-c1nc(-c2ccccc2)nc(-c2ccccc2)n1] |
| 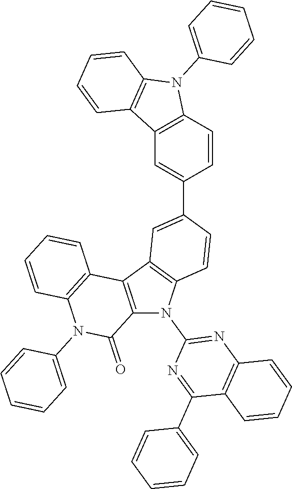 [SMILES: O=c1c2c(c3ccccc3n1-c1ccccc1)c1cc(-c3ccc4c(c3)c3ccccc3n4-c3ccccc3)ccc1n2-c1nc(-c2ccccc2)c2ccccc2n1] | 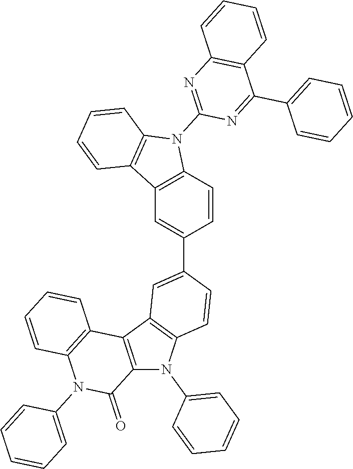 [SMILES: O=c1c2c(c3ccccc3n1-c1ccccc1)c1cc(-c3ccc4c(c3)c3ccccc3n4-c3nc(-c4ccccc4)c4ccccc4n3)ccc1n2-c1ccccc1] |
| 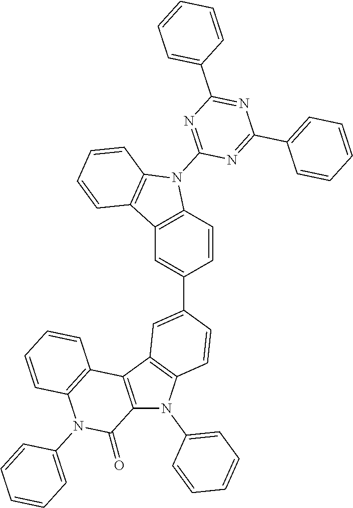 [SMILES: O=c1c2c(c3ccccc3n1-c1ccccc1)c1cc(-c3ccc4c(c3)c3ccccc3n4-c3nc(-c4ccccc4)nc(-c4ccccc4)n3)ccc1n2-c1ccccc1] | 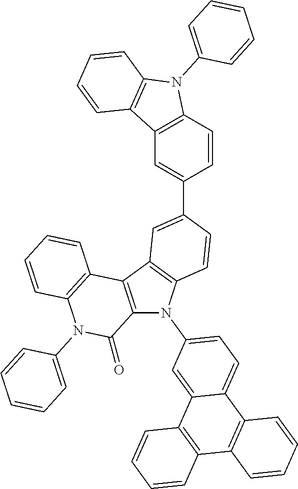 [SMILES: O=c1c2c(c3ccccc3n1-c1ccccc1)c1cc(-c3ccc4c(c3)c3ccccc3n4-c3ccccc3)ccc1n2-c1ccc2c3ccccc3c3ccccc3c2c1] |
| 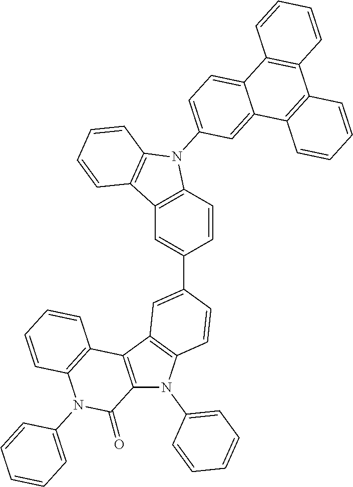 [SMILES: O=c1c2c(c3ccccc3n1-c1ccccc1)c1cc(-c3ccc4c(c3)c3ccccc3n4-c3ccc4c5ccccc5c5ccccc5c4c3)ccc1n2-c1ccccc1] | 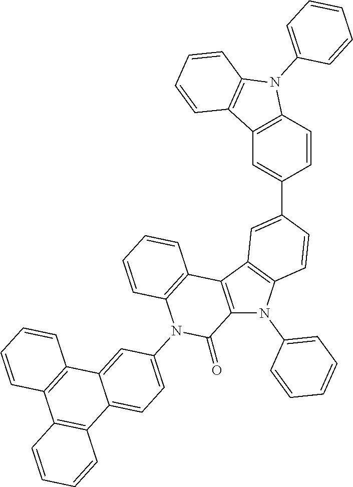 [SMILES: O=c1c2c(c3ccccc3n1-c1ccc3c4ccccc4c4ccccc4c3c1)c1cc(-c3ccc4c(c3)c3ccccc3n4-c3ccccc3)ccc1n2-c1ccccc1] |
| 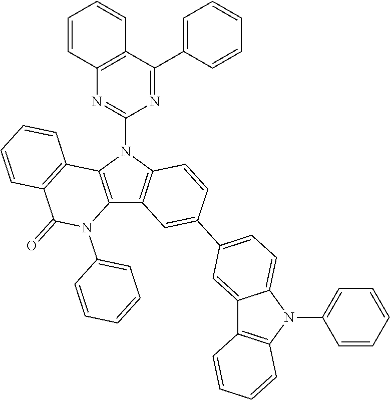 [SMILES: O=c1c2ccccc2c2c(c3cc(-c4ccc5c(c4)c4ccccc4n5-c4ccccc4)ccc3n2-c2nc(-c3ccccc3)c3ccccc3n2)n1-c1ccccc1] | 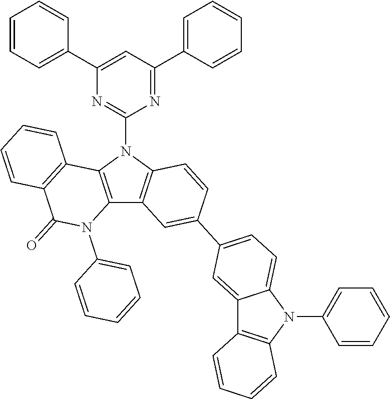 [SMILES: O=c1c2ccccc2c2c(c3cc(-c4ccc5c(c4)c4ccccc4n5-c4ccccc4)ccc3n2-c2nc(-c3ccccc3)cc(-c3ccccc3)n2)n1-c1ccccc1] |
| 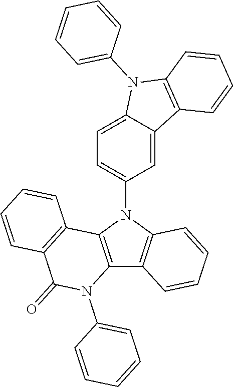 [SMILES: O=c1c2ccccc2c2c(c3ccccc3n2-c2ccc3c(c2)c2ccccc2n3-c2ccccc2)n1-c1ccccc1] | 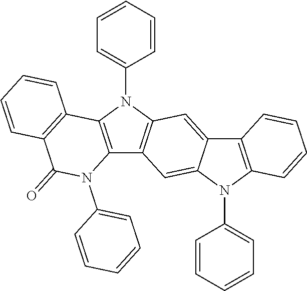 [SMILES: O=c1c2ccccc2c2c(c3cc4c(cc3n2-c2ccccc2)c2ccccc2n4-c2ccccc2)n1-c1ccccc1] |
| 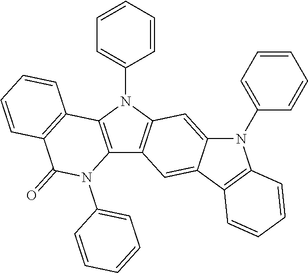 [SMILES: O=c1c2ccccc2c2c(c3cc4c5ccccc5n(-c5ccccc5)c4cc3n2-c2ccccc2)n1-c1ccccc1] | 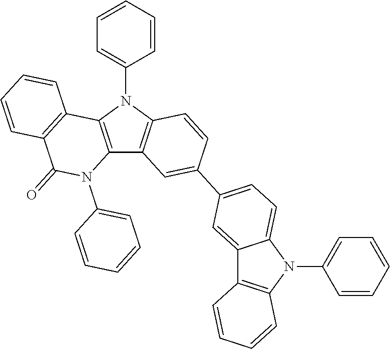 [SMILES: O=c1c2ccccc2c2c(c3cc(-c4ccc5c(c4)c4ccccc4n5-c4ccccc4)ccc3n2-c2ccccc2)n1-c1ccccc1] |
| 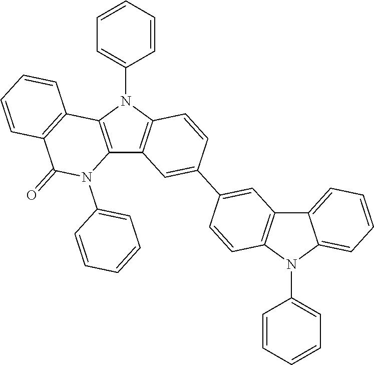 [SMILES: O=c1c2ccccc2c2c(c3cc(-c4ccc5c(c4)c4ccccc4n5-c4ccccc4)ccc3n2-c2ccccc2)n1-c1ccccc1] | 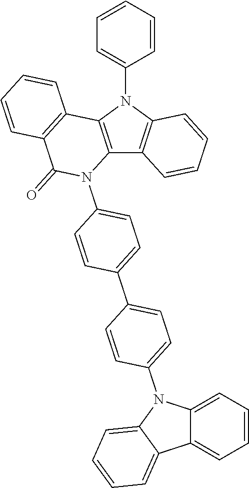 [SMILES: O=c1c2ccccc2c2c(c3ccccc3n2-c2ccccc2)n1-c1ccc(-c2ccc(-n3c4ccccc4c4ccccc43)cc2)cc1] |
| 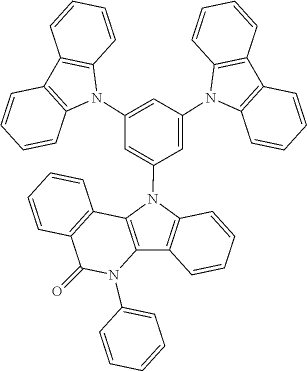 [SMILES: O=c1c2ccccc2c2c(c3ccccc3n2-c2cc(-n3c4ccccc4c4ccccc43)cc(-n3c4ccccc4c4ccccc43)c2)n1-c1ccccc1] | 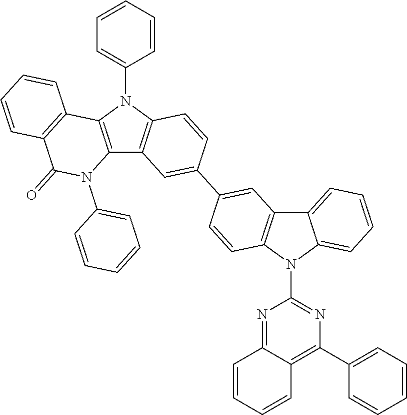 [SMILES: O=c1c2ccccc2c2c(c3cc(-c4ccc5c(c4)c4ccccc4n5-c4nc(-c5ccccc5)c5ccccc5n4)ccc3n2-c2ccccc2)n1-c1ccccc1] |
| 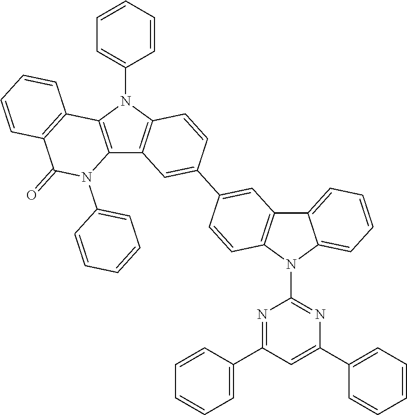 [SMILES: O=c1c2ccccc2c2c(c3cc(-c4ccc5c(c4)c4ccccc4n5-c4nc(-c5ccccc5)cc(-c5ccccc5)n4)ccc3n2-c2ccccc2)n1-c1ccccc1] | 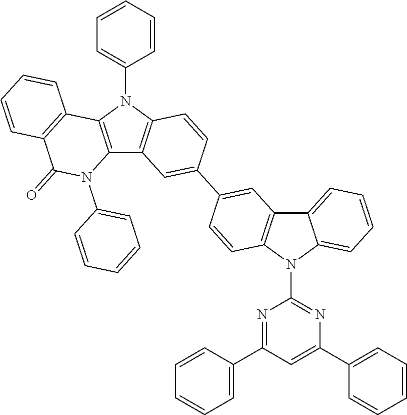 [SMILES: O=c1c2ccccc2c2c(c3cc(-c4ccc5c(c4)c4ccccc4n5-c4nc(-c5ccccc5)cc(-c5ccccc5)n4)ccc3n2-c2ccccc2)n1-c1ccccc1] |
| 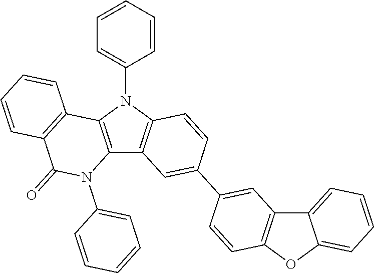 [SMILES: O=c1c2ccccc2c2c(c3cc(-c4ccc5oc6ccccc6c5c4)ccc3n2-c2ccccc2)n1-c1ccccc1] | 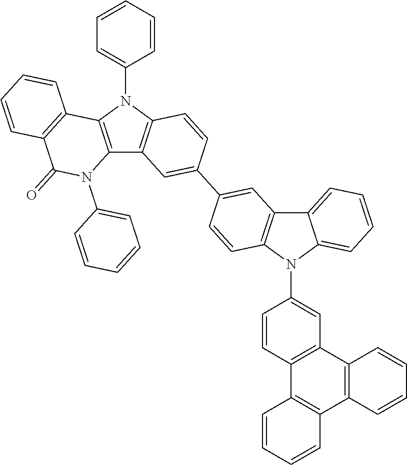 [SMILES: O=c1c2ccccc2c2c(c3cc(-c4ccc5c(c4)c4ccccc4n5-c4ccc5c6ccccc6c6ccccc6c5c4)ccc3n2-c2ccccc2)n1-c1ccccc1] |
| 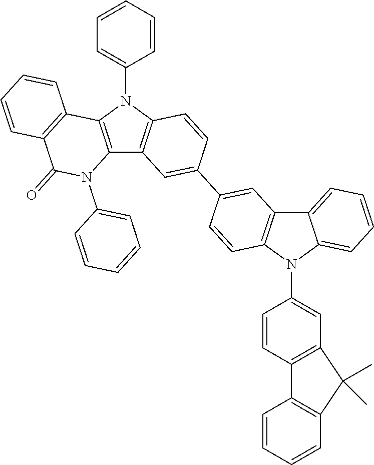 [SMILES: CC1(C)c2ccccc2-c2ccc(-n3c4ccccc4c4cc(-c5ccc6c(c5)c5c(c7ccccc7c(=O)n5-c5ccccc5)n6-c5ccccc5)ccc43)cc21] | 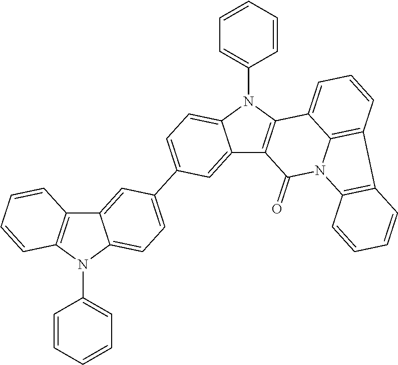 [SMILES: O=c1c2c3cc(-c4ccc5c(c4)c4ccccc4n5-c4ccccc4)ccc3n(-c3ccccc3)c2c2cccc3c4ccccc4n1c32] |
| 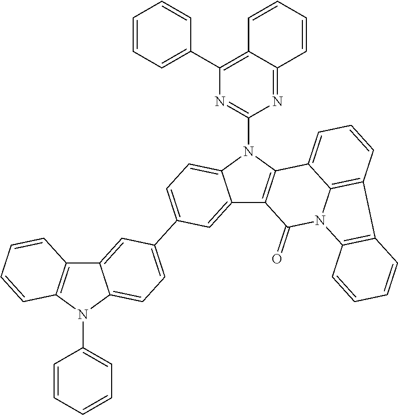 [SMILES: O=c1c2c3cc(-c4ccc5c(c4)c4ccccc4n5-c4ccccc4)ccc3n(-c3nc(-c4ccccc4)c4ccccc4n3)c2c2cccc3c4ccccc4n1c32] | 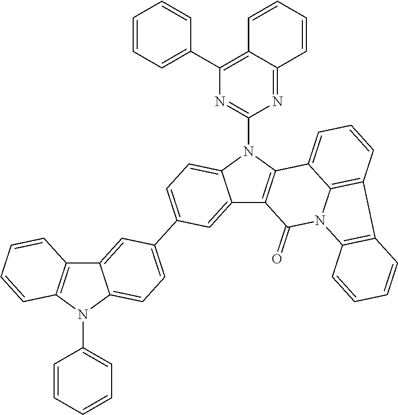 [SMILES: O=c1c2c3cc(-c4ccc5c(c4)c4ccccc4n5-c4ccccc4)ccc3n(-c3nc(-c4ccccc4)c4ccccc4n3)c2c2cccc3c4ccccc4n1c32] |

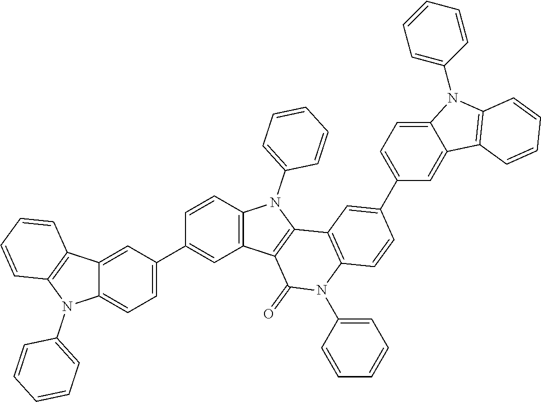 [SMILES: O=c1c2c3cc(-c4ccc5c(c4)c4ccccc4n5-c4ccccc4)ccc3n(-c3ccccc3)c2c2cc(-c3ccc4c(c3)c3ccccc3n4-c3ccccc3)ccc2n1-c1ccccc1]

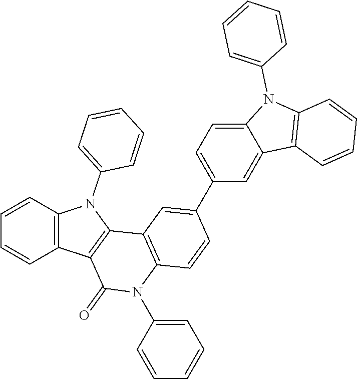 [SMILES: O=c1c2c3ccccc3n(-c3ccccc3)c2c2cc(-c3ccc4c(c3)c3ccccc3n4-c3ccccc3)ccc2n1-c1ccccc1]

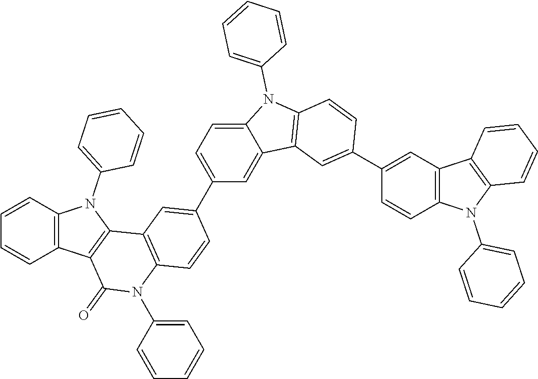 [SMILES: O=c1c2c3ccccc3n(-c3ccccc3)c2c2cc(-c3ccc4c(c3)c3cc(-c5ccc6c(c5)c5ccccc5n6-c5ccccc5)ccc3n4-c3ccccc3)ccc2n1-c1ccccc1]

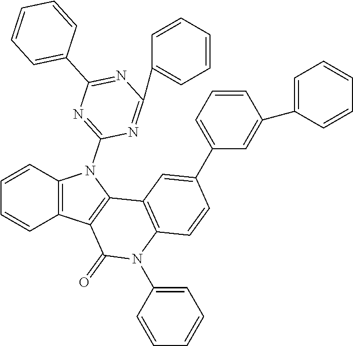 [SMILES: O=c1c2c3ccccc3n(-c3nc(-c4ccccc4)nc(-c4ccccc4)n3)c2c2cc(-c3cccc(-c4ccccc4)c3)ccc2n1-c1ccccc1]

| | |
|---|---|
| 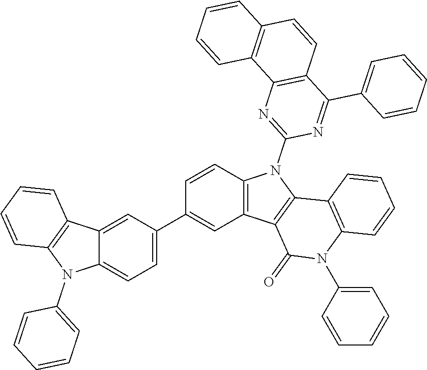 [SMILES: O=c1c2c3cc(-c4ccc5c(c4)c4ccccc4n5-c4ccccc4)ccc3n(-c3nc(-c4ccccc4)c4ccc5ccccc5c4n3)c2c2ccccc2n1-c1ccccc1] | 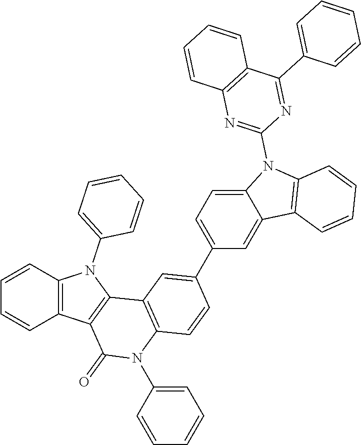 [SMILES: O=c1c2c3ccccc3n(-c3ccccc3)c2c2cc(-c3ccc4c(c3)c3ccccc3n4-c3nc(-c4ccccc4)c4ccccc4n3)ccc2n1-c1ccccc1] |
| 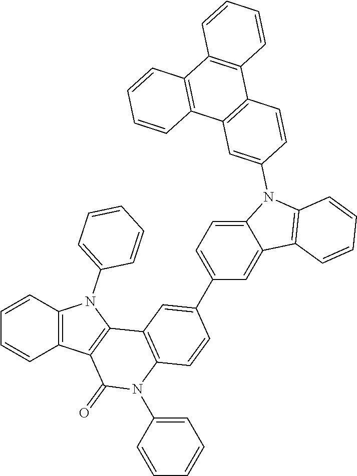 [SMILES: O=c1c2c3ccccc3n(-c3ccccc3)c2c2cc(-c3ccc4c(c3)c3ccccc3n4-c3ccc4c5ccccc5c5ccccc5c4c3)ccc2n1-c1ccccc1] | 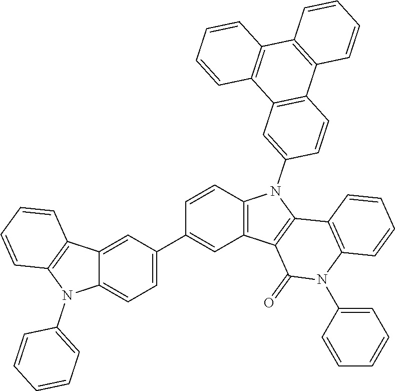 [SMILES: O=c1c2c3cc(-c4ccc5c(c4)c4ccccc4n5-c4ccccc4)ccc3n(-c3ccc4c5ccccc5c5ccccc5c4c3)c2c2ccccc2n1-c1ccccc1] |

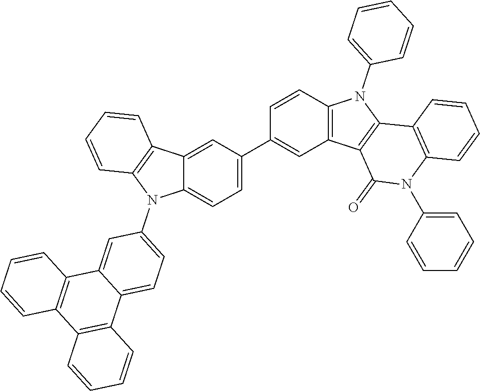 [SMILES: O=c1c2c3cc(-c4ccc5c(c4)c4ccccc4n5-c4ccc5c6ccccc6c6ccccc6c5c4)ccc3n(-c3ccccc3)c2c2ccccc2n1-c1ccccc1]

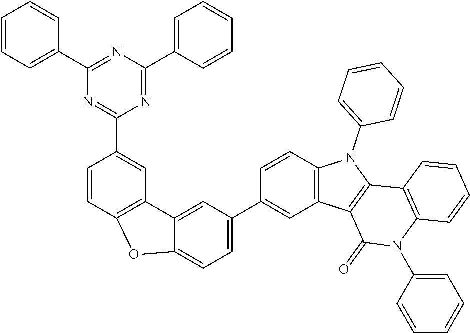 [SMILES: O=c1c2c3cc(-c4ccc5oc6ccc(-c7nc(-c8ccccc8)nc(-c8ccccc8)n7)cc6c5c4)ccc3n(-c3ccccc3)c2c2ccccc2n1-c1ccccc1]

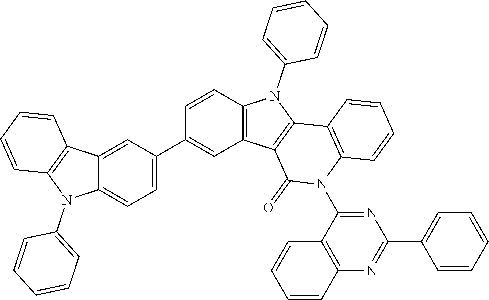 [SMILES: O=c1c2c3cc(-c4ccc5c(c4)c4ccccc4n5-c4ccccc4)ccc3n(-c3ccccc3)c2c2ccccc2n1-c1nc(-c2ccccc2)nc2ccccc12]

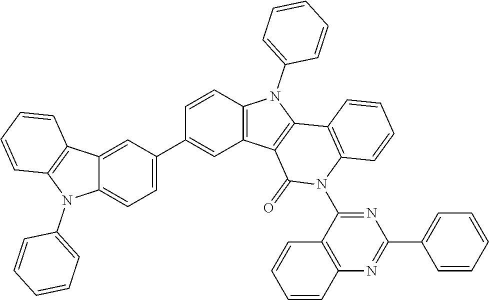 [SMILES: O=c1c2c3cc(-c4ccc5c(c4)c4ccccc4n5-c4ccccc4)ccc3n(-c3ccccc3)c2c2ccccc2n1-c1nc(-c2ccccc2)nc2ccccc12]

| | |
|---|---|
| 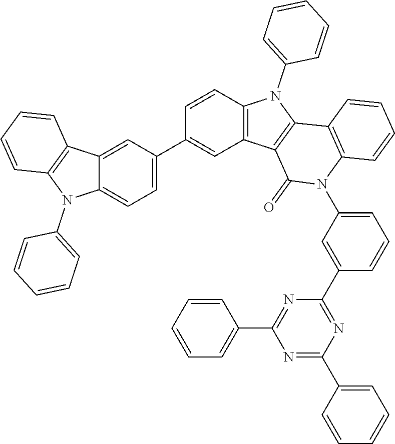 [SMILES: O=c1c2c3cc(-c4ccc5c(c4)c4ccccc4n5-c4ccccc4)ccc3n(-c3ccccc3)c2c2ccccc2n1-c1cccc(-c2nc(-c3ccccc3)nc(-c3ccccc3)n2)c1] | 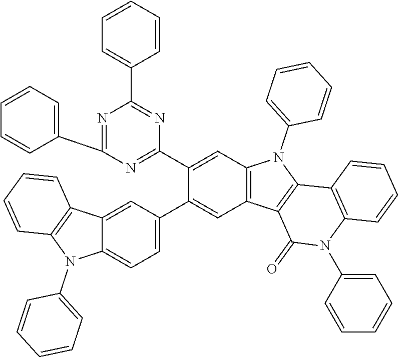 [SMILES: O=c1c2c3cc(-c4ccc5c(c4)c4ccccc4n5-c4ccccc4)c(-c4nc(-c5ccccc5)nc(-c5ccccc5)n4)cc3n(-c3ccccc3)c2c2ccccc2n1-c1ccccc1] |
| 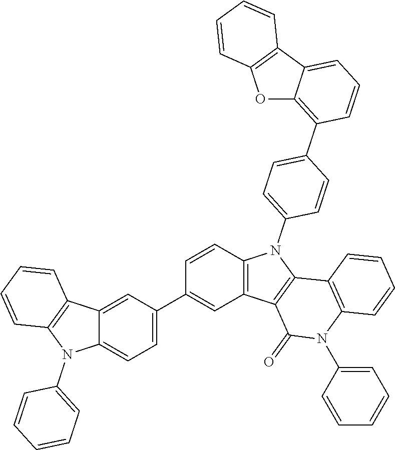 [SMILES: O=c1c2c3cc(-c4ccc5c(c4)c4ccccc4n5-c4ccccc4)ccc3n(-c3ccc(-c4cccc5c4oc4ccccc45)cc3)c2c2ccccc2n1-c1ccccc1] | 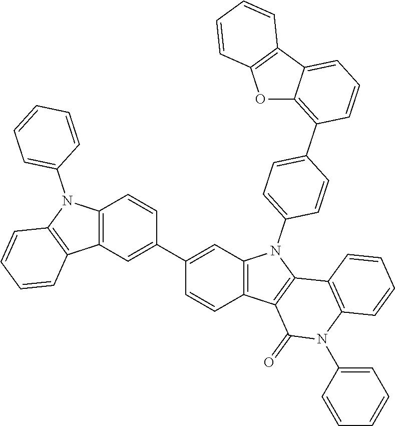 [SMILES: O=c1c2c3ccc(-c4ccc5c(c4)c4ccccc4n5-c4ccccc4)cc3n(-c3ccc(-c4cccc5c4oc4ccccc45)cc3)c2c2ccccc2n1-c1ccccc1] |
| 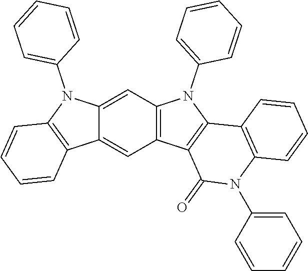 [SMILES: O=c1c2c3cc4c5ccccc5n(-c5ccccc5)c4cc3n(-c3ccccc3)c2c2ccccc2n1-c1ccccc1] | 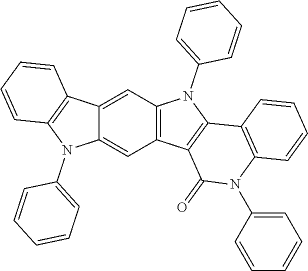 [SMILES: O=c1c2c3cc4c(cc3n(-c3ccccc3)c2c2ccccc2n1-c1ccccc1)c1ccccc1n4-c1ccccc1] |
| 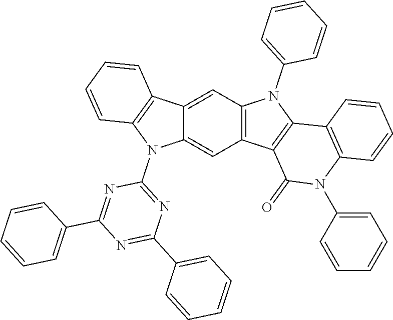 [SMILES: O=c1c2c3cc4c(cc3n(-c3ccccc3)c2c2ccccc2n1-c1ccccc1)c1ccccc1n4-c1nc(-c2ccccc2)nc(-c2ccccc2)n1] | 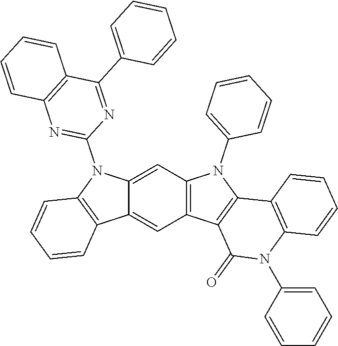 [SMILES: O=c1c2c3cc4c5ccccc5n(-c5nc(-c6ccccc6)c6ccccc6n5)c4cc3n(-c3ccccc3)c2c2ccccc2n1-c1ccccc1] |
| 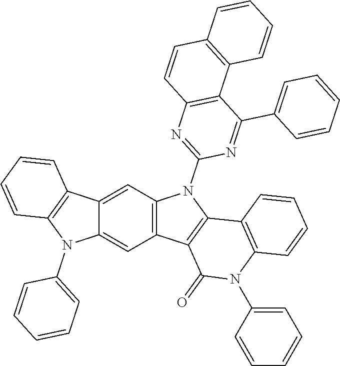 [SMILES: O=c1c2c3cc4c(cc3n(-c3nc(-c5ccccc5)c5c(ccc6ccccc65)n3)c2c2ccccc2n1-c1ccccc1)c1ccccc1n4-c1ccccc1] | 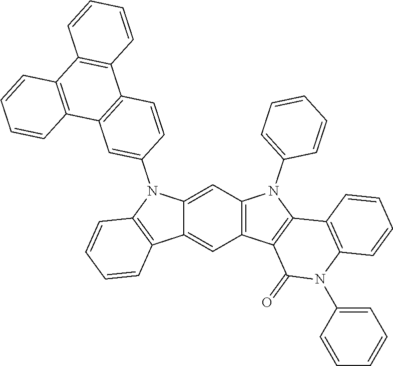 [SMILES: O=c1c2c3cc4c5ccccc5n(-c5ccc6c7ccccc7c7ccccc7c6c5)c4cc3n(-c3ccccc3)c2c2ccccc2n1-c1ccccc1] |
| 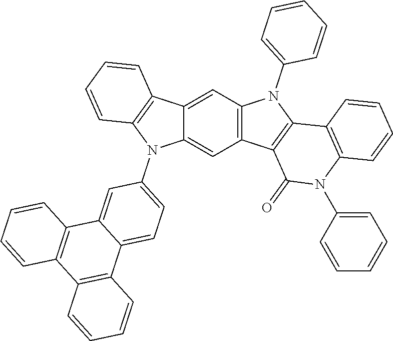 [SMILES: O=c1c2c3cc4c(cc3n(-c3ccccc3)c2c2ccccc2n1-c1ccccc1)c1ccccc1n4-c1ccc2c3ccccc3c3ccccc3c2c1] | 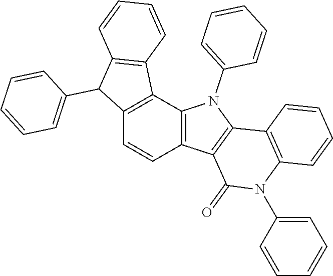 [SMILES: O=c1c2c3ccc4c(c3n(-c3ccccc3)c2c2ccccc2n1-c1ccccc1)-c1ccccc1C4c1ccccc1] |
| 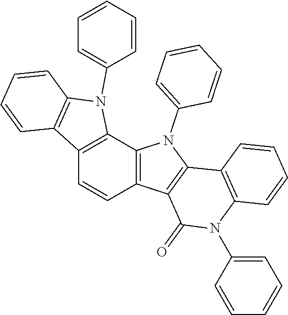 [SMILES: O=c1c2c3ccc4c5ccccc5n(-c5ccccc5)c4c3n(-c3ccccc3)c2c2ccccc2n1-c1ccccc1] | 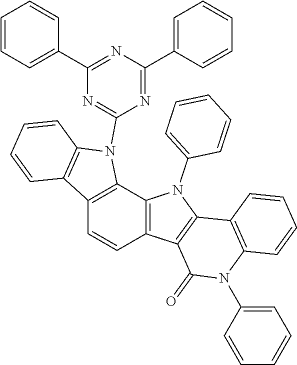 [SMILES: O=c1c2c3ccc4c5ccccc5n(-c5nc(-c6ccccc6)nc(-c6ccccc6)n5)c4c3n(-c3ccccc3)c2c2ccccc2n1-c1ccccc1] |
| 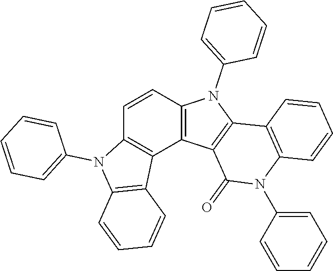 [SMILES: O=c1c2c3c4c5ccccc5n(-c5ccccc5)c4ccc3n(-c3ccccc3)c2c2ccccc2n1-c1ccccc1] | 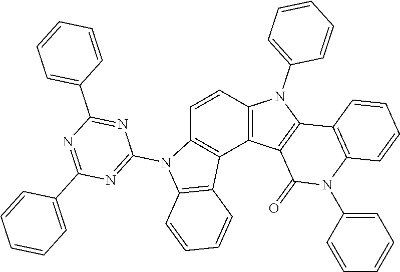 [SMILES: O=c1c2c3c4c5ccccc5n(-c5nc(-c6ccccc6)nc(-c6ccccc6)n5)c4ccc3n(-c3ccccc3)c2c2ccccc2n1-c1ccccc1] |
| 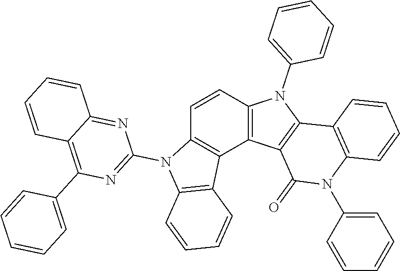 [SMILES: O=c1c2c3c4c5ccccc5n(-c5nc(-c6ccccc6)c6ccccc6n5)c4ccc3n(-c3ccccc3)c2c2ccccc2n1-c1ccccc1] | 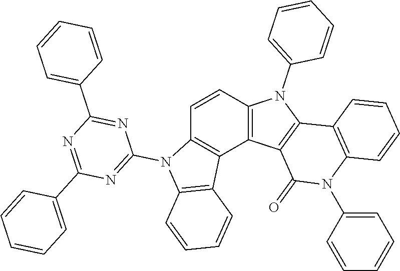 [SMILES: O=c1c2c3c4c5ccccc5n(-c5nc(-c6ccccc6)nc(-c6ccccc6)n5)c4ccc3n(-c3ccccc3)c2c2ccccc2n1-c1ccccc1] |
| 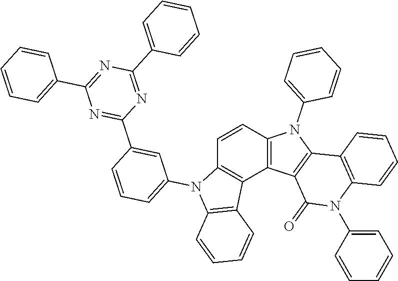 [SMILES: O=c1c2c3c4c5ccccc5n(-c5cccc(-c6nc(-c7ccccc7)nc(-c7ccccc7)n6)c5)c4ccc3n(-c3ccccc3)c2c2ccccc2n1-c1ccccc1] | 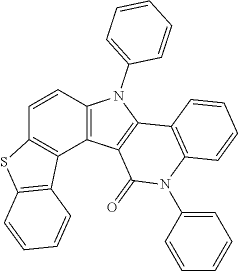 [SMILES: O=c1c2c3c4c(ccc3n(-c3ccccc3)c2c2ccccc2n1-c1ccccc1)sc1ccccc14] |
| 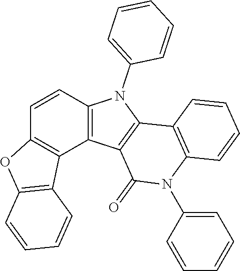 [SMILES: O=c1c2c3c4c(ccc3n(-c3ccccc3)c2c2ccccc2n1-c1ccccc1)oc1ccccc14] | 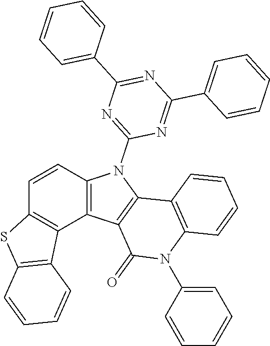 [SMILES: O=c1c2c3c4c(ccc3n(-c3nc(-c5ccccc5)nc(-c5ccccc5)n3)c2c2ccccc2n1-c1ccccc1)sc1ccccc14] |
| 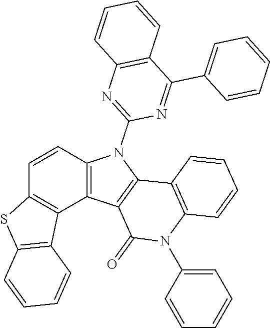 [SMILES: O=c1c2c3c4c(ccc3n(-c3nc(-c5ccccc5)c5ccccc5n3)c2c2ccccc2n1-c1ccccc1)sc1ccccc14] | 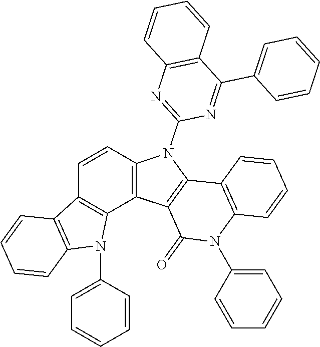 [SMILES: O=c1c2c3c(ccc4c5ccccc5n(-c5ccccc5)c43)n(-c3nc(-c4ccccc4)c4ccccc4n3)c2c2ccccc2n1-c1ccccc1] |
| 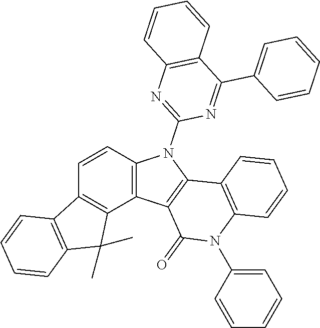 [SMILES: CC1(C)c2ccccc2-c2ccc3c(c21)c1c(=O)n(-c2ccccc2)c2ccccc2c1n3-c1nc(-c2ccccc2)c2ccccc2n1] | 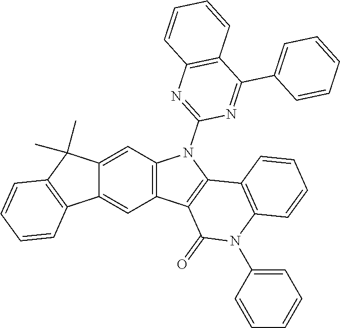 [SMILES: CC1(C)c2ccccc2-c2cc3c4c(=O)n(-c5ccccc5)c5ccccc5c4n(-c4nc(-c5ccccc5)c5ccccc5n4)c3cc21] |
| 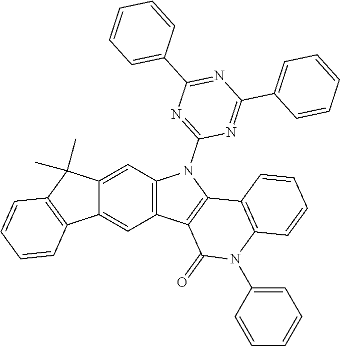 [SMILES: CC1(C)c2ccccc2-c2cc3c4c(=O)n(-c5ccccc5)c5ccccc5c4n(-c4nc(-c5ccccc5)nc(-c5ccccc5)n4)c3cc21] | 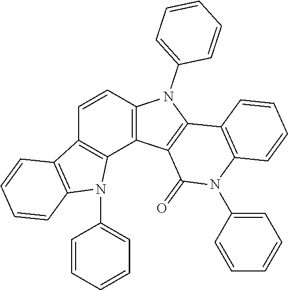 [SMILES: O=c1c2c3c(ccc4c5ccccc5n(-c5ccccc5)c43)n(-c3ccccc3)c2c2ccccc2n1-c1ccccc1] |
| 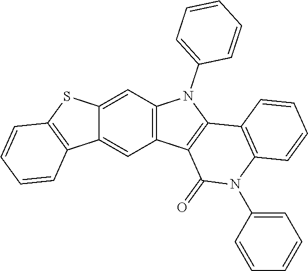 [SMILES: O=c1c2c3cc4c(cc3n(-c3ccccc3)c2c2ccccc2n1-c1ccccc1)sc1ccccc14] | 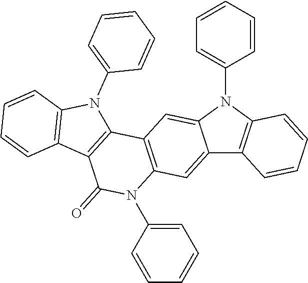 [SMILES: O=c1c2c3ccccc3n(-c3ccccc3)c2c2cc3c(cc2n1-c1ccccc1)c1ccccc1n3-c1ccccc1] |
| 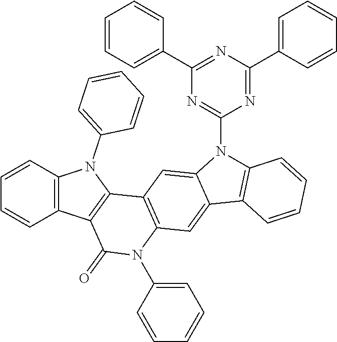 [SMILES: O=c1c2c3ccccc3n(-c3ccccc3)c2c2cc3c(cc2n1-c1ccccc1)c1ccccc1n3-c1nc(-c2ccccc2)nc(-c2ccccc2)n1] | 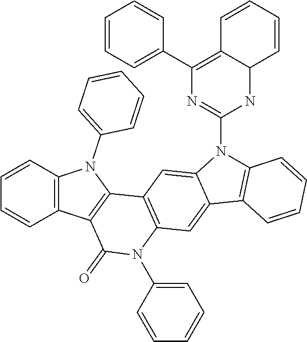 [SMILES: O=c1c2c3ccccc3n(-c3ccccc3)c2c2cc3c(cc2n1-c1ccccc1)c1ccccc1n3C1=NC(c2ccccc2)=C2C=CC=CC2N1] |
| 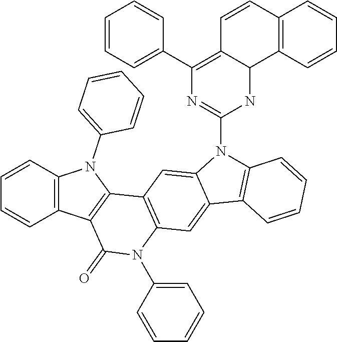 [SMILES: O=c1c2c3ccccc3n(-c3ccccc3)c2c2cc3c(cc2n1-c1ccccc1)c1ccccc1n3C1=NC(c2ccccc2)=C2C=Cc3ccccc3C2N1] | 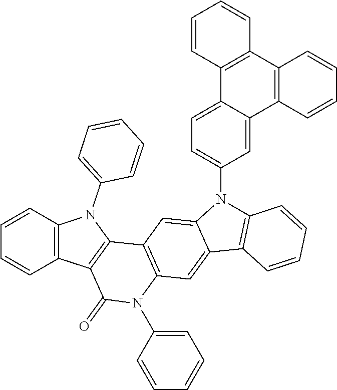 [SMILES: O=c1c2c3ccccc3n(-c3ccccc3)c2c2cc3c(cc2n1-c1ccccc1)c1ccccc1n3-c1ccc2c3ccccc3c3ccccc3c2c1] |

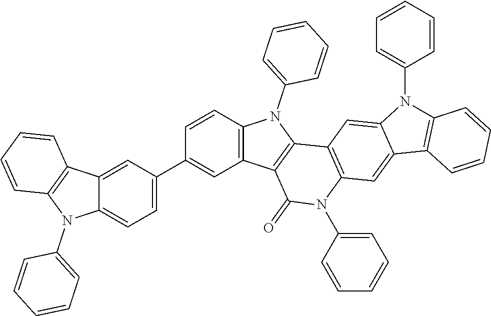 [SMILES: O=c1c2c3cc(-c4ccc5c(c4)c4ccccc4n5-c4ccccc4)ccc3n(-c3ccccc3)c2c2cc3c(cc2n1-c1ccccc1)c1ccccc1n3-c1ccccc1]

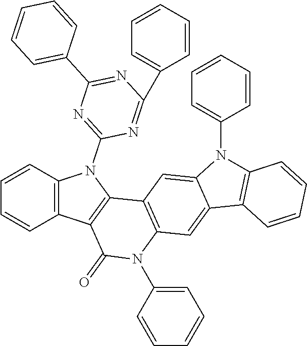 [SMILES: O=c1c2c3ccccc3n(-c3nc(-c4ccccc4)nc(-c4ccccc4)n3)c2c2cc3c(cc2n1-c1ccccc1)c1ccccc1n3-c1ccccc1]

| | |
|---|---|
| 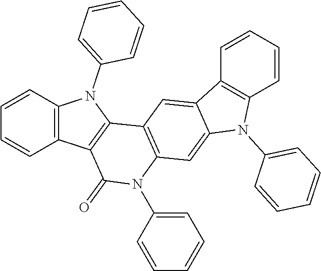 [SMILES: O=c1c2c3ccccc3n(-c3ccccc3)c2c2cc3c4ccccc4n(-c4ccccc4)c3cc2n1-c1ccccc1] | 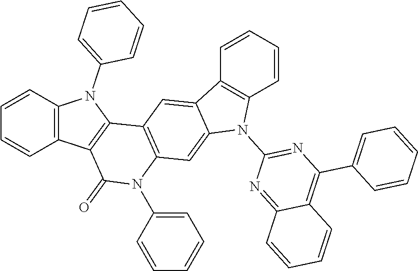 [SMILES: O=c1c2c3ccccc3n(-c3ccccc3)c2c2cc3c4ccccc4n(-c4nc(-c5ccccc5)c5ccccc5n4)c3cc2n1-c1ccccc1] |
| 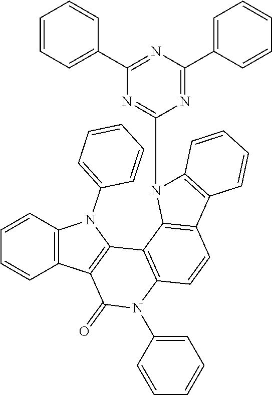 [SMILES: O=c1c2c3ccccc3n(-c3ccccc3)c2c2c(ccc3c4ccccc4n(-c4nc(-c5ccccc5)nc(-c5ccccc5)n4)c32)n1-c1ccccc1] | 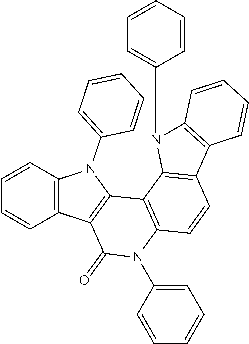 [SMILES: O=c1c2c3ccccc3n(-c3ccccc3)c2c2c(ccc3c4ccccc4n(-c4ccccc4)c32)n1-c1ccccc1] |
| 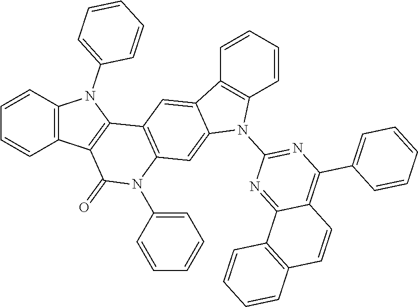 [SMILES: O=c1c2c3ccccc3n(-c3ccccc3)c2c2cc3c4ccccc4n(-c4nc(-c5ccccc5)c5ccc6ccccc6c5n4)c3cc2n1-c1ccccc1] | 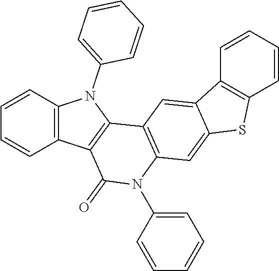 [SMILES: O=c1c2c3ccccc3n(-c3ccccc3)c2c2cc3c(cc2n1-c1ccccc1)sc1ccccc13] |
| 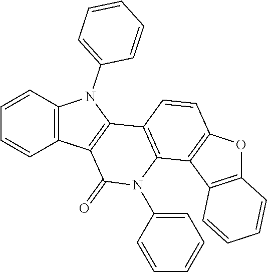 [SMILES: O=c1c2c3ccccc3n(-c3ccccc3)c2c2ccc3oc4ccccc4c3c2n1-c1ccccc1] | 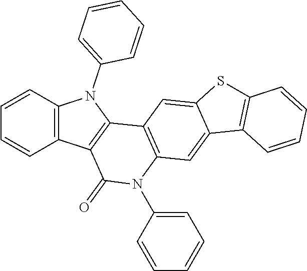 [SMILES: O=c1c2c3ccccc3n(-c3ccccc3)c2c2cc3sc4ccccc4c3cc2n1-c1ccccc1] |
| 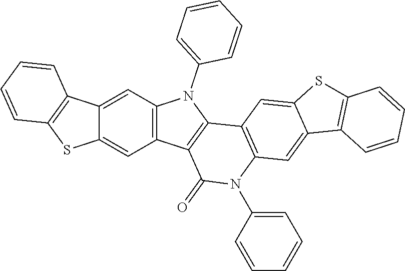 [SMILES: O=c1c2c3cc4sc5ccccc5c4cc3n(-c3ccccc3)c2c2cc3sc4ccccc4c3cc2n1-c1ccccc1] | 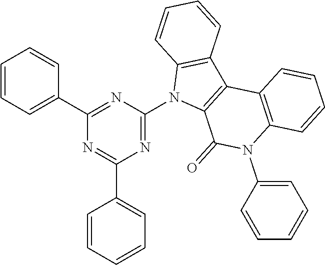 [SMILES: O=c1c2c(c3ccccc3n1-c1ccccc1)c1ccccc1n2-c1nc(-c2ccccc2)nc(-c2ccccc2)n1] |
| 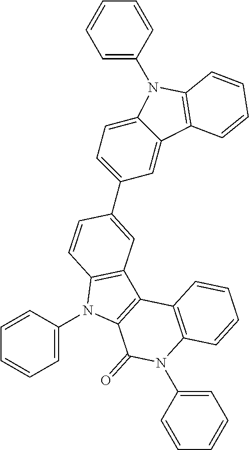 [SMILES: O=c1c2c(c3ccccc3n1-c1ccccc1)c1cc(-c3ccc4c(c3)c3ccccc3n4-c3ccccc3)ccc1n2-c1ccccc1] | 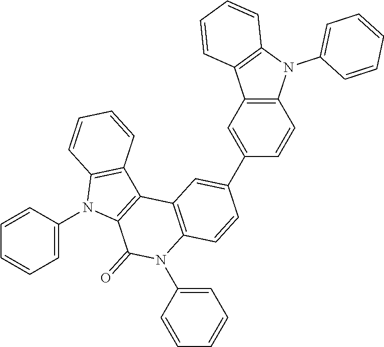 [SMILES: O=c1c2c(c3cc(-c4ccc5c(c4)c4ccccc4n5-c4ccccc4)ccc3n1-c1ccccc1)c1ccccc1n2-c1ccccc1] |
| 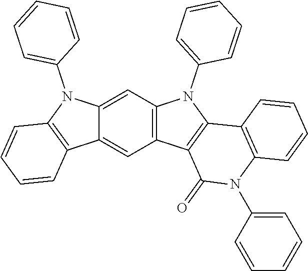 [SMILES: O=c1c2c3cc4c5ccccc5n(-c5ccccc5)c4cc3n(-c3ccccc3)c2c2ccccc2n1-c1ccccc1] | 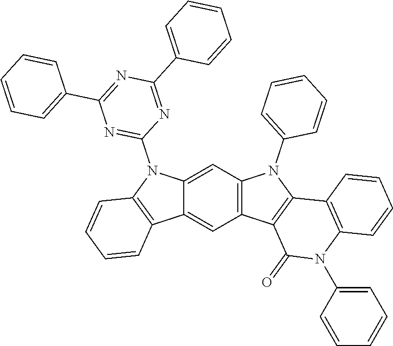 [SMILES: O=c1c2c3cc4c5ccccc5n(-c5nc(-c6ccccc6)nc(-c6ccccc6)n5)c4cc3n(-c3ccccc3)c2c2ccccc2n1-c1ccccc1] |
| 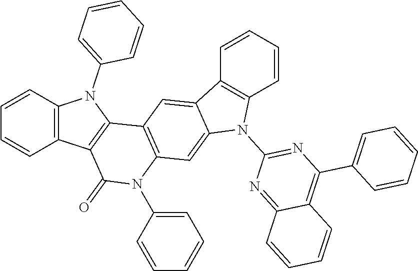 [SMILES: O=c1c2c3ccccc3n(-c3ccccc3)c2c2cc3c4ccccc4n(-c4nc(-c5ccccc5)c5ccccc5n4)c3cc2n1-c1ccccc1] | 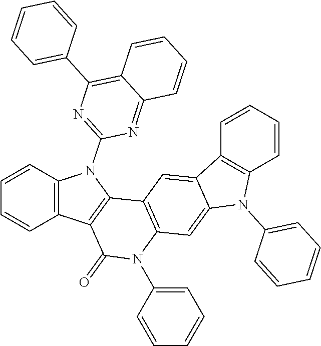 [SMILES: O=c1c2c3ccccc3n(-c3nc(-c4ccccc4)c4ccccc4n3)c2c2cc3c4ccccc4n(-c4ccccc4)c3cc2n1-c1ccccc1] |
| 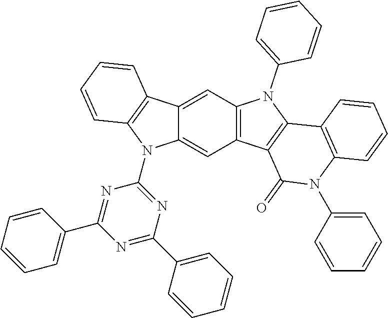 [SMILES: O=c1c2c3cc4c(cc3n(-c3ccccc3)c2c2ccccc2n1-c1ccccc1)c1ccccc1n4-c1nc(-c2ccccc2)nc(-c2ccccc2)n1] | 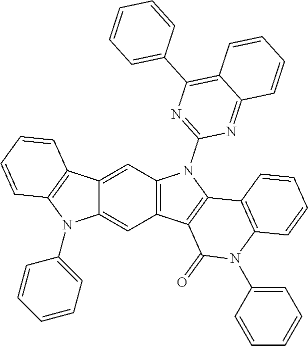 [SMILES: O=c1c2c3cc4c(cc3n(-c3nc(-c5ccccc5)c5ccccc5n3)c2c2ccccc2n1-c1ccccc1)c1ccccc1n4-c1ccccc1] |
| 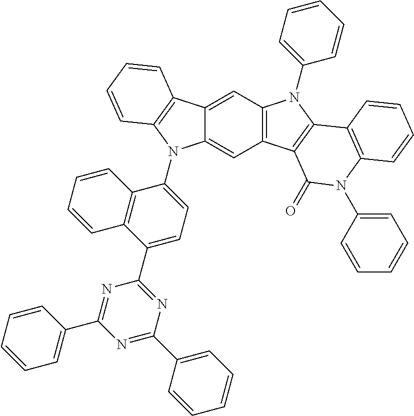 [SMILES: O=c1c2c3cc4c(cc3n(-c3ccccc3)c2c2ccccc2n1-c1ccccc1)c1ccccc1n4-c1ccc(-c2nc(-c3ccccc3)nc(-c3ccccc3)n2)c2ccccc12] | 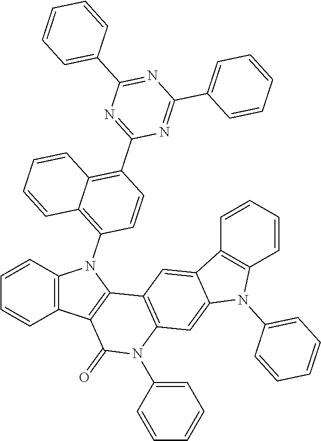 [SMILES: O=c1c2c3ccccc3n(-c3ccc(-c4nc(-c5ccccc5)nc(-c5ccccc5)n4)c4ccccc34)c2c2cc3c4ccccc4n(-c4ccccc4)c3cc2n1-c1ccccc1] |
| 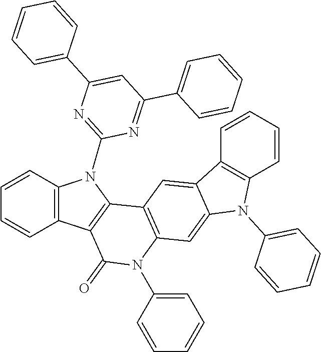 [SMILES: O=c1c2c3ccccc3n(-c3nc(-c4ccccc4)cc(-c4ccccc4)n3)c2c2cc3c4ccccc4n(-c4ccccc4)c3cc2n1-c1ccccc1] | 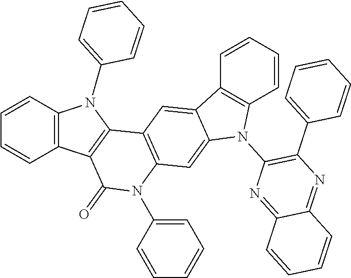 [SMILES: O=c1c2c3ccccc3n(-c3ccccc3)c2c2cc3c4ccccc4n(-c4nc5ccccc5nc4-c4ccccc4)c3cc2n1-c1ccccc1] |
| 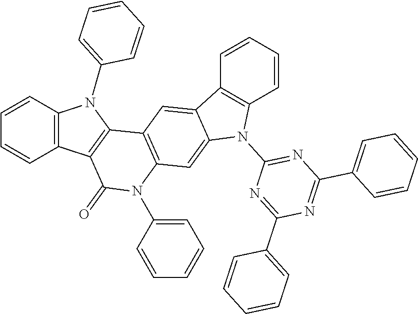 [SMILES: O=c1c2c3ccccc3n(-c3ccccc3)c2c2cc3c4ccccc4n(-c4nc(-c5ccccc5)nc(-c5ccccc5)n4)c3cc2n1-c1ccccc1] | 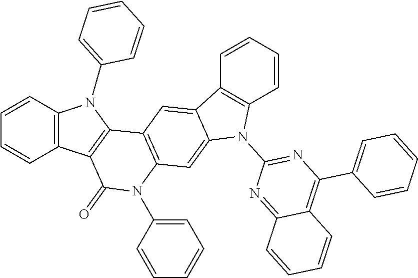 [SMILES: O=c1c2c3ccccc3n(-c3ccccc3)c2c2cc3c4ccccc4n(-c4nc(-c5ccccc5)c5ccccc5n4)c3cc2n1-c1ccccc1] |

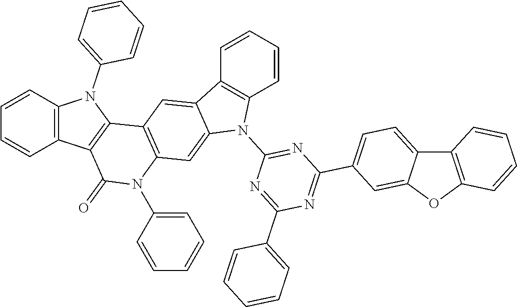 [SMILES: O=c1c2c3ccccc3n(-c3ccccc3)c2c2cc3c4ccccc4n(-c4nc(-c5ccccc5)nc(-c5ccc6c(c5)oc5ccccc56)n4)c3cc2n1-c1ccccc1]

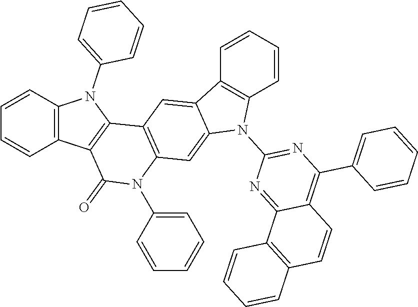 [SMILES: O=c1c2c3ccccc3n(-c3ccccc3)c2c2cc3c4ccccc4n(-c4nc(-c5ccccc5)c5ccc6ccccc6c5n4)c3cc2n1-c1ccccc1]

| | |
|---|---|
| 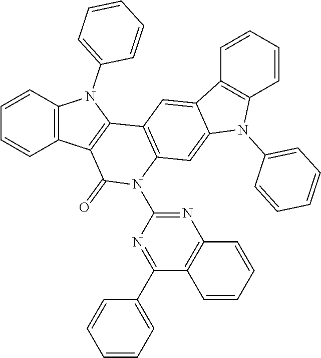 [SMILES: O=c1c2c3ccccc3n(-c3ccccc3)c2c2cc3c4ccccc4n(-c4ccccc4)c3cc2n1-c1nc(-c2ccccc2)c2ccccc2n1] | 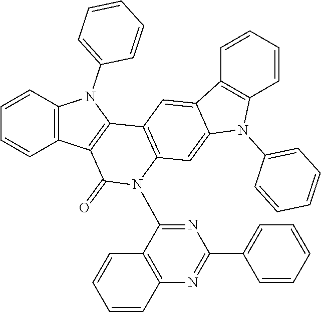 [SMILES: O=c1c2c3ccccc3n(-c3ccccc3)c2c2cc3c4ccccc4n(-c4ccccc4)c3cc2n1-c1nc(-c2ccccc2)nc2ccccc12] |
| 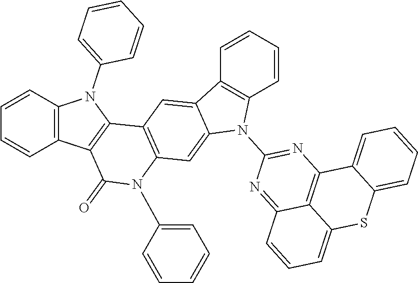 [SMILES: O=c1c2c3ccccc3n(-c3ccccc3)c2c2cc3c4ccccc4n(-c4nc5c6c(cccc6n4)Sc4ccccc4-5)c3cc2n1-c1ccccc1] | 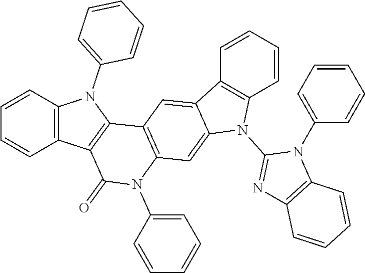 [SMILES: O=c1c2c3ccccc3n(-c3ccccc3)c2c2cc3c4ccccc4n(-c4nc5ccccc5n4-c4ccccc4)c3cc2n1-c1ccccc1] |
| 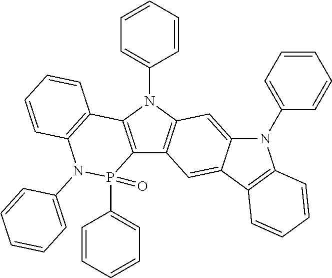 [SMILES: O=P1(c2ccccc2)c2c(n(-c3ccccc3)c3cc4c(cc23)c2ccccc2n4-c2ccccc2)-c2ccccc2N1c1ccccc1] | 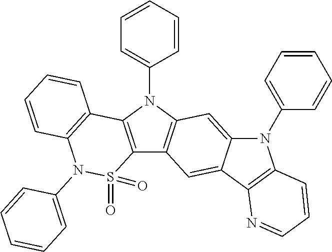 [SMILES: O=S1(=O)c2c(n(-c3ccccc3)c3cc4c(cc23)c2ncccc2n4-c2ccccc2)-c2ccccc2N1c1ccccc1] |
| 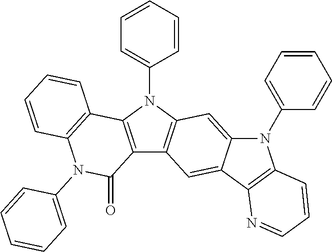 [SMILES: O=c1c2c3cc4c5ncccc5n(-c5ccccc5)c4cc3n(-c3ccccc3)c2c2ccccc2n1-c1ccccc1] | 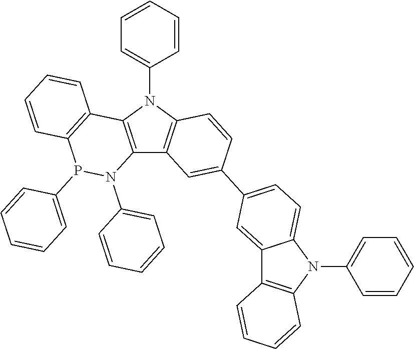 [SMILES: c1ccc(N2c3c(n(-c4ccccc4)c4ccc(-c5ccc6c(c5)c5ccccc5n6-c5ccccc5)cc34)-c3ccccc3P2c2ccccc2)cc1] |
| 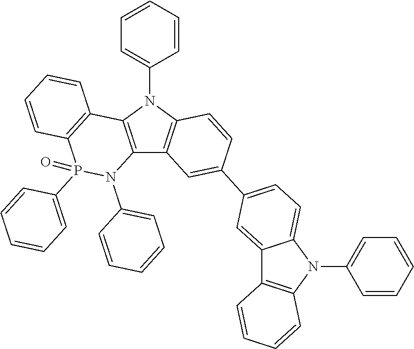 [SMILES: O=P1(c2ccccc2)c2ccccc2-c2c(c3cc(-c4ccc5c(c4)c4ccccc4n5-c4ccccc4)ccc3n2-c2ccccc2)N1c1ccccc1] | 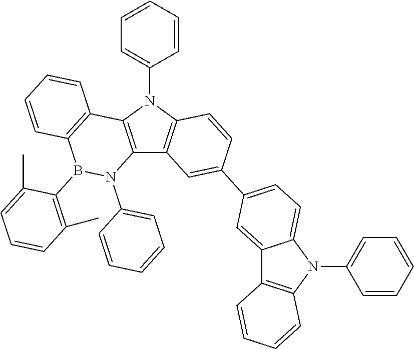 [SMILES: Cc1cccc(C)c1B1c2ccccc2-c2c(c3cc(-c4ccc5c(c4)c4ccccc4n5-c4ccccc4)ccc3n2-c2ccccc2)N1c1ccccc1] |
| 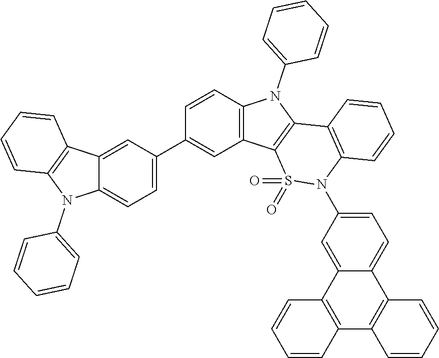 [SMILES: O=S1(=O)c2c(n(-c3ccccc3)c3ccc(-c4ccc5c(c4)c4ccccc4n5-c4ccccc4)cc23)-c2ccccc2N1c1ccc2c3ccccc3c3ccccc3c2c1] | 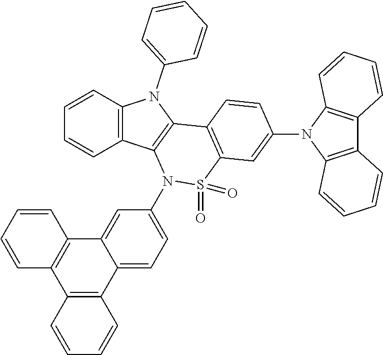 [SMILES: O=S1(=O)c2cc(-n3c4ccccc4c4ccccc43)ccc2-c2c(c3ccccc3n2-c2ccccc2)N1c1ccc2c3ccccc3c3ccccc3c2c1] |

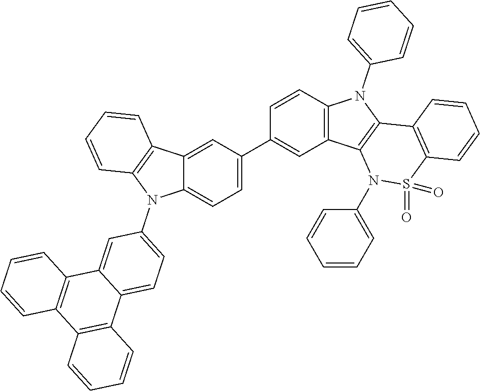 [SMILES: O=S1(=O)c2ccccc2-c2c(c3cc(-c4ccc5c(c4)c4ccccc4n5-c4ccc5c6ccccc6c6ccccc6c5c4)ccc3n2-c2ccccc2)N1c1ccccc1]

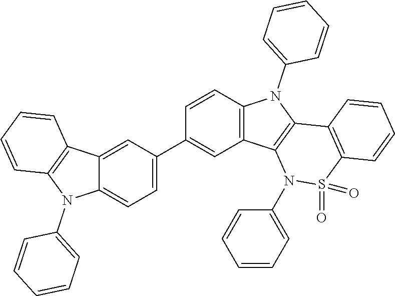 [SMILES: O=S1(=O)c2ccccc2-c2c(c3cc(-c4ccc5c(c4)c4ccccc4n5-c4ccccc4)ccc3n2-c2ccccc2)N1c1ccccc1]

| | |
|---|---|
| 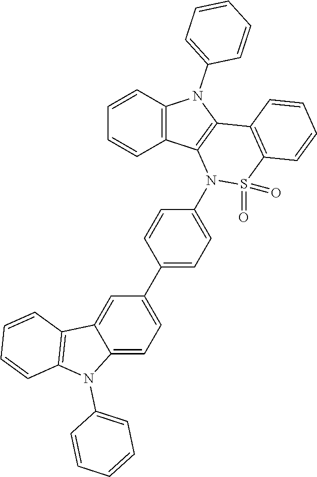 [SMILES: O=S1(=O)c2ccccc2-c2c(c3ccccc3n2-c2ccccc2)N1c1ccc(-c2ccc3c(c2)c2ccccc2n3-c2ccccc2)cc1] | 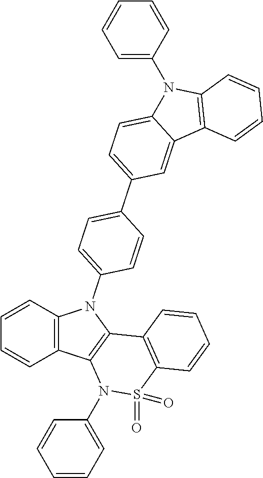 [SMILES: O=S1(=O)c2ccccc2-c2c(c3ccccc3n2-c2ccc(-c3ccc4c(c3)c3ccccc3n4-c3ccccc3)cc2)N1c1ccccc1] |
| 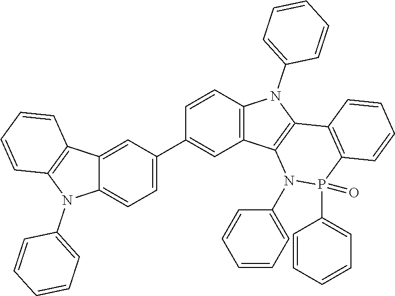 [SMILES: O=P1(c2ccccc2)c2ccccc2-c2c(c3cc(-c4ccc5c(c4)c4ccccc4n5-c4ccccc4)ccc3n2-c2ccccc2)N1c1ccccc1] | 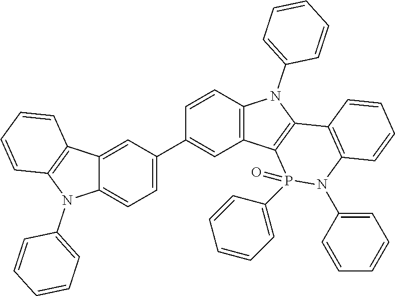 [SMILES: O=P1(c2ccccc2)c2c(n(-c3ccccc3)c3ccc(-c4ccc5c(c4)c4ccccc4n5-c4ccccc4)cc23)-c2ccccc2N1c1ccccc1] |
| 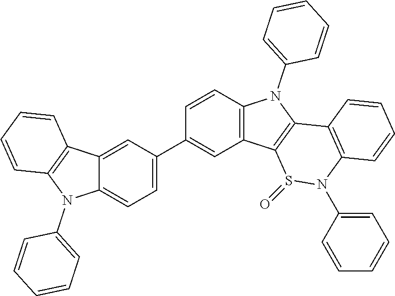 [SMILES: O=S1c2c(n(-c3ccccc3)c3ccc(-c4ccc5c(c4)c4ccccc4n5-c4ccccc4)cc23)-c2ccccc2N1c1ccccc1] | 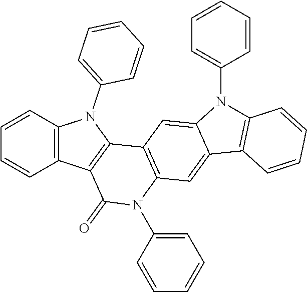 [SMILES: O=c1c2c3ccccc3n(-c3ccccc3)c2c2cc3c(cc2n1-c1ccccc1)c1ccccc1n3-c1ccccc1] |
| 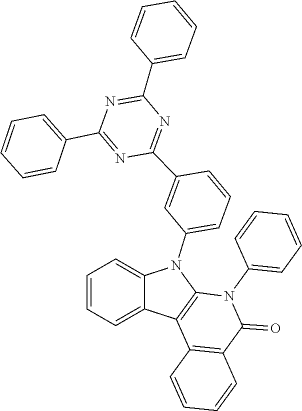 [SMILES: O=c1c2ccccc2c2c3ccccc3n(-c3cccc(-c4nc(-c5ccccc5)nc(-c5ccccc5)n4)c3)c2n1-c1ccccc1] | 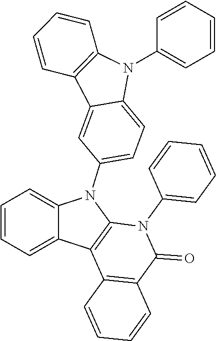 [SMILES: O=c1c2ccccc2c2c3ccccc3n(-c3ccc4c(c3)c3ccccc3n4-c3ccccc3)c2n1-c1ccccc1] |
| 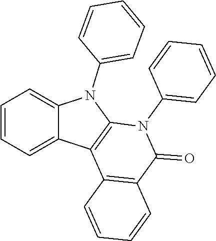 [SMILES: O=c1c2ccccc2c2c3ccccc3n(-c3ccccc3)c2n1-c1ccccc1] | 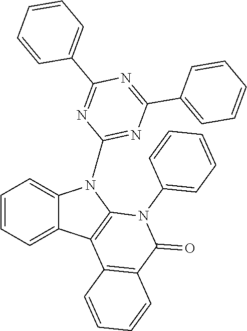 [SMILES: O=c1c2ccccc2c2c3ccccc3n(-c3nc(-c4ccccc4)nc(-c4ccccc4)n3)c2n1-c1ccccc1] |
| 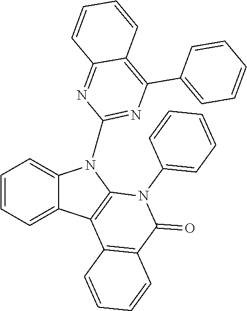 [SMILES: O=c1c2ccccc2c2c3ccccc3n(-c3nc(-c4ccccc4)c4ccccc4n3)c2n1-c1ccccc1] | 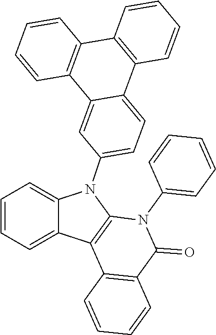 [SMILES: O=c1c2ccccc2c2c3ccccc3n(-c3ccc4c5ccccc5c5ccccc5c4c3)c2n1-c1ccccc1] |
| 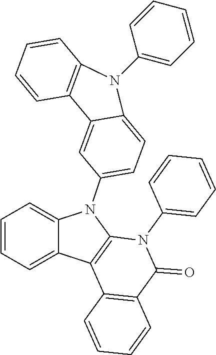 [SMILES: O=c1c2ccccc2c2c3ccccc3n(-c3ccc4c(c3)c3ccccc3n4-c3ccccc3)c2n1-c1ccccc1] | 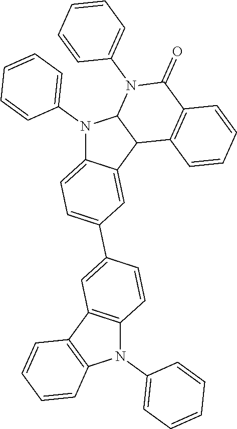 [SMILES: O=C1c2ccccc2C2c3cc(-c4ccc5c(c4)c4ccccc4n5-c4ccccc4)ccc3N(c3ccccc3)C2N1c1ccccc1] |
| 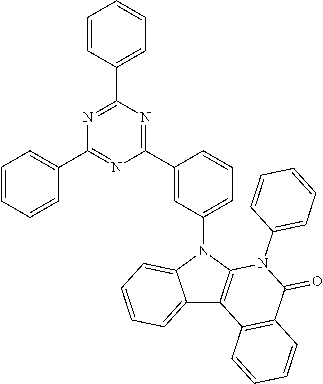 [SMILES: O=c1c2ccccc2c2c3ccccc3n(-c3cccc(-c4nc(-c5ccccc5)nc(-c5ccccc5)n4)c3)c2n1-c1ccccc1] | 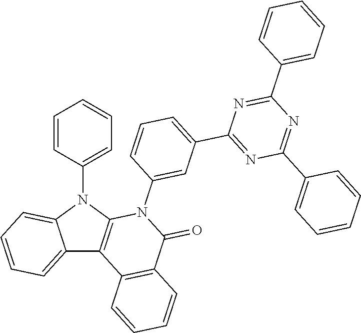 [SMILES: O=c1c2ccccc2c2c3ccccc3n(-c3ccccc3)c2n1-c1cccc(-c2nc(-c3ccccc3)nc(-c3ccccc3)n2)c1] |
| 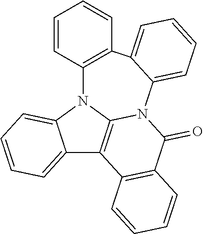 [SMILES: O=c1c2ccccc2c2c3ccccc3n3c2n1-c1ccccc1-c1ccccc1-3] | 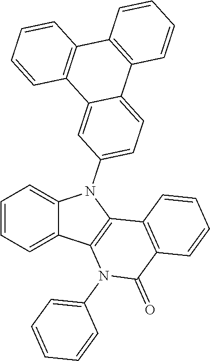 [SMILES: O=c1c2ccccc2c2c(c3ccccc3n2-c2ccc3c4ccccc4c4ccccc4c3c2)n1-c1ccccc1] |
| 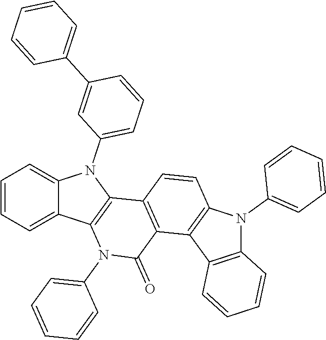 [SMILES: O=c1c2c(ccc3c2c2ccccc2n3-c2ccccc2)c2c(c3ccccc3n2-c2cccc(-c3ccccc3)c2)n1-c1ccccc1] | 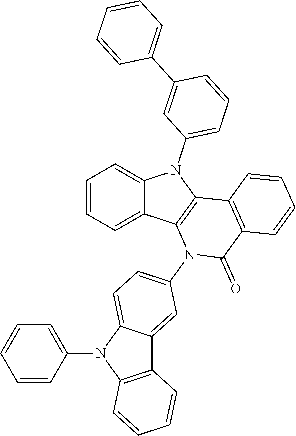 [SMILES: O=c1c2ccccc2c2c(c3ccccc3n2-c2cccc(-c3ccccc3)c2)n1-c1ccc2c(c1)c1ccccc1n2-c1ccccc1] |

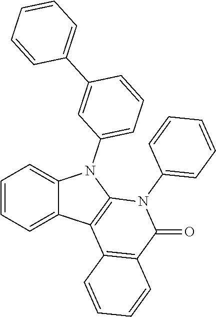 [SMILES: O=c1c2ccccc2c2c3ccccc3n(-c3cccc(-c4ccccc4)c3)c2n1-c1ccccc1]

The base structure of the compounds of the invention is known in the literature. These can be functionalized by the routes outlined in schemes 1 and 2. The indoloquinolinone base skeleton can be functionalized here by halogenation, for example with NBS, followed by a coupling reaction, for example a Suzuki coupling. Thereafter, the indole nitrogen atom and the lactam nitrogen atom may be substituted, for example by Buchwald coupling or by Ullmann coupling (scheme 1). The synthesis of compounds having a fused-on group of the formula (3) likewise proceeds from the halogenated base skeleton (scheme 2). This is coupled to an ortho-nitrobenzeneboronic acid derivative, followed by a cyclization reaction. Thereafter, the indole nitrogen atoms and the lactam nitrogen atom may be substituted, for example by Buchwald coupling or by Ullmann coupling. Further derivatives may be synthesized analogously.

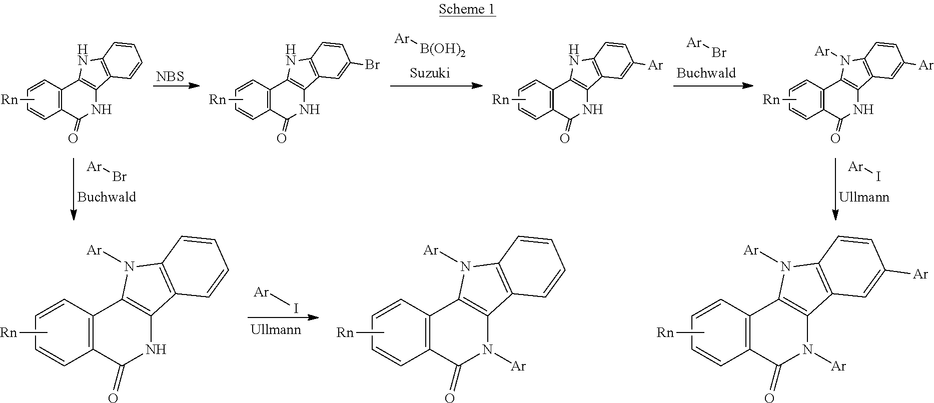

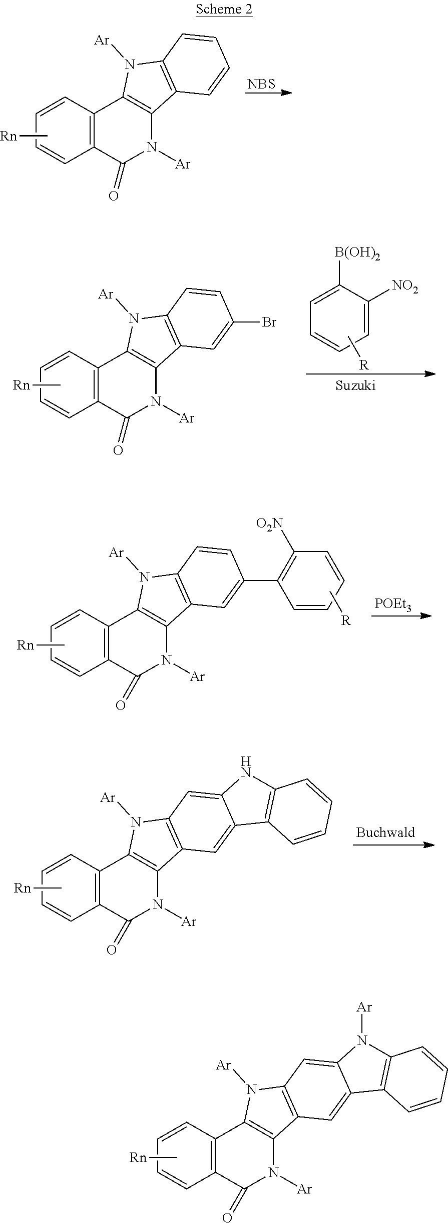

For the processing of the compounds of the invention from a liquid phase, for example by spin-coating or by printing methods, formulations of the compounds of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, α-terpineol, benzothiazole, butyl benzoate, cumene, cyclohexanol, cyclohexanone, cyclohexylbenzene, decalin, dodecylbenzene, ethyl benzoate, indane, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, 2-methylbiphenyl, 3-methylbiphenyl, 1-methylnaphthalene, 1-ethylnaphthalene, ethyl octanoate, diethyl sebacate, octyl octanoate, heptylbenzene, menthyl isovalerate, cyclohexyl hexanoate or mixtures of these solvents.

The present invention therefore further provides a formulation comprising a compound of the invention and at least one further compound. The further compound may, for example, be a solvent, especially one of the abovementioned solvents or a mixture of these solvents. The further compound may alternatively be at least one further organic or inorganic compound which is likewise used in the electronic device, for example an emitting compound and/or a further matrix material. Suitable emitting compounds and further matrix materials are listed at the back in connection with the organic electroluminescent device.

The compounds of the invention are suitable for use in an electronic device, especially in an organic electroluminescent device.

The present invention therefore further provides for the use of a compound of the invention in an electronic device, especially in an organic electroluminescent device.

The present invention still further provides an electronic device comprising at least one compound of the invention.

An electronic device in the context of the present invention is a device comprising at least one layer comprising at least one organic compound. This component may also comprise inorganic materials or else layers formed entirely from inorganic materials.

The electronic device is preferably selected from the group consisting of organic electroluminescent devices (OLEDs), organic integrated circuits (O-ICs), organic field-effect transistors (O-FETs), organic thin-film transistors (O-TFTs), organic light-emitting transistors (O-LETs), organic solar cells (O-SCs), dye-sensitized organic solar cells (DSSCs), organic optical detectors, organic photoreceptors, organic field-quench devices (O-FQDs), light-emitting electrochemical cells (LECs), organic laser diodes (O-lasers) and organic plasmon emitting devices, but preferably organic electroluminescent devices (OLEDs), more preferably phosphorescent OLEDs.

The organic electroluminescent device comprises cathode, anode and at least one emitting layer. Apart from these layers, it may also comprise further layers, for example in each case one or more hole injection layers, hole transport layers, hole blocker layers, electron transport layers, electron injection layers, exciton blocker layers, electron blocker layers and/or charge generation layers. It is likewise possible for interlayers having an exciton-blocking function, for example, to be introduced between two emitting layers. However, it should be pointed out that not necessarily every one of these layers need be present. In this case, it is possible for the organic electroluminescent device to contain an emitting layer, or for it to contain a plurality of emitting layers. If a plurality of emission layers are present, these preferably have several emission maxima between 380 nm and 750 nm overall, such that the overall result is white emission; in other words, various emitting compounds which may fluoresce or phosphoresce are used in the emitting layers. Especially preferred are systems having three emitting layers, where the three layers show blue, green and orange or red emission. The organic electroluminescent device of the invention may also be a tandem OLED, especially for white-emitting OLEDs.

The compound of the invention according to the above-detailed embodiments may be used in different layers, according to the exact structure. Preference is given to an organic electroluminescent device comprising a compound of formula (1) or the above-recited preferred embodiments in an emitting layer as matrix material for phosphorescent emitters or for emitters that exhibit TADF (thermally activated delayed fluorescence), especially for phosphorescent emitters. In this case, the organic electroluminescent device may contain an emitting layer, or it may contain a plurality of emitting layers, where at least one emitting layer contains at least one compound of the invention as matrix material. In addition, the compound of the invention can also be used in an electron transport layer and/or in a hole blocker layer and/or in a hole transport layer and/or in an exciton blocker layer.

When the compound of the invention is used as matrix material for a phosphorescent compound in an emitting layer, it is preferably used in combination with one or more phosphorescent materials (triplet emitters). Phosphorescence in the context of this invention is understood to mean luminescence from an excited state having higher spin multiplicity, i.e. a spin state >1, especially from an excited triplet state. In the context of this application, all luminescent complexes with transition metals or lanthanides, especially all iridium, platinum and copper complexes, shall be regarded as phosphorescent compounds.

The mixture of the compound of the invention and the emitting compound contains between 99% and 1% by volume, preferably between 98% and 10% by volume, more preferably between 97% and 60% by volume and especially between 95% and 80% by volume of the compound of the invention, based on the overall mixture of emitter and matrix material. Correspondingly, the mixture contains between 1% and 99% by volume, preferably between 2% and 90% by volume, more preferably between 3% and 40% by volume and especially between 5% and 20% by volume of the emitter, based on the overall mixture of emitter and matrix material.

A further preferred embodiment of the present invention is the use of the compound of the invention as matrix material for a phosphorescent emitter in combination with a further matrix material. Suitable matrix materials which can be used in combination with the inventive compounds are aromatic ketones, aromatic phosphine oxides or aromatic sulfoxides or sulfones, for example according to WO 2004/013080, WO 2004/093207, WO 2006/005627 or WO 2010/006680, triarylamines, carbazole derivatives, e.g. CBP (N,N-biscarbazolylbiphenyl) or the carbazole derivatives disclosed in WO 2005/039246, US 2005/0069729, JP 2004/288381, EP 1205527, WO 2008/086851 or WO 2013/041176, indolocarbazole derivatives, for example according to WO 2007/063754 or WO 2008/056746, indenocarbazole derivatives, for example according to WO 2010/136109, WO 2011/000455, WO 2013/041176 or WO 2013/056776, azacarbazole derivatives, for example according to EP 1617710, EP 1617711, EP 1731584, JP 2005/347160, bipolar matrix materials, for example according to WO 2007/137725, silanes, for example according to WO 2005/111172, azaboroles or boronic esters, for example according to WO 2006/117052, triazine derivatives, for example according to WO 2007/063754, WO 2008/056746, WO 2010/015306, WO 2011/057706, WO 2011/060859 or WO 2011/060877, zinc complexes, for example according to EP 652273 or WO 2009/062578, diazasilole or tetraazasilole derivatives, for example according to WO 2010/054729, diazaphosphole derivatives, for example according to WO 2010/054730, bridged carbazole derivatives, for example according to WO 2011/042107, WO 2011/060867, WO 2011/088877 and WO 2012/143080, triphenylene derivatives, for example according to WO 2012/048781, or dibenzofuran derivatives, for example according to WO 2015/169412, WO 2016/015810, WO 2016/023608, WO 2017/148564 or WO 2017/148565. It is likewise possible for a further phosphorescent emitter having shorter-wavelength emission than the actual emitter to be present as co-host in the mixture, or a compound not involved in charge transport to a significant extent, if at all, as described, for example, in WO 2010/108579.

In a preferred embodiment of the invention, the materials are used in combination with a further matrix material. If the compound of the invention is substituted by an electron-deficient heteroaromatic ring system, for example by triazine or quinazoline, preferred co-matrix materials are selected from the group of the biscarbazoles, the bridged carbazoles, the triarylamines, the dibenzofuran-carbazole derivatives or dibenzofuran-amine derivatives and the carbazoleamines.

Preferred biscarbazoles are the structures of the following formulae (30) and (31):

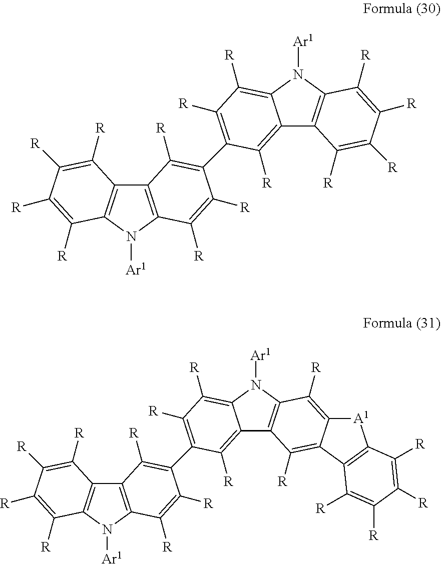

where R, Ar¹ and A¹ have the definitions given above. In a preferred embodiment of the invention, A¹ is CR₂.

Preferred embodiments of the compounds of the formulae (30) and (31) are the compounds of the following formulae (30a) and (31a):

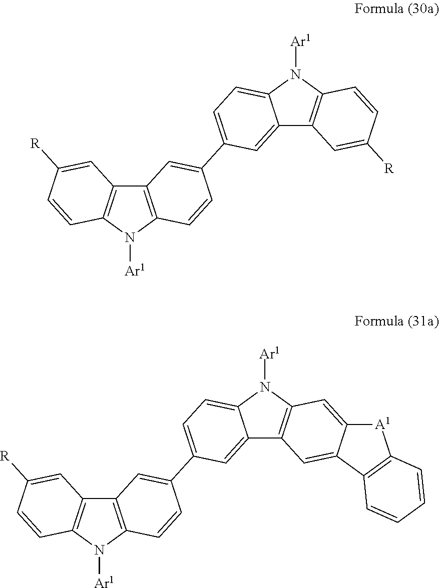

where the symbols used have the definitions given above.

Examples of suitable compounds of formulae (30) and (31) are the compounds depicted below:

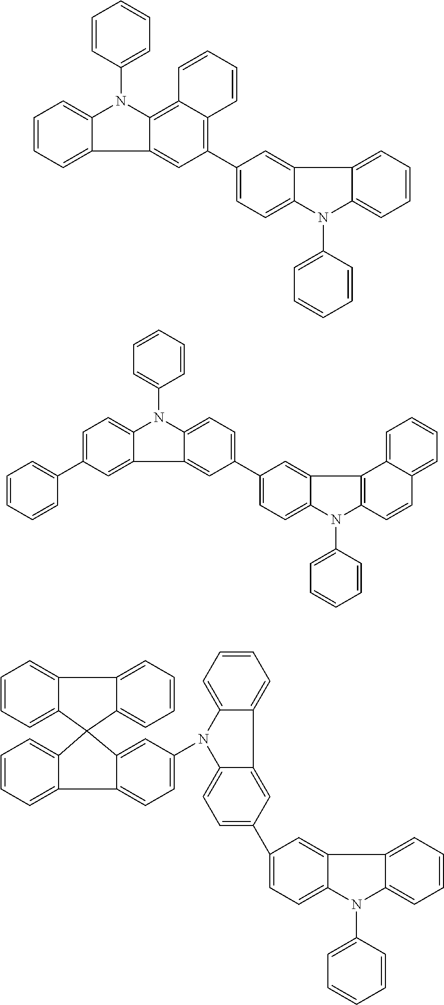 [SMILES: c1ccc(-n2c3ccccc3c3cc(-c4cc5c6ccccc6n(-c6ccccc6)c5c5ccccc45)ccc32)cc1 ; c1ccc(-c2ccc3c(c2)c2cc(-c4ccc5c(c4)c4c6ccccc6ccc4n5-c4ccccc4)ccc2n3-c2ccccc2)cc1 ; c1ccc(-n2c3ccccc3c3cc(-c4ccc5c(c4)c4ccccc4n5-c4ccc5c(c4)C4(c6ccccc6-c6ccccc64)c4ccccc4-5)ccc32)cc1]

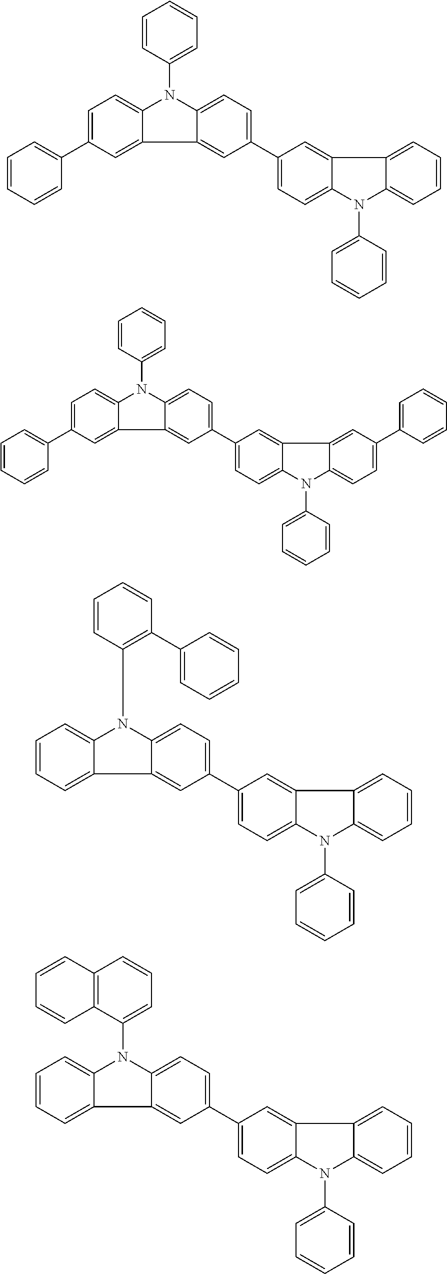 [SMILES: c1ccc(-c2ccc3c(c2)c2cc(-c4ccc5c(c4)c4ccccc4n5-c4ccccc4)ccc2n3-c2ccccc2)cc1 ; c1ccc(-c2ccc3c(c2)c2cc(-c4ccc5c(c4)c4cc(-c6ccccc6)ccc4n5-c4ccccc4)ccc2n3-c2ccccc2)cc1 ; c1ccc(-c2ccccc2-n2c3ccccc3c3cc(-c4ccc5c(c4)c4ccccc4n5-c4ccccc4)ccc32)cc1 ; c1ccc(-n2c3ccccc3c3cc(-c4ccc5c(c4)c4ccccc4n5-c4cccc5ccccc45)ccc32)cc1]

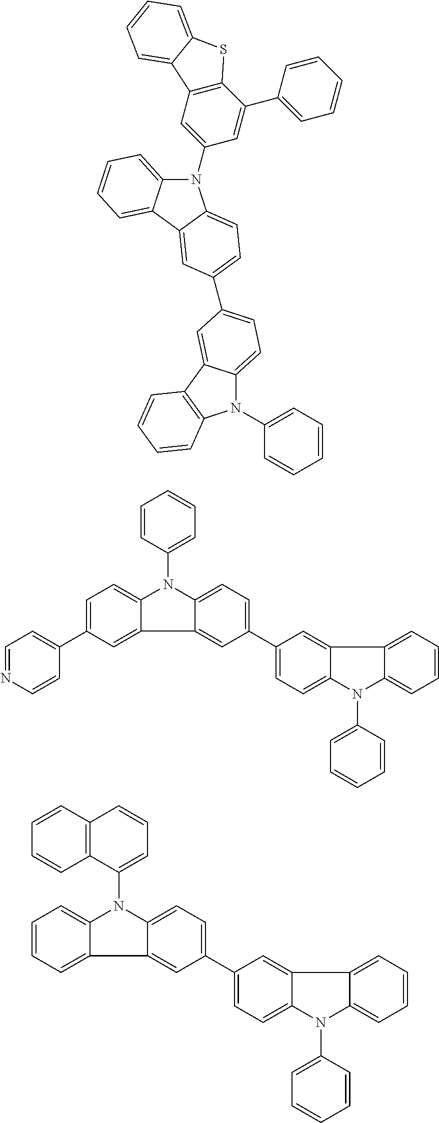

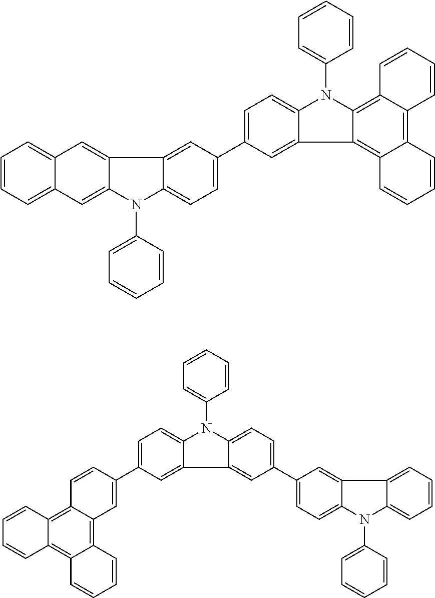 [SMILES: c1ccc(-n2c3ccc(-c4ccc5c(c4)c4c6ccccc6c6ccccc6c4n5-c4ccccc4)cc3c3cc4ccccc4cc32)cc1 ; c1ccc(-n2c3ccccc3c3cc(-c4ccc5c(c4)c4cc(-c6ccc7c8ccccc8c8ccccc8c7c6)ccc4n5-c4ccccc4)ccc32)cc1]

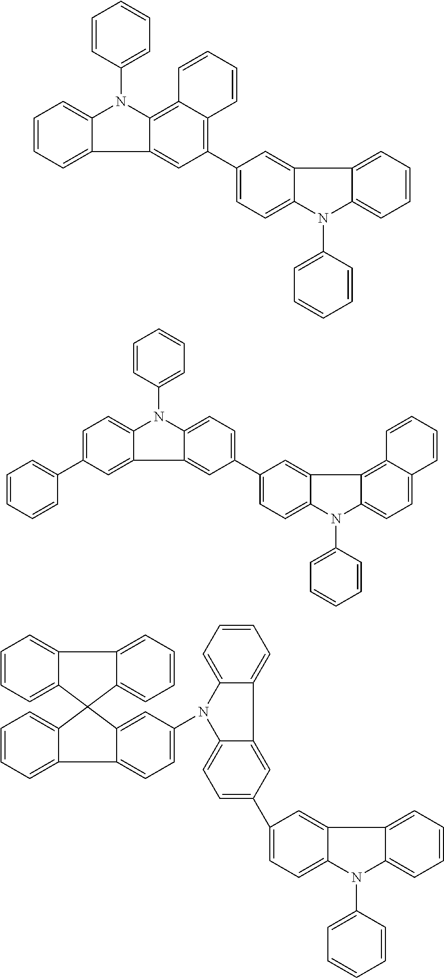 [SMILES: c1ccc(-n2c3ccccc3c3cc(-c4cc5c6ccccc6n(-c6ccccc6)c5c5ccccc45)ccc32)cc1 ; c1ccc(-c2ccc3c(c2)c2cc(-c4ccc5c(c4)c4c6ccccc6ccc4n5-c4ccccc4)ccc2n3-c2ccccc2)cc1 ; c1ccc(-n2c3ccccc3c3cc(-c4ccc5c(c4)c4ccccc4n5-c4ccc5c(c4)C4(c6ccccc6-c6ccccc64)c4ccccc4-5)ccc32)cc1]

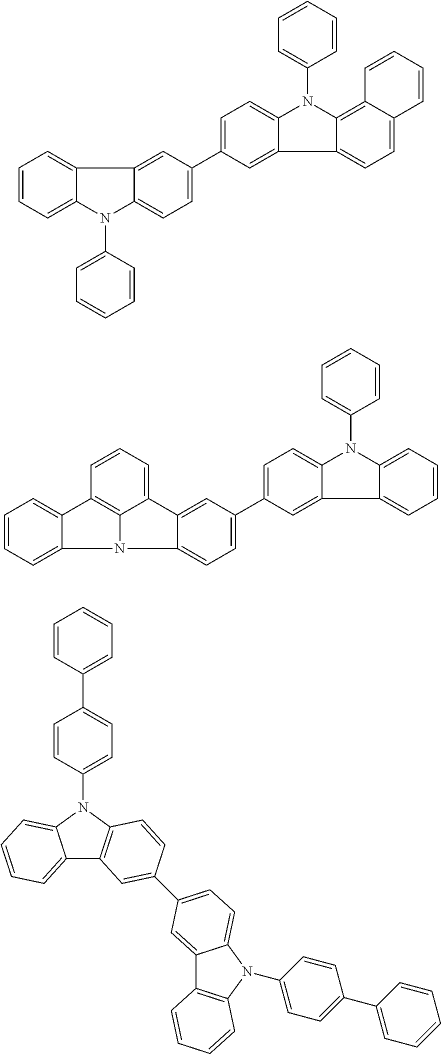 [SMILES: c1ccc(-n2c3ccccc3c3cc(-c4ccc5c(c4)c4ccc6ccccc6c4n5-c4ccccc4)ccc32)cc1 ; c1ccc(-n2c3ccccc3c3cc(-c4ccc5c(c4)c4cccc6c7ccccc7n5c64)ccc32)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

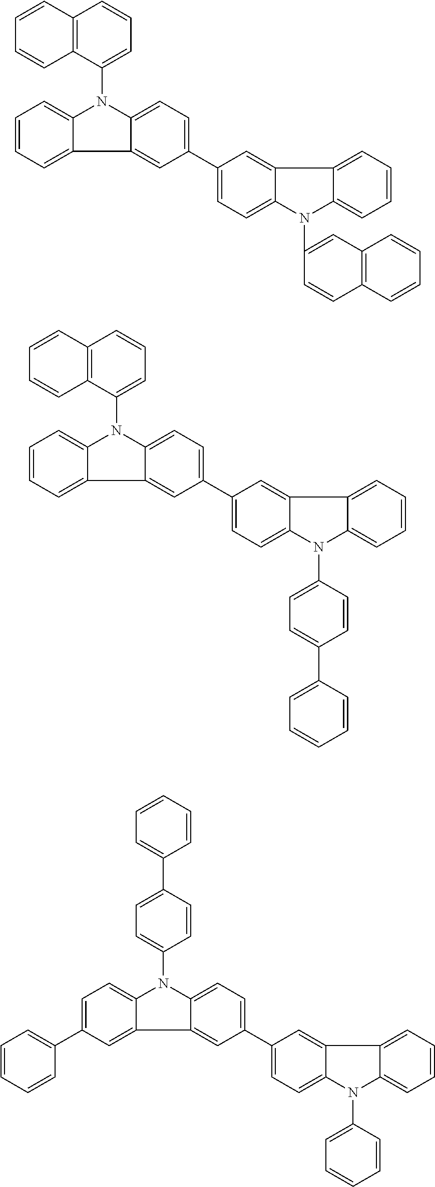 [SMILES: c1ccc2cc(-n3c4ccccc4c4cc(-c5ccc6c(c5)c5ccccc5n6-c5cccc6ccccc56)ccc43)ccc2c1 ; c1ccc(-c2ccc(-n3c4ccccc4c4cc(-c5ccc6c(c5)c5ccccc5n6-c5cccc6ccccc56)ccc43)cc2)cc1 ; c1ccc(-c2ccc(-n3c4ccc(-c5ccccc5)cc4c4cc(-c5ccc6c(c5)c5ccccc5n6-c5ccccc5)ccc43)cc2)cc1]

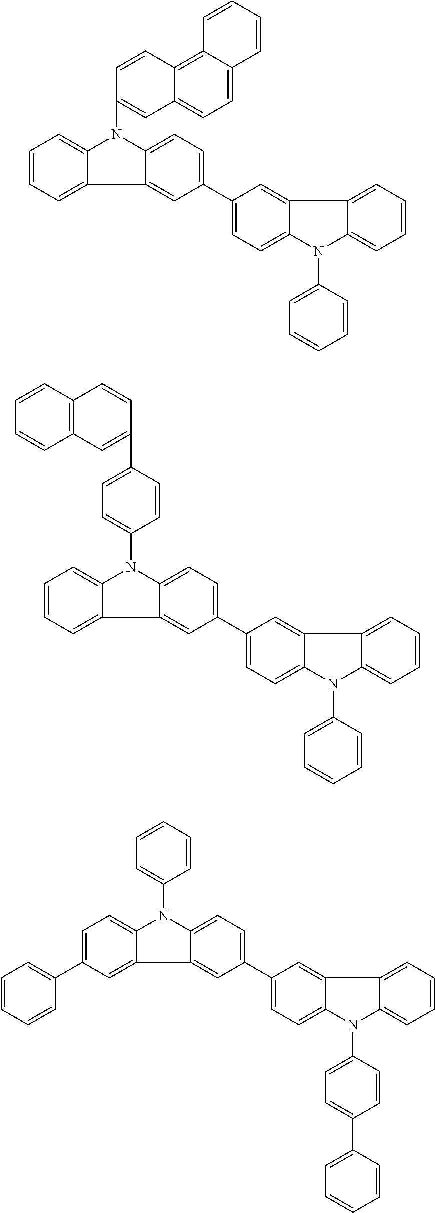 [SMILES: c1ccc(-n2c3ccccc3c3cc(-c4ccc5c(c4)c4ccccc4n5-c4ccc5c(ccc6ccccc65)c4)ccc32)cc1 ; c1ccc(-n2c3ccccc3c3cc(-c4ccc5c(c4)c4ccccc4n5-c4ccc(-c5ccc6ccccc6c5)cc4)ccc32)cc1 ; c1ccc(-c2ccc(-n3c4ccccc4c4cc(-c5ccc6c(c5)c5cc(-c7ccccc7)ccc5n6-c5ccccc5)ccc43)cc2)cc1]

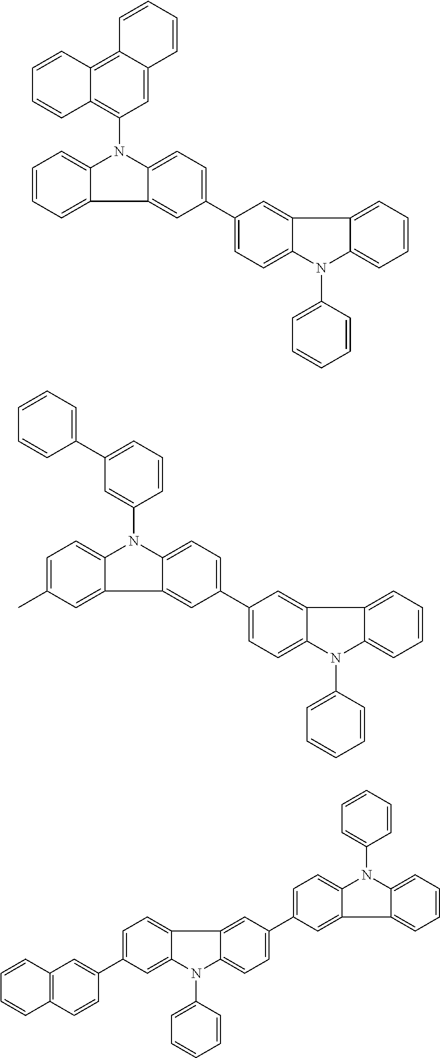

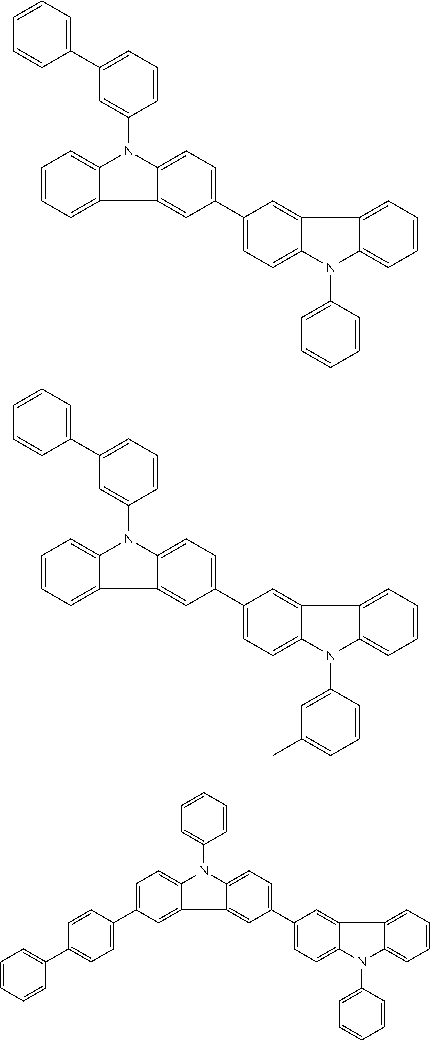 [SMILES: c1ccc(-c2cccc(-n3c4ccccc4c4cc(-c5ccc6c(c5)c5ccccc5n6-c5ccccc5)ccc43)c2)cc1 ; Cc1cccc(-n2c3ccccc3c3cc(-c4ccc5c(c4)c4ccccc4n5-c4cccc(-c5ccccc5)c4)ccc32)c1 ; c1ccc(-c2ccc(-c3ccc4c(c3)c3cc(-c5ccc6c(c5)c5ccccc5n6-c5ccccc5)ccc3n4-c3ccccc3)cc2)cc1]

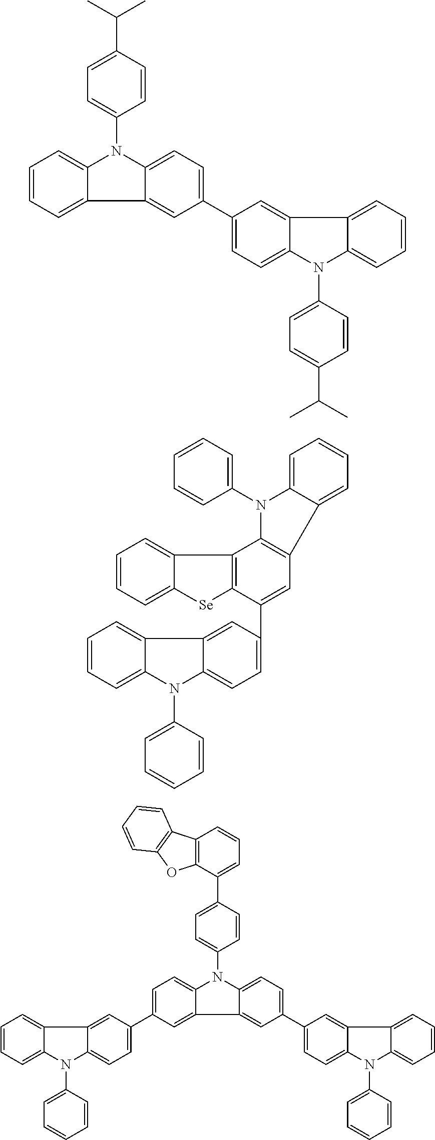

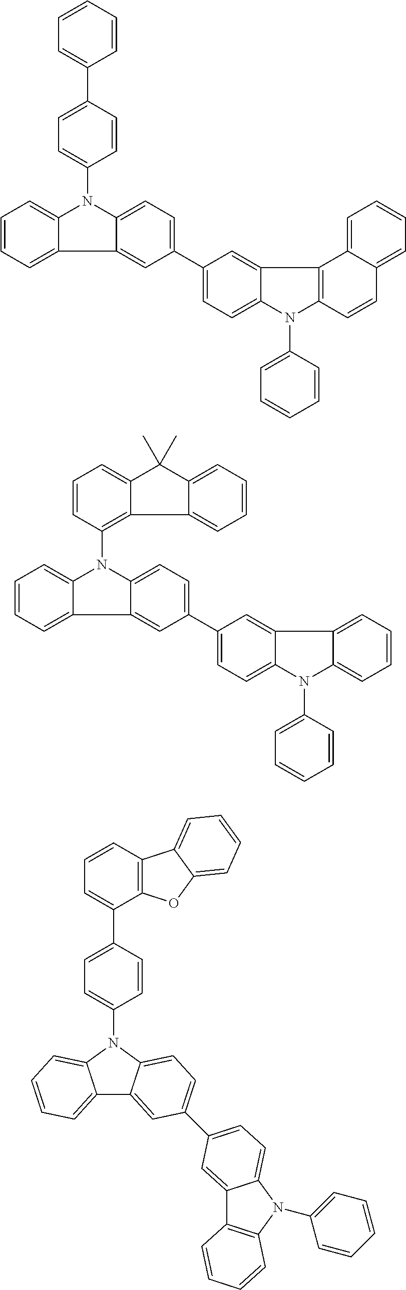 [SMILES: c1ccc(-c2ccc(-n3c4ccccc4c4cc(-c5ccc6c(c5)c5c7ccccc7ccc5n6-c5ccccc5)ccc43)cc2)cc1 ; CC1(C)c2ccccc2-c2c(-n3c4ccccc4c4cc(-c5ccc6c(c5)c5ccccc5n6-c5ccccc5)ccc43)cccc21 ; c1ccc(-n2c3ccccc3c3cc(-c4ccc5c(c4)c4ccccc4n5-c4ccc(-c5cccc6c5oc5ccccc56)cc4)ccc32)cc1]

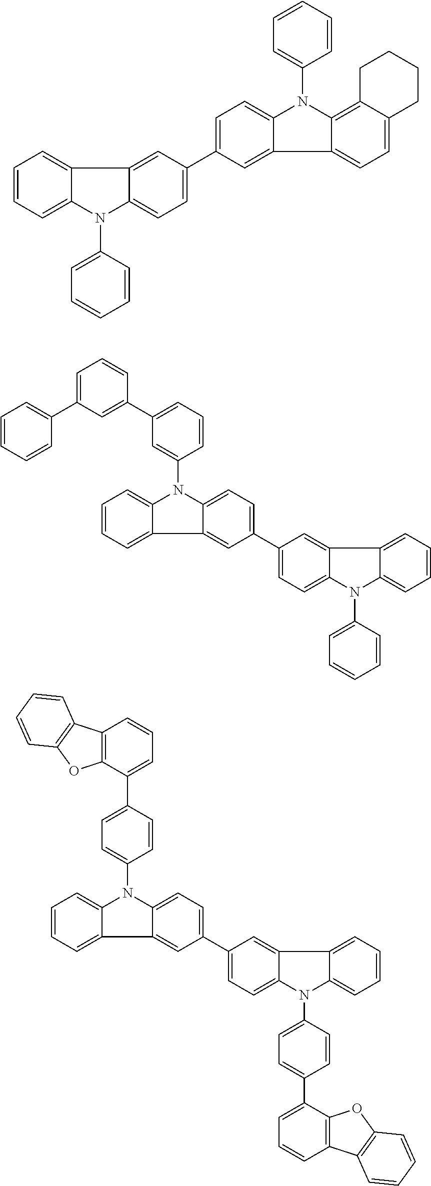

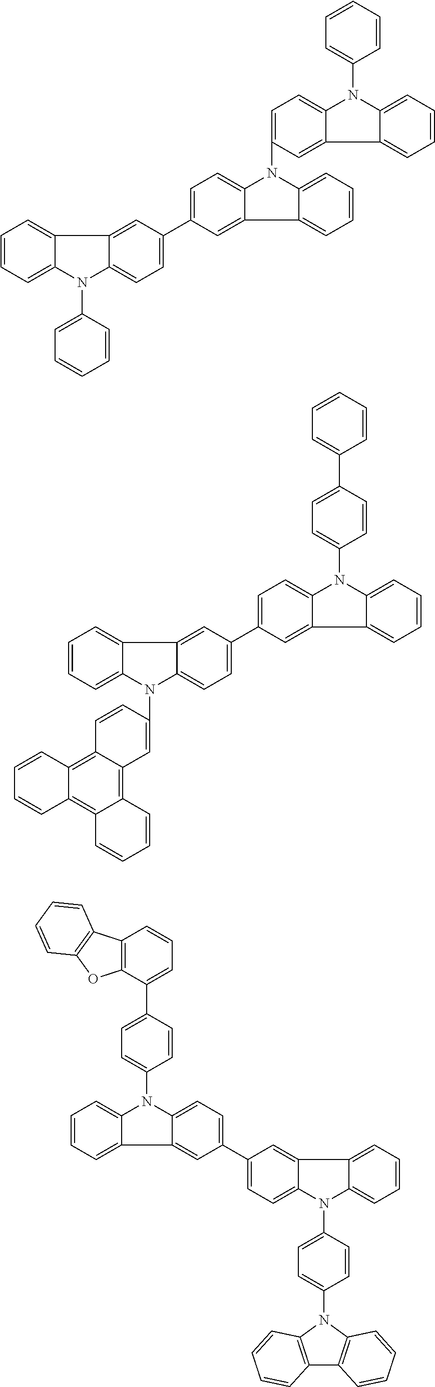 [SMILES: c1ccc(-n2c3ccccc3c3cc(-c4ccc5c(c4)c4ccccc4n5-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccc6c7ccccc7c7ccccc7c6c5)ccc43)cc2)cc1 ; c1ccc2c(c1)oc1c(-c3ccc(-n4c5ccccc5c5cc(-c6ccc7c(c6)c6ccccc6n7-c6ccc(-n7c8ccccc8c8ccccc87)cc6)ccc54)cc3)cccc12]

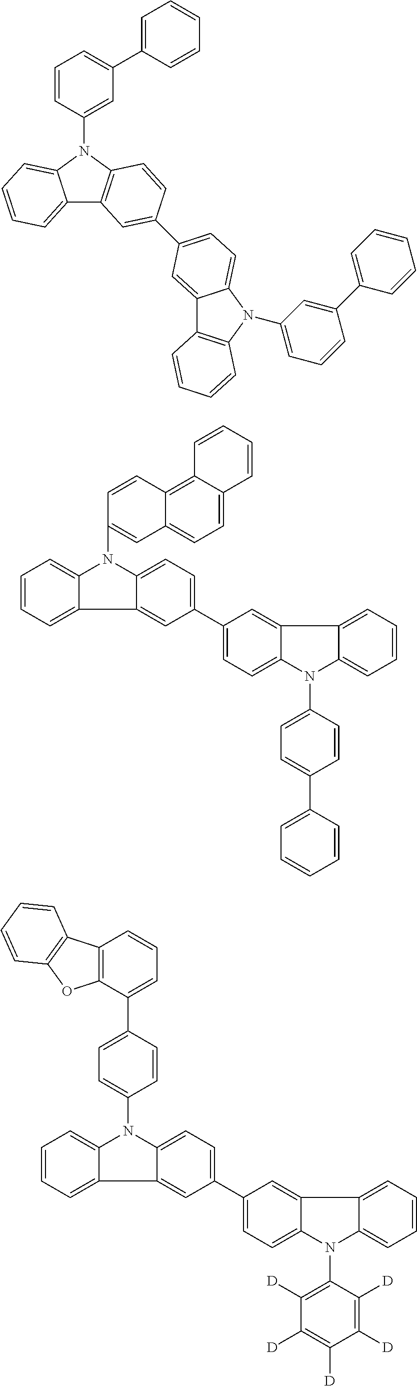

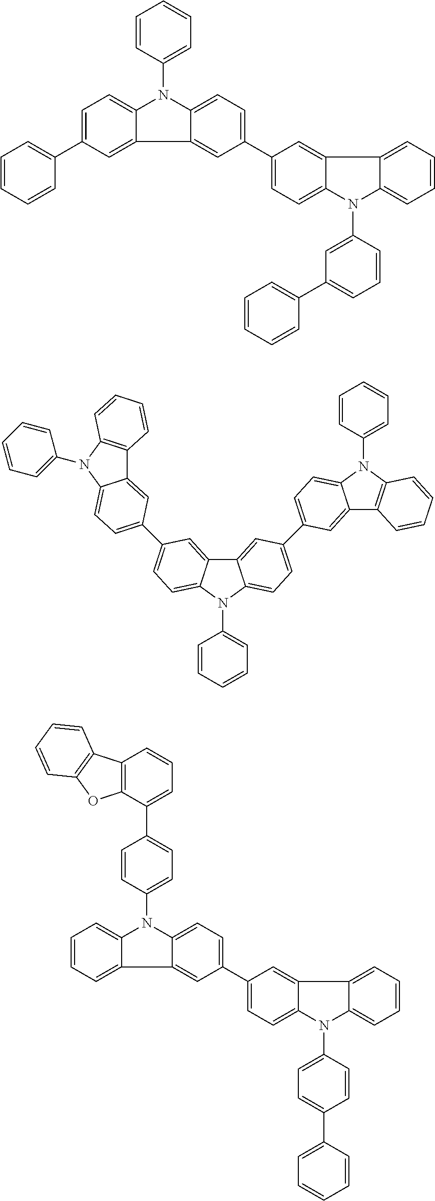 [SMILES: c1ccc(-c2cccc(-n3c4ccccc4c4cc(-c5ccc6c(c5)c5cc(-c7ccccc7)ccc5n6-c5ccccc5)ccc43)c2)cc1 ; c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1 ; c1ccc(-c2ccc(-n3c4ccccc4c4cc(-c5ccc6c(c5)c5ccccc5n6-c5ccc(-c6cccc7c6oc6ccccc67)cc5)ccc43)cc2)cc1]

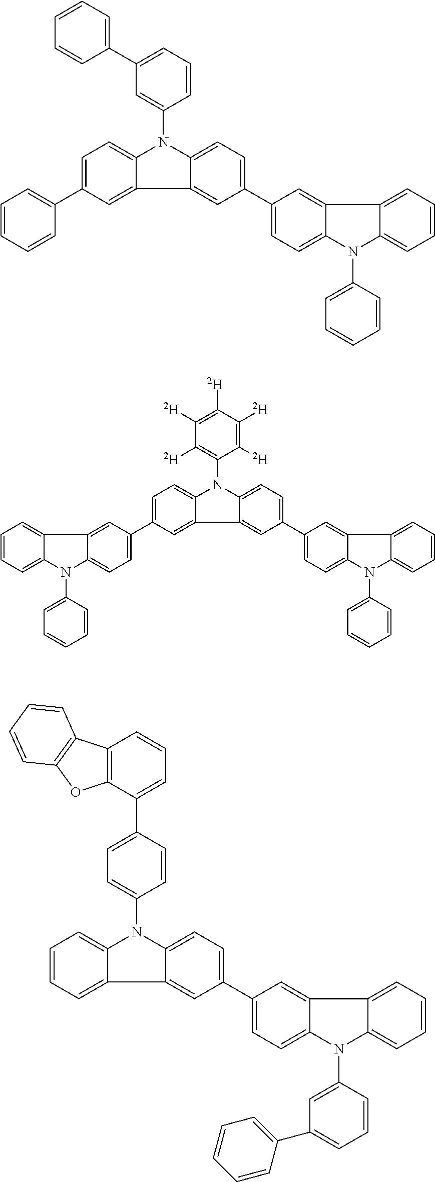 [SMILES: c1ccc(-c2cccc(-n3c4ccc(-c5ccccc5)cc4c4cc(-c5ccc6c(c5)c5ccccc5n6-c5ccccc5)ccc43)c2)cc1 ; [2H]c1c([2H])c([2H])c(-n2c3ccc(-c4ccc5c(c4)c4ccccc4n5-c4ccccc4)cc3c3cc(-c4ccc5c(c4)c4ccccc4n5-c4ccccc4)ccc32)c([2H])c1[2H] ; c1ccc(-c2cccc(-n3c4ccccc4c4cc(-c5ccc6c(c5)c5ccccc5n6-c5ccc(-c6cccc7c6oc6ccccc67)cc5)ccc43)c2)cc1]

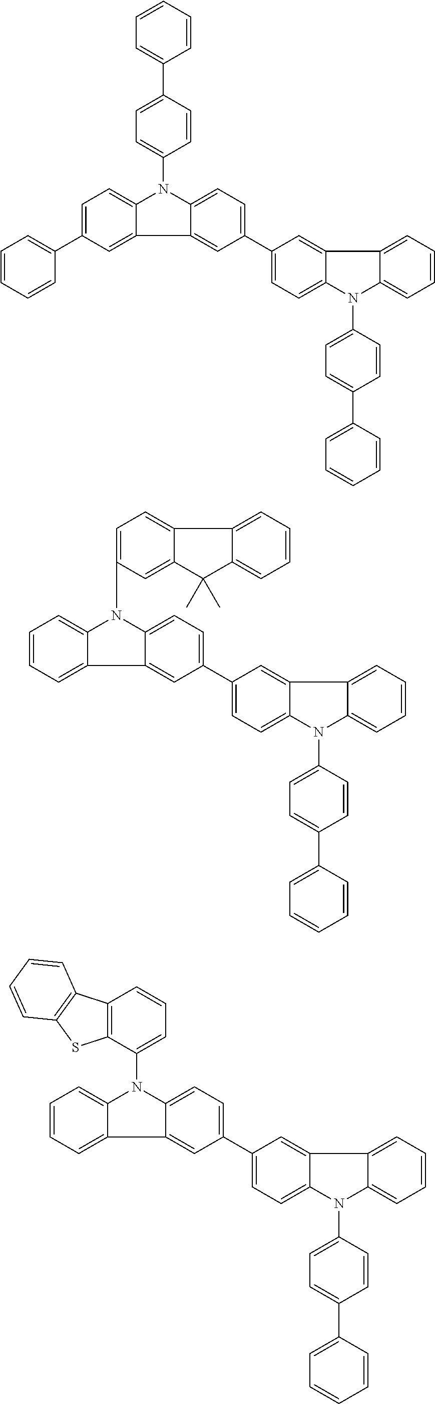 [SMILES: c1ccc(-c2ccc(-n3c4ccccc4c4cc(-c5ccc6c(c5)c5cc(-c7ccccc7)ccc5n6-c5ccc(-c6ccccc6)cc5)ccc43)cc2)cc1 ; CC1(C)c2ccccc2-c2ccc(-n3c4ccccc4c4cc(-c5ccc6c(c5)c5ccccc5n6-c5ccc(-c6ccccc6)cc5)ccc43)cc21 ; c1ccc(-c2ccc(-n3c4ccccc4c4cc(-c5ccc6c(c5)c5ccccc5n6-c5cccc6c5sc5ccccc56)ccc43)cc2)cc1]

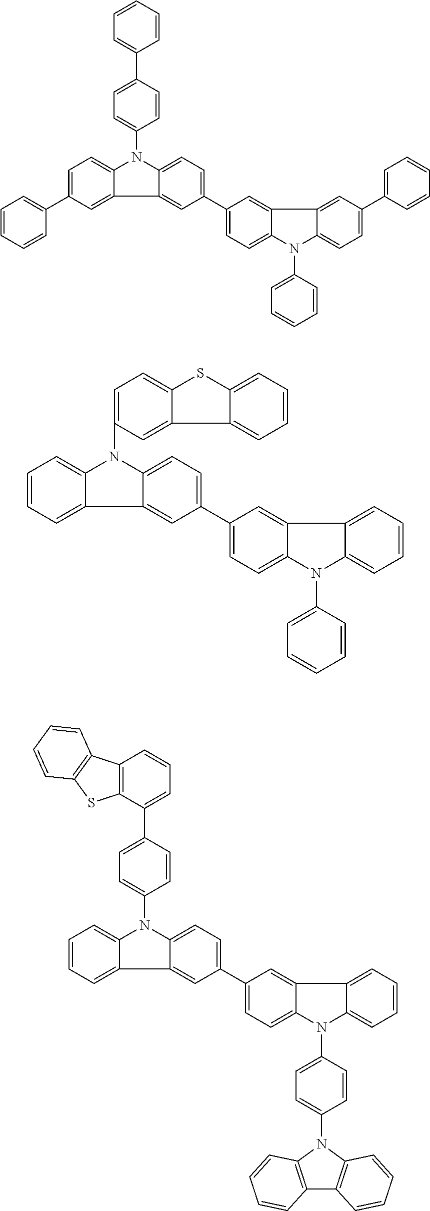 [SMILES: c1ccc(-c2ccc(-n3c4ccc(-c5ccccc5)cc4c4cc(-c5ccc6c(c5)c5cc(-c7ccccc7)ccc5n6-c5ccccc5)ccc43)cc2)cc1 ; c1ccc(-n2c3ccccc3c3cc(-c4ccc5c(c4)c4ccccc4n5-c4ccc5sc6ccccc6c5c4)ccc32)cc1 ; c1ccc2c(c1)sc1c(-c3ccc(-n4c5ccccc5c5cc(-c6ccc7c(c6)c6ccccc6n7-c6ccc(-n7c8ccccc8c8ccccc87)cc6)ccc54)cc3)cccc12]

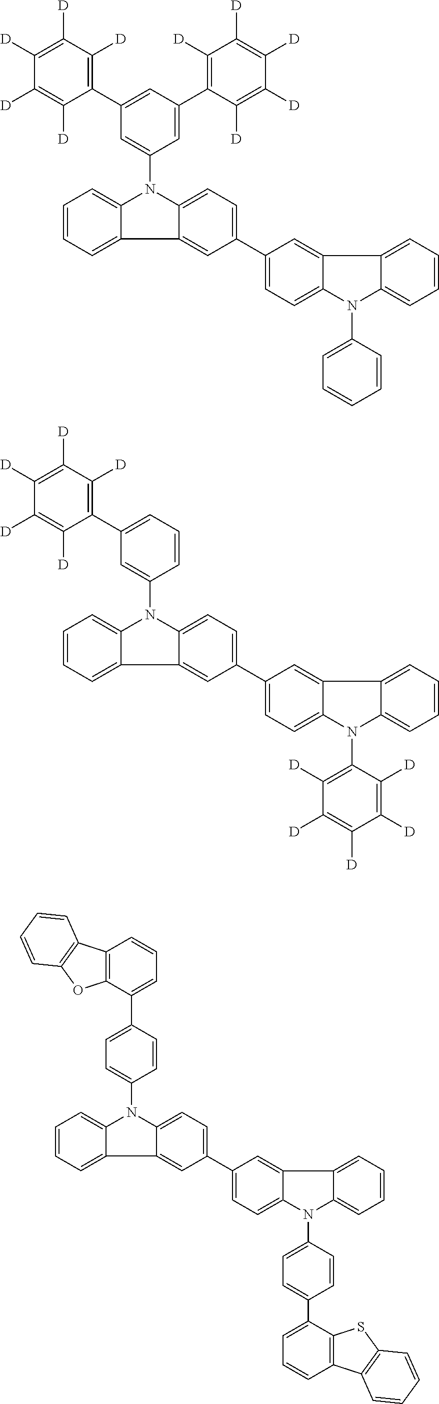 [SMILES: [2H]c1c([2H])c([2H])c(-c2cc(-c3c([2H])c([2H])c([2H])c([2H])c3[2H])cc(-n3c4ccccc4c4cc(-c5ccc6c(c5)c5ccccc5n6-c5ccccc5)ccc43)c2)c([2H])c1[2H] ; [2H]c1c([2H])c([2H])c(-c2cccc(-n3c4ccccc4c4cc(-c5ccc6c(c5)c5ccccc5n6-c5c([2H])c([2H])c([2H])c([2H])c5[2H])ccc43)c2)c([2H])c1[2H] ; c1ccc2c(c1)oc1c(-c3ccc(-n4c5ccccc5c5cc(-c6ccc7c(c6)c6ccccc6n7-c6ccc(-c7cccc8c7sc7ccccc78)cc6)ccc54)cc3)cccc12]

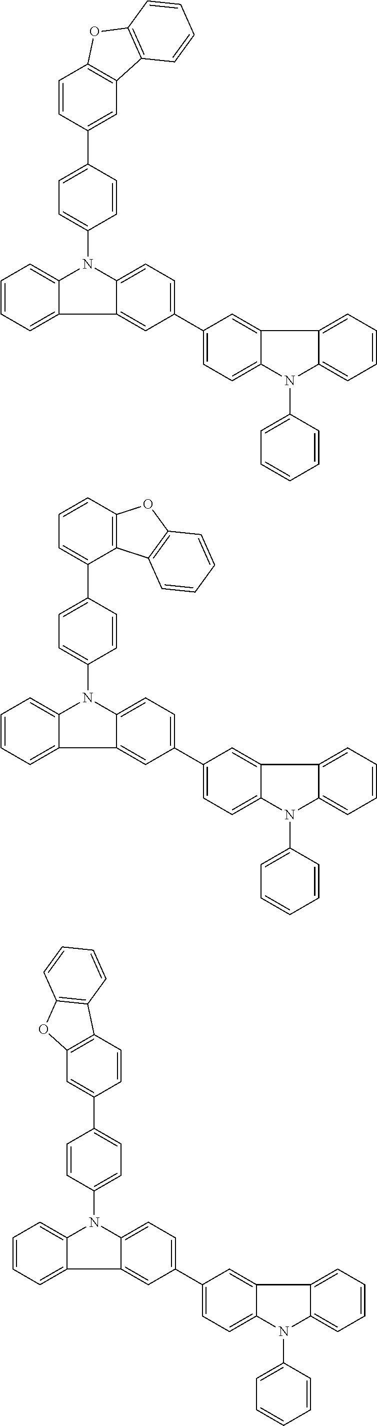 [SMILES: c1ccc(-n2c3ccccc3c3cc(-c4ccc5c(c4)c4ccccc4n5-c4ccc(-c5ccc6oc7ccccc7c6c5)cc4)ccc32)cc1 ; c1ccc(-n2c3ccccc3c3cc(-c4ccc5c(c4)c4ccccc4n5-c4ccc(-c5cccc6oc7ccccc7c56)cc4)ccc32)cc1 ; c1ccc(-n2c3ccccc3c3cc(-c4ccc5c(c4)c4ccccc4n5-c4ccc(-c5ccc6c(c5)oc5ccccc56)cc4)ccc32)cc1]

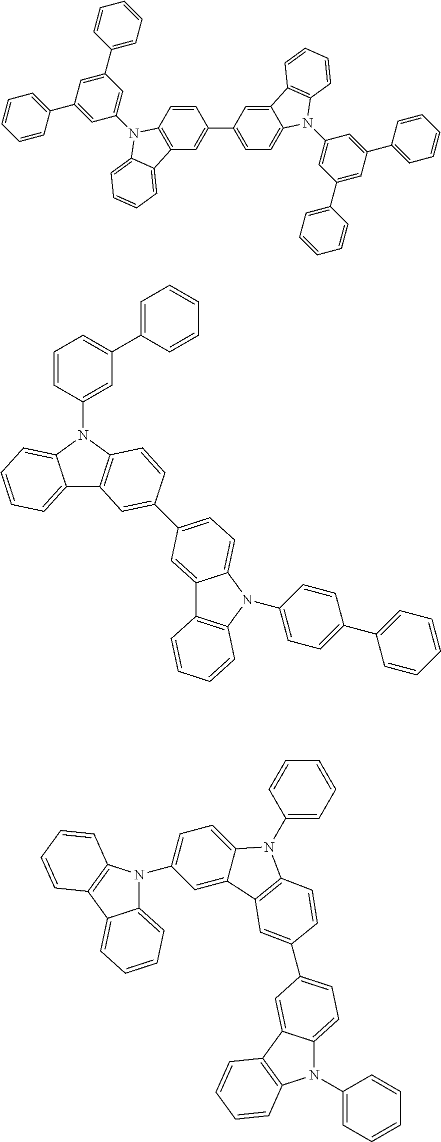 [SMILES: c1ccc(-c2cc(-c3ccccc3)cc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-n2c3ccccc3c3cc(-c4ccc5c(c4)c4cc(-n6c7ccccc7c7ccccc76)ccc4n5-c4ccccc4)ccc32)cc1]

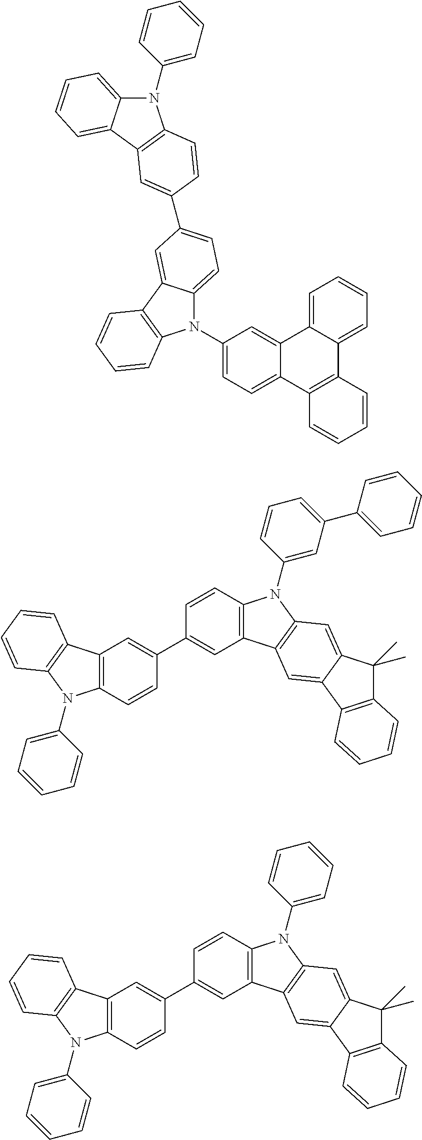

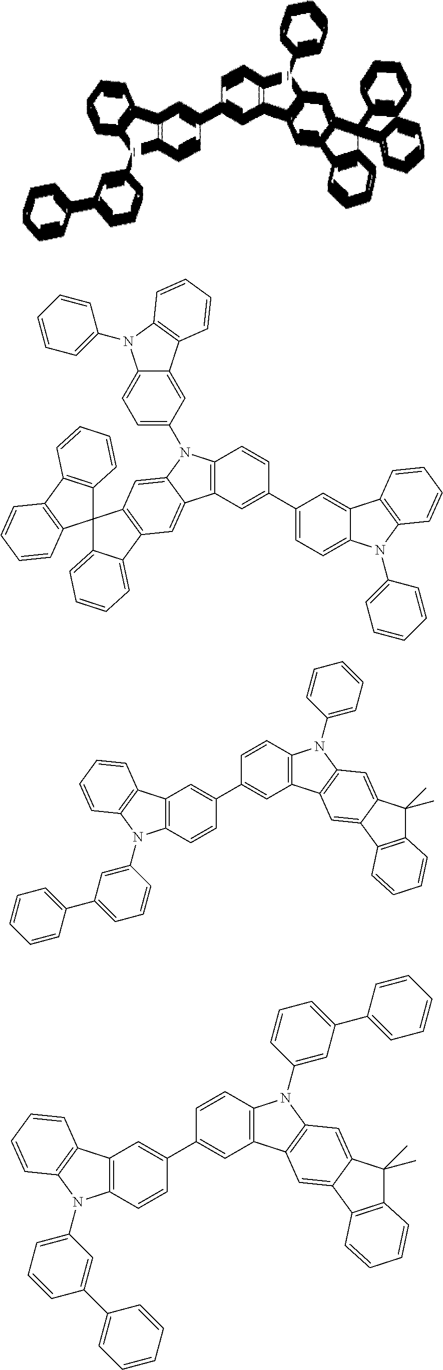

Preferred bridged carbazoles are the structures of the following formula (32):

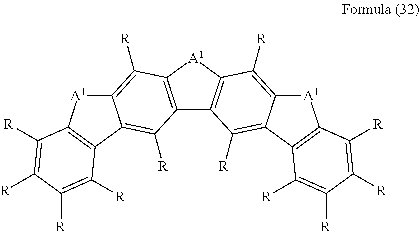

where A¹ and R have the definitions given above and A¹ is preferably the same or different at each instance and is selected from the group consisting of NAr¹ and CR₂.

Preferred dibenzofuran derivatives are the compounds of the following formula (33):

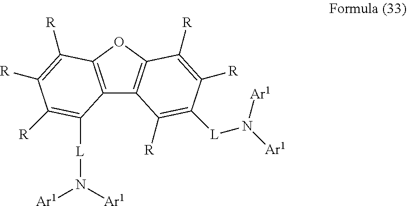

where the oxygen may also be replaced by sulfur so as to form a dibenzothiophene, L is a single bond or an aromatic or heteroaromatic ring system which has 5 to 30 aromatic ring atoms and may also be substituted by one or more R radicals, and R and Ar¹ have the definitions given above. It is also possible here for the two Ar¹ groups that bind to the same nitrogen atom, or for one Ar¹ group and one L group that bind to the same nitrogen atom, to be bonded to one another, for example to give a carbazole.

Examples of suitable dibenzofuran derivatives are the compounds depicted below.

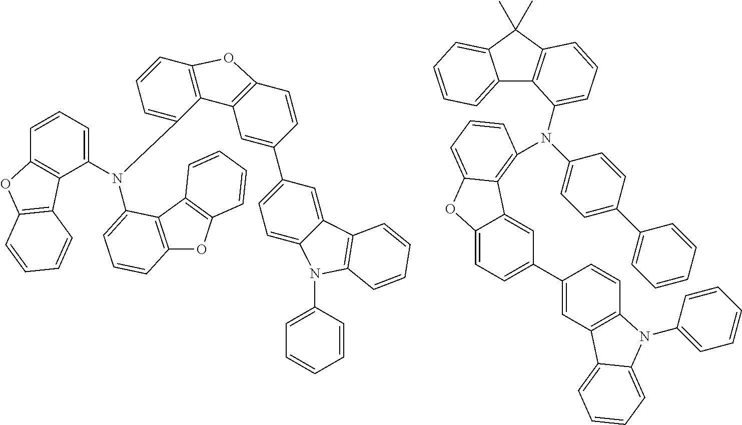 [SMILES: c1ccc(-n2c3ccccc3c3cc(-c4ccc5oc6cccc(N(c7cccc8oc9ccccc9c78)c7cccc8oc9ccccc9c78)c6c5c4)ccc32)cc1 ; CC1(C)c2ccccc2-c2c(N(c3ccc(-c4ccccc4)cc3)c3cccc4oc5ccc(-c6ccc7c(c6)c6ccccc6n7-c6ccccc6)cc5c34)cccc21]

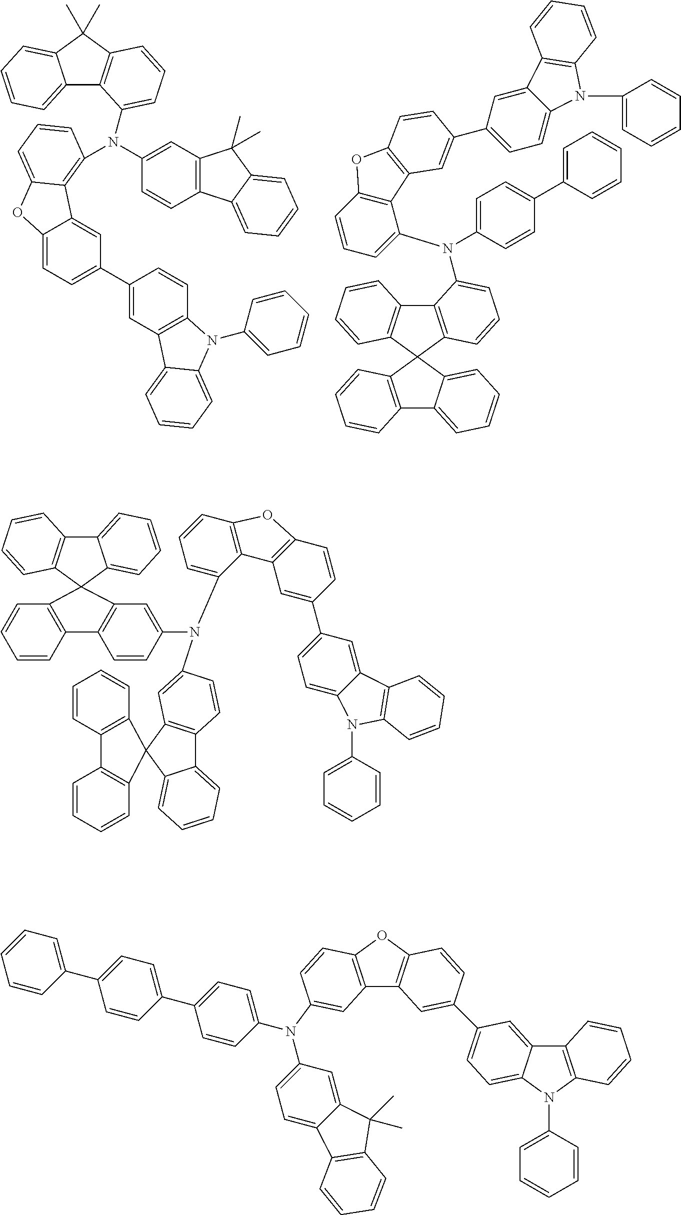

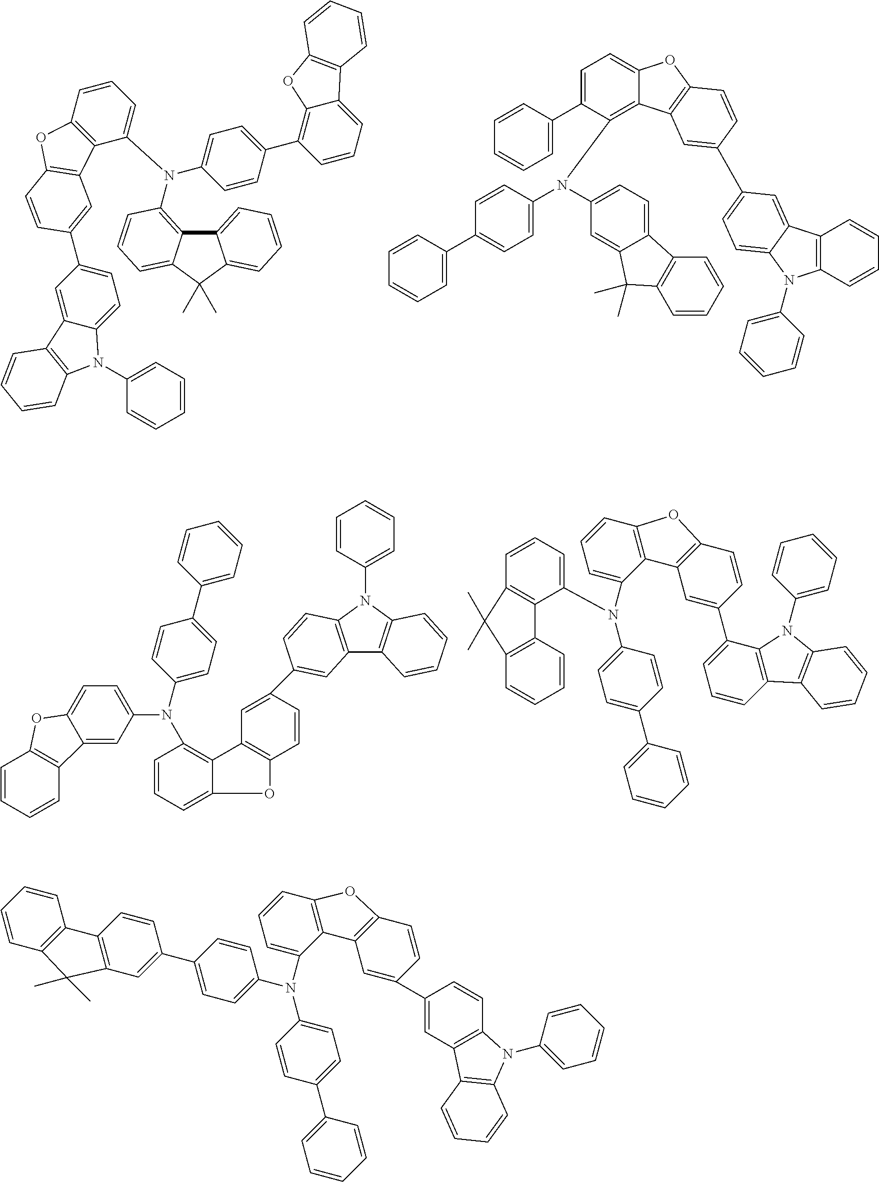

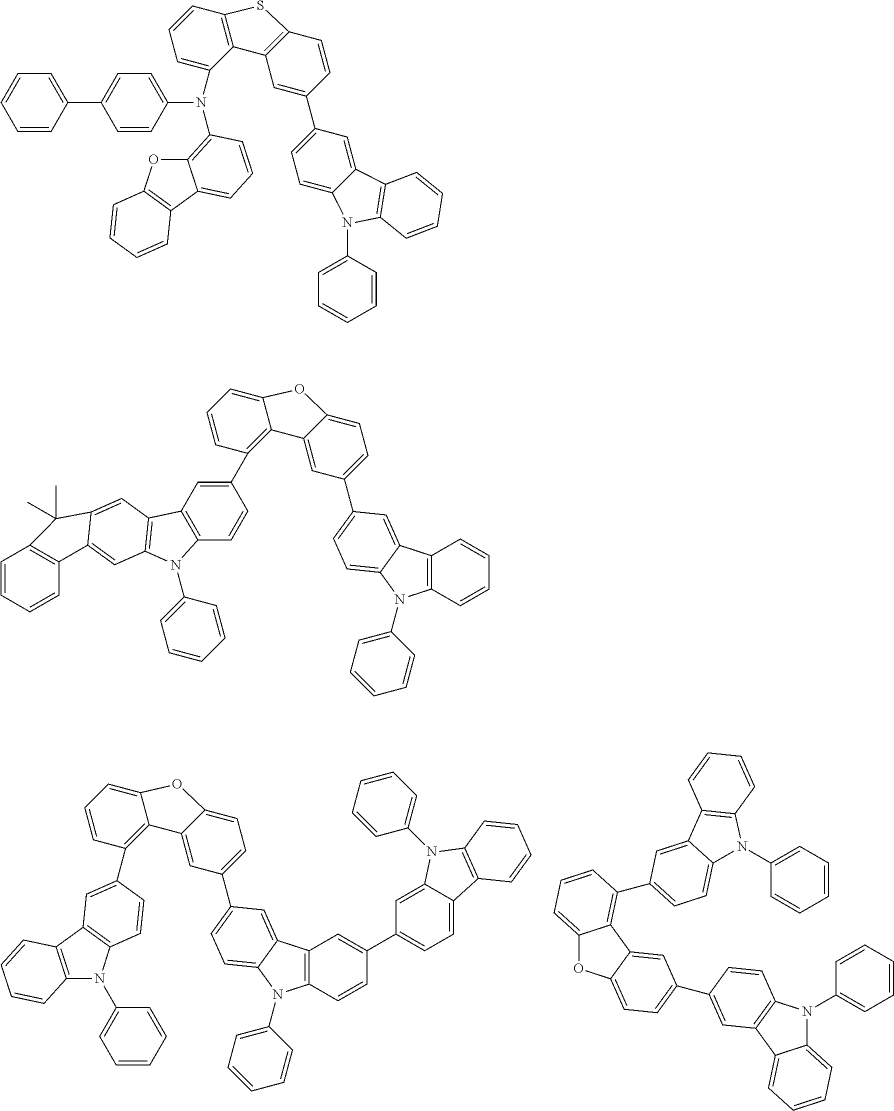

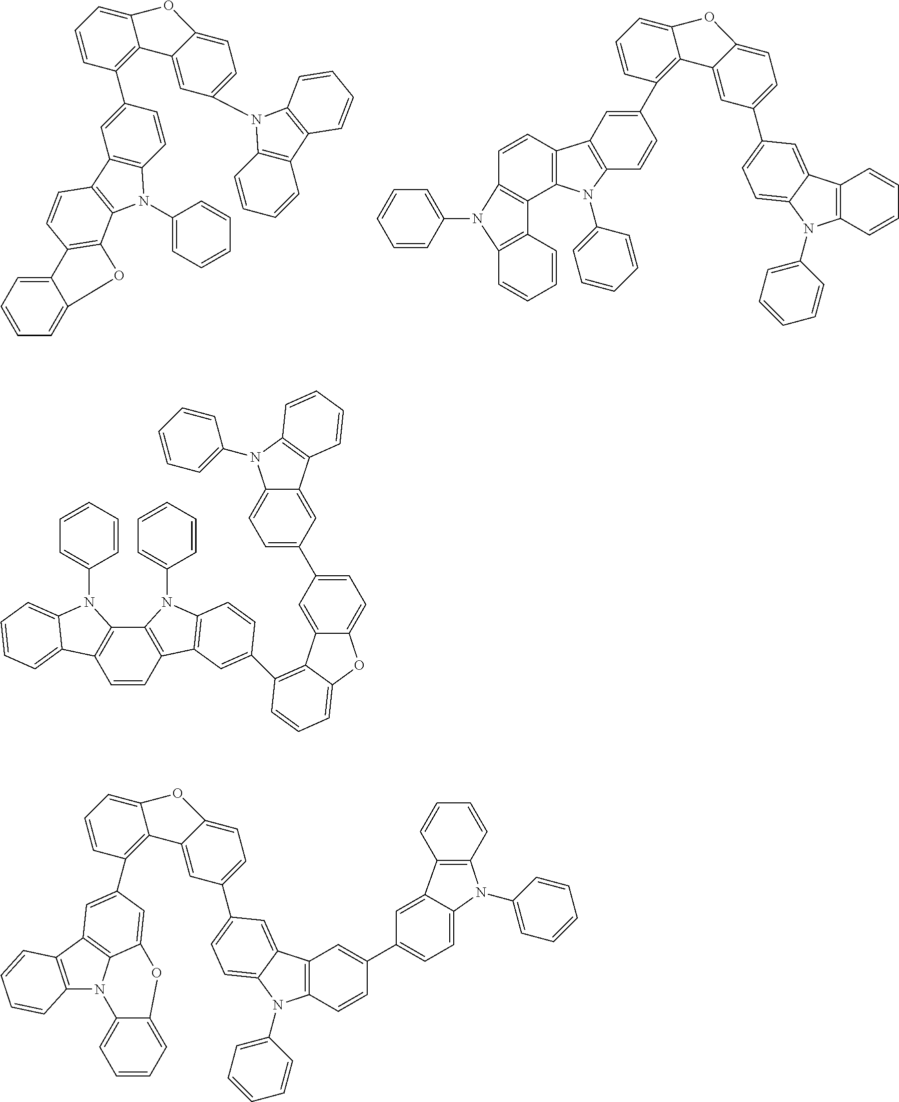 [SMILES: c1ccc(-n2c3ccc(-c4cccc5oc6ccc(-n7c8ccccc8c8ccccc87)cc6c45)cc3c3ccc4c5ccccc5oc4c32)cc1 ; c1ccc(-n2c3ccccc3c3cc(-c4ccc5oc6cccc(-c7ccc8c(c7)c7ccc9c(c%10ccccc%10n9-c9ccccc9)c7n8-c7ccccc7)c6c5c4)ccc32)cc1 ; c1ccc(-n2c3ccccc3c3cc(-c4ccc5oc6cccc(-c7ccc8c(c7)c7ccc9c%10ccccc%10n(-c%10ccccc%10)c9c7n8-c7ccccc7)c6c5c4)ccc32)cc1 ; c1ccc(-n2c3ccccc3c3cc(-c4ccc5c(c4)c4cc(-c6ccc7oc8cccc(-c9cc%10c%11c(c9)c9ccccc9n%11-c9ccccc9O%10)c8c7c6)ccc4n5-c4ccccc4)ccc32)cc1]

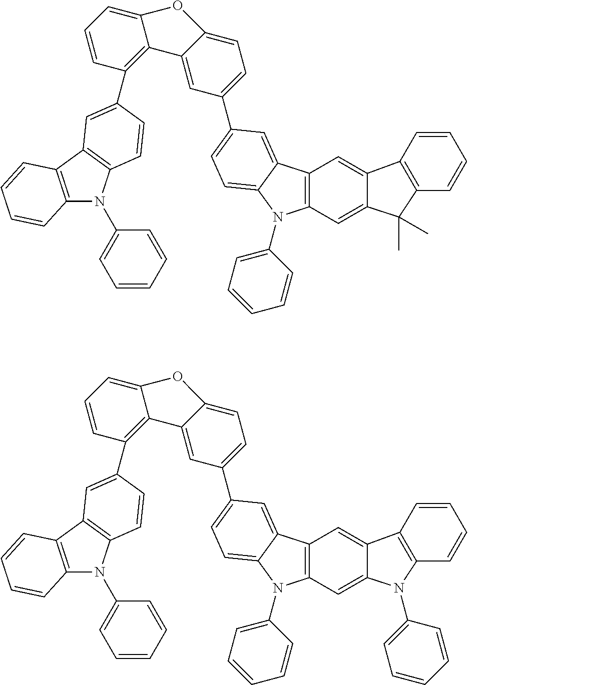 [SMILES: CC1(C)c2ccccc2-c2cc3c4cc(-c5ccc6oc7cccc(-c8ccc9c(c8)c8ccccc8n9-c8ccccc8)c7c6c5)ccc4n(-c4ccccc4)c3cc21 ; c1ccc(-n2c3ccccc3c3cc(-c4cccc5oc6ccc(-c7ccc8c(c7)c7cc9c%10ccccc%10n(-c%10ccccc%10)c9cc7n8-c7ccccc7)cc6c45)ccc32)cc1]

Preferred carbazoleamines are the structures of the following formulae (34), (35) and (36):

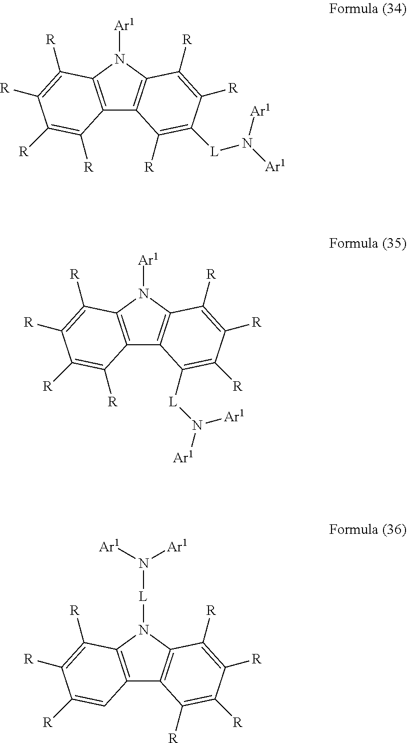

where L is an aromatic or heteroaromatic ring system which has 5 to 30 aromatic ring atoms and may be substituted by one or more R radicals, and R and Ar¹ have the definitions given above.

Examples of suitable carbazoleamine derivatives are the compounds depicted below.

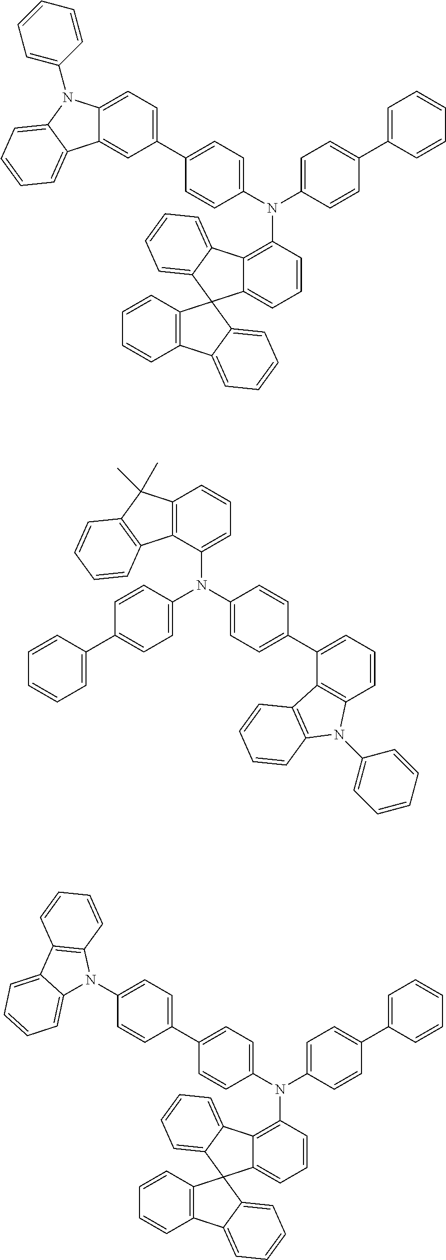 [SMILES: c1ccc(-c2ccc(N(c3ccc(-c4ccc5c(c4)c4ccccc4n5-c4ccccc4)cc3)c3cccc4c3-c3ccccc3C43c4ccccc4-c4ccccc43)cc2)cc1 ; CC1(C)c2ccccc2-c2c(N(c3ccc(-c4ccccc4)cc3)c3ccc(-c4cccc5c4c4ccccc4n5-c4ccccc4)cc3)cccc21 ; c1ccc(-c2ccc(N(c3ccc(-c4ccc(-n5c6ccccc6c6ccccc65)cc4)cc3)c3cccc4c3-c3ccccc3C43c4ccccc4-c4ccccc43)cc2)cc1]

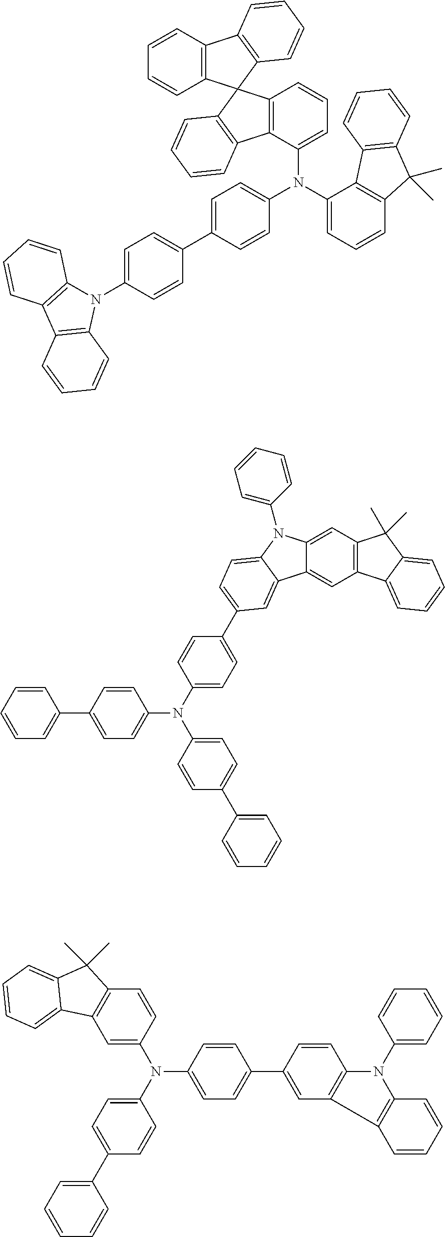 [SMILES: CC1(C)c2ccccc2-c2c(N(c3ccc(-c4ccc(-n5c6ccccc6c6ccccc65)cc4)cc3)c3cccc4c3-c3ccccc3C43c4ccccc4-c4ccccc43)cccc21 ; CC1(C)c2ccccc2-c2cc3c4cc(-c5ccc(N(c6ccc(-c7ccccc7)cc6)c6ccc(-c7ccccc7)cc6)cc5)ccc4n(-c4ccccc4)c3cc21 ; CC1(C)c2ccccc2-c2cc(N(c3ccc(-c4ccccc4)cc3)c3ccc(-c4ccc5c(c4)c4ccccc4n5-c4ccccc4)cc3)ccc21]

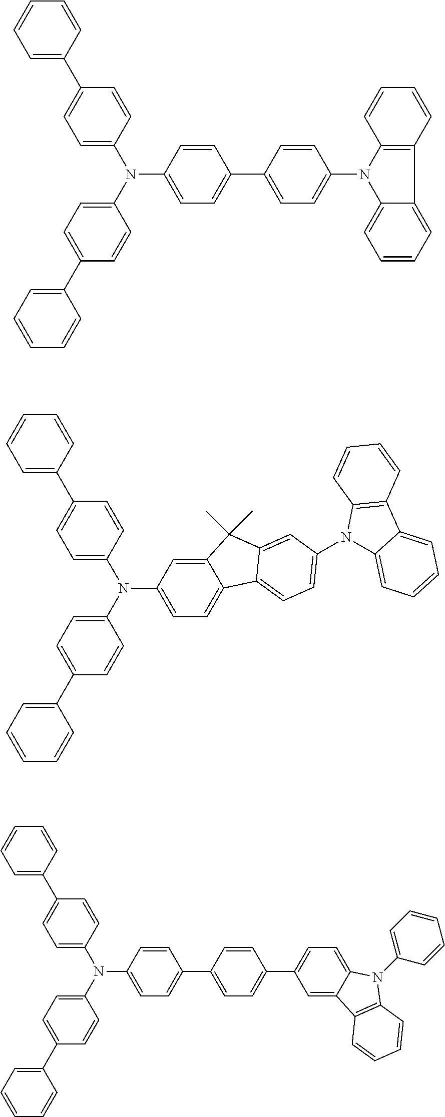 [SMILES: c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2cc(N(c3ccc(-c4ccccc4)cc3)c3ccc(-c4ccccc4)cc3)ccc2-c2ccc(-n3c4ccccc4c4ccccc43)cc21 ; c1ccc(-c2ccc(N(c3ccc(-c4ccccc4)cc3)c3ccc(-c4ccc(-c5ccc6c(c5)c5ccccc5n6-c5ccccc5)cc4)cc3)cc2)cc1]

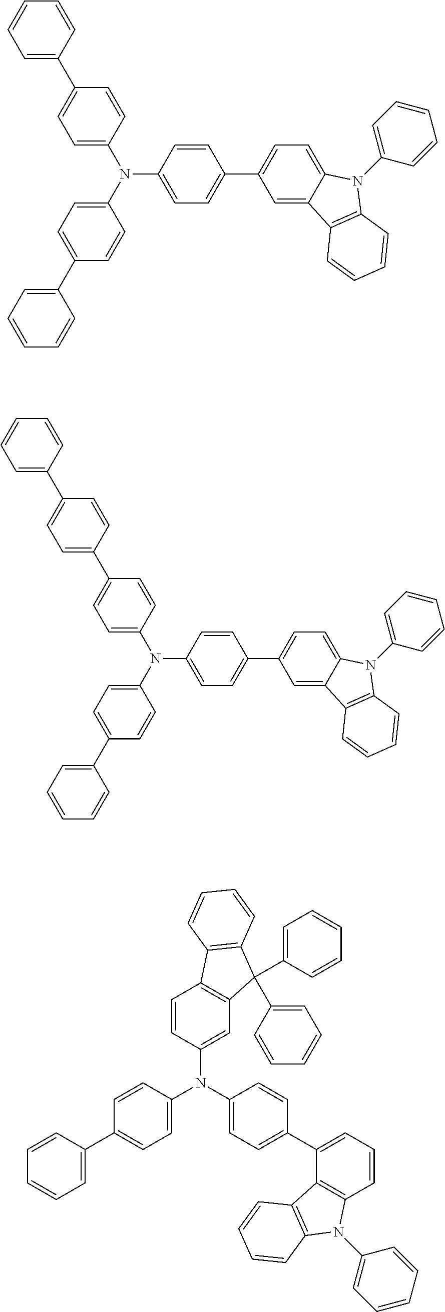 [SMILES: c1ccc(-c2ccc(N(c3ccc(-c4ccccc4)cc3)c3ccc(-c4ccc5c(c4)c4ccccc4n5-c4ccccc4)cc3)cc2)cc1 ; c1ccc(-c2ccc(-c3ccc(N(c4ccc(-c5ccccc5)cc4)c4ccc(-c5ccc6c(c5)c5ccccc5n6-c5ccccc5)cc4)cc3)cc2)cc1 ; c1ccc(-c2ccc(N(c3ccc(-c4cccc5c4c4ccccc4n5-c4ccccc4)cc3)c3ccc4c(c3)C(c3ccccc3)(c3ccccc3)c3ccccc3-4)cc2)cc1]

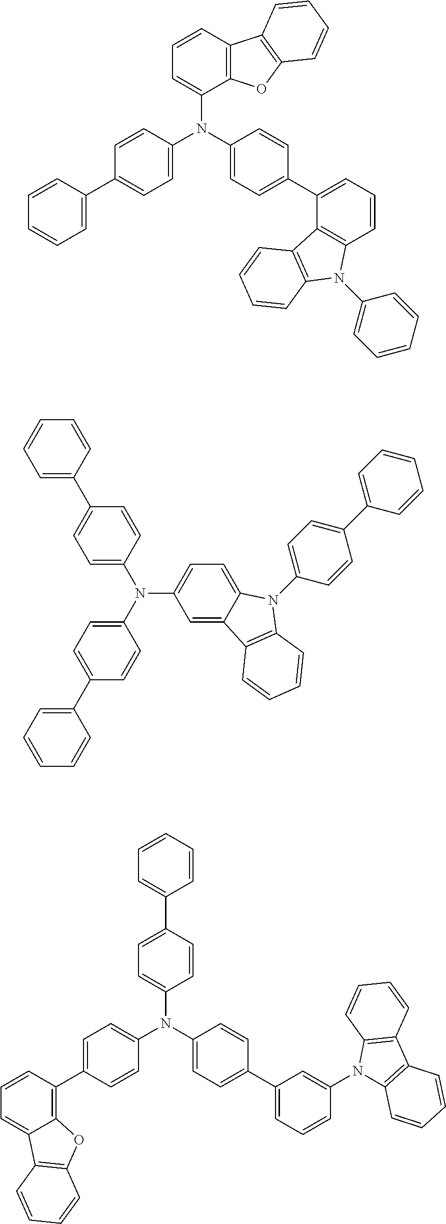 [SMILES: c1ccc(-c2ccc(N(c3ccc(-c4cccc5c4c4ccccc4n5-c4ccccc4)cc3)c3cccc4c3oc3ccccc34)cc2)cc1 ; c1ccc(-c2ccc(N(c3ccc(-c4ccccc4)cc3)c3ccc4c(c3)c3ccccc3n4-c3ccc(-c4ccccc4)cc3)cc2)cc1 ; c1ccc(-c2ccc(N(c3ccc(-c4cccc(-n5c6ccccc6c6ccccc65)c4)cc3)c3ccc(-c4cccc5c4oc4ccccc45)cc3)cc2)cc1]

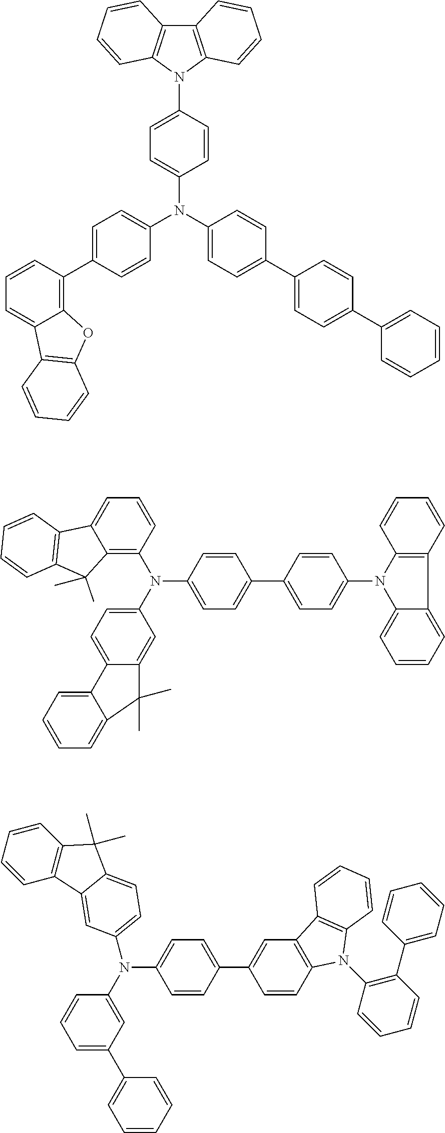 [SMILES: c1ccc(-c2ccc(-c3ccc(N(c4ccc(-c5cccc6c5oc5ccccc56)cc4)c4ccc(-n5c6ccccc6c6ccccc65)cc4)cc3)cc2)cc1 ; CC1(C)c2ccccc2-c2ccc(N(c3ccc(-c4ccc(-n5c6ccccc6c6ccccc65)cc4)cc3)c3cccc4c3C(C)(C)c3ccccc3-4)cc21 ; CC1(C)c2ccccc2-c2cc(N(c3ccc(-c4ccc5c(c4)c4ccccc4n5-c4ccccc4-c4ccccc4)cc3)c3cccc(-c4ccccc4)c3)ccc21]

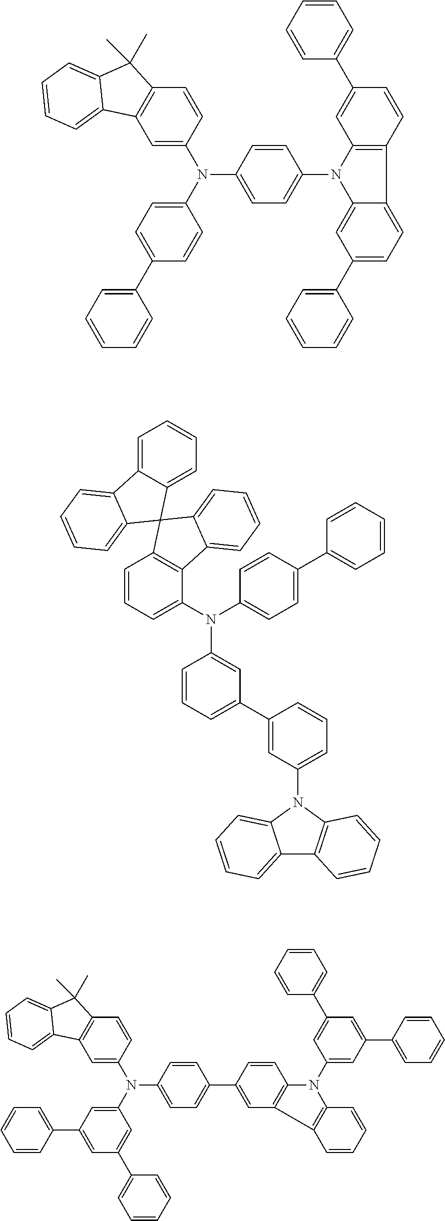 [SMILES: CC1(C)c2ccccc2-c2cc(N(c3ccc(-c4ccccc4)cc3)c3ccc(-n4c5cc(-c6ccccc6)ccc5c5ccc(-c6ccccc6)cc54)cc3)ccc21 ; c1ccc(-c2ccc(N(c3cccc(-c4cccc(-n5c6ccccc6c6ccccc65)c4)c3)c3cccc4c3-c3ccccc3C43c4ccccc4-c4ccccc43)cc2)cc1 ; CC1(C)c2ccccc2-c2cc(N(c3ccc(-c4ccc5c(c4)c4ccccc4n5-c4cc(-c5ccccc5)cc(-c5ccccc5)c4)cc3)c3cc(-c4ccccc4)cc(-c4ccccc4)c3)ccc21]

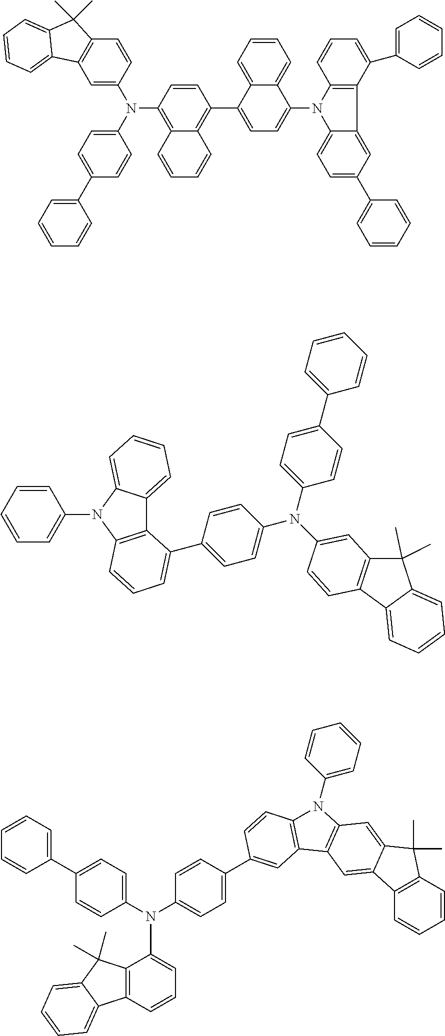 [SMILES: CC1(C)c2ccccc2-c2cc(N(c3ccc(-c4ccccc4)cc3)c3ccc(-c4ccc(-n5c6ccc(-c7ccccc7)cc6c6c(-c7ccccc7)cccc65)c5ccccc45)c4ccccc34)ccc21 ; CC1(C)c2ccccc2-c2ccc(N(c3ccc(-c4ccccc4)cc3)c3ccc(-c4cccc5c4c4ccccc4n5-c4ccccc4)cc3)cc21 ; CC1(C)c2ccccc2-c2cc3c4cc(-c5ccc(N(c6ccc(-c7ccccc7)cc6)c6cccc7c6C(C)(C)c6ccccc6-7)cc5)ccc4n(-c4ccccc4)c3cc21]

Especially when the compound of the invention is substituted by an aromatic ring system or an electron-rich heteroaromatic ring system, for example a carbazole group, or has a group of the formula (3), preferred co-matrix materials are selected from the group consisting of triazine derivatives, pyrimidine derivatives and quinazoline derivatives. Preferred triazine, quinazoline or pyrimidine derivatives that can be used as a mixture together with the compounds of the invention are the compounds of the following formulae (37), (38) and (39):

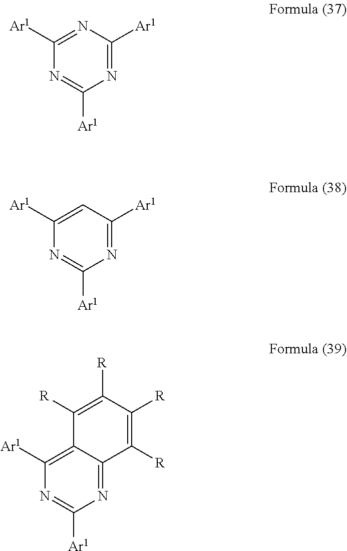

where Ar¹ and R have the definitions given above.

Particular preference is given to the triazine derivatives of the formula (37) and the quinazoline derivatives of the formula (39), especially the triazine derivatives of the formula (37).

In a preferred embodiment of the invention, Ar¹ in the formulae (37), (38) and (39) is the same or different at each instance and is an aromatic or heteroaromatic ring system which has 6 to 30 aromatic ring atoms, especially 6 to 24 aromatic ring atoms, and may be substituted by one or more R radicals. Suitable aromatic or heteroaromatic ring systems Ar¹ here are the same as set out above as embodiments for Ar¹, Ar² and Ar³, especially the structures Ar-1 to Ar-83.

Examples of suitable triazine compounds that may be used as matrix materials together with the compounds of the invention are the compounds depicted in the following table:

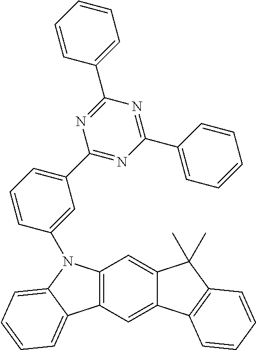 [SMILES: CC1(C)c2ccccc2-c2cc3c4ccccc4n(-c4cccc(-c5nc(-c6ccccc6)nc(-c6ccccc6)n5)c4)c3cc21]

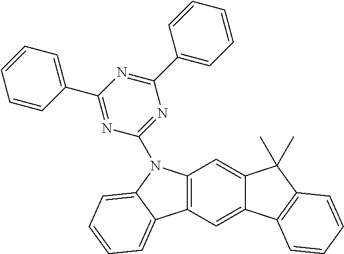 [SMILES: CC1(C)c2ccccc2-c2cc3c4ccccc4n(-c4nc(-c5ccccc5)nc(-c5ccccc5)n4)c3cc21]

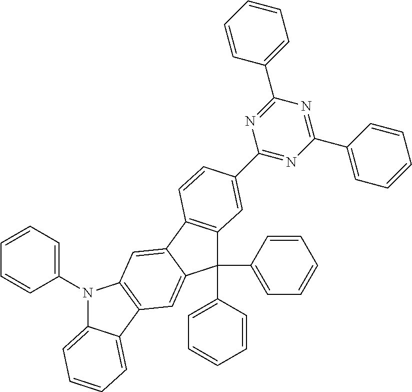 [SMILES: c1ccc(-c2nc(-c3ccccc3)nc(-c3ccc4c(c3)C(c3ccccc3)(c3ccccc3)c3cc5c6ccccc6n(-c6ccccc6)c5cc3-4)n2)cc1]

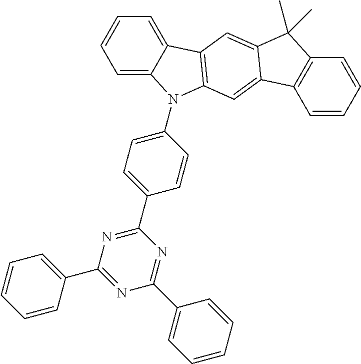 [SMILES: CC1(C)c2ccccc2-c2cc3c(cc21)c1ccccc1n3-c1ccc(-c2nc(-c3ccccc3)nc(-c3ccccc3)n2)cc1]

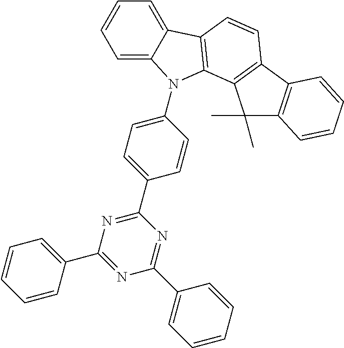 [SMILES: CC1(C)c2ccccc2-c2ccc3c4ccccc4n(-c4ccc(-c5nc(-c6ccccc6)nc(-c6ccccc6)n5)cc4)c3c21]

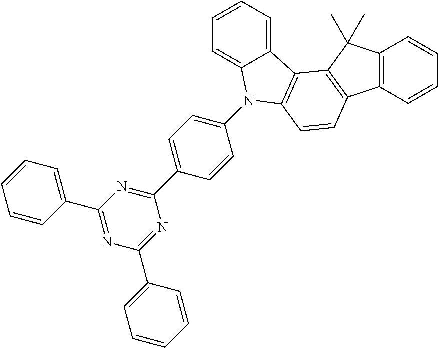 [SMILES: CC1(C)c2ccccc2-c2ccc3c(c21)c1ccccc1n3-c1ccc(-c2nc(-c3ccccc3)nc(-c3ccccc3)n2)cc1]

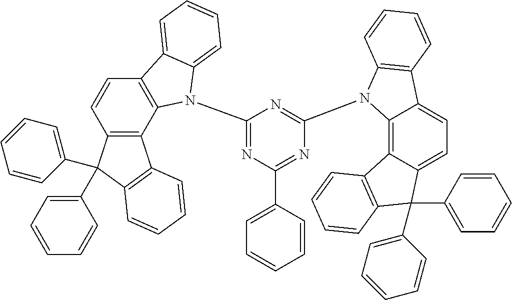 [SMILES: c1ccc(-c2nc(-n3c4ccccc4c4ccc5c(c43)-c3ccccc3C5(c3ccccc3)c3ccccc3)nc(-n3c4ccccc4c4ccc5c(c43)-c3ccccc3C5(c3ccccc3)c3ccccc3)n2)cc1]

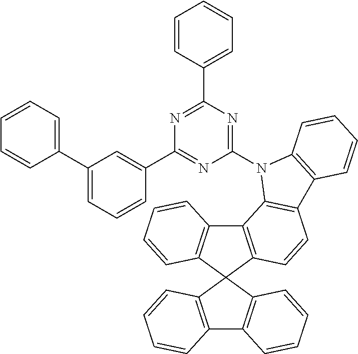 [SMILES: c1ccc(-c2cccc(-c3nc(-c4ccccc4)nc(-n4c5ccccc5c5ccc6c(c54)-c4ccccc4C64c5ccccc5-c5ccccc54)n3)c2)cc1]

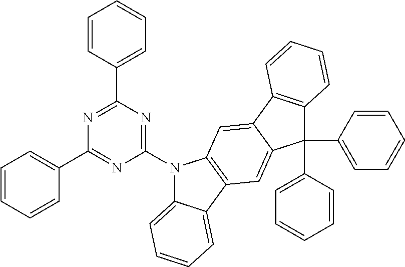 [SMILES: c1ccc(-c2nc(-c3ccccc3)nc(-n3c4ccccc4c4cc5c(cc43)-c3ccccc3C5(c3ccccc3)c3ccccc3)n2)cc1]

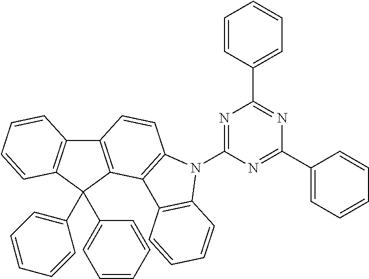 [SMILES: c1ccc(-c2nc(-c3ccccc3)nc(-n3c4ccccc4c4c5c(ccc43)-c3ccccc3C5(c3ccccc3)c3ccccc3)n2)cc1]

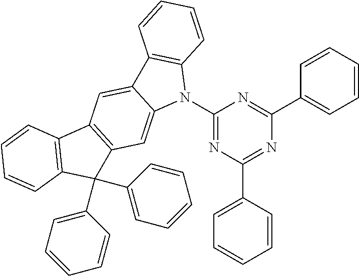 [SMILES: c1ccc(-c2nc(-c3ccccc3)nc(-n3c4ccccc4c4cc5c(cc43)C(c3ccccc3)(c3ccccc3)c3ccccc3-5)n2)cc1]

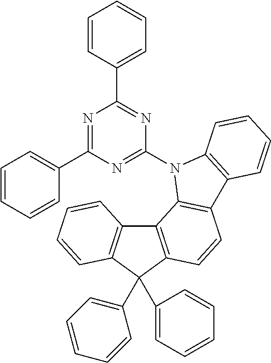 [SMILES: c1ccc(-c2nc(-c3ccccc3)nc(-n3c4ccccc4c4ccc5c(c43)-c3ccccc3C5(c3ccccc3)c3ccccc3)n2)cc1]

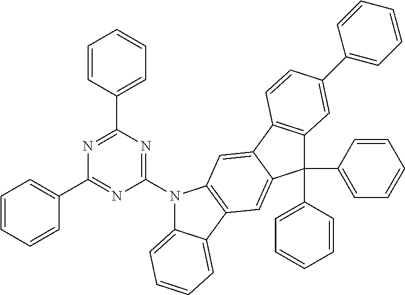 [SMILES: c1ccc(-c2ccc3c(c2)C(c2ccccc2)(c2ccccc2)c2cc4c5ccccc5n(-c5nc(-c6ccccc6)nc(-c6ccccc6)n5)c4cc2-3)cc1]

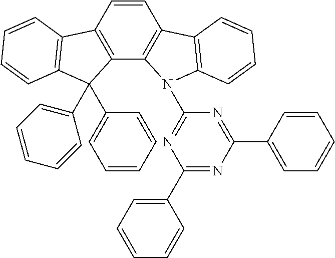 [SMILES: c1ccc(-c2nc(-c3ccccc3)nc(-n3c4ccccc4c4ccc5c(c43)C(c3ccccc3)(c3ccccc3)c3ccccc3-5)n2)cc1]

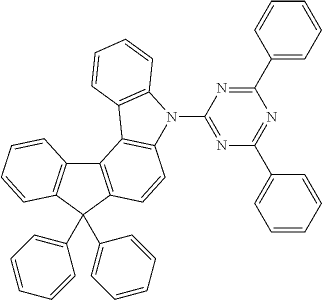 [SMILES: c1ccc(-c2nc(-c3ccccc3)nc(-n3c4ccccc4c4c5c(ccc43)C(c3ccccc3)(c3ccccc3)c3ccccc3-5)n2)cc1]

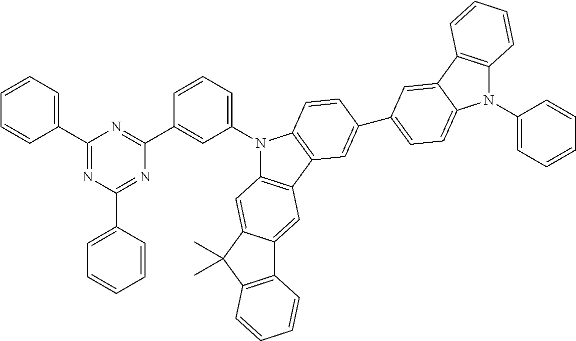 [SMILES: CC1(C)c2ccccc2-c2cc3c4cc(-c5ccc6c(c5)c5ccccc5n6-c5ccccc5)ccc4n(-c4cccc(-c5nc(-c6ccccc6)nc(-c6ccccc6)n5)c4)c3cc21]

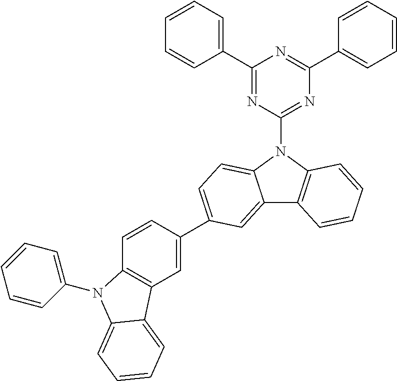 [SMILES: c1ccc(-c2nc(-c3ccccc3)nc(-n3c4ccccc4c4cc(-c5ccc6c(c5)c5ccccc5n6-c5ccccc5)ccc43)n2)cc1]

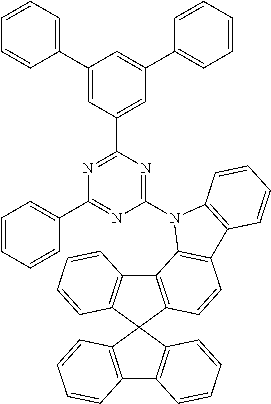 [SMILES: c1ccc(-c2cc(-c3ccccc3)cc(-c3nc(-c4ccccc4)nc(-n4c5ccccc5c5ccc6c(c54)-c4ccccc4C64c5ccccc5-c5ccccc54)n3)c2)cc1]

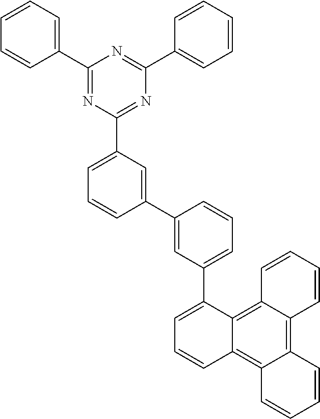 [SMILES: c1ccc(-c2nc(-c3ccccc3)nc(-c3cccc(-c4cccc(-c5cccc6c7ccccc7c7ccccc7c56)c4)c3)n2)cc1]

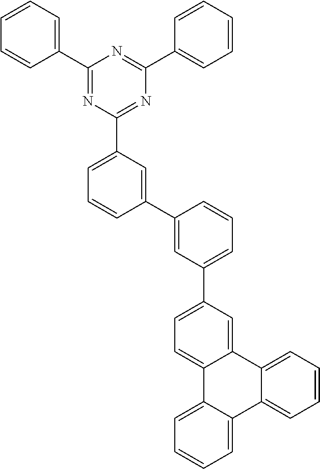 [SMILES: c1ccc(-c2nc(-c3ccccc3)nc(-c3cccc(-c4cccc(-c5ccc6c7ccccc7c7ccccc7c6c5)c4)c3)n2)cc1]

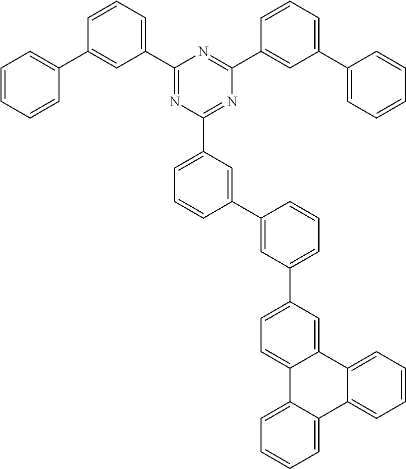 [SMILES: c1ccc(-c2cccc(-c3nc(-c4cccc(-c5ccccc5)c4)nc(-c4cccc(-c5cccc(-c6ccc7c8ccccc8c8ccccc8c7c6)c5)c4)n3)c2)cc1]

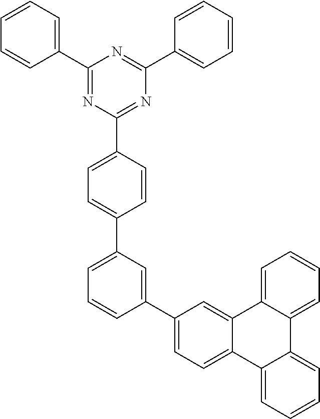 [SMILES: c1ccc(-c2nc(-c3ccccc3)nc(-c3ccc(-c4cccc(-c5ccc6c7ccccc7c7ccccc7c6c5)c4)cc3)n2)cc1]

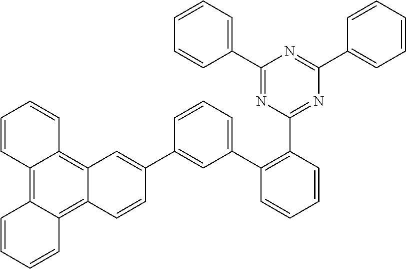 [SMILES: c1ccc(-c2nc(-c3ccccc3)nc(-c3ccccc3-c3cccc(-c4ccc5c6ccccc6c6ccccc6c5c4)c3)n2)cc1]

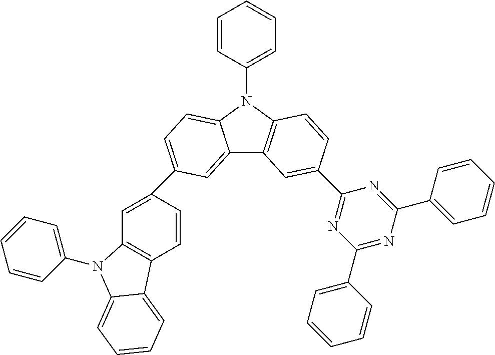 [SMILES: c1ccc(-c2nc(-c3ccccc3)nc(-c3ccc4c(c3)c3cc(-c5ccc6c7ccccc7n(-c7ccccc7)c6c5)ccc3n4-c3ccccc3)n2)cc1]

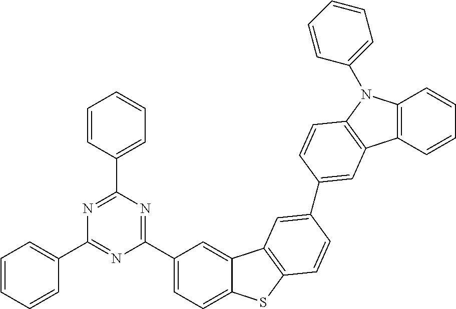 [SMILES: c1ccc(-c2nc(-c3ccccc3)nc(-c3ccc4sc5ccc(-c6ccc7c(c6)c6ccccc6n7-c6ccccc6)cc5c4c3)n2)cc1]

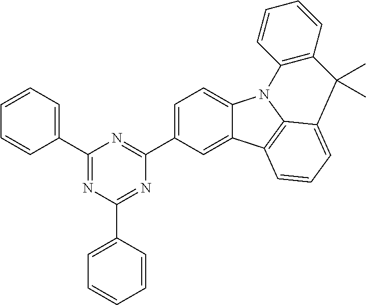 [SMILES: CC1(C)c2ccccc2-n2c3ccc(-c4nc(-c5ccccc5)nc(-c5ccccc5)n4)cc3c3cccc1c32]

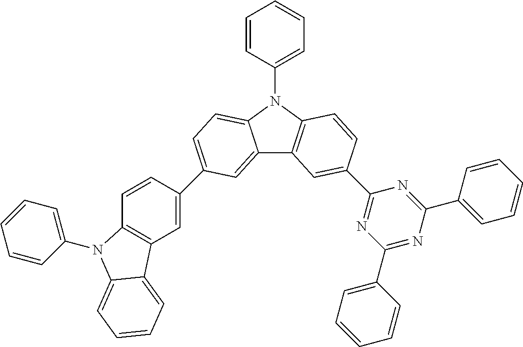 [SMILES: c1ccc(-c2nc(-c3ccccc3)nc(-c3ccc4c(c3)c3cc(-c5ccc6c(c5)c5ccccc5n6-c5ccccc5)ccc3n4-c3ccccc3)n2)cc1]

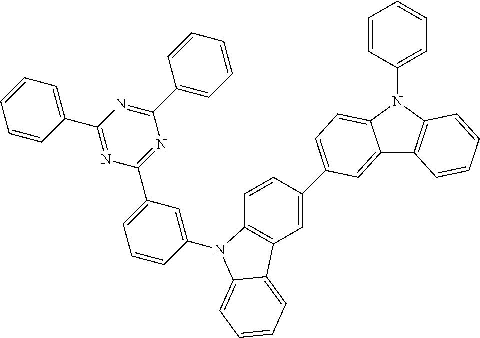 [SMILES: c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1]

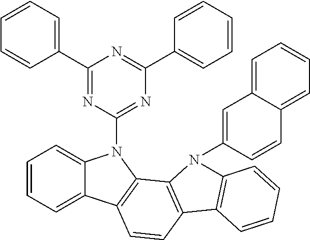 [SMILES: c1ccc(-c2nc(-c3ccccc3)nc(-n3c4ccccc4c4ccc5c6ccccc6n(-c6ccc7ccccc7c6)c5c43)n2)cc1]

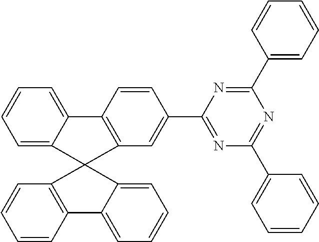 [SMILES: c1ccc(-c2nc(-c3ccccc3)nc(-c3ccc4c(c3)C3(c5ccccc5-c5ccccc53)c3ccccc3-4)n2)cc1]

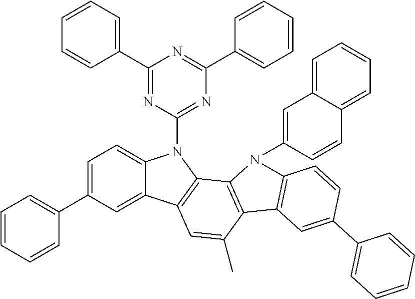 [SMILES: Cc1cc2c3cc(-c4ccccc4)ccc3n(-c3nc(-c4ccccc4)nc(-c4ccccc4)n3)c2c2c1c1cc(-c3ccccc3)ccc1n2-c1ccc2ccccc2c1]

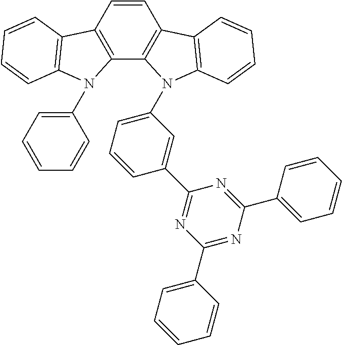 [SMILES: c1ccc(-c2nc(-c3ccccc3)nc(-c3cccc(-n4c5ccccc5c5ccc6c7ccccc7n(-c7ccccc7)c6c54)c3)n2)cc1]

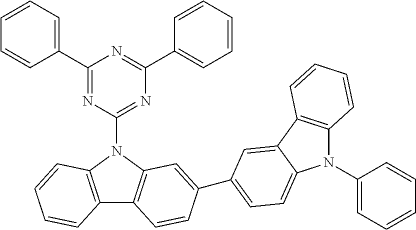 [SMILES: c1ccc(-c2nc(-c3ccccc3)nc(-n3c4ccccc4c4ccc(-c5ccc6c(c5)c5ccccc5n6-c5ccccc5)cc43)n2)cc1]

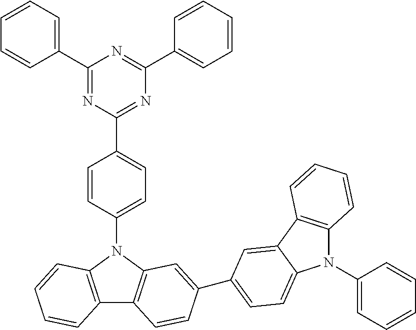 [SMILES: c1ccc(-c2nc(-c3ccccc3)nc(-c3ccc(-n4c5ccccc5c5ccc(-c6ccc7c(c6)c6ccccc6n7-c6ccccc6)cc54)cc3)n2)cc1]

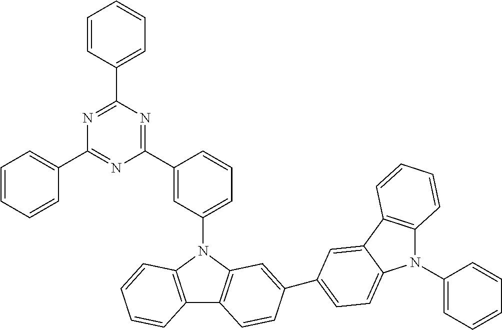 [SMILES: c1ccc(-c2nc(-c3ccccc3)nc(-c3cccc(-n4c5ccccc5c5ccc(-c6ccc7c(c6)c6ccccc6n7-c6ccccc6)cc54)c3)n2)cc1]

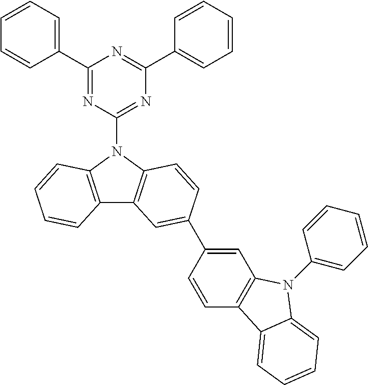 [SMILES: c1ccc(-c2nc(-c3ccccc3)nc(-n3c4ccccc4c4cc(-c5ccc6c7ccccc7n(-c7ccccc7)c6c5)ccc43)n2)cc1]

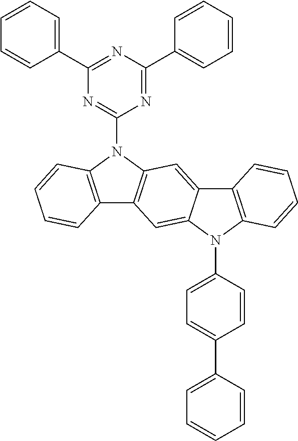 [SMILES: c1ccc(-c2ccc(-n3c4ccccc4c4cc5c(cc43)c3ccccc3n5-c3nc(-c4ccccc4)nc(-c4ccccc4)n3)cc2)cc1]

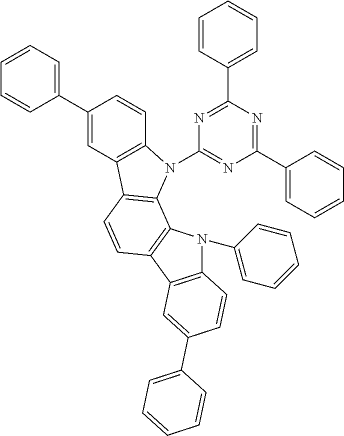 [SMILES: c1ccc(-c2ccc3c(c2)c2ccc4c5cc(-c6ccccc6)ccc5n(-c5nc(-c6ccccc6)nc(-c6ccccc6)n5)c4c2n3-c2ccccc2)cc1]

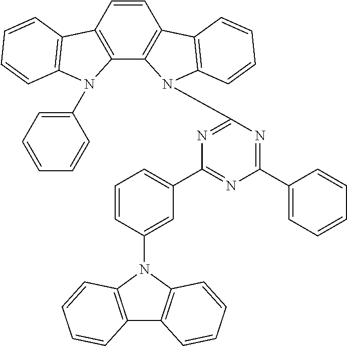 [SMILES: c1ccc(-c2nc(-c3cccc(-n4c5ccccc5c5ccccc54)c3)nc(-n3c4ccccc4c4ccc5c6ccccc6n(-c6ccccc6)c5c43)n2)cc1]

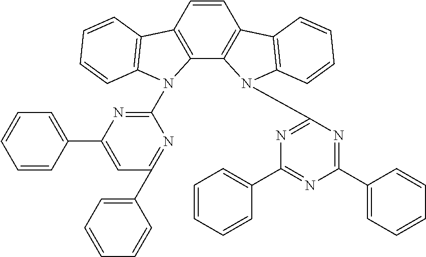 [SMILES: c1ccc(-c2cc(-c3ccccc3)nc(-n3c4ccccc4c4ccc5c6ccccc6n(-c6nc(-c7ccccc7)nc(-c7ccccc7)n6)c5c43)n2)cc1]

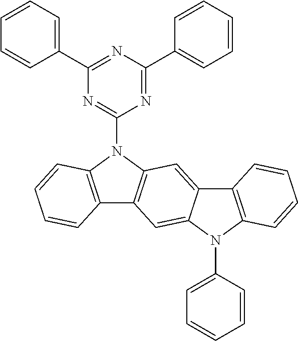 [SMILES: c1ccc(-c2nc(-c3ccccc3)nc(-n3c4ccccc4c4cc5c(cc43)c3ccccc3n5-c3ccccc3)n2)cc1]

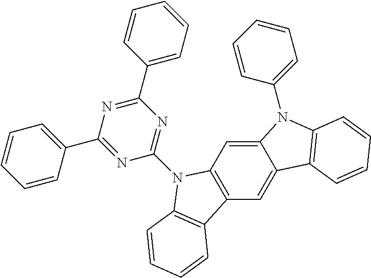 [SMILES: c1ccc(-c2nc(-c3ccccc3)nc(-n3c4ccccc4c4cc5c6ccccc6n(-c6ccccc6)c5cc43)n2)cc1]

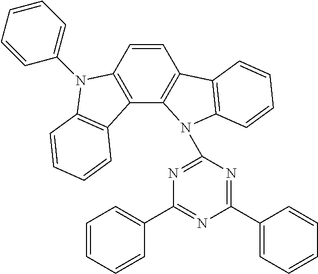 [SMILES: c1ccc(-c2nc(-c3ccccc3)nc(-n3c4ccccc4c4ccc5c(c6ccccc6n5-c5ccccc5)c43)n2)cc1]

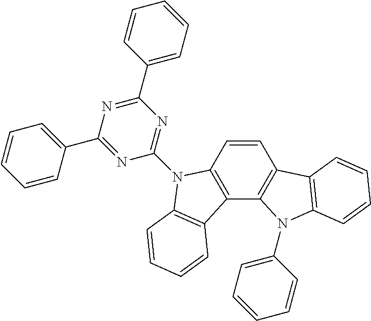 [SMILES: c1ccc(-c2nc(-c3ccccc3)nc(-n3c4ccccc4c4c3ccc3c5ccccc5n(-c5ccccc5)c34)n2)cc1]

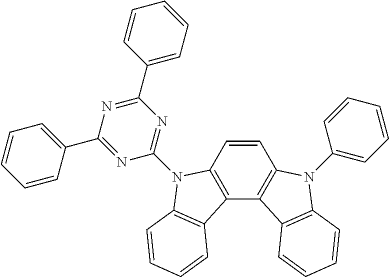 [SMILES: c1ccc(-c2nc(-c3ccccc3)nc(-n3c4ccccc4c4c5c6ccccc6n(-c6ccccc6)c5ccc43)n2)cc1]

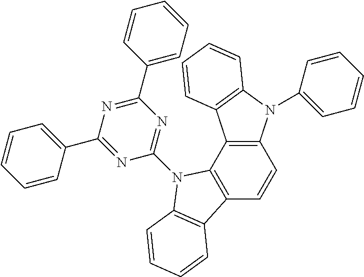 [SMILES: c1ccc(-c2nc(-c3ccccc3)nc(-n3c4ccccc4c4ccc5c(c6ccccc6n5-c5ccccc5)c43)n2)cc1]

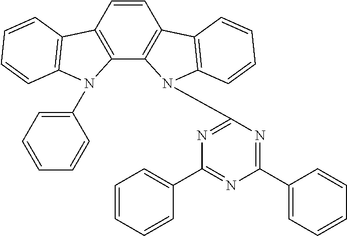 [SMILES: c1ccc(-c2nc(-c3ccccc3)nc(-n3c4ccccc4c4ccc5c6ccccc6n(-c6ccccc6)c5c43)n2)cc1]

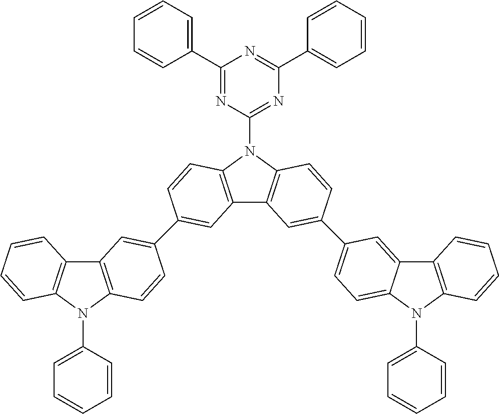 [SMILES: c1ccc(-c2nc(-c3ccccc3)nc(-n3c4ccc(-c5ccc6c(c5)c5ccccc5n6-c5ccccc5)cc4c4cc(-c5ccc6c(c5)c5ccccc5n6-c5ccccc5)ccc43)n2)cc1]

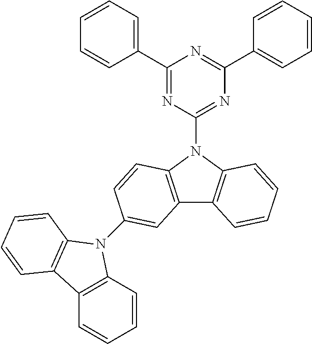 [SMILES: c1ccc(-c2nc(-c3ccccc3)nc(-n3c4ccccc4c4cc(-n5c6ccccc6c6ccccc65)ccc43)n2)cc1]

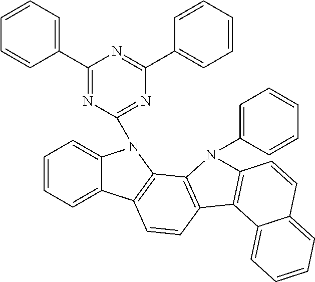 [SMILES: c1ccc(-c2nc(-c3ccccc3)nc(-n3c4ccccc4c4ccc5c6c7ccccc7ccc6n(-c6ccccc6)c5c43)n2)cc1]

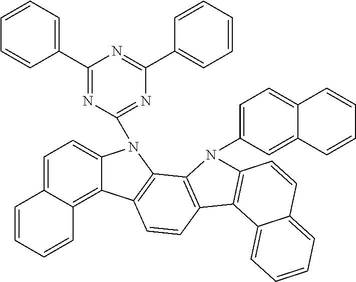 [SMILES: c1ccc(-c2nc(-c3ccccc3)nc(-n3c4ccc5ccccc5c4c4ccc5c6c7ccccc7ccc6n(-c6ccc7ccccc7c6)c5c43)n2)cc1]

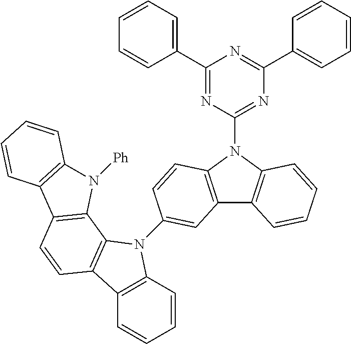 [SMILES: c1ccc(-c2nc(-c3ccccc3)nc(-n3c4ccccc4c4cc(-n5c6ccccc6c6ccc7c8ccccc8n(-c8ccccc8)c7c65)ccc43)n2)cc1]

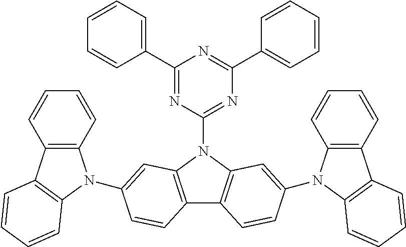 [SMILES: c1ccc(-c2nc(-c3ccccc3)nc(-n3c4cc(-n5c6ccccc6c6ccccc65)ccc4c4ccc(-n5c6ccccc6c6ccccc65)cc43)n2)cc1]

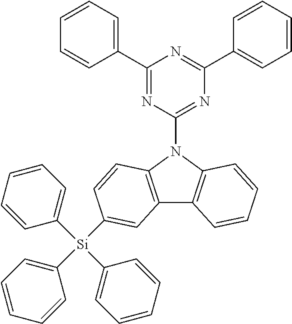 [SMILES: c1ccc(-c2nc(-c3ccccc3)nc(-n3c4ccccc4c4cc([Si](c5ccccc5)(c5ccccc5)c5ccccc5)ccc43)n2)cc1]

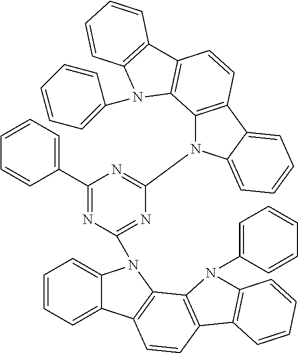 [SMILES: c1ccc(-c2nc(-n3c4ccccc4c4ccc5c6ccccc6n(-c6ccccc6)c5c43)nc(-n3c4ccccc4c4ccc5c6ccccc6n(-c6ccccc6)c5c43)n2)cc1]

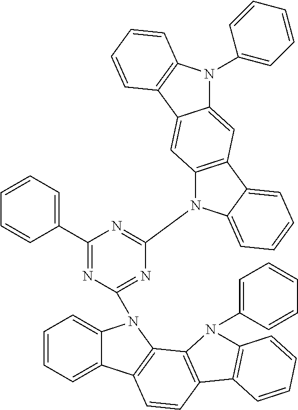 [SMILES: c1ccc(-c2nc(-n3c4ccccc4c4cc5c(cc43)c3ccccc3n5-c3ccccc3)nc(-n3c4ccccc4c4ccc5c6ccccc6n(-c6ccccc6)c5c43)n2)cc1]

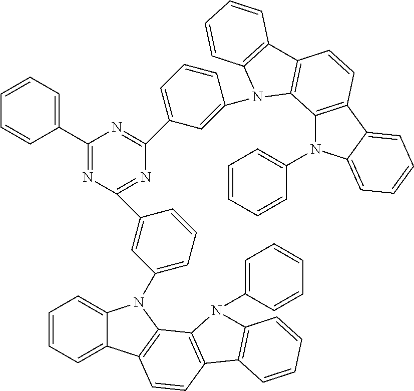 [SMILES: c1ccc(-c2nc(-c3cccc(-n4c5ccccc5c5ccc6c7ccccc7n(-c7ccccc7)c6c54)c3)nc(-c3cccc(-n4c5ccccc5c5ccc6c7ccccc7n(-c7ccccc7)c6c54)c3)n2)cc1]

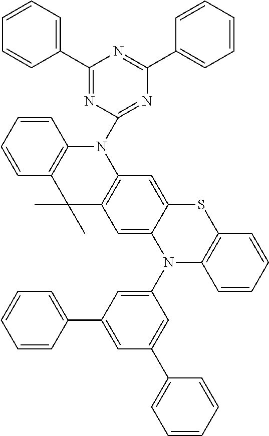 [SMILES: CC1(C)c2ccccc2N(c2nc(-c3ccccc3)nc(-c3ccccc3)n2)c2cc3c(cc21)N(c1cc(-c2ccccc2)cc(-c2ccccc2)c1)c1ccccc1S3]

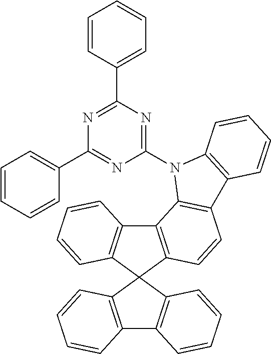 [SMILES: c1ccc(-c2nc(-c3ccccc3)nc(-n3c4ccccc4c4ccc5c(c43)-c3ccccc3C53c4ccccc4-c4ccccc43)n2)cc1]

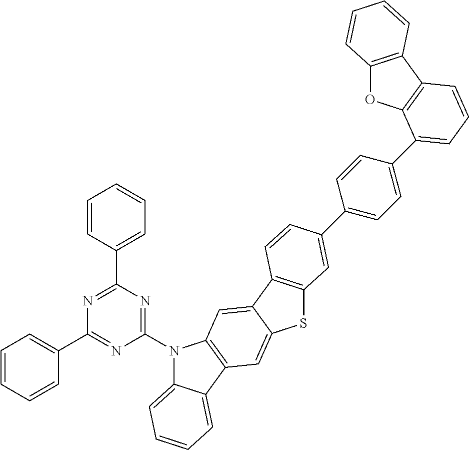 [SMILES: c1ccc(-c2nc(-c3ccccc3)nc(-n3c4ccccc4c4cc5sc6cc(-c7ccc(-c8cccc9c8oc8ccccc89)cc7)ccc6c5cc43)n2)cc1]

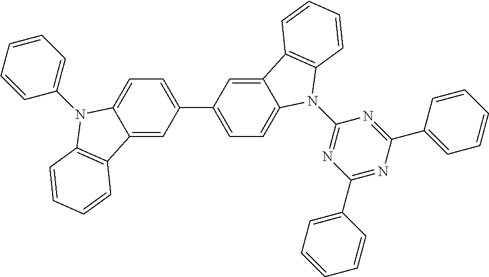 [SMILES: c1ccc(-c2nc(-c3ccccc3)nc(-n3c4ccccc4c4cc(-c5ccc6c(c5)c5ccccc5n6-c5ccccc5)ccc43)n2)cc1]

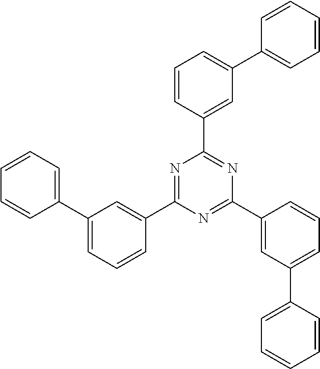 [SMILES: c1ccc(-c2cccc(-c3nc(-c4cccc(-c5ccccc5)c4)nc(-c4cccc(-c5ccccc5)c4)n3)c2)cc1]

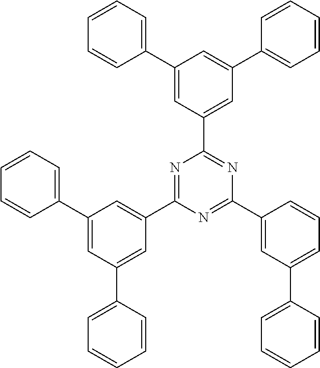 [SMILES: c1ccc(-c2cccc(-c3nc(-c4cc(-c5ccccc5)cc(-c5ccccc5)c4)nc(-c4cc(-c5ccccc5)cc(-c5ccccc5)c4)n3)c2)cc1]

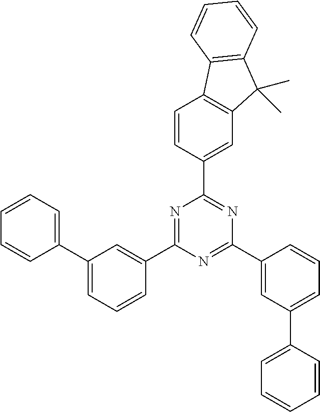 [SMILES: CC1(C)c2ccccc2-c2ccc(-c3nc(-c4cccc(-c5ccccc5)c4)nc(-c4cccc(-c5ccccc5)c4)n3)cc21]

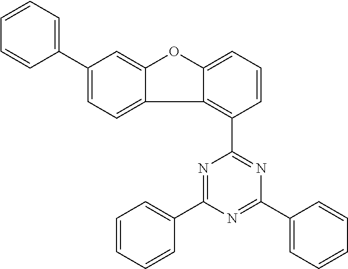 [SMILES: c1ccc(-c2ccc3c(c2)oc2cccc(-c4nc(-c5ccccc5)nc(-c5ccccc5)n4)c23)cc1]

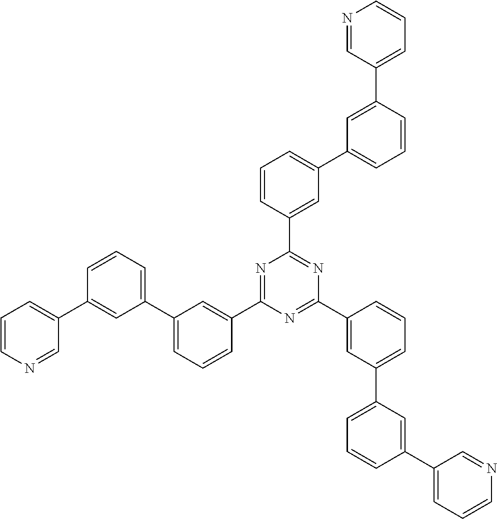 [SMILES: c1cncc(-c2cccc(-c3cccc(-c4nc(-c5cccc(-c6cccc(-c7cccnc7)c6)c5)nc(-c5cccc(-c6cccc(-c7cccnc7)c6)c5)n4)c3)c2)c1]

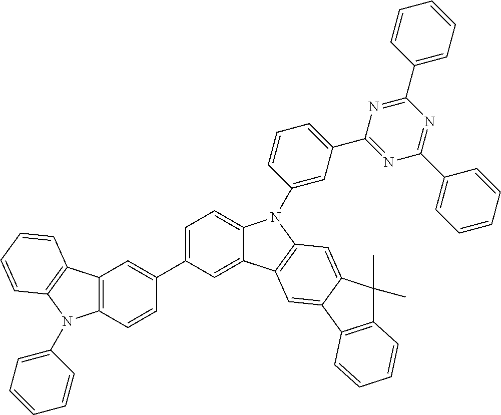 [SMILES: CC1(C)c2ccccc2-c2cc3c4cc(-c5ccc6c(c5)c5ccccc5n6-c5ccccc5)ccc4n(-c4cccc(-c5nc(-c6ccccc6)nc(-c6ccccc6)n5)c4)c3cc21]

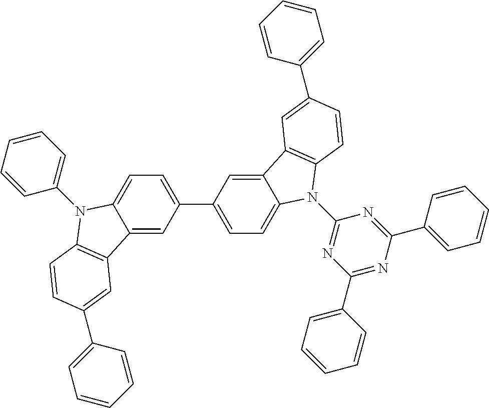 [SMILES: c1ccc(-c2ccc3c(c2)c2cc(-c4ccc5c(c4)c4cc(-c6ccccc6)ccc4n5-c4nc(-c5ccccc5)nc(-c5ccccc5)n4)ccc2n3-c2ccccc2)cc1]

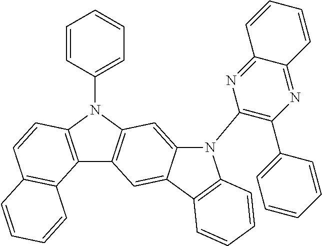 [SMILES: c1ccc(-c2nc3ccccc3nc2-n2c3ccccc3c3cc4c5c6ccccc6ccc5n(-c5ccccc5)c4cc32)cc1]

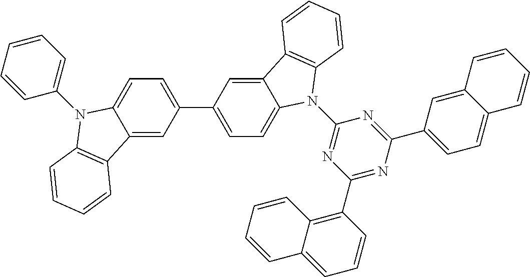 [SMILES: c1ccc(-n2c3ccccc3c3cc(-c4ccc5c(c4)c4ccccc4n5-c4nc(-c5ccc6ccccc6c5)nc(-c5cccc6ccccc56)n4)ccc32)cc1]

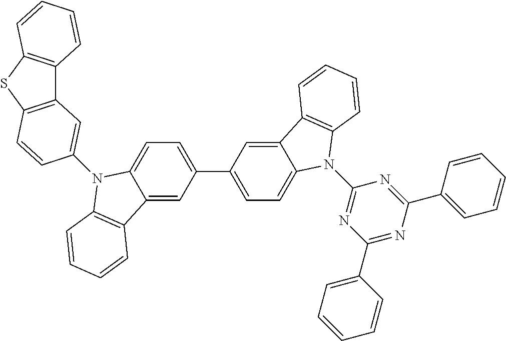 [SMILES: c1ccc(-c2nc(-c3ccccc3)nc(-n3c4ccccc4c4cc(-c5ccc6c(c5)c5ccccc5n6-c5ccc6sc7ccccc7c6c5)ccc43)n2)cc1]

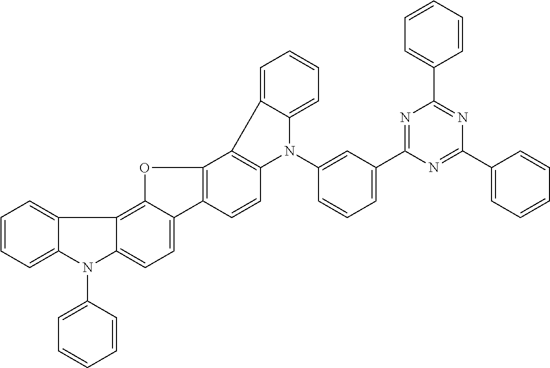 [SMILES: c1ccc(-c2nc(-c3ccccc3)nc(-c3cccc(-n4c5ccccc5c5c6oc7c(ccc8c7c7ccccc7n8-c7ccccc7)c6ccc54)c3)n2)cc1]

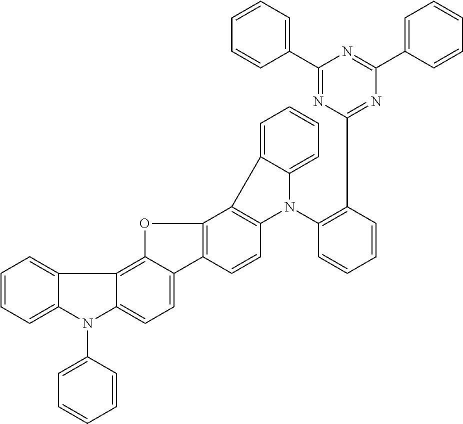 [SMILES: c1ccc(-c2nc(-c3ccccc3)nc(-c3ccccc3-n3c4ccccc4c4c5oc6c(ccc7c6c6ccccc6n7-c6ccccc6)c5ccc43)n2)cc1]

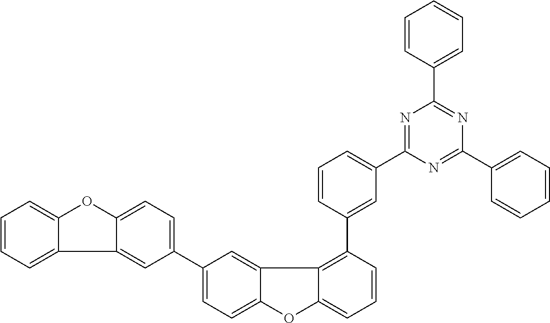 [SMILES: c1ccc(-c2nc(-c3ccccc3)nc(-c3cccc(-c4cccc5oc6ccc(-c7ccc8oc9ccccc9c8c7)cc6c45)c3)n2)cc1]

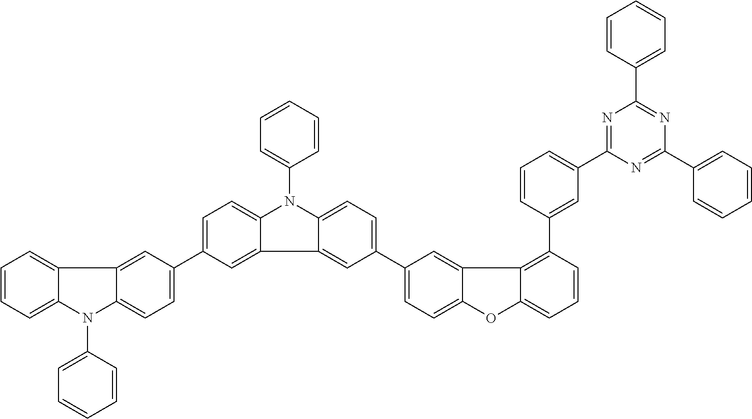 [SMILES: c1ccc(-c2nc(-c3ccccc3)nc(-c3cccc(-c4cccc5oc6ccc(-c7ccc8c(c7)c7cc(-c9ccc%10c(c9)c9ccccc9n%10-c9ccccc9)ccc7n8-c7ccccc7)cc6c45)c3)n2)cc1]

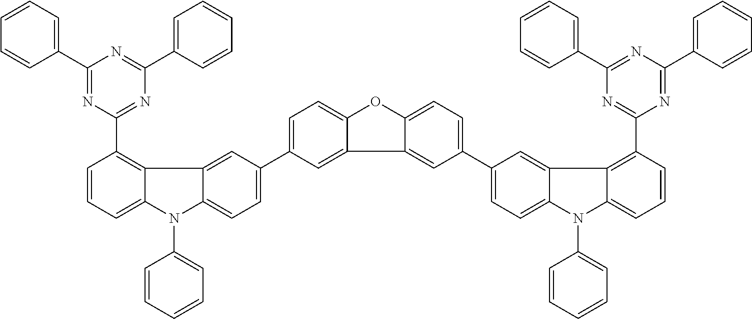 [SMILES: c1ccc(-c2nc(-c3ccccc3)nc(-c3cccc4c3c3cc(-c5ccc6oc7ccc(-c8ccc9c(c8)c8c(-c%10nc(-c%11ccccc%11)nc(-c%11ccccc%11)n%10)cccc8n9-c8ccccc8)cc7c6c5)ccc3n4-c3ccccc3)n2)cc1]

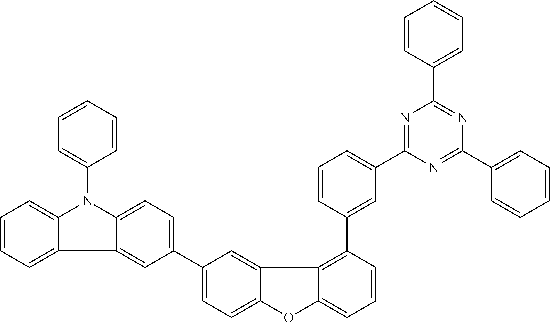 [SMILES: c1ccc(-c2nc(-c3ccccc3)nc(-c3cccc(-c4cccc5oc6ccc(-c7ccc8c(c7)c7ccccc7n8-c7ccccc7)cc6c45)c3)n2)cc1]

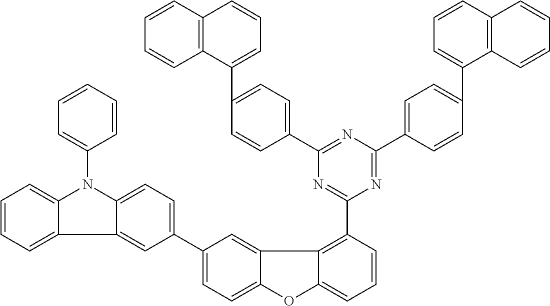 [SMILES: c1ccc(-n2c3ccccc3c3cc(-c4ccc5oc6cccc(-c7nc(-c8ccc(-c9cccc%10ccccc9%10)cc8)nc(-c8ccc(-c9cccc%10ccccc9%10)cc8)n7)c6c5c4)ccc32)cc1]

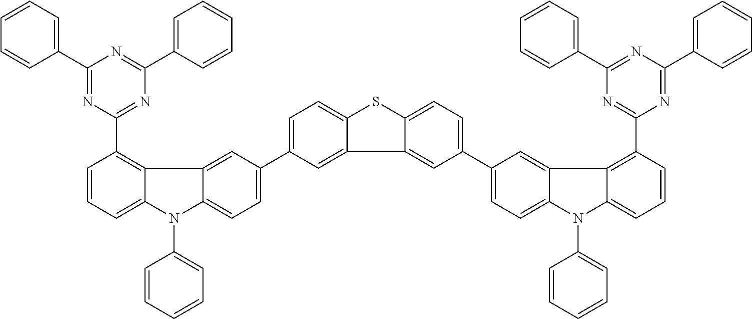 [SMILES: c1ccc(-c2nc(-c3ccccc3)nc(-c3cccc4c3c3cc(-c5ccc6sc7ccc(-c8ccc9c(c8)c8c(-c%10nc(-c%11ccccc%11)nc(-c%11ccccc%11)n%10)cccc8n9-c8ccccc8)cc7c6c5)ccc3n4-c3ccccc3)n2)cc1]

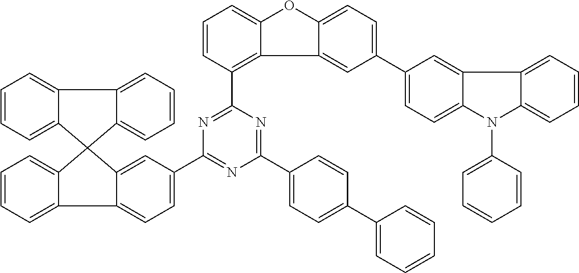 [SMILES: c1ccc(-c2ccc(-c3nc(-c4ccc5c(c4)C4(c6ccccc6-c6ccccc64)c4ccccc4-5)nc(-c4cccc5oc6ccc(-c7ccc8c(c7)c7ccccc7n8-c7ccccc7)cc6c45)n3)cc2)cc1]

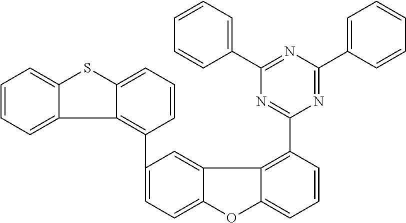 [SMILES: c1ccc(-c2nc(-c3ccccc3)nc(-c3cccc4oc5ccc(-c6cccc7sc8ccccc8c67)cc5c34)n2)cc1]

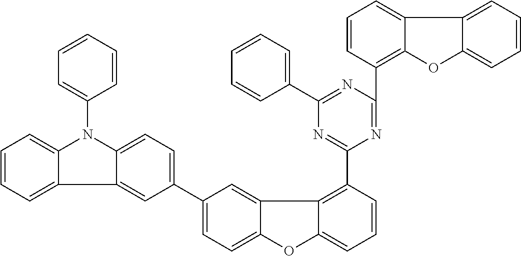 [SMILES: c1ccc(-c2nc(-c3cccc4c3oc3ccccc34)nc(-c3cccc4oc5ccc(-c6ccc7c(c6)c6ccccc6n7-c6ccccc6)cc5c34)n2)cc1]

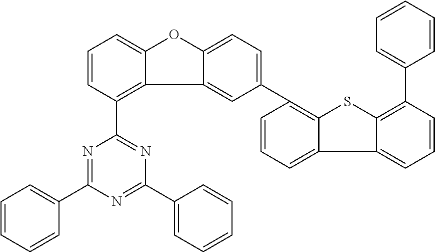 [SMILES: c1ccc(-c2nc(-c3ccccc3)nc(-c3cccc4oc5ccc(-c6cccc7c6sc6c(-c8ccccc8)cccc67)cc5c34)n2)cc1]

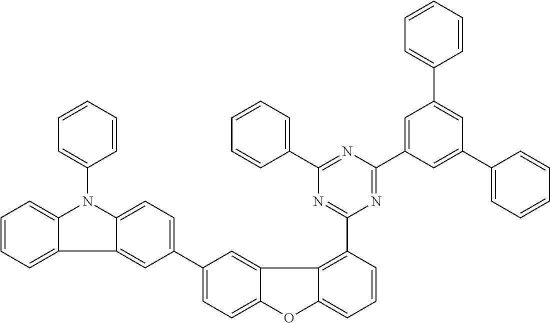 [SMILES: c1ccc(-c2cc(-c3ccccc3)cc(-c3nc(-c4ccccc4)nc(-c4cccc5oc6ccc(-c7ccc8c(c7)c7ccccc7n8-c7ccccc7)cc6c45)n3)c2)cc1]

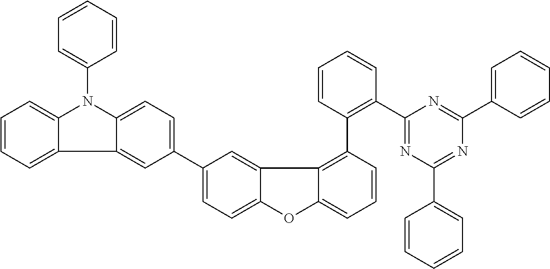 [SMILES: c1ccc(-c2nc(-c3ccccc3)nc(-c3ccccc3-c3cccc4oc5ccc(-c6ccc7c(c6)c6ccccc6n7-c6ccccc6)cc5c34)n2)cc1]

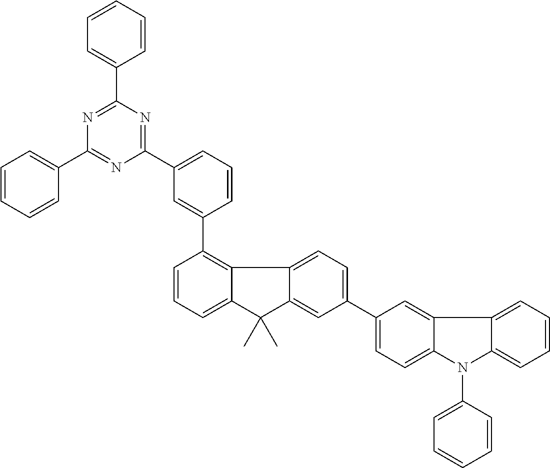 [SMILES: CC1(C)c2cc(-c3ccc4c(c3)c3ccccc3n4-c3ccccc3)ccc2-c2c(-c3cccc(-c4nc(-c5ccccc5)nc(-c5ccccc5)n4)c3)cccc21]

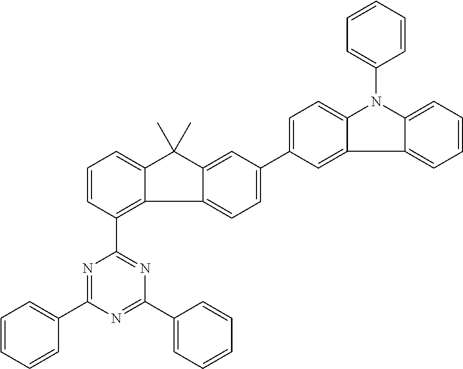 [SMILES: CC1(C)c2cc(-c3ccc4c(c3)c3ccccc3n4-c3ccccc3)ccc2-c2c(-c3nc(-c4ccccc4)nc(-c4ccccc4)n3)cccc21]

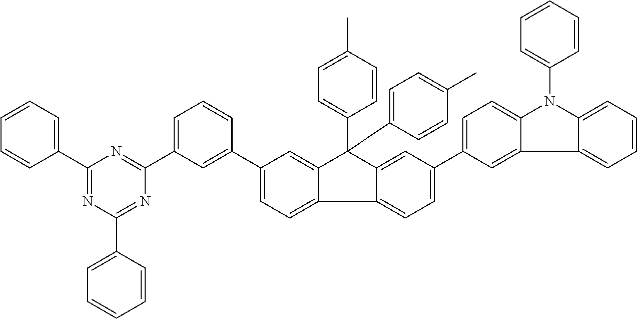 [SMILES: Cc1ccc(C2(c3ccc(C)cc3)c3cc(-c4cccc(-c5nc(-c6ccccc6)nc(-c6ccccc6)n5)c4)ccc3-c3ccc(-c4ccc5c(c4)c4ccccc4n5-c4ccccc4)cc32)cc1]

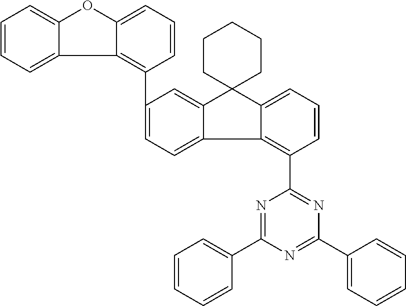 [SMILES: c1ccc(-c2nc(-c3ccccc3)nc(-c3cccc4c3-c3ccc(-c5cccc6oc7ccccc7c56)cc3C43CCCCC3)n2)cc1]

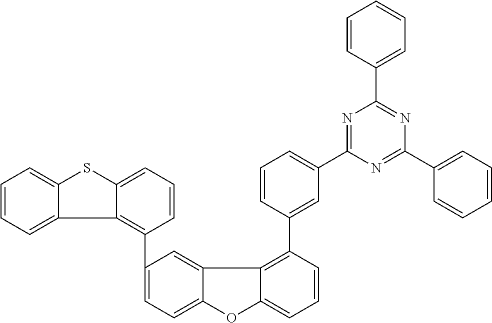 [SMILES: c1ccc(-c2nc(-c3ccccc3)nc(-c3cccc(-c4cccc5oc6ccc(-c7cccc8sc9ccccc9c78)cc6c45)c3)n2)cc1]

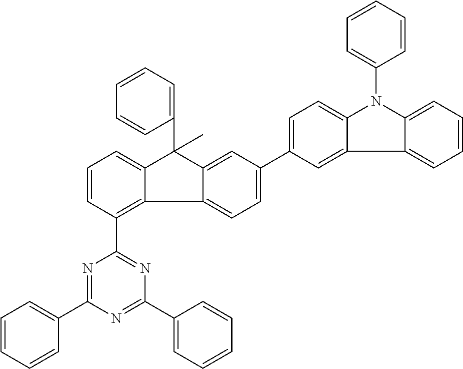 [SMILES: CC1(c2ccccc2)c2cc(-c3ccc4c(c3)c3ccccc3n4-c3ccccc3)ccc2-c2c(-c3nc(-c4ccccc4)nc(-c4ccccc4)n3)cccc21]

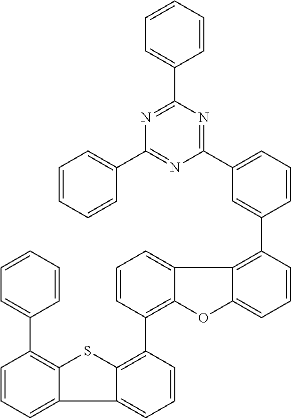 [SMILES: c1ccc(-c2nc(-c3ccccc3)nc(-c3cccc(-c4cccc5oc6c(-c7cccc8c7sc7c(-c9ccccc9)cccc78)cccc6c45)c3)n2)cc1]

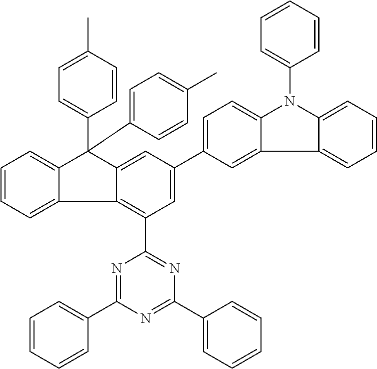 [SMILES: Cc1ccc(C2(c3ccc(C)cc3)c3ccccc3-c3c(-c4nc(-c5ccccc5)nc(-c5ccccc5)n4)cc(-c4ccc5c(c4)c4ccccc4n5-c4ccccc4)cc32)cc1]

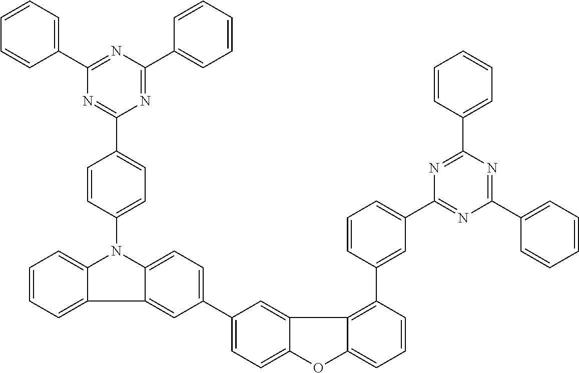 [SMILES: c1ccc(-c2nc(-c3ccccc3)nc(-c3ccc(-n4c5ccccc5c5cc(-c6ccc7oc8cccc(-c9cccc(-c%10nc(-c%11ccccc%11)nc(-c%11ccccc%11)n%10)c9)c8c7c6)ccc54)cc3)n2)cc1]

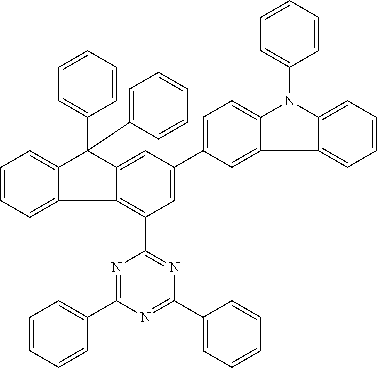 [SMILES: c1ccc(-c2nc(-c3ccccc3)nc(-c3cc(-c4ccc5c(c4)c4ccccc4n5-c4ccccc4)cc4c3-c3ccccc3C4(c3ccccc3)c3ccccc3)n2)cc1]

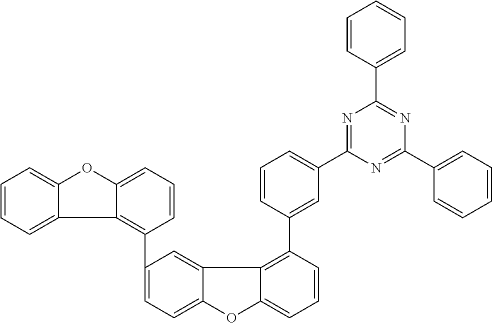 [SMILES: c1ccc(-c2nc(-c3ccccc3)nc(-c3cccc(-c4cccc5oc6ccc(-c7cccc8oc9ccccc9c78)cc6c45)c3)n2)cc1]

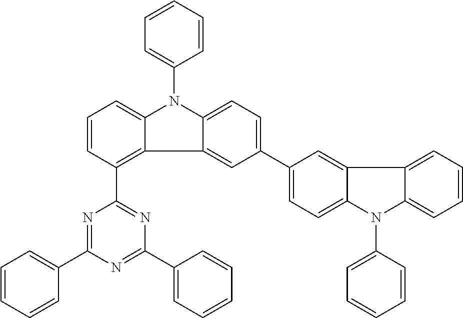 [SMILES: c1ccc(-c2nc(-c3ccccc3)nc(-c3cccc4c3c3cc(-c5ccc6c(c5)c5ccccc5n6-c5ccccc5)ccc3n4-c3ccccc3)n2)cc1]

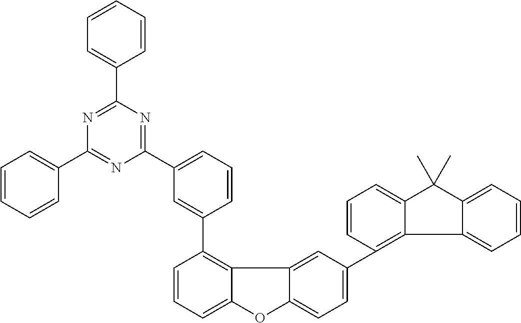 [SMILES: CC1(C)c2ccccc2-c2c(-c3ccc4oc5cccc(-c6cccc(-c7nc(-c8ccccc8)nc(-c8ccccc8)n7)c6)c5c4c3)cccc21]

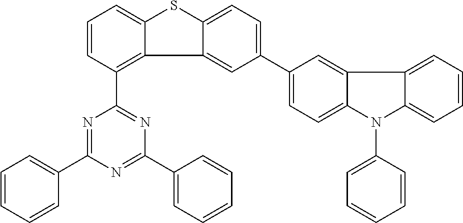 [SMILES: c1ccc(-c2nc(-c3ccccc3)nc(-c3cccc4sc5ccc(-c6ccc7c(c6)c6ccccc6n7-c6ccccc6)cc5c34)n2)cc1]

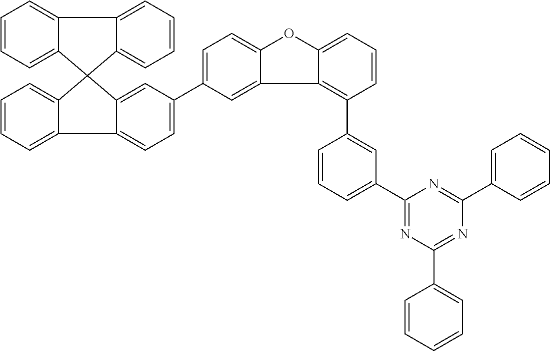 [SMILES: c1ccc(-c2nc(-c3ccccc3)nc(-c3cccc(-c4cccc5oc6ccc(-c7ccc8c(c7)C7(c9ccccc9-c9ccccc97)c7ccccc7-8)cc6c45)c3)n2)cc1]

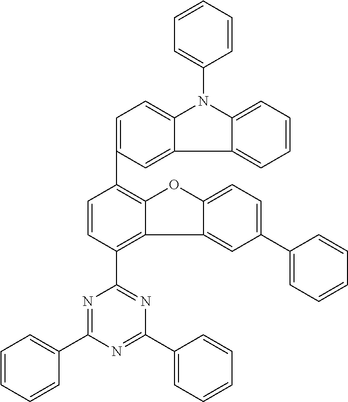 [SMILES: c1ccc(-c2ccc3oc4c(-c5ccc6c(c5)c5ccccc5n6-c5ccccc5)ccc(-c5nc(-c6ccccc6)nc(-c6ccccc6)n5)c4c3c2)cc1]

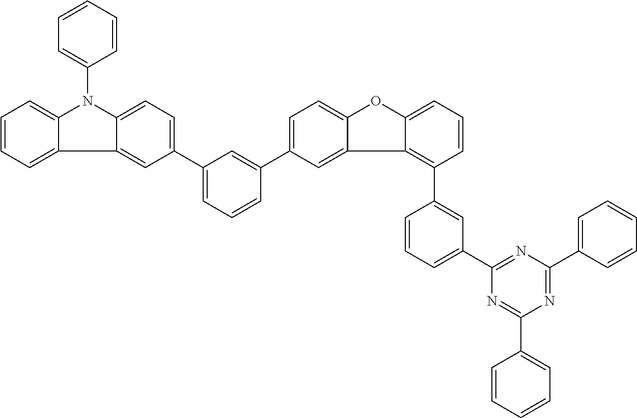 [SMILES: c1ccc(-c2nc(-c3ccccc3)nc(-c3cccc(-c4cccc5oc6ccc(-c7cccc(-c8ccc9c(c8)c8ccccc8n9-c8ccccc8)c7)cc6c45)c3)n2)cc1]

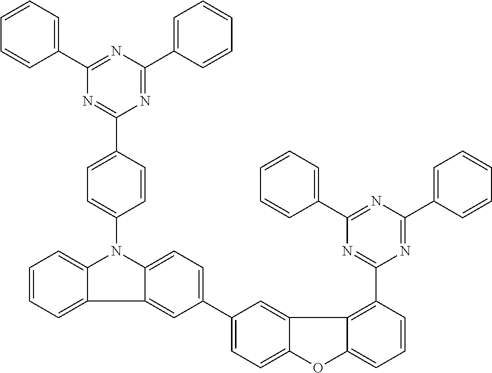 [SMILES: c1ccc(-c2nc(-c3ccccc3)nc(-c3ccc(-n4c5ccccc5c5cc(-c6ccc7oc8cccc(-c9nc(-c%10ccccc%10)nc(-c%10ccccc%10)n9)c8c7c6)ccc54)cc3)n2)cc1]

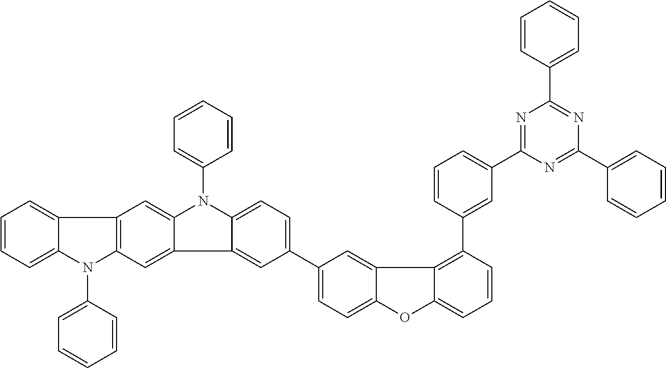 [SMILES: c1ccc(-c2nc(-c3ccccc3)nc(-c3cccc(-c4cccc5oc6ccc(-c7ccc8c(c7)c7cc9c(cc7n8-c7ccccc7)c7ccccc7n9-c7ccccc7)cc6c45)c3)n2)cc1]

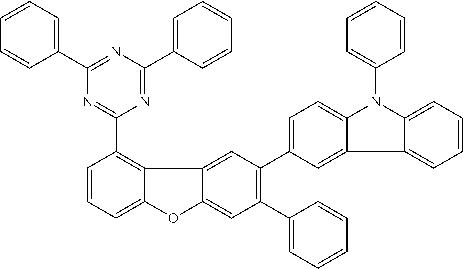 [SMILES: c1ccc(-c2nc(-c3ccccc3)nc(-c3cccc4oc5cc(-c6ccccc6)c(-c6ccc7c(c6)c6ccccc6n7-c6ccccc6)cc5c34)n2)cc1]

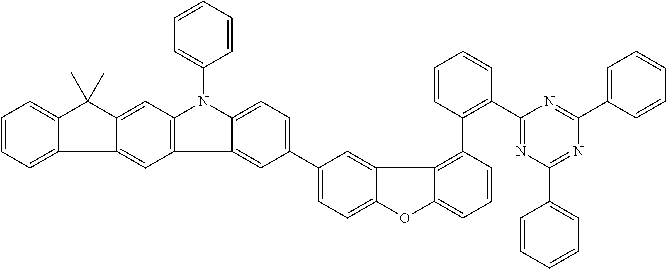 [SMILES: CC1(C)c2ccccc2-c2cc3c4cc(-c5ccc6oc7cccc(-c8ccccc8-c8nc(-c9ccccc9)nc(-c9ccccc9)n8)c7c6c5)ccc4n(-c4ccccc4)c3cc21]

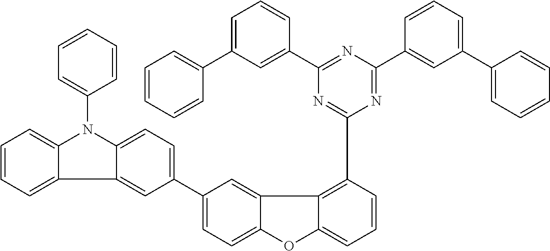 [SMILES: c1ccc(-c2cccc(-c3nc(-c4cccc(-c5ccccc5)c4)nc(-c4cccc5oc6ccc(-c7ccc8c(c7)c7ccccc7n8-c7ccccc7)cc6c45)n3)c2)cc1]

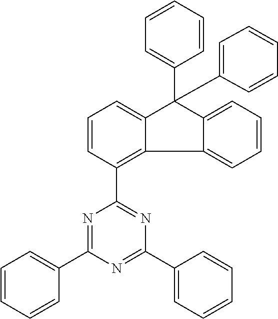 [SMILES: c1ccc(-c2nc(-c3ccccc3)nc(-c3cccc4c3-c3ccccc3C4(c3ccccc3)c3ccccc3)n2)cc1]

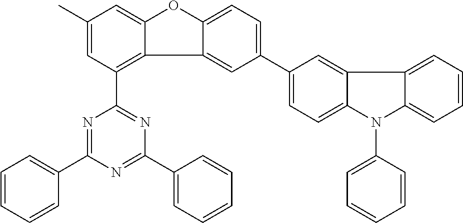 [SMILES: Cc1cc(-c2nc(-c3ccccc3)nc(-c3ccccc3)n2)c2c(c1)oc1ccc(-c3ccc4c(c3)c3ccccc3n4-c3ccccc3)cc12]

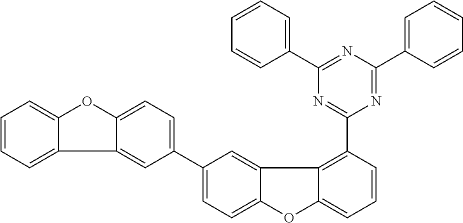 [SMILES: c1ccc(-c2nc(-c3ccccc3)nc(-c3cccc4oc5ccc(-c6ccc7oc8ccccc8c7c6)cc5c34)n2)cc1]

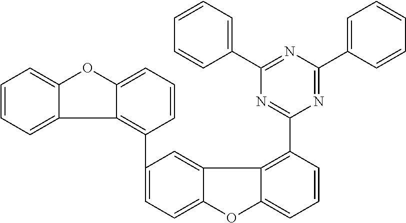 [SMILES: c1ccc(-c2nc(-c3ccccc3)nc(-c3cccc4oc5ccc(-c6cccc7oc8ccccc8c67)cc5c34)n2)cc1]

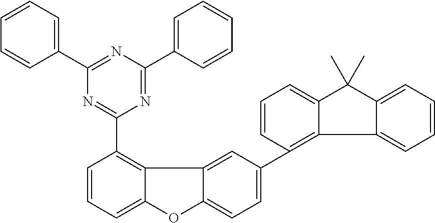 [SMILES: CC1(C)c2ccccc2-c2c(-c3ccc4oc5cccc(-c6nc(-c7ccccc7)nc(-c7ccccc7)n6)c5c4c3)cccc21]

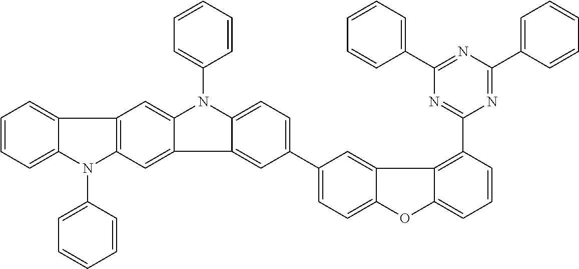 [SMILES: c1ccc(-c2nc(-c3ccccc3)nc(-c3cccc4oc5ccc(-c6ccc7c(c6)c6cc8c(cc6n7-c6ccccc6)c6ccccc6n8-c6ccccc6)cc5c34)n2)cc1]

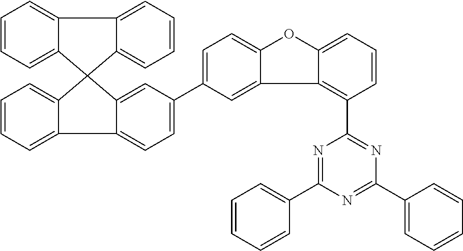 [SMILES: c1ccc(-c2nc(-c3ccccc3)nc(-c3cccc4oc5ccc(-c6ccc7c(c6)C6(c8ccccc8-c8ccccc86)c6ccccc6-7)cc5c34)n2)cc1]

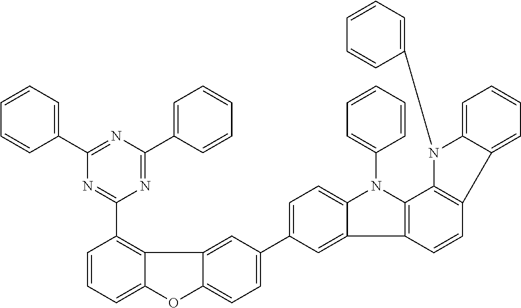 [SMILES: c1ccc(-c2nc(-c3ccccc3)nc(-c3cccc4oc5ccc(-c6ccc7c(c6)c6ccc8c9ccccc9n(-c9ccccc9)c8c6n7-c6ccccc6)cc5c34)n2)cc1]

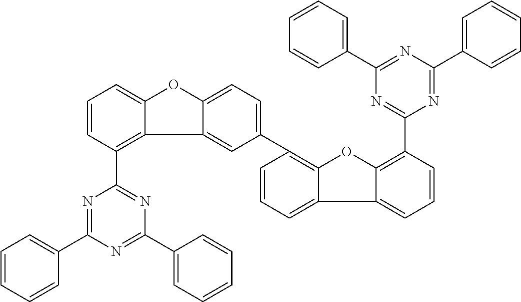 [SMILES: c1ccc(-c2nc(-c3ccccc3)nc(-c3cccc4c3oc3c(-c5ccc6oc7cccc(-c8nc(-c9ccccc9)nc(-c9ccccc9)n8)c7c6c5)cccc34)n2)cc1]

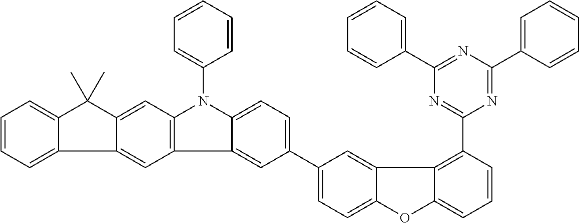 [SMILES: CC1(C)c2ccccc2-c2cc3c4cc(-c5ccc6oc7cccc(-c8nc(-c9ccccc9)nc(-c9ccccc9)n8)c7c6c5)ccc4n(-c4ccccc4)c3cc21]

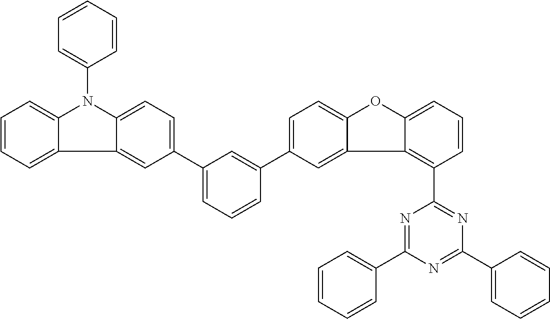 [SMILES: c1ccc(-c2nc(-c3ccccc3)nc(-c3cccc4oc5ccc(-c6cccc(-c7ccc8c(c7)c7ccccc7n8-c7ccccc7)c6)cc5c34)n2)cc1]

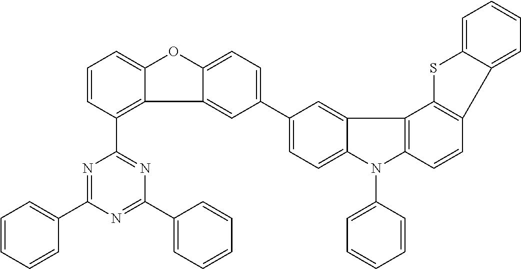 [SMILES: c1ccc(-c2nc(-c3ccccc3)nc(-c3cccc4oc5ccc(-c6ccc7c(c6)c6c8sc9ccccc9c8ccc6n7-c6ccccc6)cc5c34)n2)cc1]

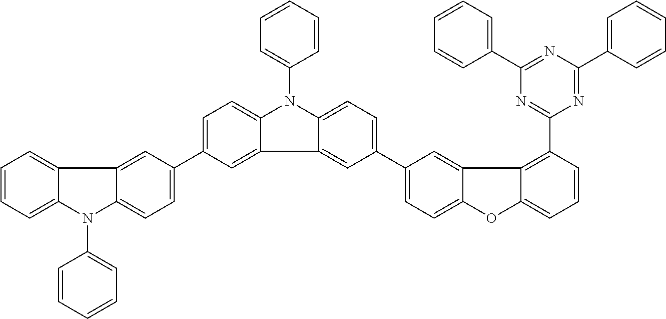 [SMILES: c1ccc(-c2nc(-c3ccccc3)nc(-c3cccc4oc5ccc(-c6ccc7c(c6)c6cc(-c8ccc9c(c8)c8ccccc8n9-c8ccccc8)ccc6n7-c6ccccc6)cc5c34)n2)cc1]

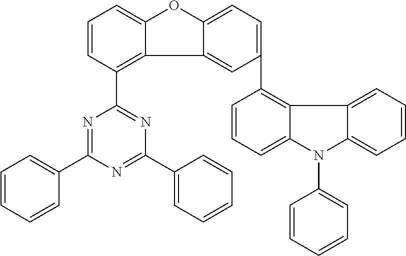 [SMILES: c1ccc(-c2nc(-c3ccccc3)nc(-c3cccc4oc5ccc(-c6cccc7c6c6ccccc6n7-c6ccccc6)cc5c34)n2)cc1]

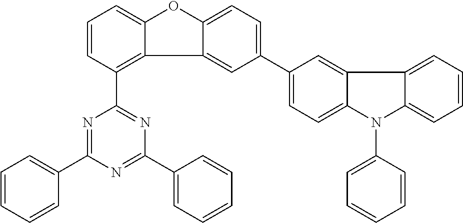 [SMILES: c1ccc(-c2nc(-c3ccccc3)nc(-c3cccc4oc5ccc(-c6ccc7c(c6)c6ccccc6n7-c6ccccc6)cc5c34)n2)cc1]

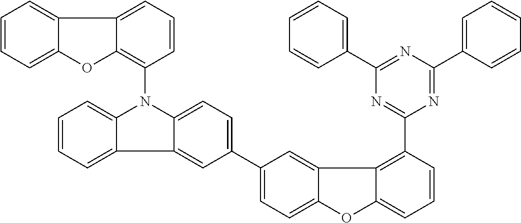 [SMILES: c1ccc(-c2nc(-c3ccccc3)nc(-c3cccc4oc5ccc(-c6ccc7c(c6)c6ccccc6n7-c6cccc7c6oc6ccccc67)cc5c34)n2)cc1]

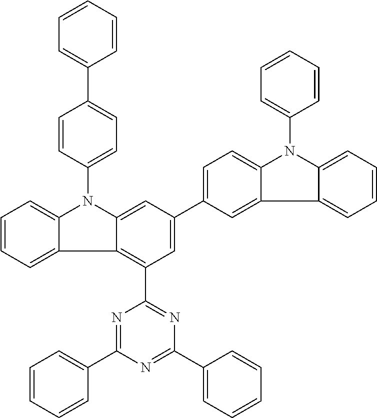 [SMILES: c1ccc(-c2ccc(-n3c4ccccc4c4c(-c5nc(-c6ccccc6)nc(-c6ccccc6)n5)cc(-c5ccc6c(c5)c5ccccc5n6-c5ccccc5)cc43)cc2)cc1]

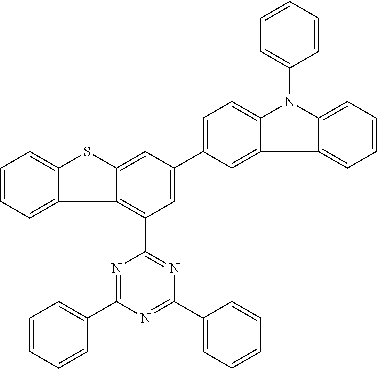 [SMILES: c1ccc(-c2nc(-c3ccccc3)nc(-c3cc(-c4ccc5c(c4)c4ccccc4n5-c4ccccc4)cc4sc5ccccc5c34)n2)cc1]

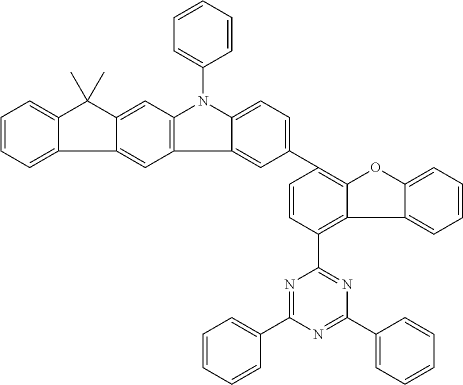 [SMILES: CC1(C)c2ccccc2-c2cc3c4cc(-c5ccc(-c6nc(-c7ccccc7)nc(-c7ccccc7)n6)c6c5oc5ccccc56)ccc4n(-c4ccccc4)c3cc21]

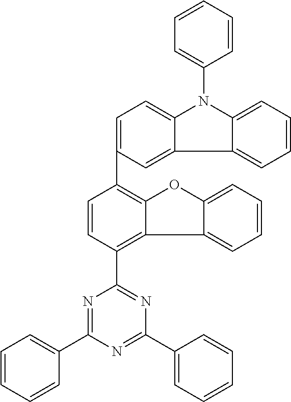 [SMILES: c1ccc(-c2nc(-c3ccccc3)nc(-c3ccc(-c4ccc5c(c4)c4ccccc4n5-c4ccccc4)c4oc5ccccc5c34)n2)cc1]

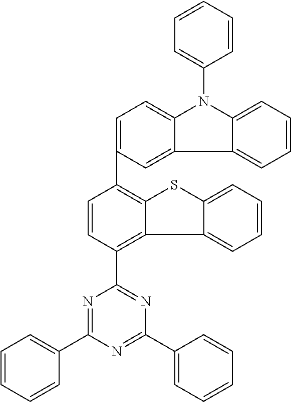 [SMILES: c1ccc(-c2nc(-c3ccccc3)nc(-c3ccc(-c4ccc5c(c4)c4ccccc4n5-c4ccccc4)c4sc5ccccc5c34)n2)cc1]

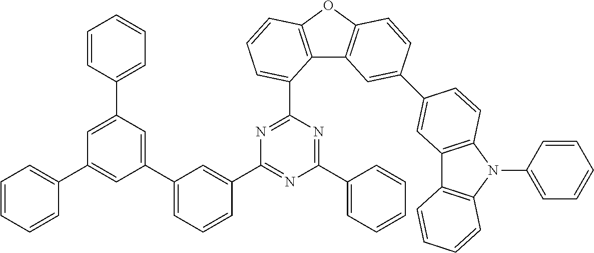 [SMILES: c1ccc(-c2cc(-c3ccccc3)cc(-c3cccc(-c4nc(-c5ccccc5)nc(-c5cccc6oc7ccc(-c8ccc9c(c8)c8ccccc8n9-c8ccccc8)cc7c56)n4)c3)c2)cc1]

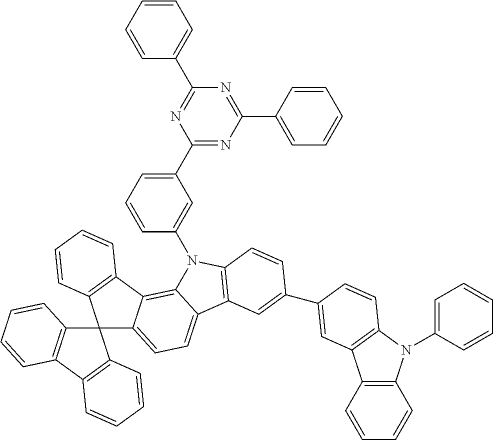 [SMILES: c1ccc(-c2nc(-c3ccccc3)nc(-c3cccc(-n4c5ccc(-c6ccc7c(c6)c6ccccc6n7-c6ccccc6)cc5c5ccc6c(c54)-c4ccccc4C64c5ccccc5-c5ccccc54)c3)n2)cc1]

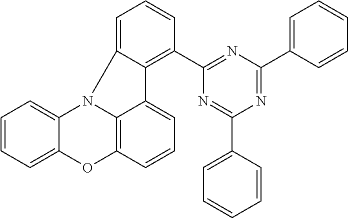 [SMILES: c1ccc(-c2nc(-c3ccccc3)nc(-c3cccc4c3c3cccc5c3n4-c3ccccc3O5)n2)cc1]

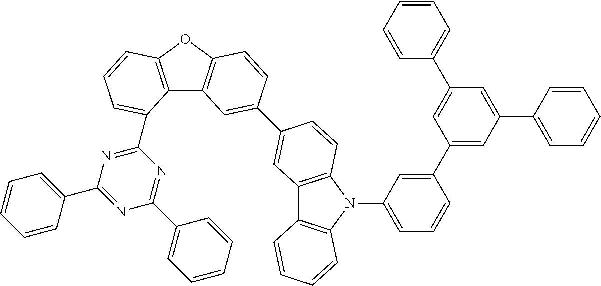 [SMILES: c1ccc(-c2cc(-c3ccccc3)cc(-c3cccc(-n4c5ccccc5c5cc(-c6ccc7oc8cccc(-c9nc(-c%10ccccc%10)nc(-c%10ccccc%10)n9)c8c7c6)ccc54)c3)c2)cc1]

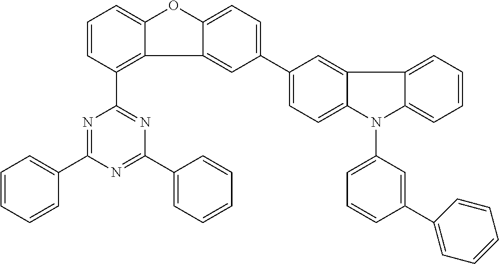 [SMILES: c1ccc(-c2cccc(-n3c4ccccc4c4cc(-c5ccc6oc7cccc(-c8nc(-c9ccccc9)nc(-c9ccccc9)n8)c7c6c5)ccc43)c2)cc1]

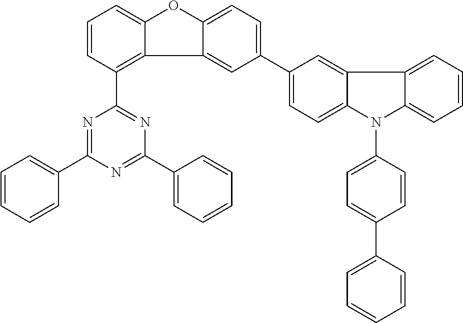 [SMILES: c1ccc(-c2ccc(-n3c4ccccc4c4cc(-c5ccc6oc7cccc(-c8nc(-c9ccccc9)nc(-c9ccccc9)n8)c7c6c5)ccc43)cc2)cc1]

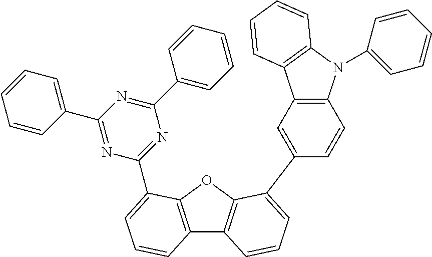 [SMILES: c1ccc(-c2nc(-c3ccccc3)nc(-c3cccc4c3oc3c(-c5ccc6c(c5)c5ccccc5n6-c5ccccc5)cccc34)n2)cc1]

Examples of suitable quinazoline compounds are the compounds depicted in the following table:

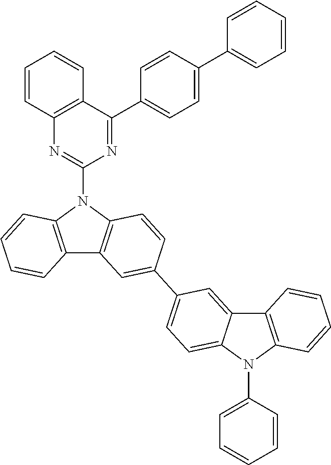 [SMILES: c1ccc(-c2ccc(-c3nc(-n4c5ccccc5c5cc(-c6ccc7c(c6)c6ccccc6n7-c6ccccc6)ccc54)nc4ccccc34)cc2)cc1]

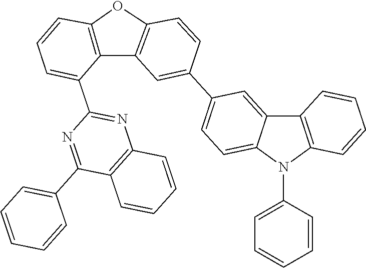 [SMILES: c1ccc(-c2nc(-c3cccc4oc5ccc(-c6ccc7c(c6)c6ccccc6n7-c6ccccc6)cc5c34)nc3ccccc23)cc1]

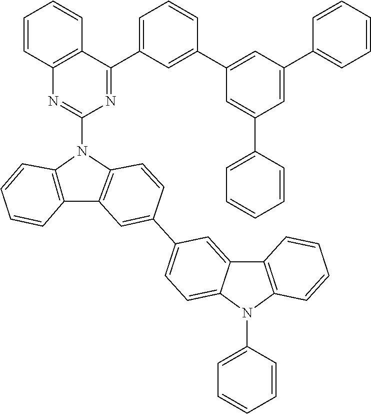 [SMILES: c1ccc(-c2cc(-c3ccccc3)cc(-c3cccc(-c4nc(-n5c6ccccc6c6cc(-c7ccc8c(c7)c7ccccc7n8-c7ccccc7)ccc65)nc5ccccc45)c3)c2)cc1]

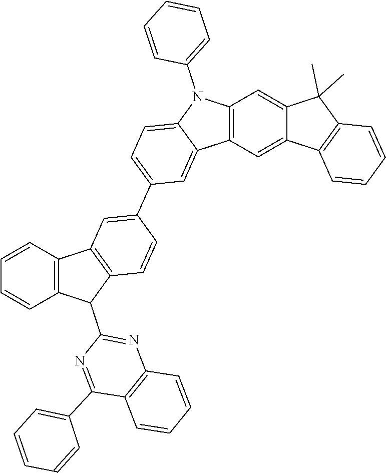 [SMILES: C=C/C(=C\C=C/C)c1nc(C2c3ccccc3-c3cc(-c4ccc5c(c4)c4cc6c(cc4n5-c4ccccc4)C(C)(C)c4ccccc4-6)ccc32)nc2ccccc12]

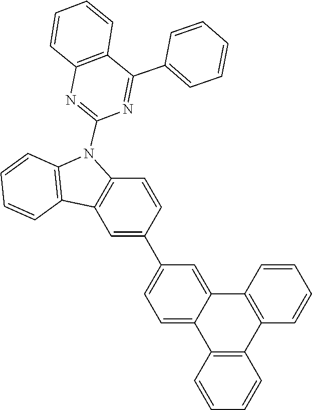 [SMILES: c1ccc(-c2nc(-n3c4ccccc4c4cc(-c5ccc6c7ccccc7c7ccccc7c6c5)ccc43)nc3ccccc23)cc1]

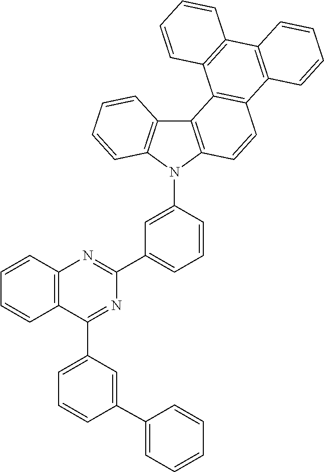 [SMILES: c1ccc(-c2cccc(-c3nc(-c4cccc(-n5c6ccccc6c6c7c8ccccc8c8ccccc8c7ccc65)c4)nc4ccccc34)c2)cc1]

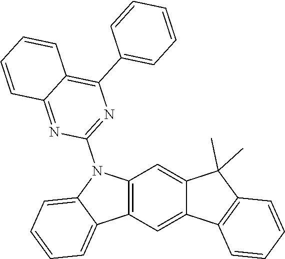 [SMILES: CC1(C)c2ccccc2-c2cc3c4ccccc4n(-c4nc(-c5ccccc5)c5ccccc5n4)c3cc21]

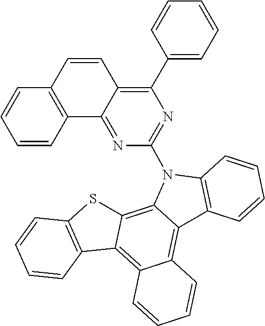 [SMILES: c1ccc(-c2nc(-n3c4ccccc4c4c5ccccc5c5c6ccccc6sc5c43)nc3c2ccc2ccccc23)cc1]

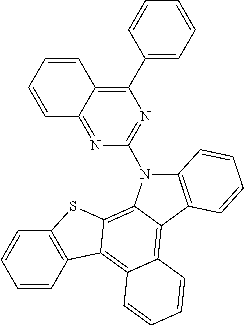 [SMILES: c1ccc(-c2nc(-n3c4ccccc4c4c5ccccc5c5c6ccccc6sc5c43)nc3ccccc23)cc1]

Suitable phosphorescent compounds (=triplet emitters) are especially compounds which, when suitably excited, emit light, preferably in the visible region, and also contain at least one atom of atomic number greater than 20, preferably greater than 38 and less than 84, more preferably greater than 56 and less than 80, especially a metal having this atomic number. Preferred phosphorescence emitters used are compounds containing copper, molybdenum, tungsten, rhenium, ruthenium, osmium, rhodium, iridium, palladium, platinum, silver, gold or europium, especially compounds containing iridium or platinum.

Examples of the emitters described above can be found in applications WO 00/70655, WO 2001/41512, WO 2002/02714, WO 2002/15645, EP 1191613, EP 1191612, EP 1191614, WO 05/033244, WO 05/019373, US 2005/0258742, WO 2009/146770, WO 2010/015307, WO 2010/031485, WO 2010/054731, WO 2010/054728, WO 2010/086089, WO 2010/099852, WO 2010/102709, WO 2011/032626, WO 2011/066898, WO 2011/157339, WO 2012/007086, WO 2014/008982, WO 2014/023377, WO 2014/094961, WO 2014/094960, WO 2015/036074, WO 2015/104045, WO 2015/117718, WO 2016/015815, WO 2016/124304, WO 2017/032439, WO 2018/011186 and WO 2018/041769, WO 2019/020538, WO 2018/178001 and as yet unpublished patent applications EP 17206950.2 and EP 18156388.3. In general, all phosphorescent complexes as used for phosphorescent OLEDs according to the prior art and as known to those skilled in the art in the field of organic electroluminescence are suitable, and the person skilled in the art will be able to use further phosphorescent complexes without exercising inventive skill.

Examples of phosphorescent dopants are adduced below.

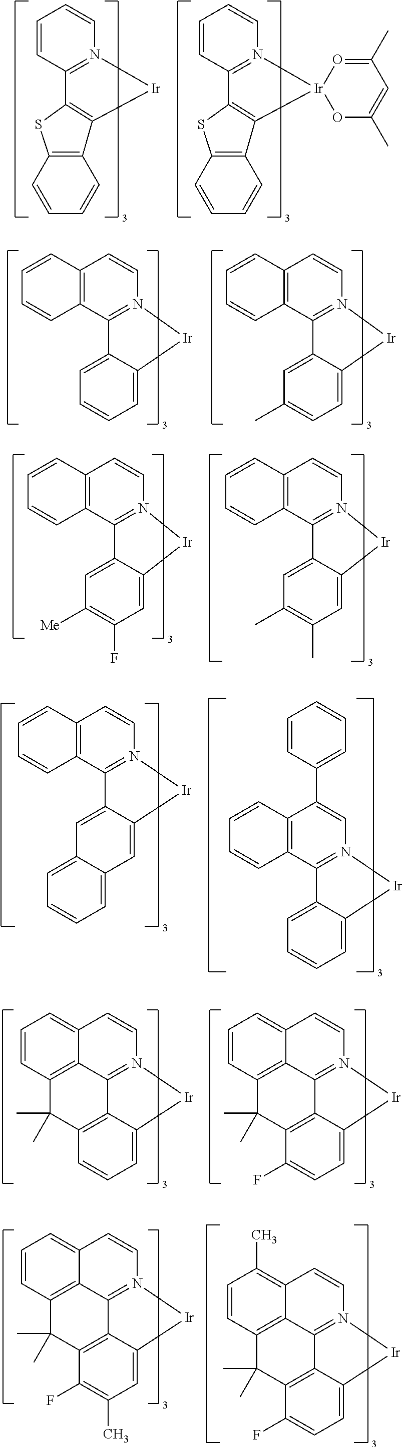

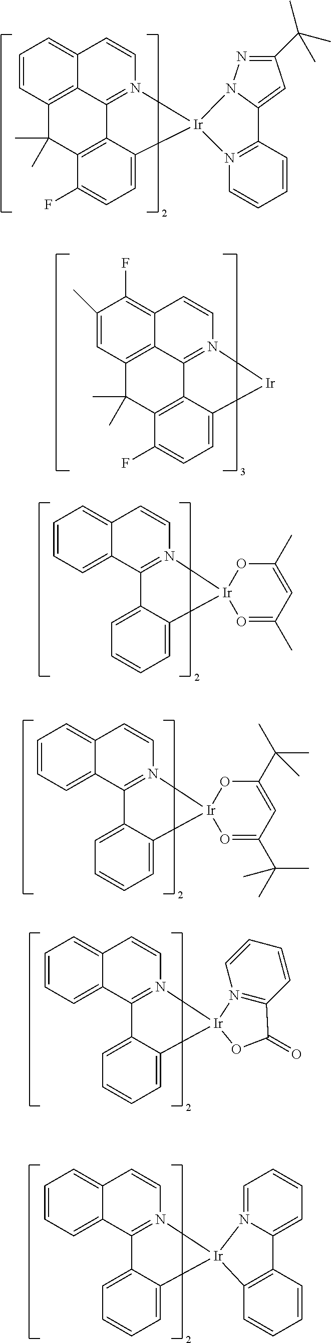

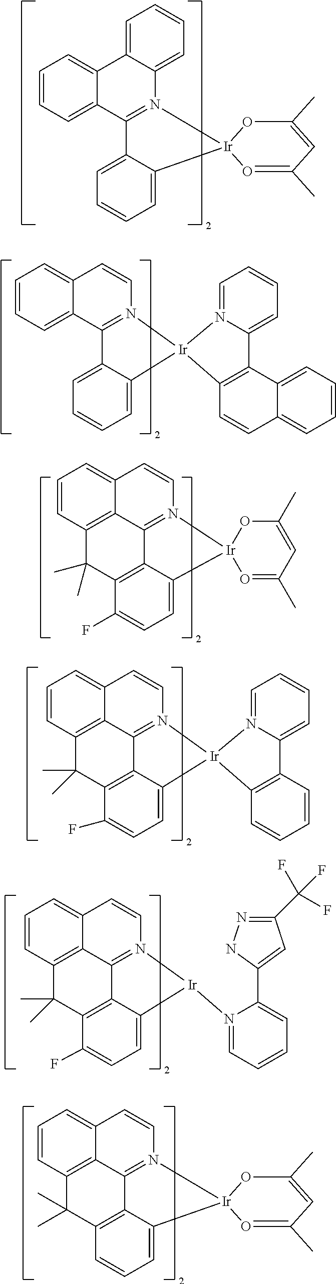

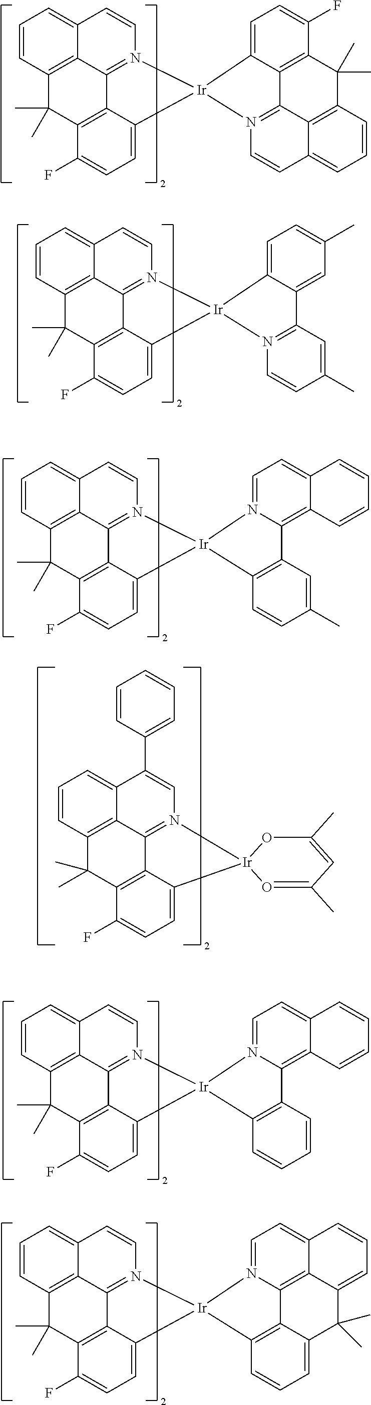

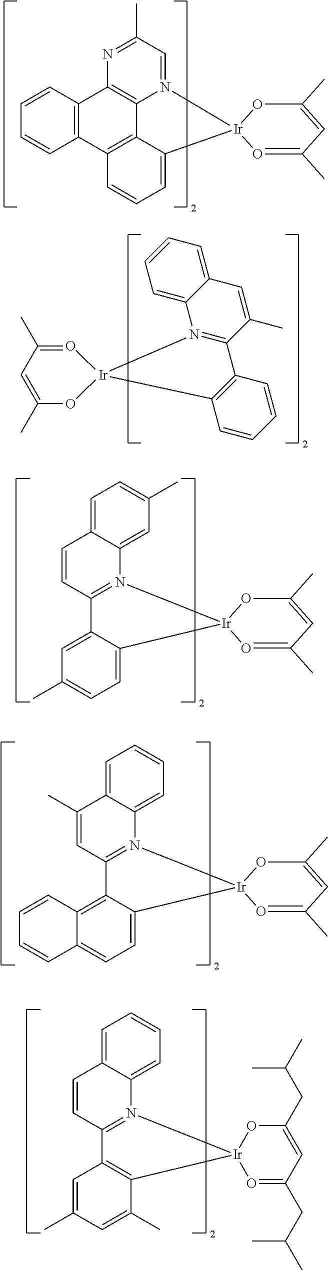

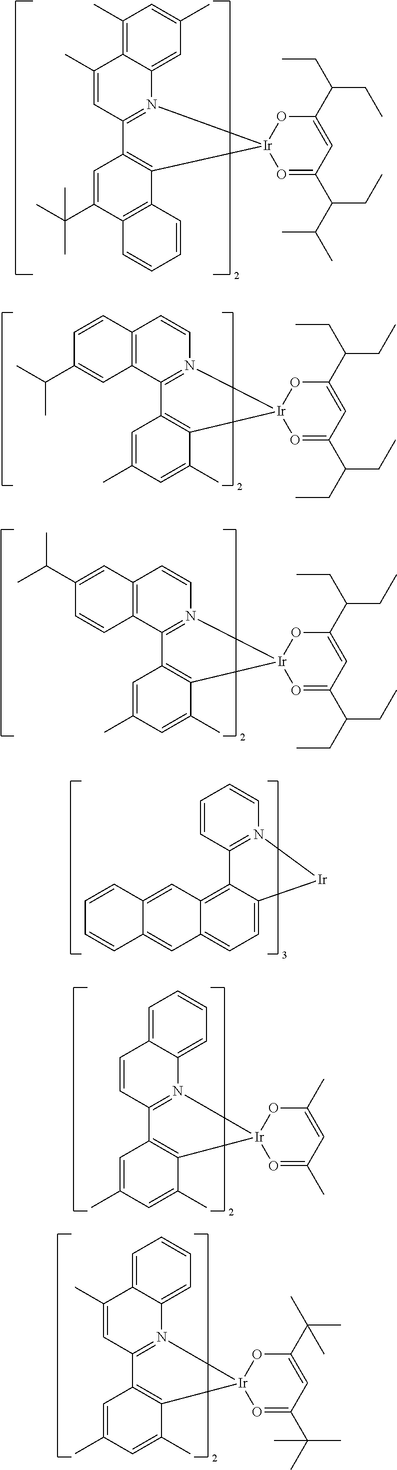

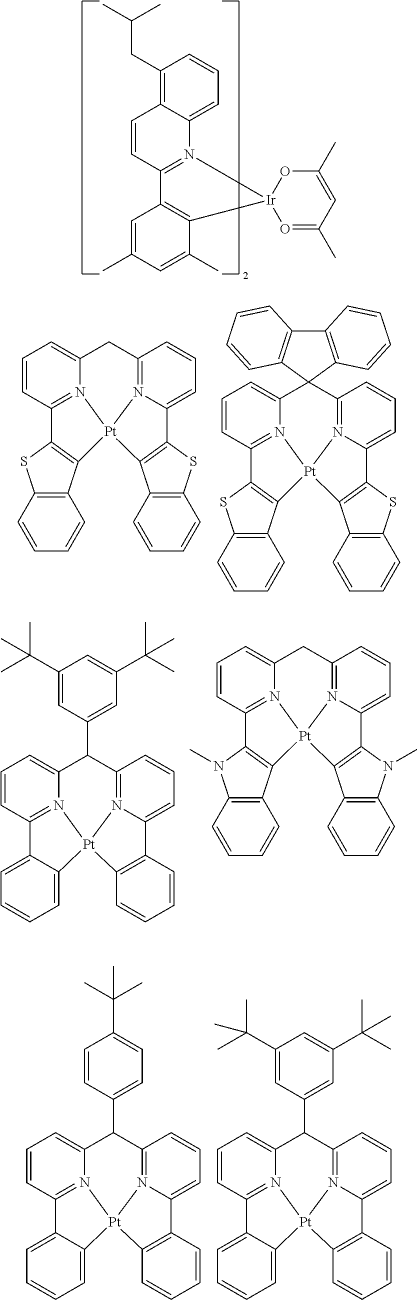

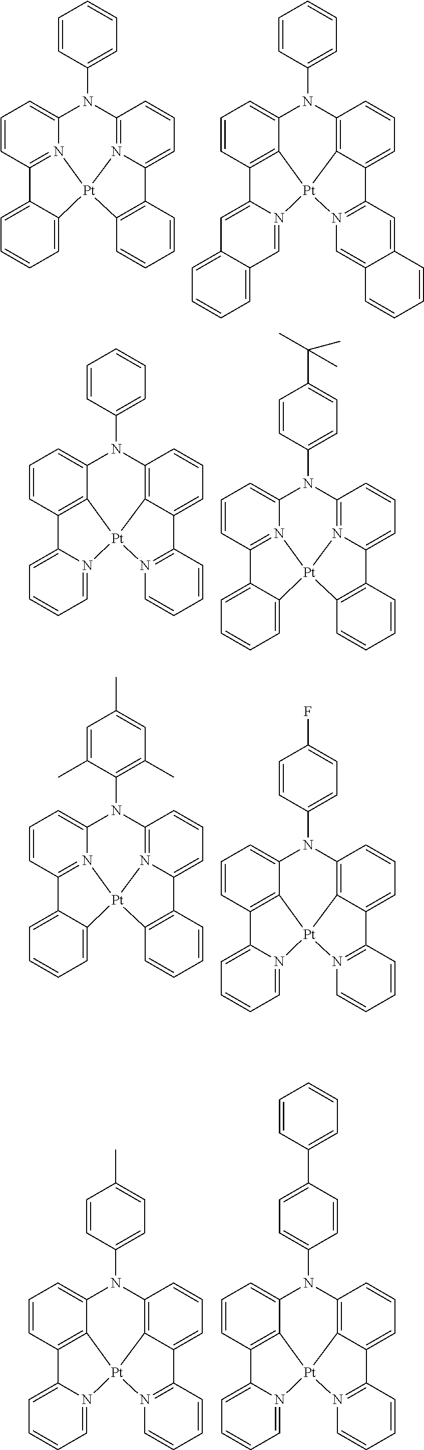

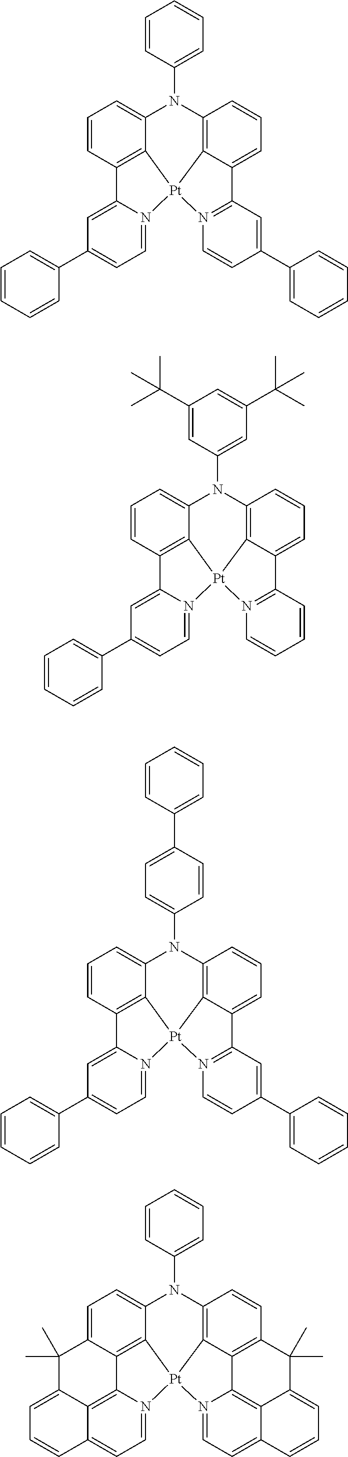

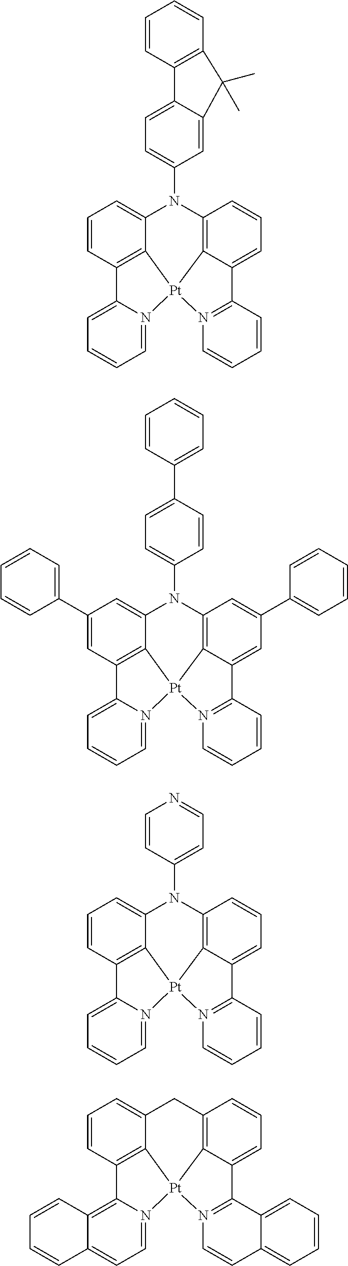

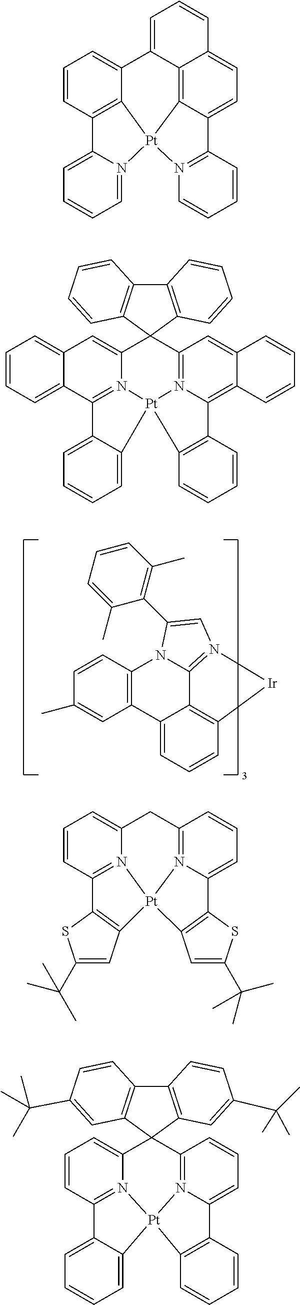

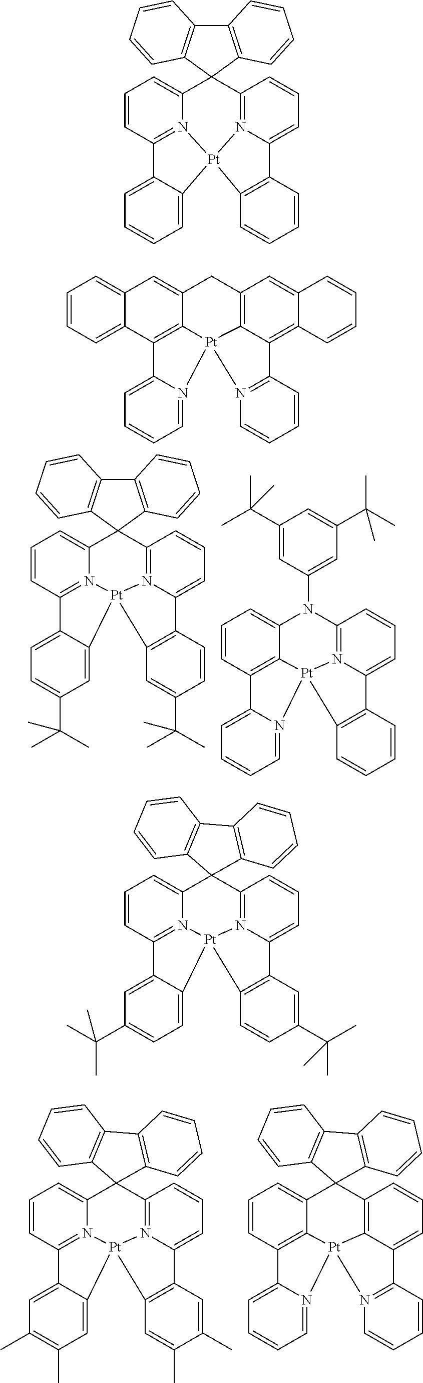

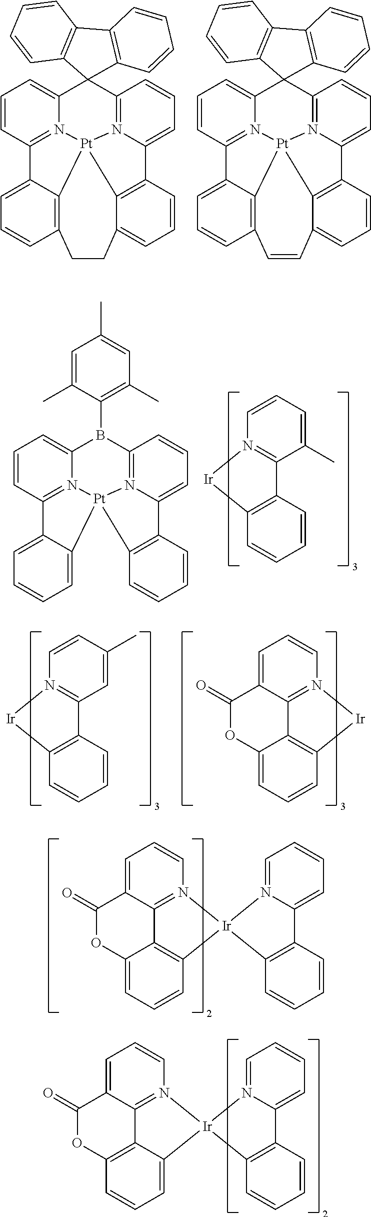

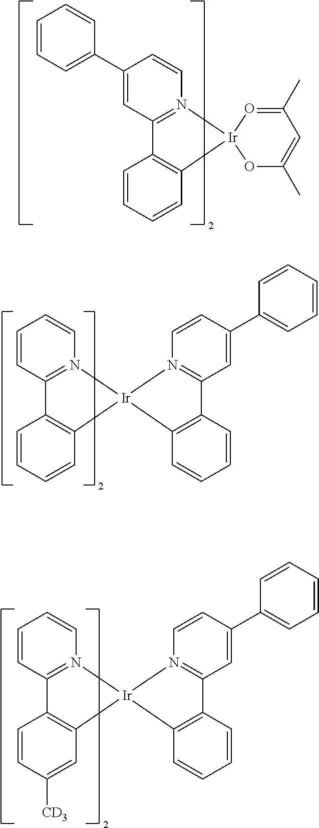

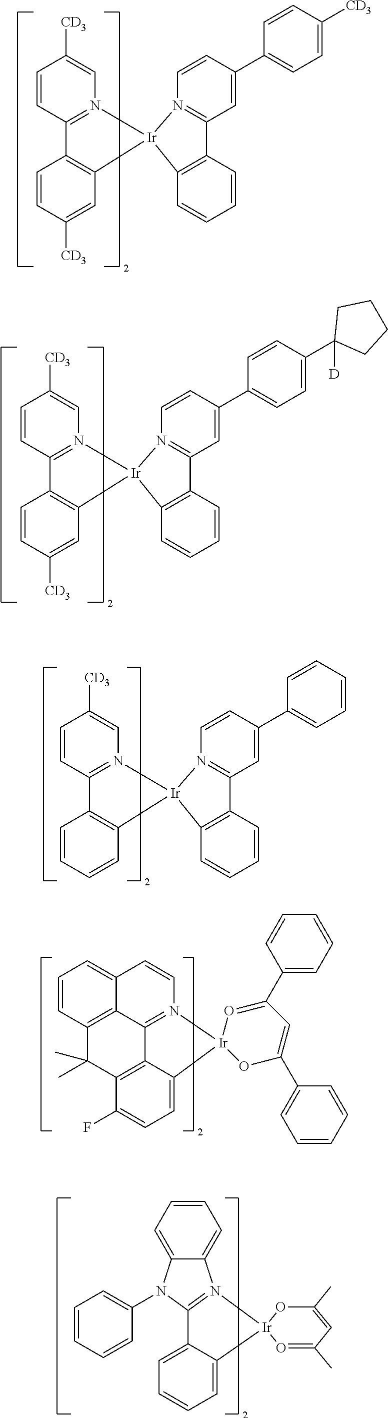

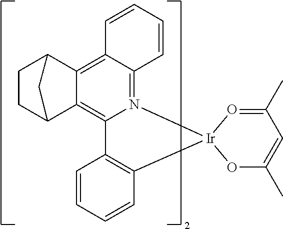

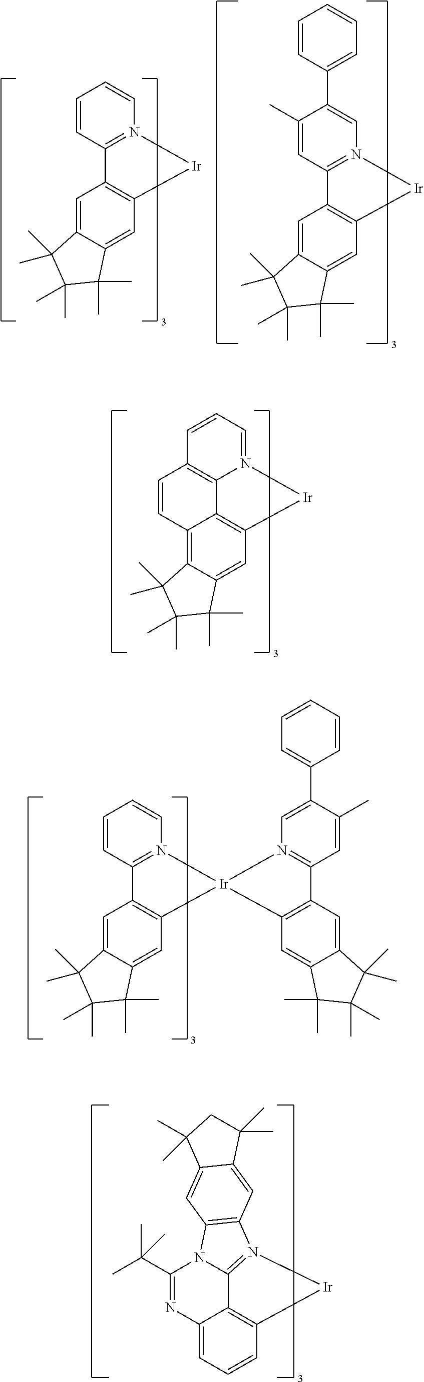

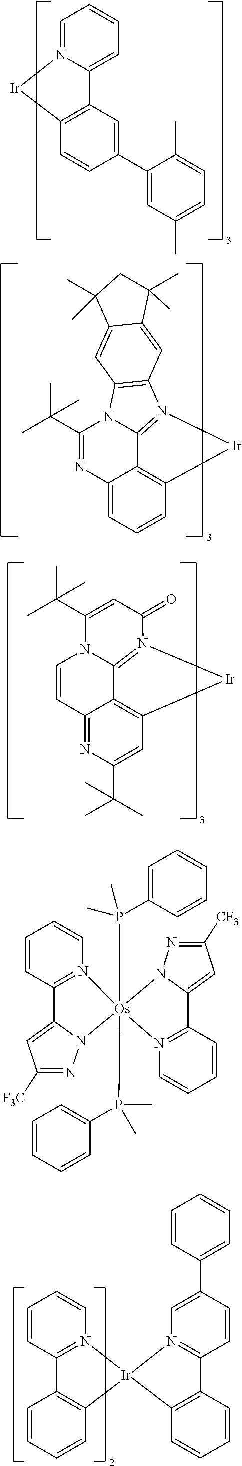

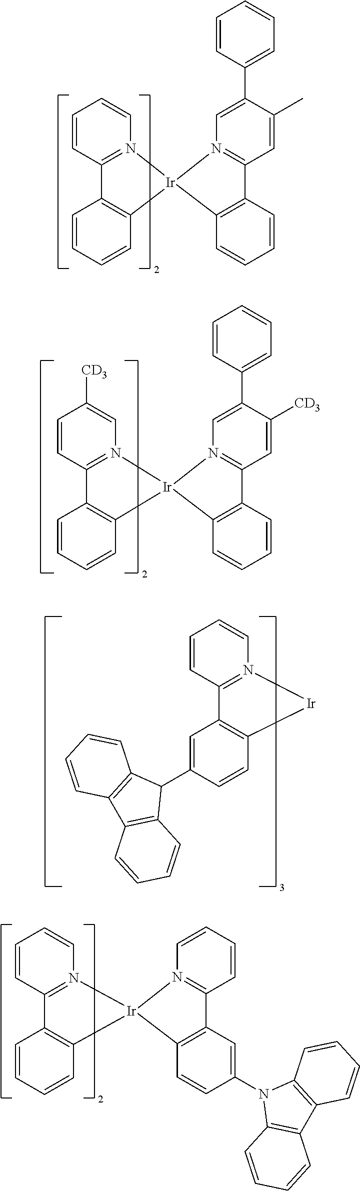

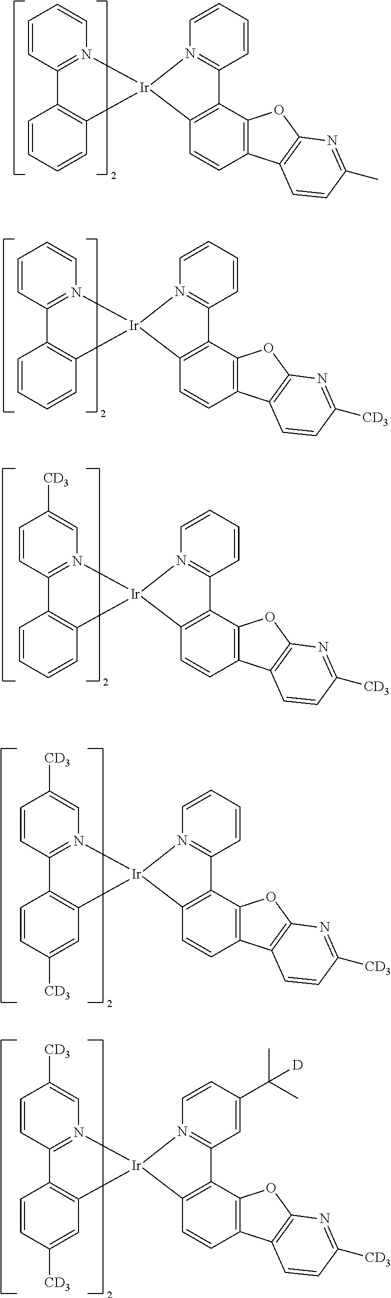

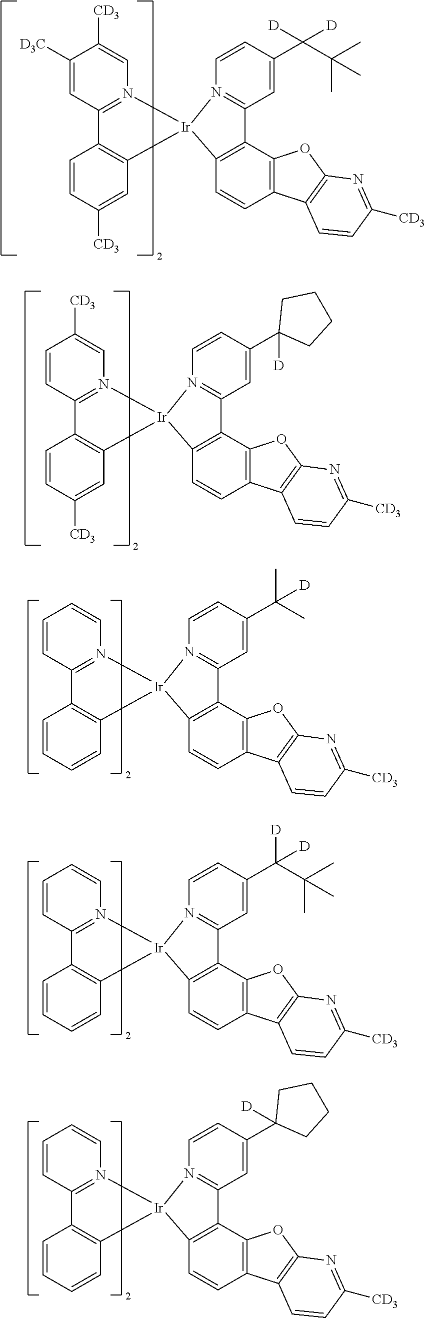

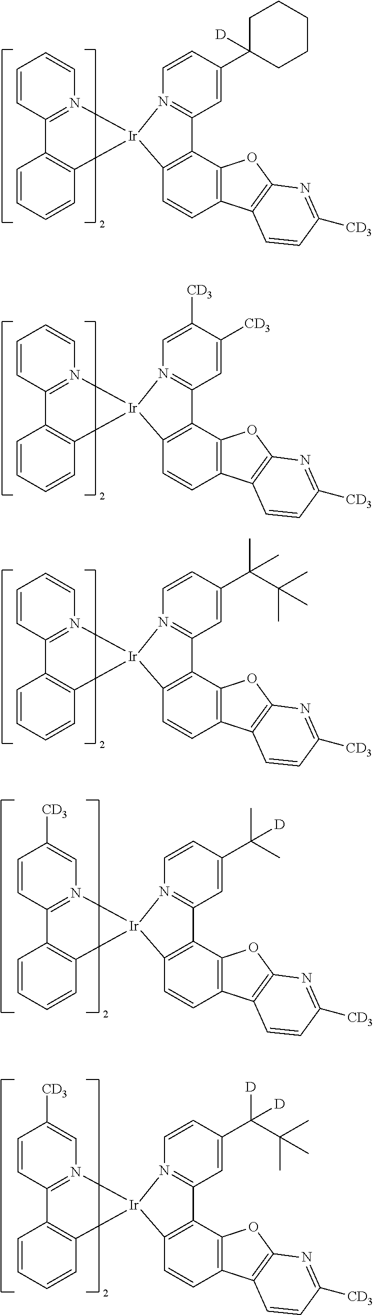

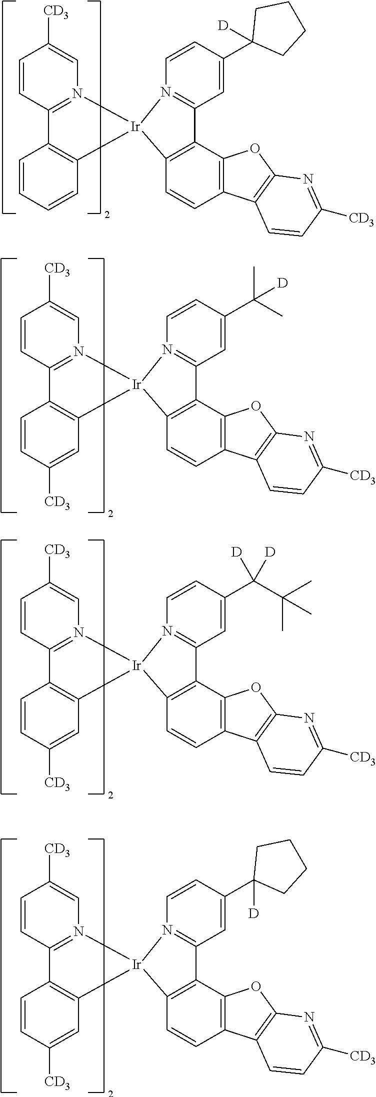

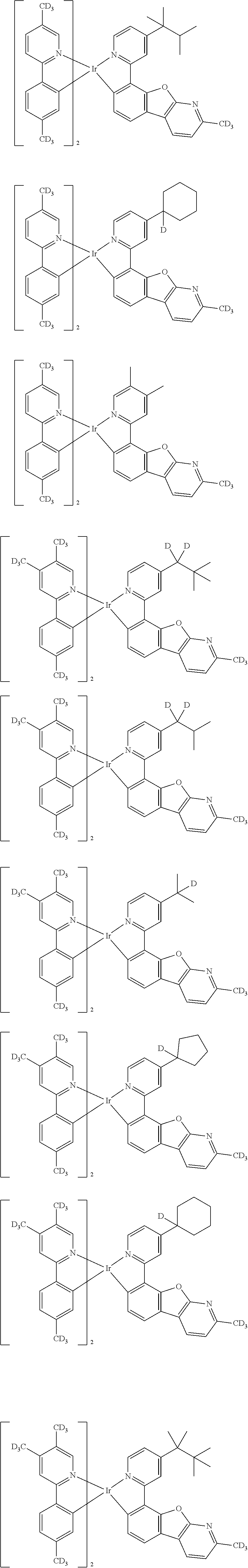

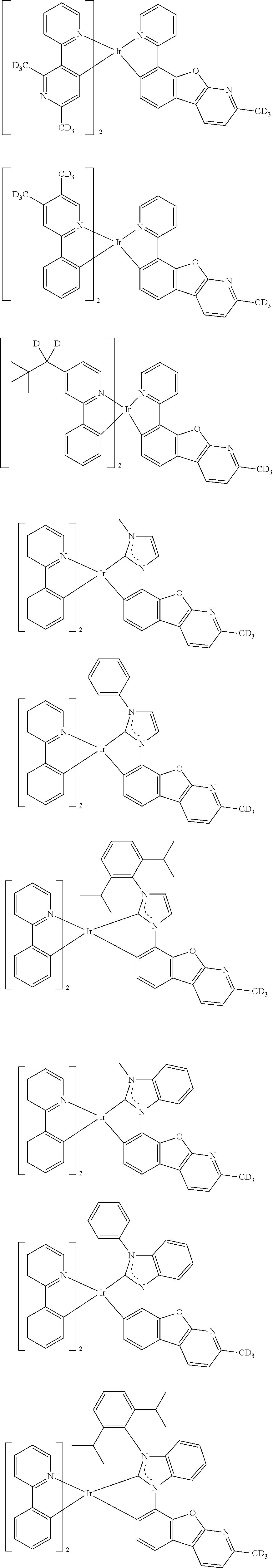

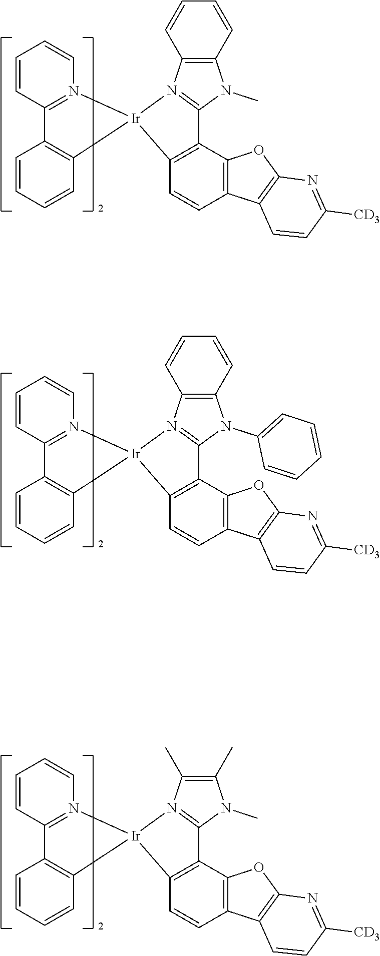

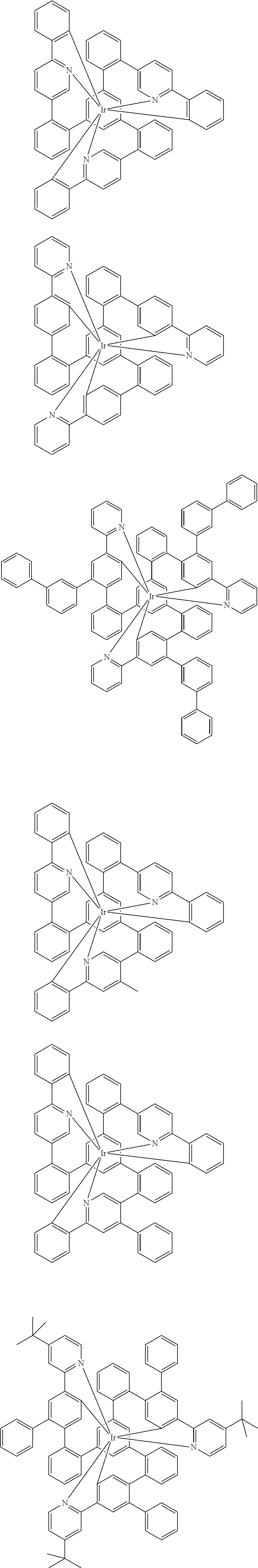

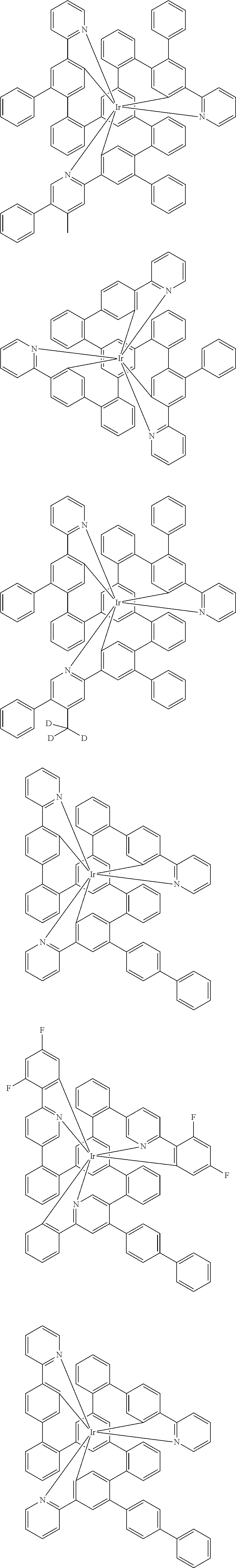

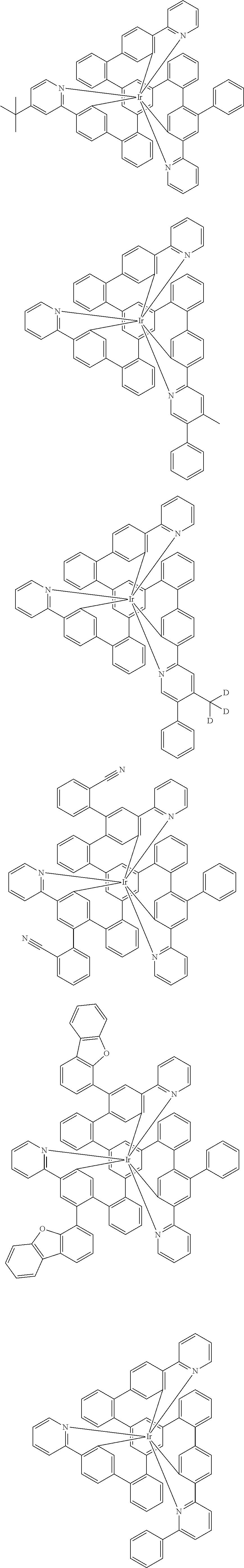

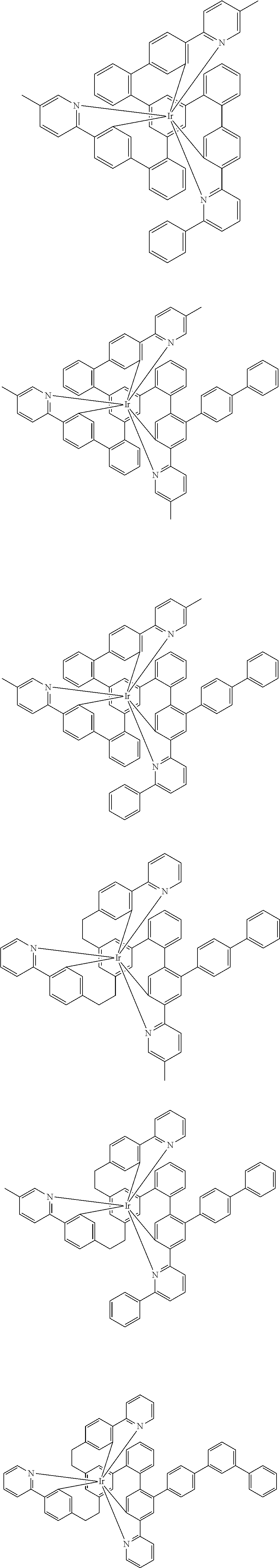

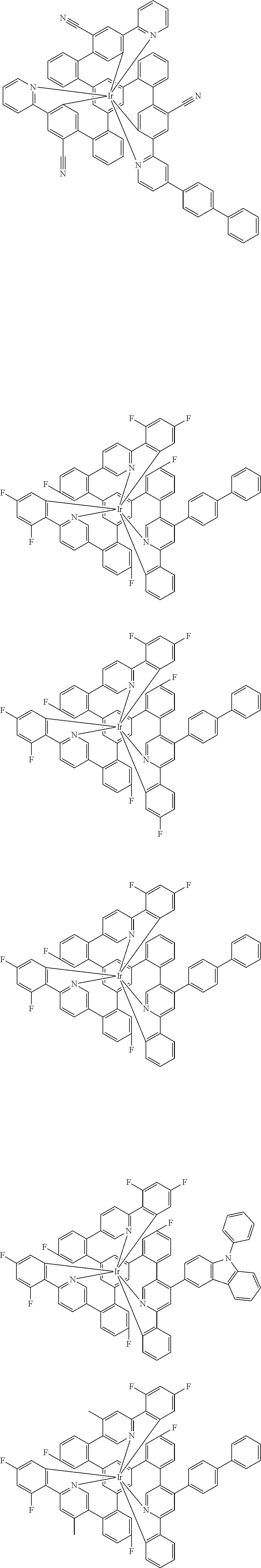

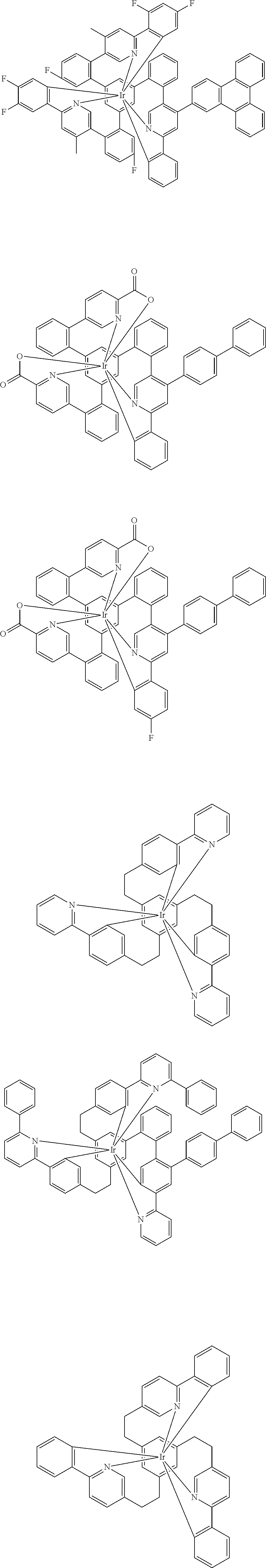

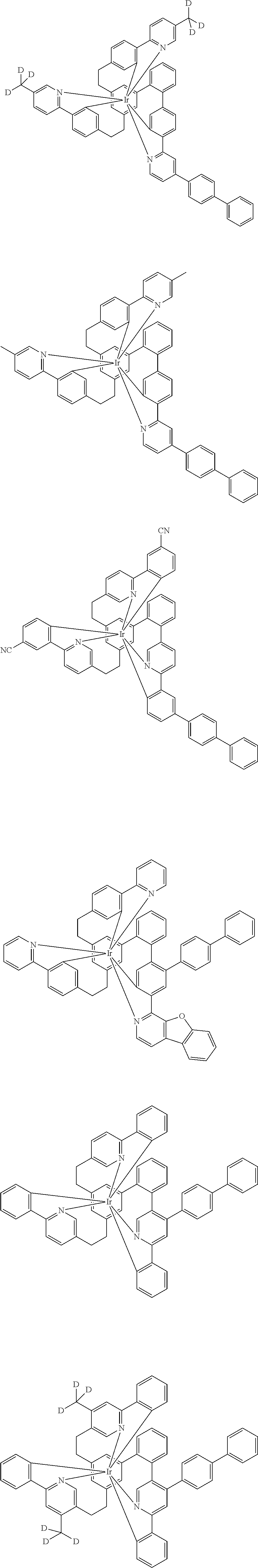

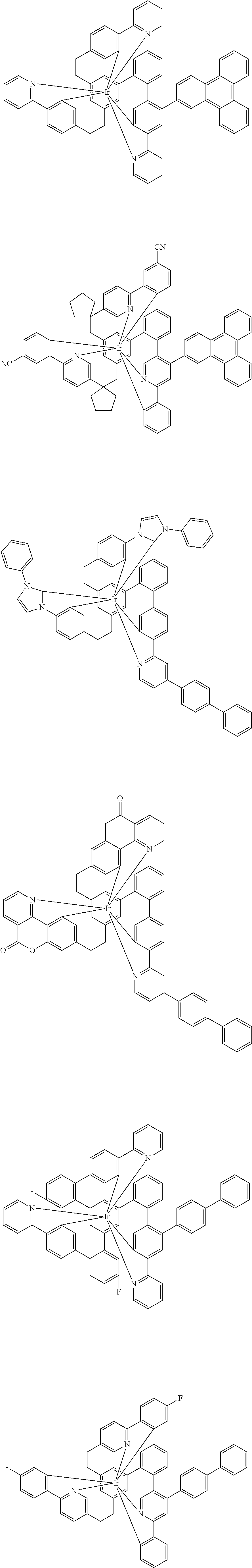

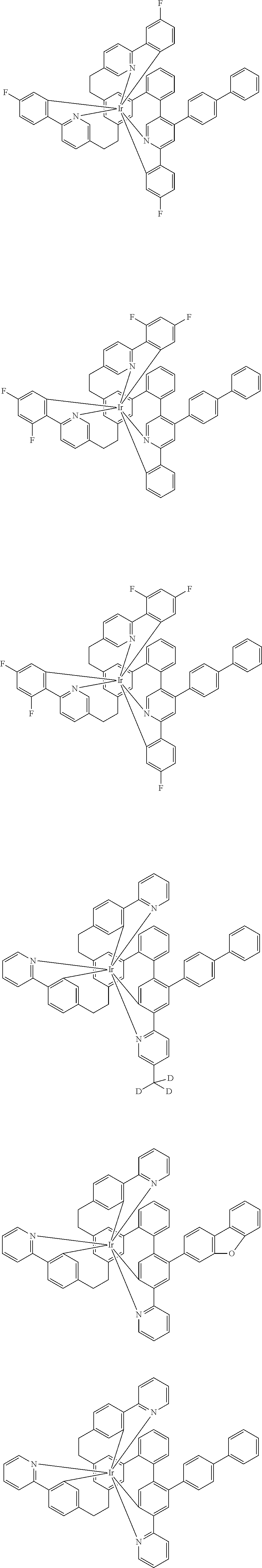

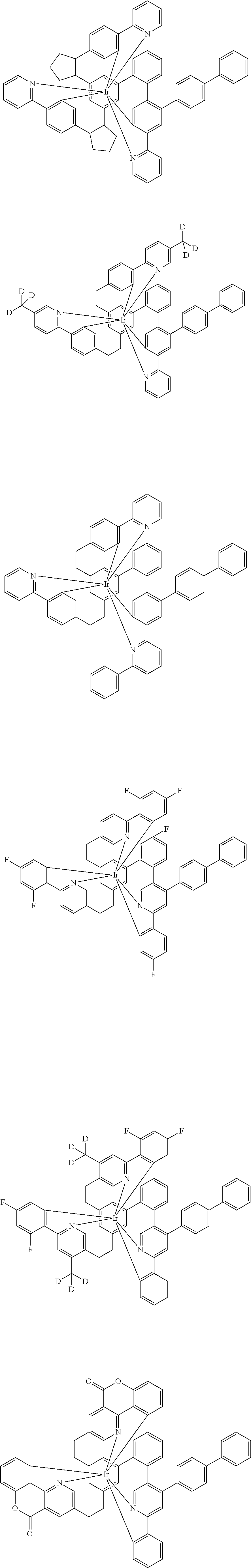

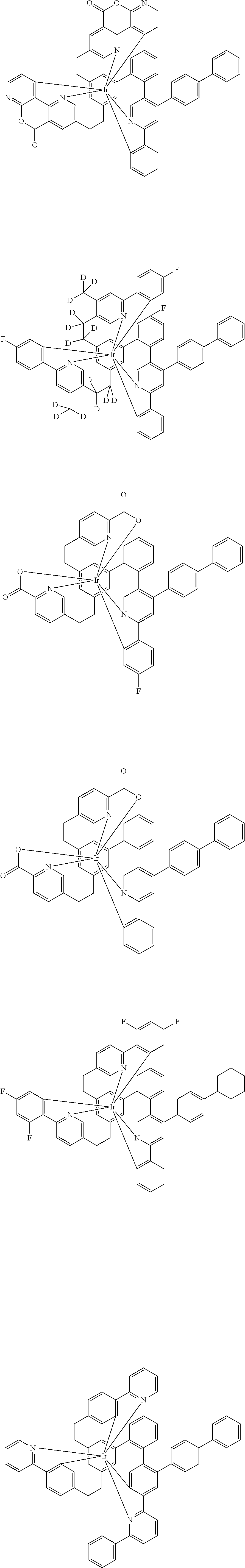

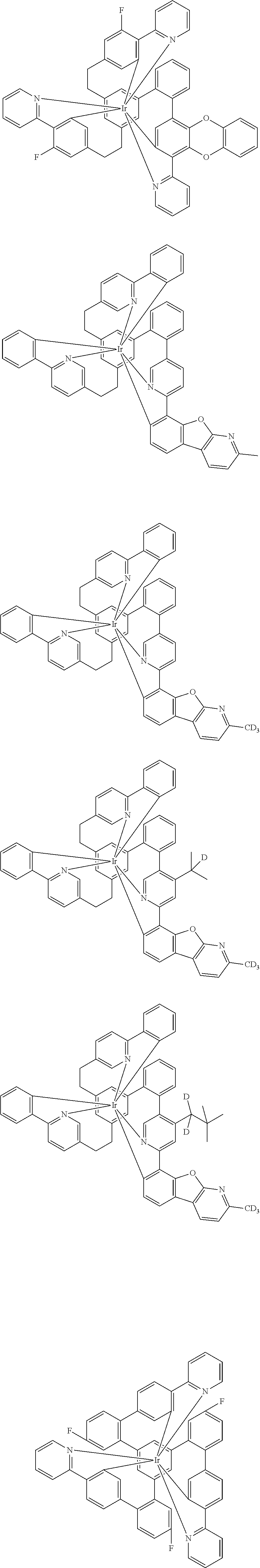

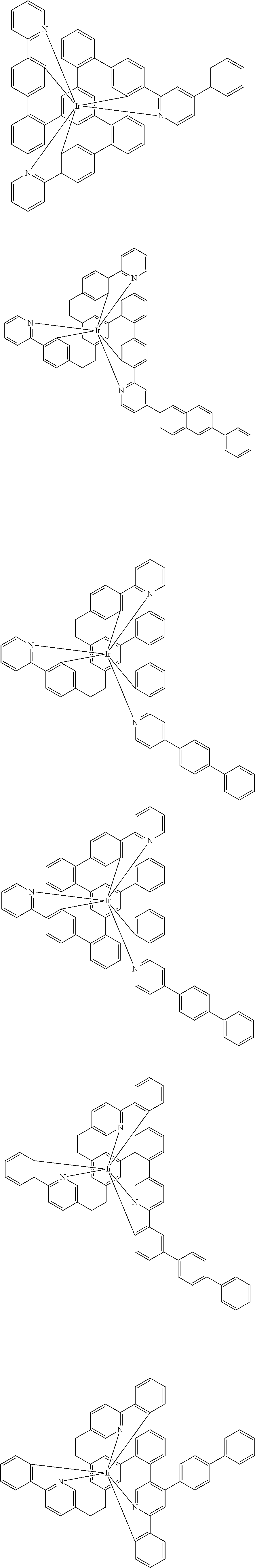

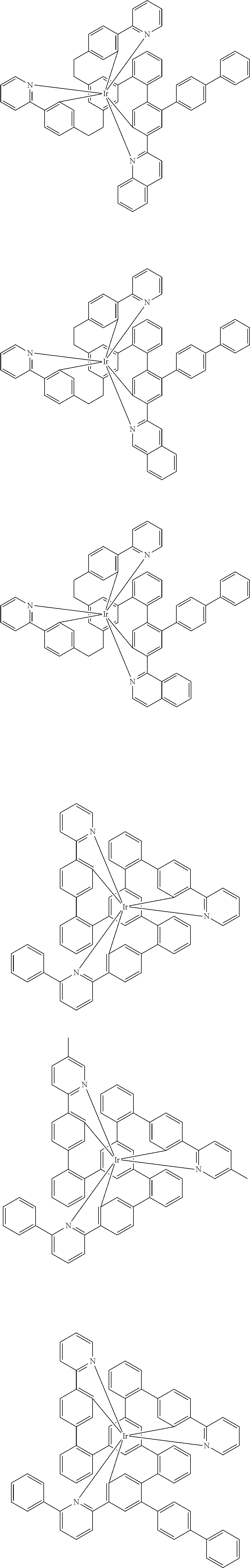

In the further layers of the organic electroluminescent device of the invention, it is possible to use any materials as typically used according to the prior art. The person skilled in the art will therefore be able, without exercising inventive skill, to use any materials known for organic electroluminescent devices in combination with the inventive compounds of formula (1) or the above-recited preferred embodiments.

Additionally preferred is an organic electroluminescent device, characterized in that one or more layers are coated by a sublimation process. In this case, the materials are applied by vapor deposition in vacuum sublimation systems at an initial pressure of less than 10-5 mbar, preferably less than 10-6 mbar. However, it is also possible that the initial pressure is even lower, for example less than 10-7 mbar.

Preference is likewise given to an organic electroluminescent device, characterized in that one or more lavers are coated by the OVPD (organic vapor phase deposition) method or with the aid of a carrier gas sublimation. In this case, the materials are applied at a pressure between 10⁵ mbar and 1 bar. A special case of this method is the OVJP (organic vapor jet printing) method, in which the materials are applied directly by a nozzle and thus structured.

Preference is additionally given to an organic electroluminescent device, characterized in that one or more layers are produced from solution, for example by spin-coating, or by any printing method, for example screen printing, flexographic printing, offset printing, LITI (light-induced thermal imaging, thermal transfer printing), inkjet printing or nozzle printing. For this purpose, soluble compounds are needed, which are obtained, for example, through suitable substitution.

In addition, hybrid methods are possible, in which, for example, one or more layers are applied from solution and one or more further layers are applied by vapor deposition.

These methods are known in general terms to those skilled in the art and can be applied by those skilled in the art without exercising inventive skill to organic electroluminescent devices comprising the compounds of the invention.

The compounds of the invention and the organic electroluminescent devices of the invention are notable for one or more of the following surprising advantages over the prior art:
1. The compounds of the invention, used as matrix material for phosphorescent emitters, lead to long lifetimes.
2. The compounds of the invention lead to high efficiencies. This is especially true when the compounds are used as matrix material for a phosphorescent emitter. More particularly, efficiency is better than in compounds that are comparable but do not have any heteroaromatic substituents or any group of the formula (3).
3. The compounds of the invention lead to low operating voltages. This is especially true when the compounds are used as matrix material for a phosphorescent emitter. More particularly, operating voltage is lower than in compounds that are comparable but do not have any heteroaromatic substituents or any group of the formula (3).

The invention is illustrated in more detail by the examples which follow, without any intention of restricting it thereby. The person skilled in the art will be able to use the information given to execute the invention over the entire scope disclosed and to prepare further compounds of the invention without exercising inventive skill and to use them in electronic devices or to employ the process of the invention.

### EXAMPLES

### Synthesis Examples

The syntheses which follow, unless stated otherwise, are conducted under a protective gas atmosphere in dried solvents. The solvents and reagents can be purchased from ALDRICH or ABCR. The numbers given for the reactants that are not commercially available are the corresponding CAS numbers.

### a) 10-Bromo-5,7-dihydroindolo[2,3-c]quinolin-6-one

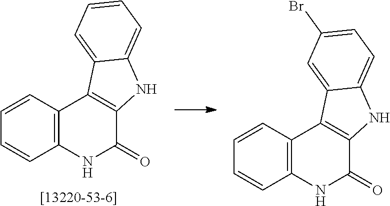 [SMILES: O=c1[nH]c2ccccc2c2c1[nH]c1ccccc12 ; O=c1[nH]c2ccccc2c2c1[nH]c1ccc(Br)cc12]

To a solution of 36 g (154 mmol) of 5,7-dihydroindolo[2,3-c]quinolin-6-one in chloroform (1000 ml) is added, at 0° C. in the dark, 24.7 g (139 mmol) of N-bromosuccinimide in portions, and the mixture is stirred at this temperature for 2 h. The reaction is ended by addition of sodium sulfite solution and the mixture is stirred at room temperature for a further 30 min. After phase separation, the organic phase is washed with water and the aqueous phase is extracted with dichloromethane. The combined organic phases are dried over sodium sulfate and concentrated under reduced pressure. The residue is dissolved in toluene and filtered through silica gel. Subsequently, the crude product is recrystallized from toluene/heptane. Yield: 28.8 g (92 mmol), 60% of theory, colorless solid.

The following compounds can be obtained analogously:

| | Reactant | Product | Yield |
|---|---|---|---|
| | 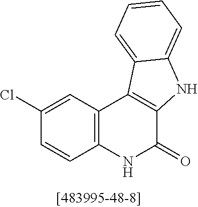 [SMILES: O=c1[nH]c2ccc(Cl)cc2c2c1[nH]c1ccccc12] | 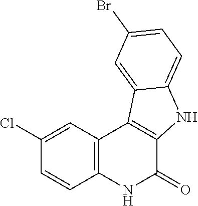 [SMILES: O=c1[nH]c2ccc(Cl)cc2c2c1[nH]c1ccc(Br)cc12] | 64% |
| 2a | 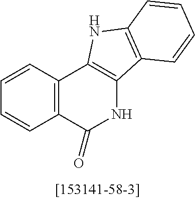 [SMILES: O=c1[nH]c2c3ccccc3[nH]c2c2ccccc12] | 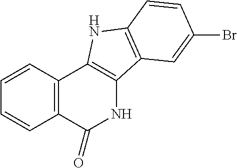 [SMILES: O=c1[nH]c2c3cc(Br)ccc3[nH]c2c2ccccc12] | 64% |
| 3a | 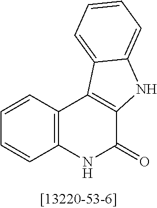 [SMILES: O=c1[nH]c2ccccc2c2c1[nH]c1ccccc12] | 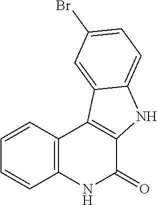 [SMILES: O=c1[nH]c2ccccc2c2c1[nH]c1ccc(Br)cc12] | 67% |
| 4a | 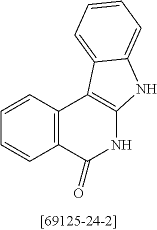 [SMILES: O=c1[nH]c2[nH]c3ccccc3c2c2ccccc12] | 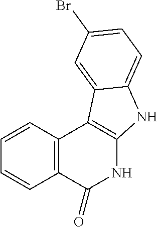 [SMILES: O=c1[nH]c2[nH]c3ccc(Br)cc3c2c2ccccc12] | 71% |
| 5a | 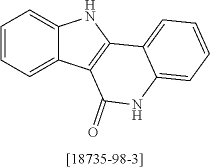 [SMILES: O=c1[nH]c2ccccc2c2[nH]c3ccccc3c12] | 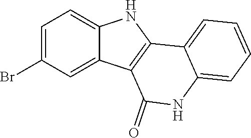 [SMILES: O=c1[nH]c2ccccc2c2[nH]c3ccc(Br)cc3c12] | 63% |
| 6a | 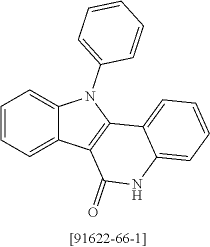 [SMILES: O=c1[nH]c2ccccc2c2c1c1ccccc1n2-c1ccccc1] | 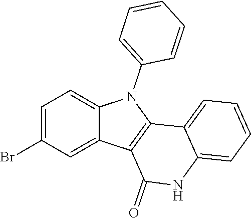 [SMILES: O=c1[nH]c2ccccc2c2c1c1cc(Br)ccc1n2-c1ccccc1] | 45% |

### b) 10-(9-Phenylcarbazol-3-yl)-5,7-dihydroindolo[2,3-c]quinolin-6-one

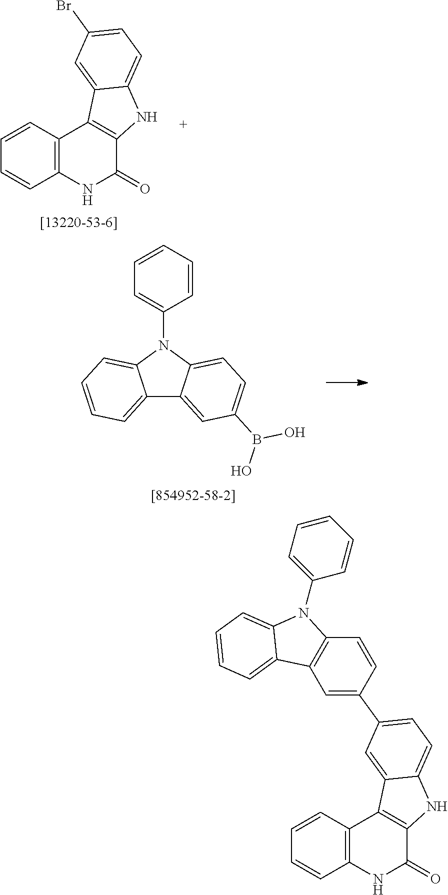 [SMILES: O=c1[nH]c2ccccc2c2c1[nH]c1ccc(Br)cc12 ; OB(O)c1ccc2c(c1)c1ccccc1n2-c1ccccc1 ; O=c1[nH]c2ccccc2c2c1[nH]c1ccc(-c3ccc4c(c3)c3ccccc3n4-c3ccccc3)cc12]

21.9 g (70 mmol) of 10-bromo-5,7-dihydroindolo[2,3-c]quinolin-6-one, 20.8 g (75 mmol) of phenylcarbazole-3-boronic acid and 14.7 g (139 mmol) of sodium carbonate are suspended in 200 ml of toluene, 52 ml of ethanol and 100 ml of water. 80 mg (0.69 mmol) of tetrakistriphenylphosphinepalladium(0) is added to this suspension, and the reaction mixture is heated under reflux for 16 h. After cooling, the organic phase is removed, filtered through silica gel, washed three times with 200 ml of water and then concentrated to dryness. The residue is recrystallized from heptane/dichloromethane. The yield is 26.5 g (56 mmol), 80% of theory.

The following compounds can be obtained in an analogous manner:

| | Reactant 1 | Reactant 2 | Product | Yield |
|---|---|---|---|---|
| | 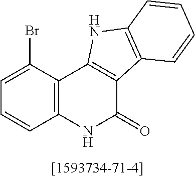 [SMILES: O=c1[nH]c2cccc(Br)c2c2[nH]c3ccccc3c12] | 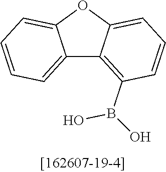 [SMILES: OB(O)c1cccc2oc3ccccc3c12] | 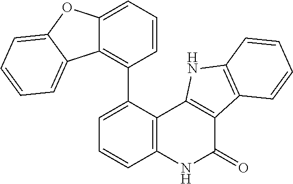 [SMILES: O=c1[nH]c2cccc(-c3cccc4oc5ccccc5c34)c2c2[nH]c3ccccc3c12] | 75% |
| 2b | 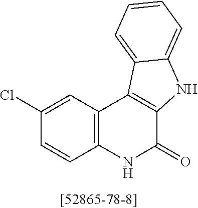 [SMILES: O=c1[nH]c2ccc(Cl)cc2c2c1[nH]c1ccccc12] | 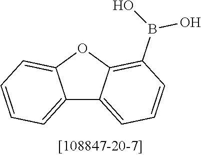 [SMILES: OB(O)c1cccc2c1oc1ccccc12] | 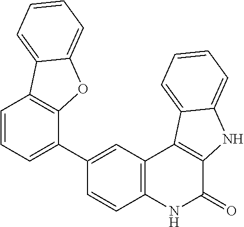 [SMILES: O=c1[nH]c2ccc(-c3cccc4c3oc3ccccc34)cc2c2c1[nH]c1ccccc12] | 64% |
| 3b | 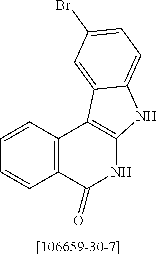 [SMILES: O=c1[nH]c2[nH]c3ccc(Br)cc3c2c2ccccc12] | 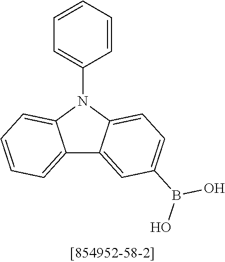 [SMILES: OB(O)c1ccc2c(c1)c1ccccc1n2-c1ccccc1] | 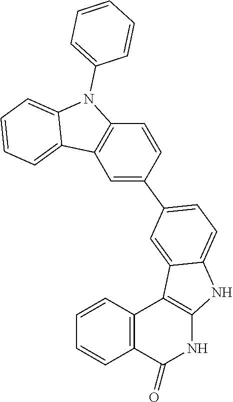 [SMILES: O=c1[nH]c2[nH]c3ccc(-c4ccc5c(c4)c4ccccc4n5-c4ccccc4)cc3c2c2ccccc12] | 76% |
| 4b | 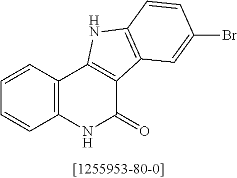 [SMILES: O=c1[nH]c2ccccc2c2[nH]c3ccc(Br)cc3c12] | 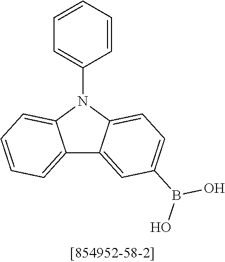 [SMILES: OB(O)c1ccc2c(c1)c1ccccc1n2-c1ccccc1] | 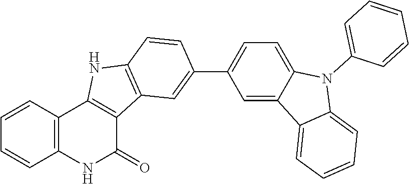 [SMILES: O=c1[nH]c2ccccc2c2[nH]c3ccc(-c4ccc5c(c4)c4ccccc4n5-c4ccccc4)cc3c12] | 81% |
| 5b | 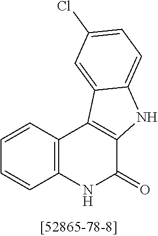 [SMILES: O=c1[nH]c2ccccc2c2c1[nH]c1ccc(Cl)cc12] | 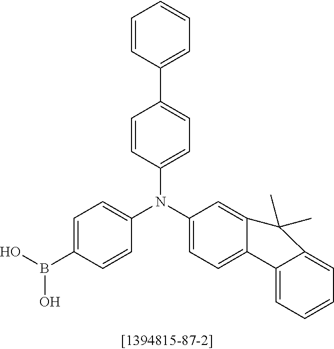 [SMILES: CC1(C)c2ccccc2-c2ccc(N(c3ccc(B(O)O)cc3)c3ccc(-c4ccccc4)cc3)cc21] | 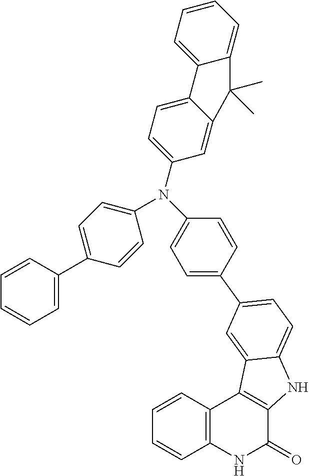 [SMILES: CC1(C)c2ccccc2-c2ccc(N(c3ccc(-c4ccccc4)cc3)c3ccc(-c4ccc5[nH]c6c(=O)[nH]c7ccccc7c6c5c4)cc3)cc21] | 63% |
| 6b | 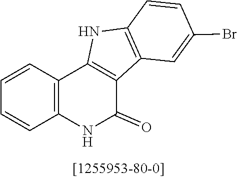 [SMILES: O=c1[nH]c2ccccc2c2[nH]c3ccc(Br)cc3c12] | 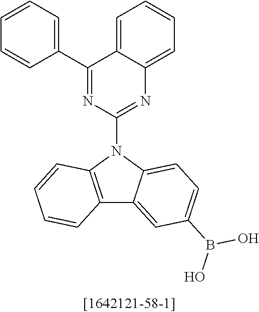 [SMILES: OB(O)c1ccc2c(c1)c1ccccc1n2-c1nc(-c2ccccc2)c2ccccc2n1] | 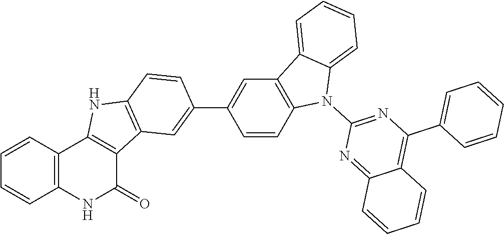 [SMILES: O=c1[nH]c2ccccc2c2[nH]c3ccc(-c4ccc5c(c4)c4ccccc4n5-c4nc(-c5ccccc5)c5ccccc5n4)cc3c12] | 76% |
| 7b | 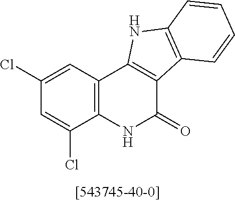 [SMILES: O=c1[nH]c2c(Cl)cc(Cl)cc2c2[nH]c3ccccc3c12] | 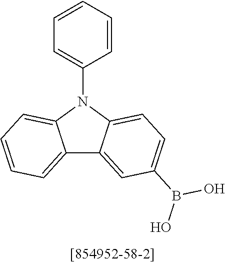 [SMILES: OB(O)c1ccc2c(c1)c1ccccc1n2-c1ccccc1] | 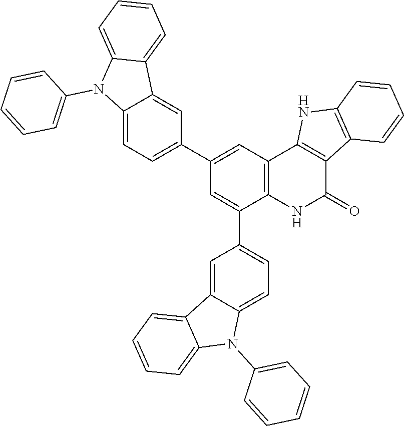 [SMILES: O=c1[nH]c2c(-c3ccc4c(c3)c3ccccc3n4-c3ccccc3)cc(-c3ccc4c(c3)c3ccccc3n4-c3ccccc3)cc2c2[nH]c3ccccc3c12] | 80% |
| 8b | 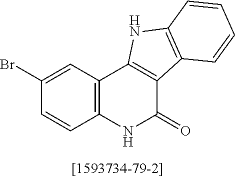 [SMILES: O=c1[nH]c2ccc(Br)cc2c2[nH]c3ccccc3c12] | 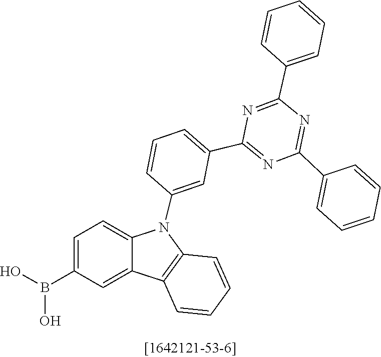 [SMILES: OB(O)c1ccc2c(c1)c1ccccc1n2-c1cccc(-c2nc(-c3ccccc3)nc(-c3ccccc3)n2)c1] | 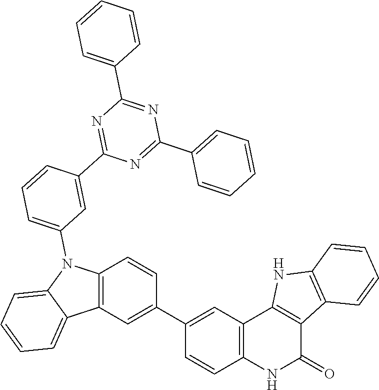 [SMILES: O=c1[nH]c2ccc(-c3ccc4c(c3)c3ccccc3n4-c3cccc(-c4nc(-c5ccccc5)nc(-c5ccccc5)n4)c3)cc2c2[nH]c3ccccc3c12] | 74% |
| 9b | 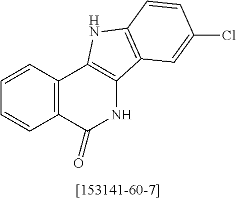 [SMILES: O=c1[nH]c2c3cc(Cl)ccc3[nH]c2c2ccccc12] | 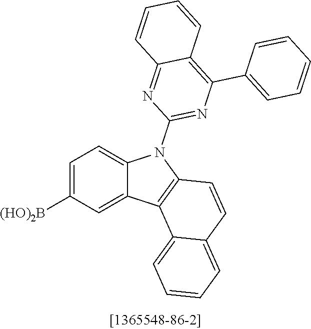 [SMILES: OB(O)c1ccc2c(c1)c1c3ccccc3ccc1n2-c1nc(-c2ccccc2)c2ccccc2n1] | 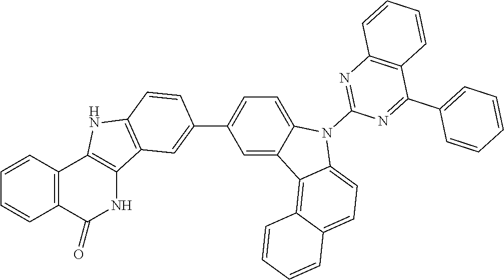 [SMILES: O=c1[nH]c2c3cc(-c4ccc5c(c4)c4c6ccccc6ccc4n5-c4nc(-c5ccccc5)c5ccccc5n4)ccc3[nH]c2c2ccccc12] | 77% |
| 10b | 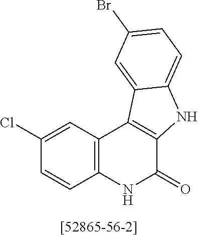 [SMILES: O=c1[nH]c2ccc(Cl)cc2c2c1[nH]c1ccc(Br)cc12] | 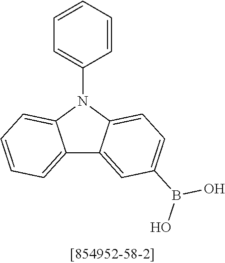 [SMILES: OB(O)c1ccc2c(c1)c1ccccc1n2-c1ccccc1] | 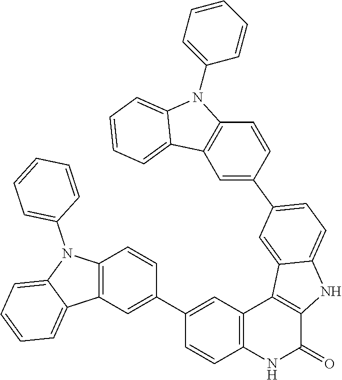 [SMILES: O=c1[nH]c2ccc(-c3ccc4c(c3)c3ccccc3n4-c3ccccc3)cc2c2c1[nH]c1ccc(-c3ccc4c(c3)c3ccccc3n4-c3ccccc3)cc12] | 54% |
| 11b | 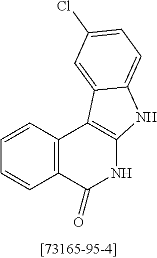 [SMILES: O=c1[nH]c2[nH]c3ccc(Cl)cc3c2c2ccccc12] | 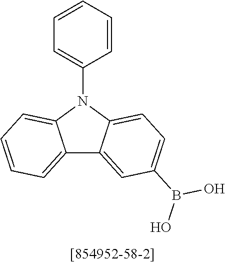 [SMILES: OB(O)c1ccc2c(c1)c1ccccc1n2-c1ccccc1] | 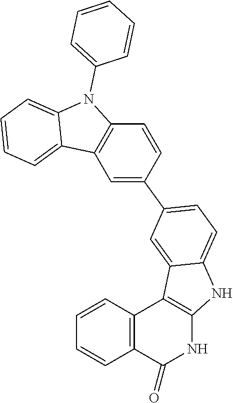 [SMILES: O=c1[nH]c2[nH]c3ccc(-c4ccc5c(c4)c4ccccc4n5-c4ccccc4)cc3c2c2ccccc12] | 76% |
| 12b | 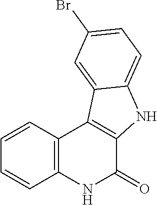 [SMILES: O=c1[nH]c2ccccc2c2c1[nH]c1ccc(Br)cc12] | 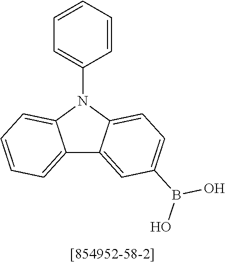 [SMILES: OB(O)c1ccc2c(c1)c1ccccc1n2-c1ccccc1] | 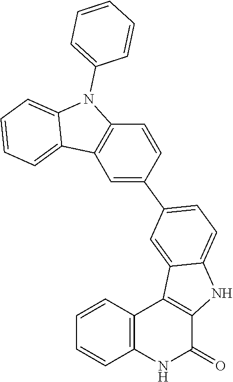 [SMILES: O=c1[nH]c2ccccc2c2c1[nH]c1ccc(-c3ccc4c(c3)c3ccccc3n4-c3ccccc3)cc12] | 70% |
| 13b | 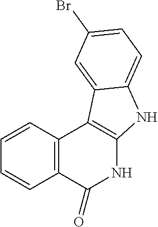 [SMILES: O=c1[nH]c2[nH]c3ccc(Br)cc3c2c2ccccc12] | 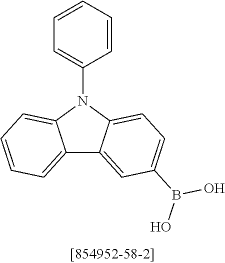 [SMILES: OB(O)c1ccc2c(c1)c1ccccc1n2-c1ccccc1] | 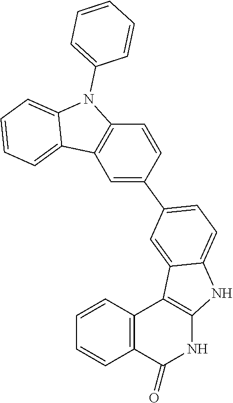 [SMILES: O=c1[nH]c2[nH]c3ccc(-c4ccc5c(c4)c4ccccc4n5-c4ccccc4)cc3c2c2ccccc12] | 68% |
| 14b | 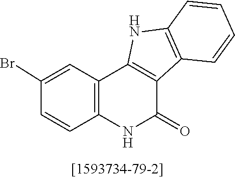 [SMILES: O=c1[nH]c2ccc(Br)cc2c2[nH]c3ccccc3c12] | 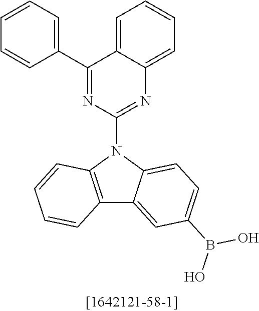 [SMILES: OB(O)c1ccc2c(c1)c1ccccc1n2-c1nc(-c2ccccc2)c2ccccc2n1] | 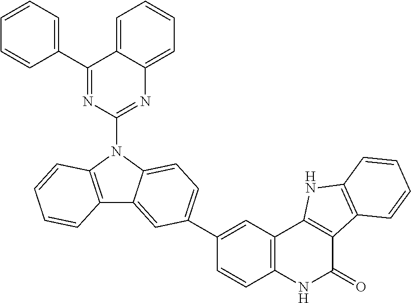 [SMILES: O=c1[nH]c2ccc(-c3ccc4c(c3)c3ccccc3n4-c3nc(-c4ccccc4)c4ccccc4n3)cc2c2[nH]c3ccccc3c12] | 67% |
| 15b | 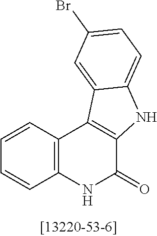 [SMILES: O=c1[nH]c2ccccc2c2c1[nH]c1ccc(Br)cc12] | 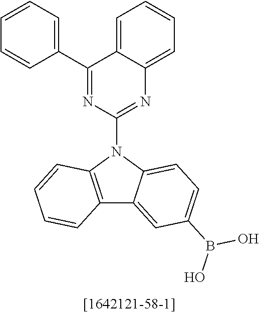 [SMILES: OB(O)c1ccc2c(c1)c1ccccc1n2-c1nc(-c2ccccc2)c2ccccc2n1] | 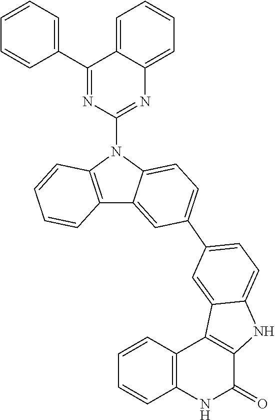 [SMILES: O=c1[nH]c2ccccc2c2c1[nH]c1ccc(-c3ccc4c(c3)c3ccccc3n4-c3nc(-c4ccccc4)c4ccccc4n3)cc12] | 66% |
| 16b | 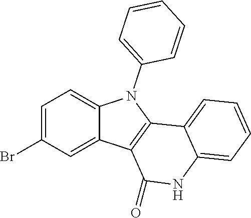 [SMILES: O=c1[nH]c2ccccc2c2c1c1cc(Br)ccc1n2-c1ccccc1] | 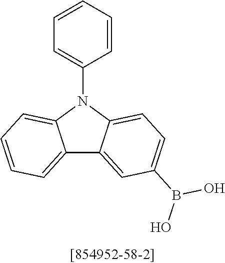 [SMILES: OB(O)c1ccc2c(c1)c1ccccc1n2-c1ccccc1] | 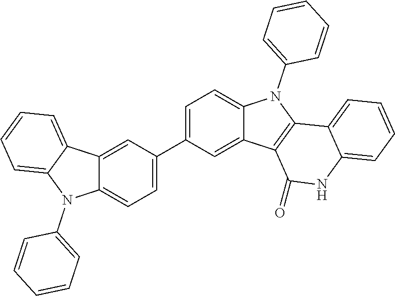 [SMILES: O=c1[nH]c2ccccc2c2c1c1cc(-c3ccc4c(c3)c3ccccc3n4-c3ccccc3)ccc1n2-c1ccccc1] | 73% |

### c) 8-(2-Nitrophenyl)-11-phenyl-5H-indolo[3,2-c]quinolin-6-one

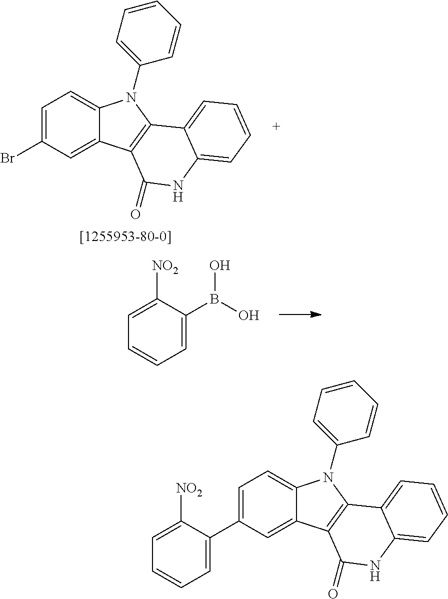 [SMILES: O=c1[nH]c2ccccc2c2c1c1cc(Br)ccc1n2-c1ccccc1 ; O=[N+]([O-])c1ccccc1B(O)O ; O=c1[nH]c2ccccc2c2c1c1cc(-c3ccccc3[N+](=O)[O-])ccc1n2-c1ccccc1]

To a well-stirred, degassed suspension of 30 g (184 mmol) of 2-nitrophenylboronic acid, 70 g (180 mmol) of 8-bromo-11-phenyl-5H-indolo[3,2-c]quinolin-6-one and 66.5 g (212.7 mmol) of potassium carbonate in a mixture of 250 ml of water and 250 ml of THE is added 1.7 g (1.49 mmol) of Pd(PPh₃)₄, and the mixture is heated under reflux for 17 h. After cooling, the organic phase is removed, washed three times with 200 ml of water and once with 200 ml of saturated aqueous sodium chloride solution, dried over magnesium sulfate and concentrated to dryness by rotary evaporation. The gray residue is recrystallized from hexane. The precipitated crystals are filtered off with suction, washed with a little MeOH and dried under reduced pressure. Yield: 58 g (134 mmol); 75% of theory.

The following compounds can be obtained in an analogous manner:

| | Reactant 1 | Reactant 2 | Product | Yield |
|---|---|---|---|---|
| | 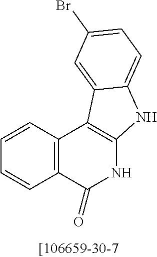 [SMILES: O=c1[nH]c2[nH]c3ccc(Br)cc3c2c2ccccc12] | 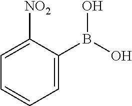 [SMILES: O=[N+]([O-])c1ccccc1B(O)O] | 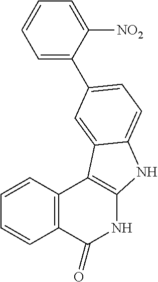 [SMILES: O=c1[nH]c2[nH]c3ccc(-c4ccccc4[N+](=O)[O-])cc3c2c2ccccc12] | 76% |
| 2c | 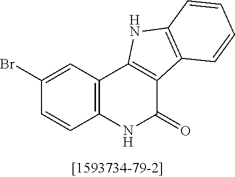 [SMILES: O=c1[nH]c2ccc(Br)cc2c2[nH]c3ccccc3c12] | 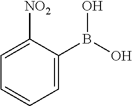 [SMILES: O=[N+]([O-])c1ccccc1B(O)O] | 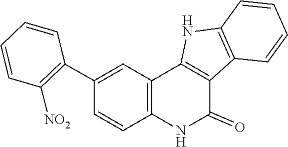 [SMILES: O=c1[nH]c2ccc(-c3ccccc3[N+](=O)[O-])cc2c2[nH]c3ccccc3c12] | 74% |
| 3c | 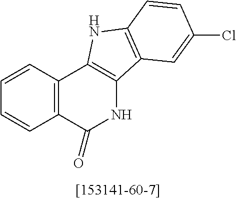 [SMILES: O=c1[nH]c2c3cc(Cl)ccc3[nH]c2c2ccccc12] | 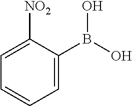 [SMILES: O=[N+]([O-])c1ccccc1B(O)O] | 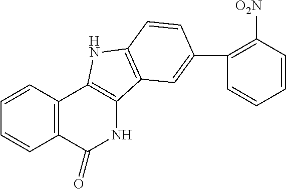 [SMILES: O=c1[nH]c2c3cc(-c4ccccc4[N+](=O)[O-])ccc3[nH]c2c2ccccc12] | 63% |
| 4c | 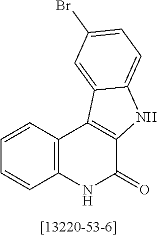 [SMILES: O=c1[nH]c2ccccc2c2c1[nH]c1ccc(Br)cc12] | 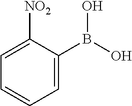 [SMILES: O=[N+]([O-])c1ccccc1B(O)O] | 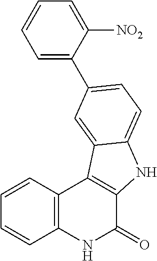 [SMILES: O=c1[nH]c2ccccc2c2c1[nH]c1ccc(-c3ccccc3[N+](=O)[O-])cc12] | 75% |
| 5c | 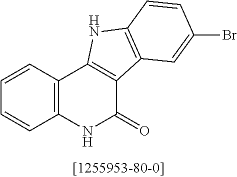 [SMILES: O=c1[nH]c2ccccc2c2[nH]c3ccc(Br)cc3c12] | 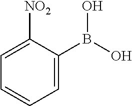 [SMILES: O=[N+]([O-])c1ccccc1B(O)O] | 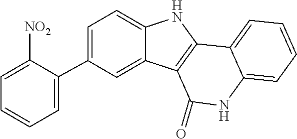 [SMILES: O=c1[nH]c2ccccc2c2[nH]c3ccc(-c4ccccc4[N+](=O)[O-])cc3c12] | 79% |

### d) Cyclization

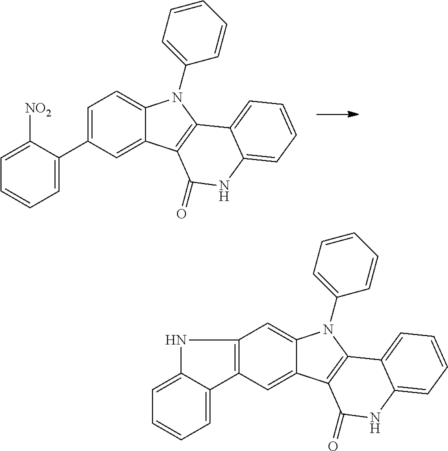 [SMILES: O=c1[nH]c2ccccc2c2c1c1cc(-c3ccccc3[N+](=O)[O-])ccc1n2-c1ccccc1 ; O=c1[nH]c2ccccc2c2c1c1cc3c(cc1n2-c1ccccc1)[nH]c1ccccc13]

A mixture of 94 g (220 mmol) of 8-(2-nitrophenyl)-11-phenyl-5H-indolo[3,2-c]quinolin-6-one and 290.3 ml (1669 mmol) of triethyl phosphite is heated under reflux for 12 h. Subsequently, the rest of the triethyl phosphite is distilled off (72-76° C./9 mmHg). Water/MeOH (1:1) is added to the residue, and the solids are filtered off and recrystallized. Yield: 62 g (156 mmol); 71% of theory.

The following compounds can be obtained in an analogous manner:

| | Reactant | Product | Yield |
|---|---|---|---|
| | 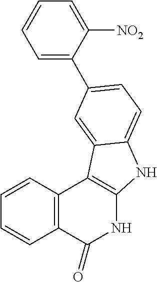 [SMILES: O=c1[nH]c2[nH]c3ccc(-c4ccccc4[N+](=O)[O-])cc3c2c2ccccc12] | 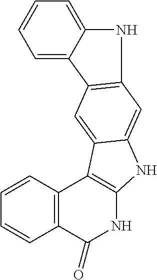 [SMILES: O=c1[nH]c2[nH]c3cc4[nH]c5ccccc5c4cc3c2c2ccccc12] | 75% |
| 2d | 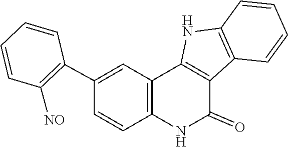 | 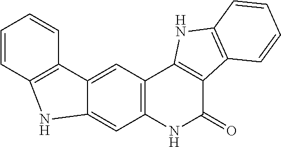 [SMILES: O=c1[nH]c2cc3[nH]c4ccccc4c3cc2c2[nH]c3ccccc3c12] | 64% |
| 3d | 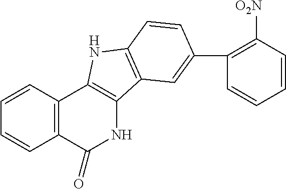 [SMILES: O=c1[nH]c2c3cc(-c4ccccc4[N+](=O)[O-])ccc3[nH]c2c2ccccc12] | 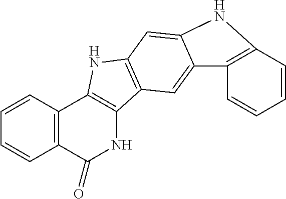 [SMILES: O=c1[nH]c2c3cc4c(cc3[nH]c2c2ccccc12)[nH]c1ccccc14] | 78% |
| 4d | 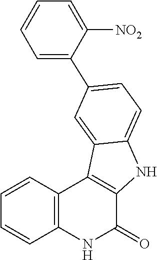 [SMILES: O=c1[nH]c2ccccc2c2c1[nH]c1ccc(-c3ccccc3[N+](=O)[O-])cc12] | 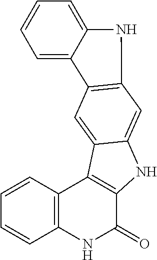 [SMILES: O=c1[nH]c2ccccc2c2c1[nH]c1cc3[nH]c4ccccc4c3cc12] | 70% |
| 5d | 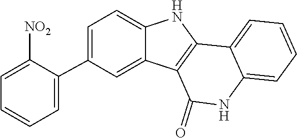 [SMILES: O=c1[nH]c2ccccc2c2[nH]c3ccc(-c4ccccc4[N+](=O)[O-])cc3c12] | 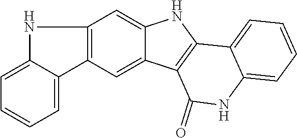 [SMILES: O=c1[nH]c2ccccc2c2[nH]c3cc4[nH]c5ccccc5c4cc3c12] | 76% |

### e) 7-(4,6-Diphenyl-1,3,5-triazin-2-yl)-10-(9-phenylcarbazol-3-yl)-5H-indolo[2,3-c]quinolin-6-one

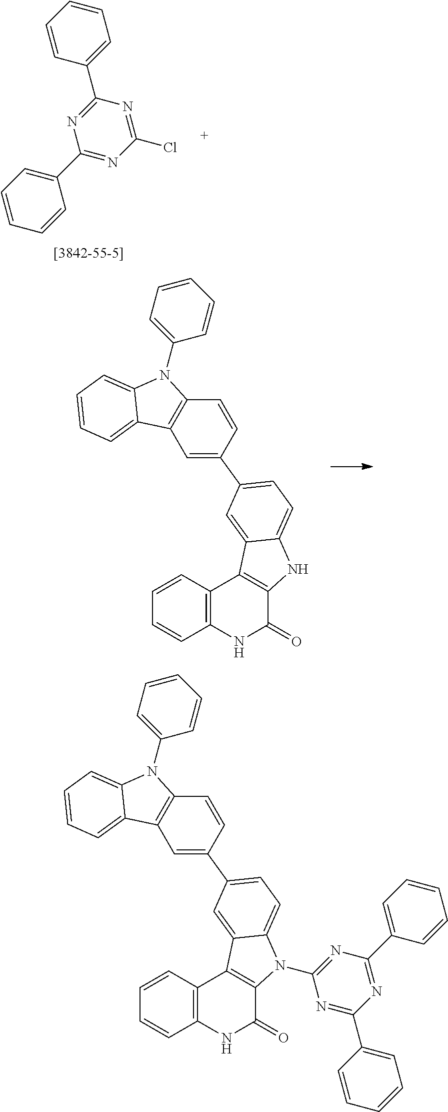

25 g (50 mmol) of 10-(9-phenylcarbazol-3-yl)-5,7-dihydroindolo[2,3-c]quinolin-6-one and 16 g (60 mmol) of 2-chloro-4,6-diphenyl-[1,3,5]triazine are dissolved in 400 ml of toluene under an argon atmosphere. 1.0 g (5 mmol) of tri-tert-butylphosphine is added and the mixture is stirred under an argon atmosphere. 0.6 g (2 mmol) of Pd(OAc)₂ is added and the mixture is stirred under an argon atmosphere, and then 9.5 g (99 mmol) of sodium tert-butoxide are added. The reaction mixture is stirred under reflux for 24 h. After cooling, the organic phase is separated, washed three times with 200 ml of water, dried over MgSO₄ and filtered, and the solvent is removed under reduced pressure. The residue is purified by column chromatography using silica gel (eluent: DCM/heptane (1:4)). Yield 47 g (66 mmol); 63% of theory.

In the case of 23c, 24c and 25c, the residue is recrystallized from toluene and finally sublimed under high vacuum (p=5×10⁻⁵ mbar). The purity is 99.9%.

The following compounds can be obtained analogously:

| | Reactant 1 | Reactant 2 |
|---|---|---|
| | 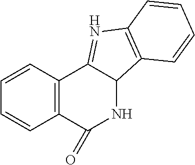 | 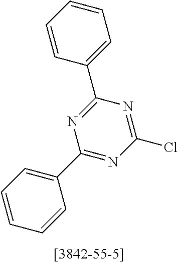 [SMILES: Clc1nc(-c2ccccc2)nc(-c2ccccc2)n1] |
| 2e | 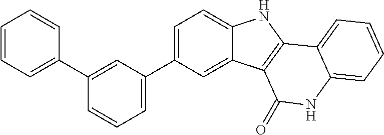 [SMILES: O=c1[nH]c2ccccc2c2[nH]c3ccc(-c4cccc(-c5ccccc5)c4)cc3c12] | 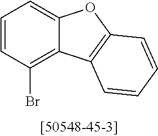 [SMILES: Brc1cccc2oc3ccccc3c12] |
| 3e | 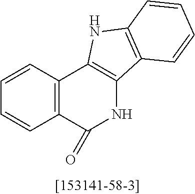 [SMILES: O=c1[nH]c2c3ccccc3[nH]c2c2ccccc12] | 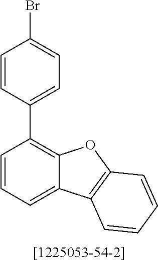 [SMILES: Brc1ccc(-c2cccc3c2oc2ccccc23)cc1] |
| 4e | 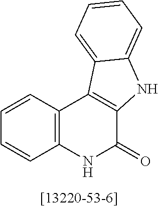 [SMILES: O=c1[nH]c2ccccc2c2c1[nH]c1ccccc12] | 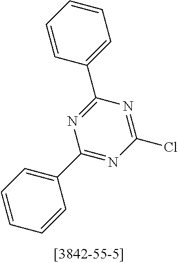 [SMILES: Clc1nc(-c2ccccc2)nc(-c2ccccc2)n1] |
| 5e | 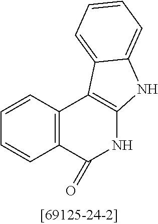 [SMILES: O=c1[nH]c2[nH]c3ccccc3c2c2ccccc12] | 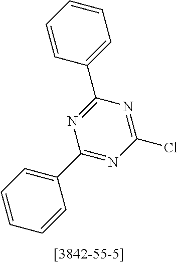 [SMILES: Clc1nc(-c2ccccc2)nc(-c2ccccc2)n1] |
| 6e | 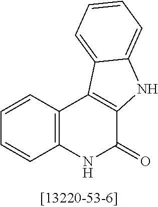 [SMILES: O=c1[nH]c2ccccc2c2c1[nH]c1ccccc12] | 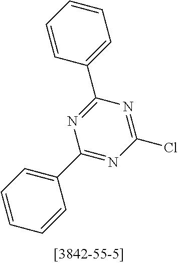 [SMILES: Clc1nc(-c2ccccc2)nc(-c2ccccc2)n1] |
| 7e | 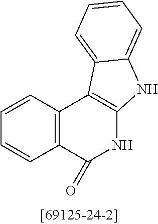 [SMILES: O=c1[nH]c2[nH]c3ccccc3c2c2ccccc12] | 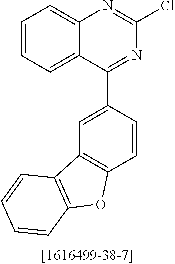 [SMILES: Clc1nc(-c2ccc3oc4ccccc4c3c2)c2ccccc2n1] |
| 8e | 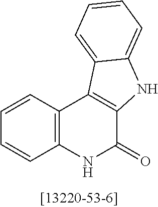 [SMILES: O=c1[nH]c2ccccc2c2c1[nH]c1ccccc12] | 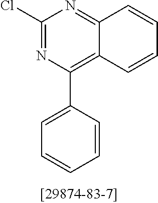 [SMILES: Clc1nc(-c2ccccc2)c2ccccc2n1] |
| 9e | 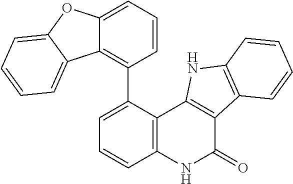 [SMILES: O=c1[nH]c2cccc(-c3cccc4oc5ccccc5c34)c2c2[nH]c3ccccc3c12] | 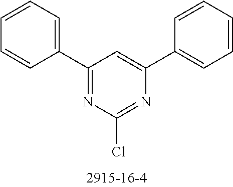 [SMILES: Clc1nc(-c2ccccc2)cc(-c2ccccc2)n1] |
| 10e | 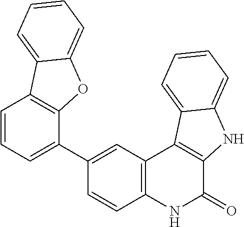 [SMILES: O=c1[nH]c2ccc(-c3cccc4c3oc3ccccc34)cc2c2c1[nH]c1ccccc12] | 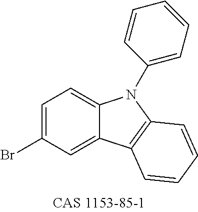 [SMILES: Brc1ccc2c(c1)c1ccccc1n2-c1ccccc1] |
| 11e | 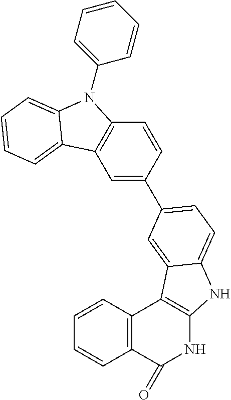 [SMILES: O=c1[nH]c2[nH]c3ccc(-c4ccc5c(c4)c4ccccc4n5-c4ccccc4)cc3c2c2ccccc12] | 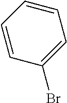 |
| 12e | 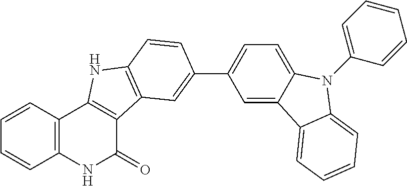 [SMILES: O=c1[nH]c2ccccc2c2[nH]c3ccc(-c4ccc5c(c4)c4ccccc4n5-c4ccccc4)cc3c12] | 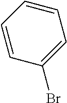 |
| 13e | 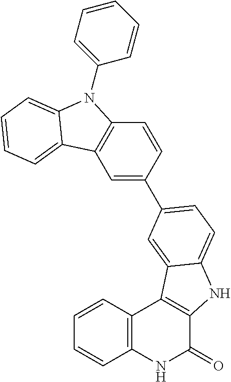 [SMILES: O=c1[nH]c2ccccc2c2c1[nH]c1ccc(-c3ccc4c(c3)c3ccccc3n4-c3ccccc3)cc12] | 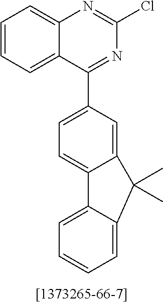 [SMILES: CC1(C)c2ccccc2-c2ccc(-c3nc(Cl)nc4ccccc34)cc21] |
| 14e | 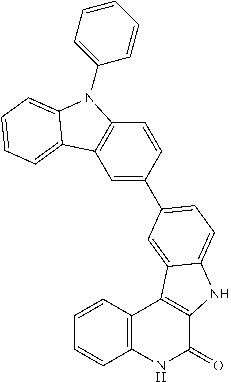 [SMILES: O=c1[nH]c2ccccc2c2c1[nH]c1ccc(-c3ccc4c(c3)c3ccccc3n4-c3ccccc3)cc12] | 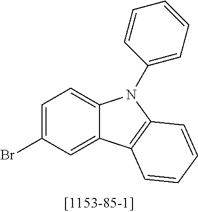 [SMILES: Brc1ccc2c(c1)c1ccccc1n2-c1ccccc1] |
| 15e | 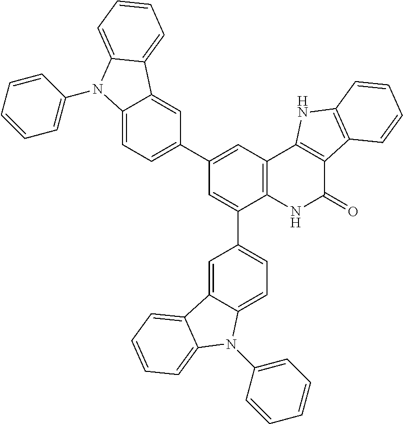 [SMILES: O=c1[nH]c2c(-c3ccc4c(c3)c3ccccc3n4-c3ccccc3)cc(-c3ccc4c(c3)c3ccccc3n4-c3ccccc3)cc2c2[nH]c3ccccc3c12] | 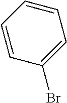 |
| 16e | 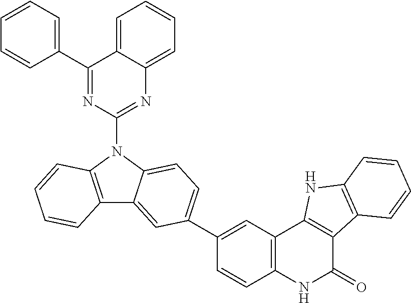 [SMILES: O=c1[nH]c2ccc(-c3ccc4c(c3)c3ccccc3n4-c3nc(-c4ccccc4)c4ccccc4n3)cc2c2[nH]c3ccccc3c12] | 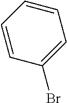 |
| 17e | 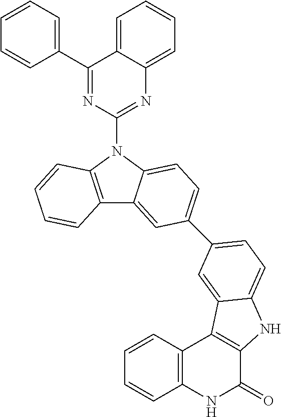 [SMILES: O=c1[nH]c2ccccc2c2c1[nH]c1ccc(-c3ccc4c(c3)c3ccccc3n4-c3nc(-c4ccccc4)c4ccccc4n3)cc12] | 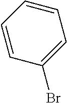 |
| 18e | 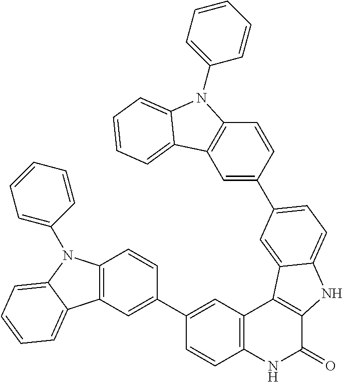 [SMILES: O=c1[nH]c2ccc(-c3ccc4c(c3)c3ccccc3n4-c3ccccc3)cc2c2c1[nH]c1ccc(-c3ccc4c(c3)c3ccccc3n4-c3ccccc3)cc12] | 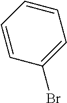 |
| 19e | 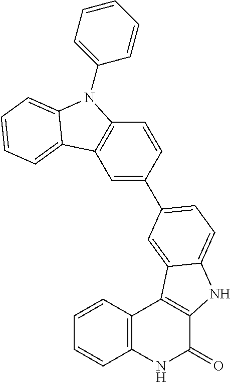 [SMILES: O=c1[nH]c2ccccc2c2c1[nH]c1ccc(-c3ccc4c(c3)c3ccccc3n4-c3ccccc3)cc12] | 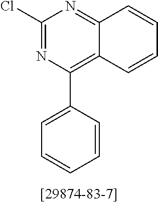 [SMILES: Clc1nc(-c2ccccc2)c2ccccc2n1] |
| 20e | 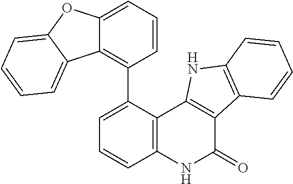 [SMILES: O=c1[nH]c2cccc(-c3cccc4oc5ccccc5c34)c2c2[nH]c3ccccc3c12] | 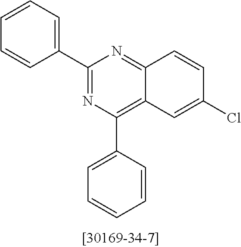 [SMILES: Clc1ccc2nc(-c3ccccc3)nc(-c3ccccc3)c2c1] |
| 21e | 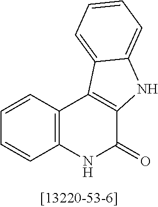 [SMILES: O=c1[nH]c2ccccc2c2c1[nH]c1ccccc12] | 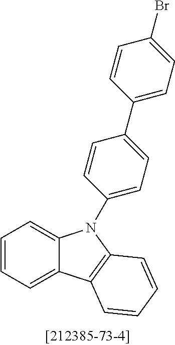 [SMILES: Brc1ccc(-c2ccc(-n3c4ccccc4c4ccccc43)cc2)cc1] |
| 22e | 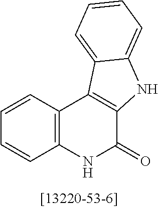 [SMILES: O=c1[nH]c2ccccc2c2c1[nH]c1ccccc12] | 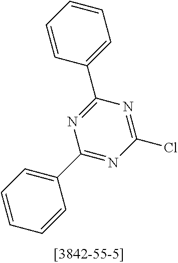 [SMILES: Clc1nc(-c2ccccc2)nc(-c2ccccc2)n1] |
| 23e | 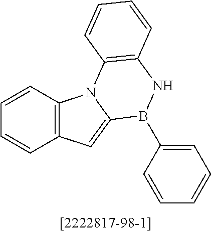 [SMILES: c1ccc(B2Nc3ccccc3-n3c2cc2ccccc23)cc1] | 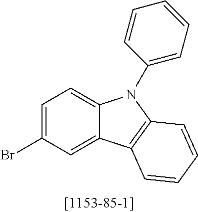 [SMILES: Brc1ccc2c(c1)c1ccccc1n2-c1ccccc1] |
| 24e | 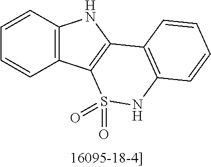 [SMILES: O=S1(=O)Nc2ccccc2-c2[nH]c3ccccc3c21] | 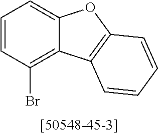 [SMILES: Brc1cccc2oc3ccccc3c12] |
| 25e | 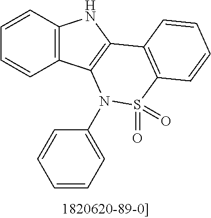 [SMILES: O=S1(=O)c2ccccc2-c2[nH]c3ccccc3c2N1c1ccccc1] | 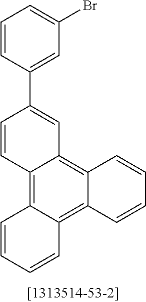 [SMILES: Brc1cccc(-c2ccc3c4ccccc4c4ccccc4c3c2)c1] |
| 26e | 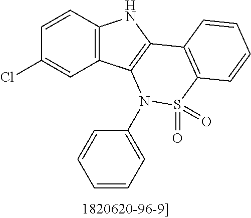 [SMILES: O=S1(=O)c2ccccc2-c2[nH]c3ccc(Cl)cc3c2N1c1ccccc1] | 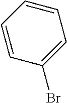 |
| 27e | 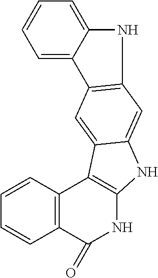 [SMILES: O=c1[nH]c2[nH]c3cc4[nH]c5ccccc5c4cc3c2c2ccccc12] | 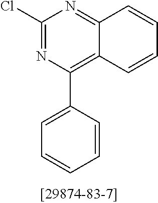 [SMILES: Clc1nc(-c2ccccc2)c2ccccc2n1] |
| 28e | 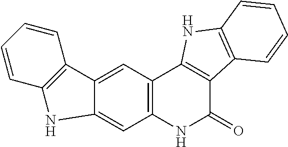 [SMILES: O=c1[nH]c2cc3[nH]c4ccccc4c3cc2c2[nH]c3ccccc3c12] | 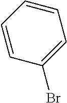 |
| 29e | 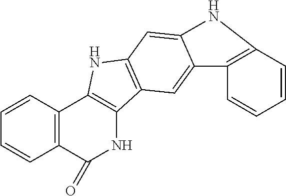 [SMILES: O=c1[nH]c2c3cc4c(cc3[nH]c2c2ccccc12)[nH]c1ccccc14] | 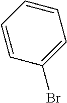 |
| 30e | 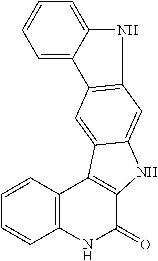 [SMILES: O=c1[nH]c2ccccc2c2c1[nH]c1cc3[nH]c4ccccc4c3cc12] | 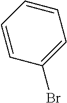 |
| 31e | 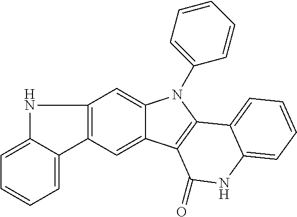 [SMILES: O=c1[nH]c2ccccc2c2c1c1cc3c(cc1n2-c1ccccc1)[nH]c1ccccc13] | 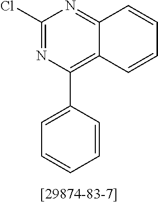 [SMILES: Clc1nc(-c2ccccc2)c2ccccc2n1] |
| 32e | 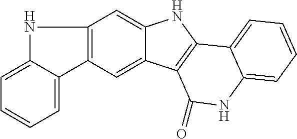 [SMILES: O=c1[nH]c2ccccc2c2[nH]c3cc4[nH]c5ccccc5c4cc3c12] | 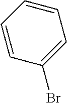 |
| | Product | Yield |
| | 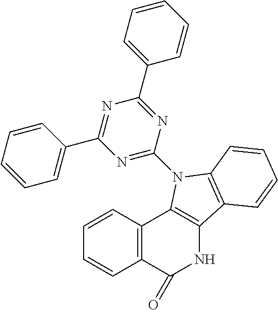 [SMILES: O=c1[nH]c2c3ccccc3n(-c3nc(-c4ccccc4)nc(-c4ccccc4)n3)c2c2ccccc12] | 61% |
| 2e | 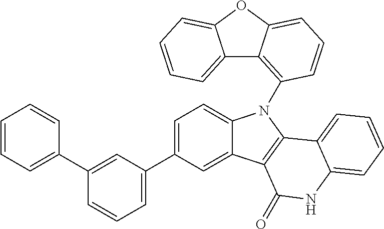 [SMILES: O=c1[nH]c2ccccc2c2c1c1cc(-c3cccc(-c4ccccc4)c3)ccc1n2-c1cccc2oc3ccccc3c12] | 64% |
| 3e | 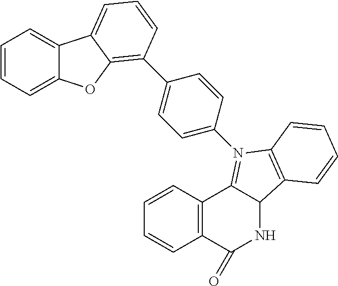 | 67% |
| 4e | 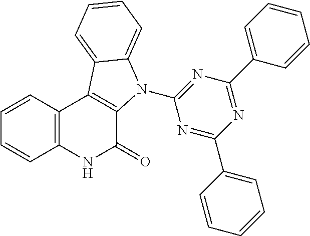 [SMILES: O=c1[nH]c2ccccc2c2c3ccccc3n(-c3nc(-c4ccccc4)nc(-c4ccccc4)n3)c12] | 71% |
| 5e | 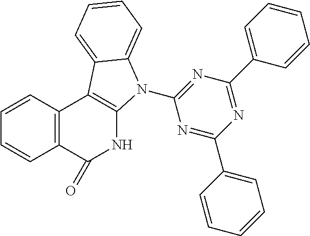 [SMILES: O=c1[nH]c2c(c3ccccc13)c1ccccc1n2-c1nc(-c2ccccc2)nc(-c2ccccc2)n1] | 70% |
| 6e | 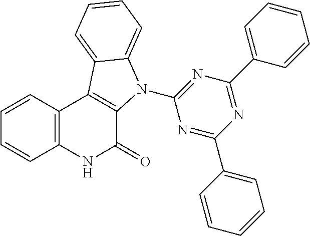 [SMILES: O=c1[nH]c2ccccc2c2c3ccccc3n(-c3nc(-c4ccccc4)nc(-c4ccccc4)n3)c12] | 68% |
| 7e | 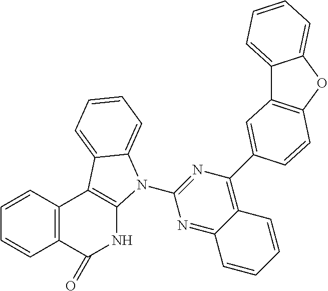 [SMILES: O=c1[nH]c2c(c3ccccc13)c1ccccc1n2-c1nc(-c2ccc3oc4ccccc4c3c2)c2ccccc2n1] | 59% |
| 8e | 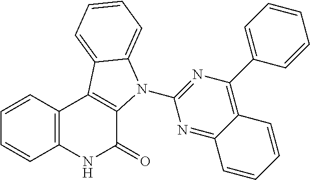 [SMILES: O=c1[nH]c2ccccc2c2c3ccccc3n(-c3nc(-c4ccccc4)c4ccccc4n3)c12] | 55% |
| 9e | 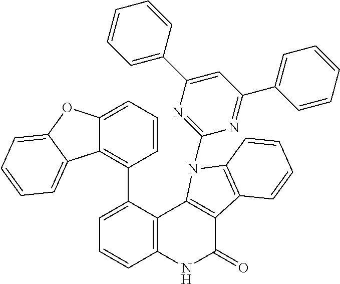 [SMILES: O=c1[nH]c2cccc(-c3cccc4oc5ccccc5c34)c2c2c1c1ccccc1n2-c1nc(-c2ccccc2)cc(-c2ccccc2)n1] | 60% |
| 10e | 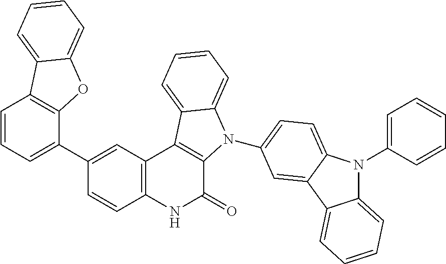 [SMILES: O=c1[nH]c2ccc(-c3cccc4c3oc3ccccc34)cc2c2c3ccccc3n(-c3ccc4c(c3)c3ccccc3n4-c3ccccc3)c12] | 58% |
| 11e | 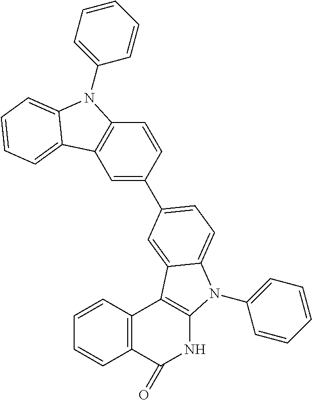 [SMILES: O=c1[nH]c2c(c3ccccc13)c1cc(-c3ccc4c(c3)c3ccccc3n4-c3ccccc3)ccc1n2-c1ccccc1] | 62% |
| 12e | 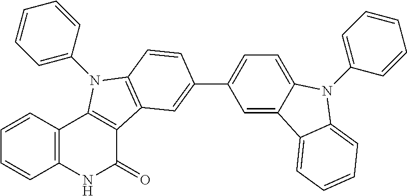 [SMILES: O=c1[nH]c2ccccc2c2c1c1cc(-c3ccc4c(c3)c3ccccc3n4-c3ccccc3)ccc1n2-c1ccccc1] | 61% |
| 13e | 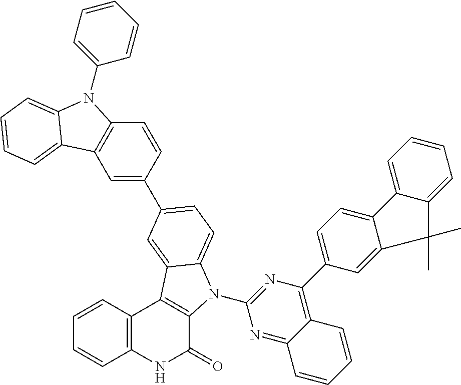 [SMILES: CC1(C)c2ccccc2-c2ccc(-c3nc(-n4c5ccc(-c6ccc7c(c6)c6ccccc6n7-c6ccccc6)cc5c5c6ccccc6[nH]c(=O)c54)nc4ccccc34)cc21] | 65% |
| 14e | 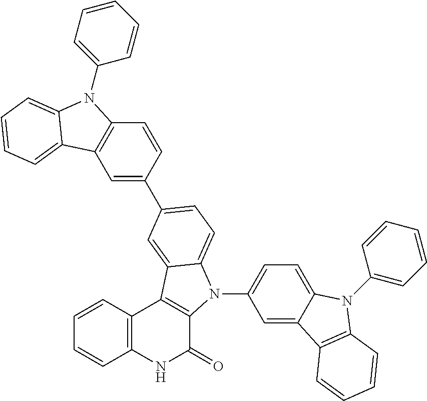 [SMILES: O=c1[nH]c2ccccc2c2c3cc(-c4ccc5c(c4)c4ccccc4n5-c4ccccc4)ccc3n(-c3ccc4c(c3)c3ccccc3n4-c3ccccc3)c12] | 64% |
| 15e | 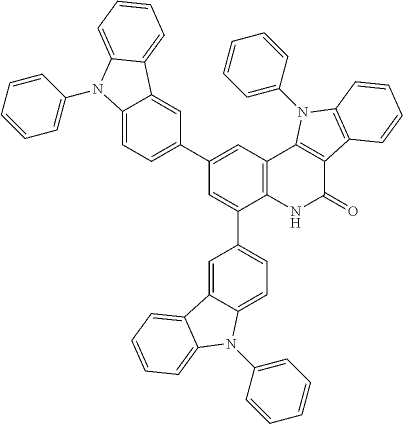 [SMILES: O=c1[nH]c2c(-c3ccc4c(c3)c3ccccc3n4-c3ccccc3)cc(-c3ccc4c(c3)c3ccccc3n4-c3ccccc3)cc2c2c1c1ccccc1n2-c1ccccc1] | 65% |
| 16e | 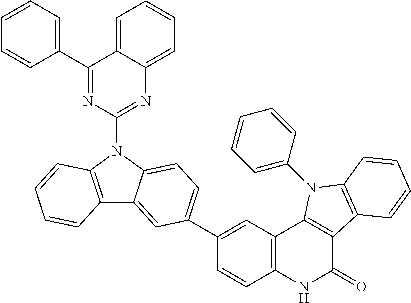 [SMILES: O=c1[nH]c2ccc(-c3ccc4c(c3)c3ccccc3n4-c3nc(-c4ccccc4)c4ccccc4n3)cc2c2c1c1ccccc1n2-c1ccccc1] | |
| 17e | 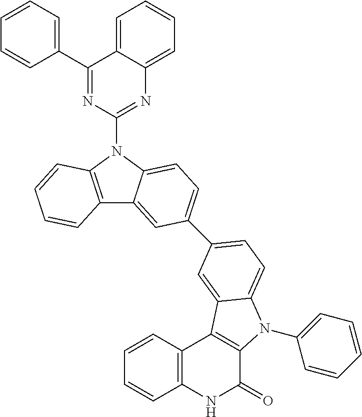 [SMILES: O=c1[nH]c2ccccc2c2c3cc(-c4ccc5c(c4)c4ccccc4n5-c4nc(-c5ccccc5)c5ccccc5n4)ccc3n(-c3ccccc3)c12] | 63% |
| 18e | 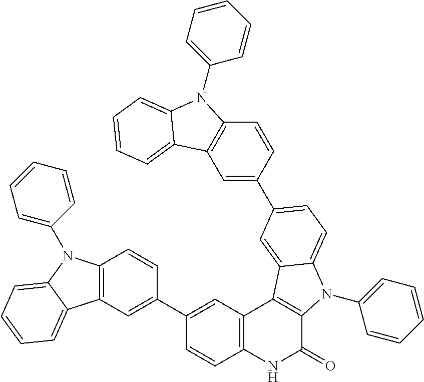 [SMILES: O=c1[nH]c2ccc(-c3ccc4c(c3)c3ccccc3n4-c3ccccc3)cc2c2c3cc(-c4ccc5c(c4)c4ccccc4n5-c4ccccc4)ccc3n(-c3ccccc3)c12] | 67% |
| 19e | 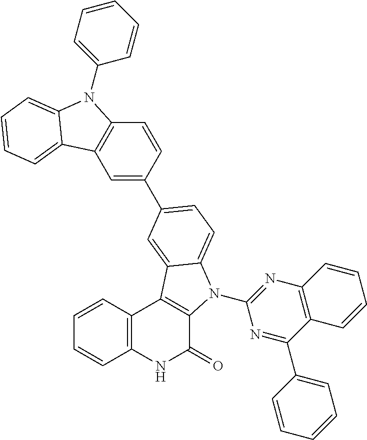 [SMILES: O=c1[nH]c2ccccc2c2c3cc(-c4ccc5c(c4)c4ccccc4n5-c4ccccc4)ccc3n(-c3nc(-c4ccccc4)c4ccccc4n3)c12] | 61% |
| 20e | 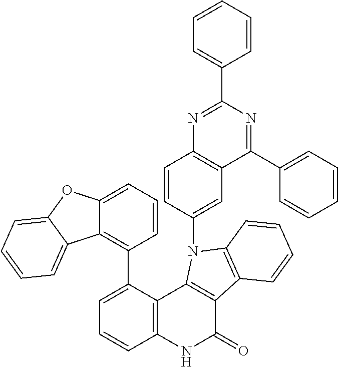 [SMILES: O=c1[nH]c2cccc(-c3cccc4oc5ccccc5c34)c2c2c1c1ccccc1n2-c1ccc2nc(-c3ccccc3)nc(-c3ccccc3)c2c1] | 60% |
| 21e | 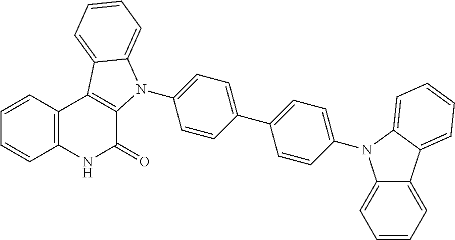 [SMILES: O=c1[nH]c2ccccc2c2c3ccccc3n(-c3ccc(-c4ccc(-n5c6ccccc6c6ccccc65)cc4)cc3)c12] | 68% |
| 22e | 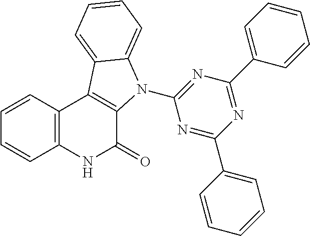 [SMILES: O=c1[nH]c2ccccc2c2c3ccccc3n(-c3nc(-c4ccccc4)nc(-c4ccccc4)n3)c12] | 68% |
| 23e | 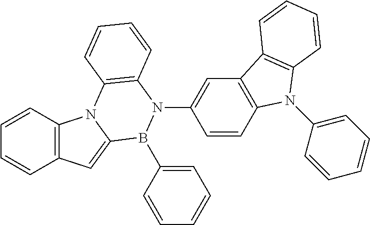 [SMILES: c1ccc(B2c3cc4ccccc4n3-c3ccccc3N2c2ccc3c(c2)c2ccccc2n3-c2ccccc2)cc1] | 57% |
| 24e | 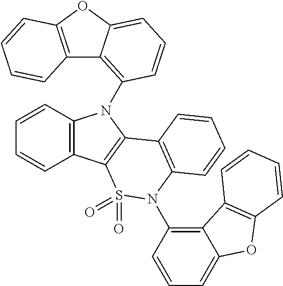 [SMILES: O=S1(=O)c2c(n(-c3cccc4oc5ccccc5c34)c3ccccc23)-c2ccccc2N1c1cccc2oc3ccccc3c12] | 61% |
| 25e | 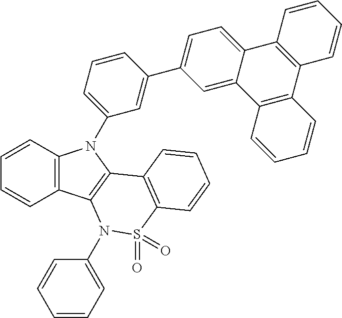 [SMILES: O=S1(=O)c2ccccc2-c2c(c3ccccc3n2-c2cccc(-c3ccc4c5ccccc5c5ccccc5c4c3)c2)N1c1ccccc1] | 64% |
| 26e | 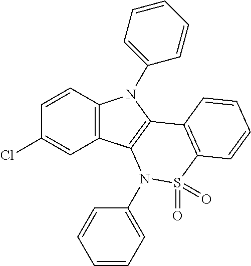 [SMILES: O=S1(=O)c2ccccc2-c2c(c3cc(Cl)ccc3n2-c2ccccc2)N1c1ccccc1] | 59% |
| 27e | 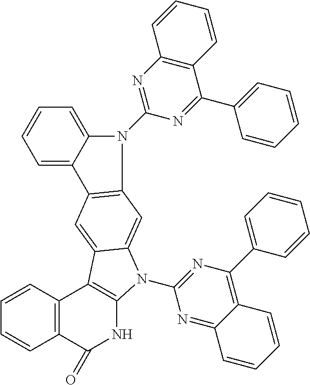 [SMILES: O=c1[nH]c2c(c3ccccc13)c1cc3c4ccccc4n(-c4nc(-c5ccccc5)c5ccccc5n4)c3cc1n2-c1nc(-c2ccccc2)c2ccccc2n1] | 52% |
| 28e | 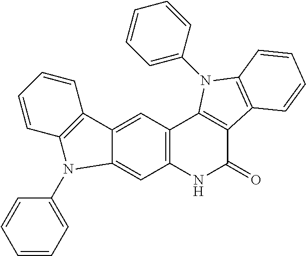 [SMILES: O=c1[nH]c2cc3c(cc2c2c1c1ccccc1n2-c1ccccc1)c1ccccc1n3-c1ccccc1] | 78% |
| 29e | 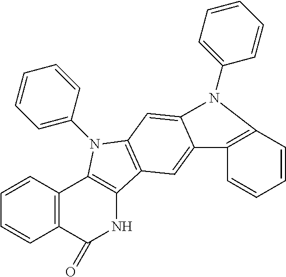 [SMILES: O=c1[nH]c2c3cc4c5ccccc5n(-c5ccccc5)c4cc3n(-c3ccccc3)c2c2ccccc12] | 80% |
| 30e | 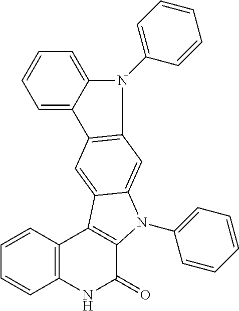 [SMILES: O=c1[nH]c2ccccc2c2c3cc4c5ccccc5n(-c5ccccc5)c4cc3n(-c3ccccc3)c12] | 69% |
| 31e | 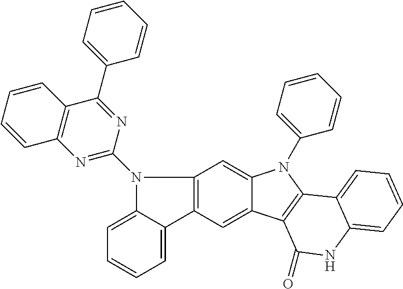 [SMILES: O=c1[nH]c2ccccc2c2c1c1cc3c4ccccc4n(-c4nc(-c5ccccc5)c5ccccc5n4)c3cc1n2-c1ccccc1] | 81% |
| 32e | 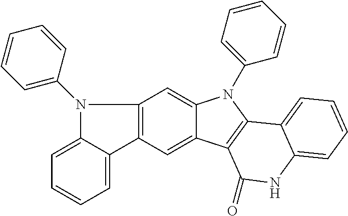 [SMILES: O=c1[nH]c2ccccc2c2c1c1cc3c4ccccc4n(-c4ccccc4)c3cc1n2-c1ccccc1] | |

### f) 7-(4,6-Diphenyl-1,3,5-triazin-2-yl)-5-phenyl-10-(9-phenylcarbazol-3-yl)indolo[2,3-c]quinolin-6-one

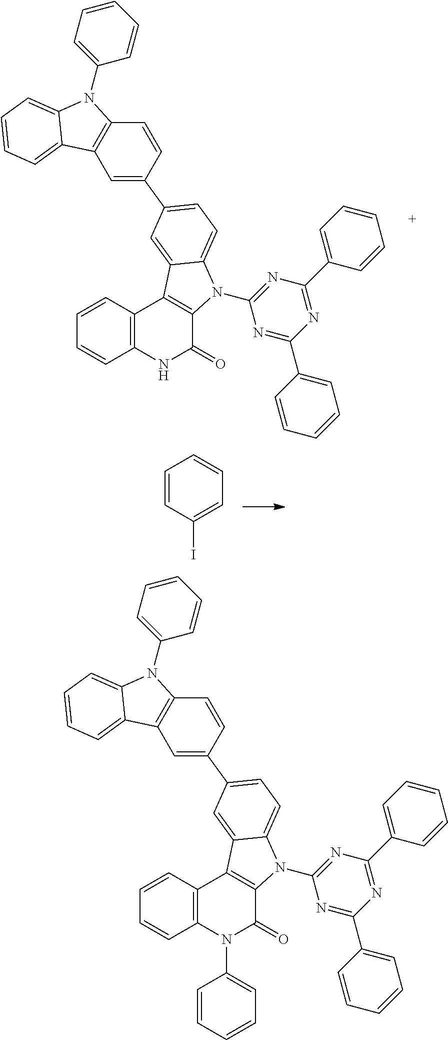

28.2 g (40 mmol) of 7-(4,6-diphenyl-1,3,5-triazin-2-yl)-10-(9-phenylcarbazol-3-yl)-5H-indolo[2,3-c]quinolin-6-one, 61.2 g (85 mmol) of 4-iodobenzene and 44.7 g (320 mmol) of potassium carbonate, 3 g (16 mmol) of copper(I) iodide and 3.6 g (16 mmol) of 1,3-di(pyridin-2-yl)propane-1,3-dione are stirred in 100 ml of DMF at 15000 for 30 h. The solution is diluted with water and extracted twice with ethyl acetate. The combined organic phases are dried over Na₂SO₄ and concentrated by rotary evaporation. The residue is purified by chromatography (EtOAc/hexane: 2/3), recrystallized from toluene and finally sublimed under high vacuum (p=5×10⁻⁵ mbar). The purity is 99.9%. The yield is 22.5 g (28 mmol), 72% of theory.

The following compounds can be obtained analogously:

| | Reactant 1 | Reactant 2 |
|---|---|---|
| | 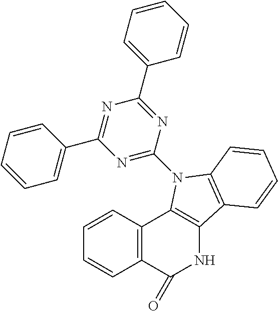 [SMILES: O=c1[nH]c2c3ccccc3n(-c3nc(-c4ccccc4)nc(-c4ccccc4)n3)c2c2ccccc12] | 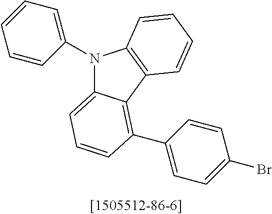 [SMILES: Brc1ccc(-c2cccc3c2c2ccccc2n3-c2ccccc2)cc1] |
| 2f | 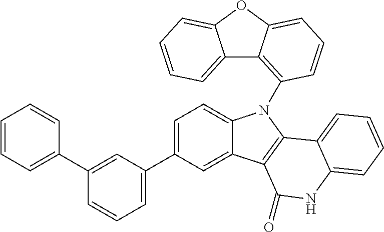 [SMILES: O=c1[nH]c2ccccc2c2c1c1cc(-c3cccc(-c4ccccc4)c3)ccc1n2-c1cccc2oc3ccccc3c12] | 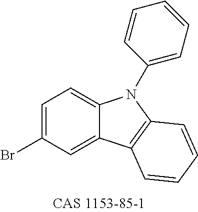 [SMILES: Brc1ccc2c(c1)c1ccccc1n2-c1ccccc1] |
| 3f | 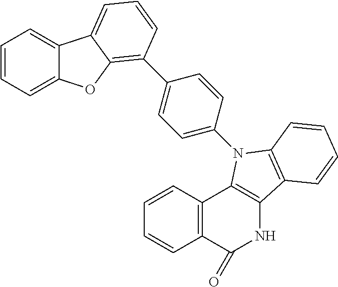 [SMILES: O=c1[nH]c2c3ccccc3n(-c3ccc(-c4cccc5c4oc4ccccc45)cc3)c2c2ccccc12] | 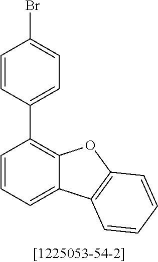 [SMILES: Brc1ccc(-c2cccc3c2oc2ccccc23)cc1] |
| 4f | 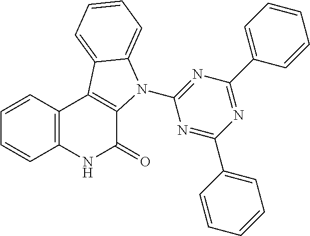 [SMILES: O=c1[nH]c2ccccc2c2c3ccccc3n(-c3nc(-c4ccccc4)nc(-c4ccccc4)n3)c12] | 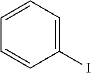 |
| 5f | 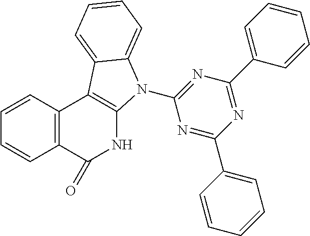 [SMILES: O=c1[nH]c2c(c3ccccc13)c1ccccc1n2-c1nc(-c2ccccc2)nc(-c2ccccc2)n1] | 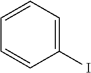 |
| 6f | 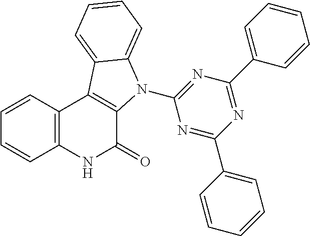 [SMILES: O=c1[nH]c2ccccc2c2c3ccccc3n(-c3nc(-c4ccccc4)nc(-c4ccccc4)n3)c12] | 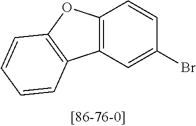 [SMILES: Brc1ccc2oc3ccccc3c2c1] |
| 7f | 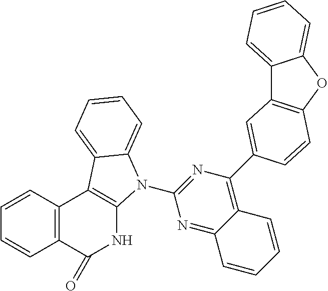 [SMILES: O=c1[nH]c2c(c3ccccc13)c1ccccc1n2-c1nc(-c2ccc3oc4ccccc4c3c2)c2ccccc2n1] | 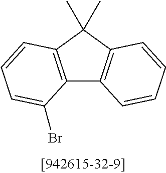 [SMILES: CC1(C)c2ccccc2-c2c(Br)cccc21] |
| 8f | 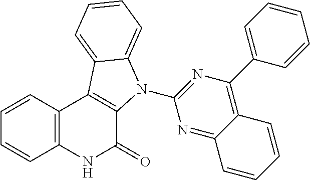 [SMILES: O=c1[nH]c2ccccc2c2c3ccccc3n(-c3nc(-c4ccccc4)c4ccccc4n3)c12] | 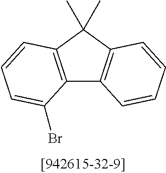 [SMILES: CC1(C)c2ccccc2-c2c(Br)cccc21] |
| 9f | 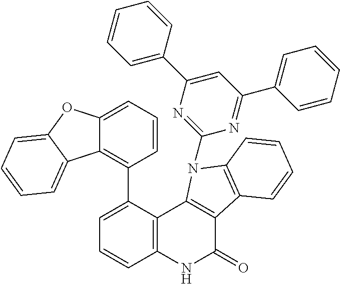 [SMILES: O=c1[nH]c2cccc(-c3cccc4oc5ccccc5c34)c2c2c1c1ccccc1n2-c1nc(-c2ccccc2)cc(-c2ccccc2)n1] | 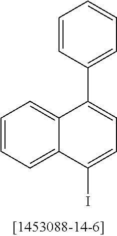 [SMILES: Ic1ccc(-c2ccccc2)c2ccccc12] |
| 10f | 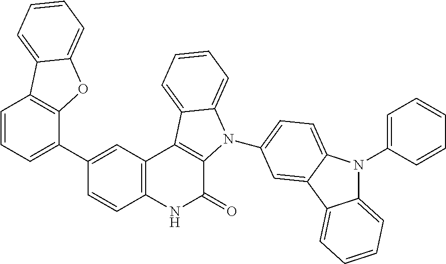 [SMILES: O=c1[nH]c2ccc(-c3cccc4c3oc3ccccc34)cc2c2c3ccccc3n(-c3ccc4c(c3)c3ccccc3n4-c3ccccc3)c12] | 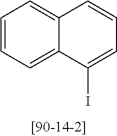 [SMILES: Ic1cccc2ccccc12] |
| 11f | 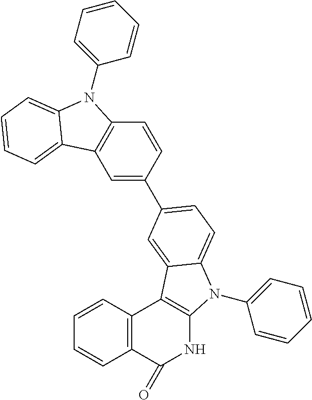 [SMILES: O=c1[nH]c2c(c3ccccc13)c1cc(-c3ccc4c(c3)c3ccccc3n4-c3ccccc3)ccc1n2-c1ccccc1] | 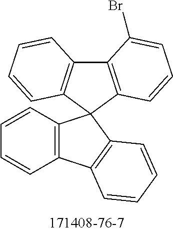 [SMILES: Brc1cccc2c1-c1ccccc1C21c2ccccc2-c2ccccc21] |
| 12f | 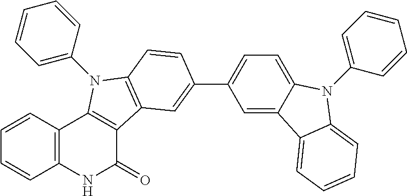 [SMILES: O=c1[nH]c2ccccc2c2c1c1cc(-c3ccc4c(c3)c3ccccc3n4-c3ccccc3)ccc1n2-c1ccccc1] | 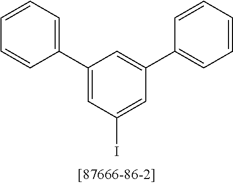 [SMILES: Ic1cc(-c2ccccc2)cc(-c2ccccc2)c1] |
| 13f | 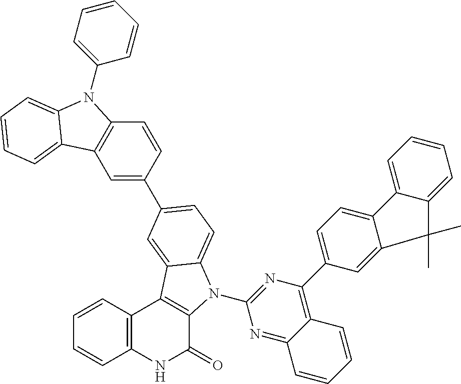 [SMILES: CC1(C)c2ccccc2-c2ccc(-c3nc(-n4c5ccc(-c6ccc7c(c6)c6ccccc6n7-c6ccccc6)cc5c5c6ccccc6[nH]c(=O)c54)nc4ccccc34)cc21] | 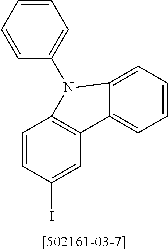 [SMILES: Ic1ccc2c(c1)c1ccccc1n2-c1ccccc1] |
| 14f | 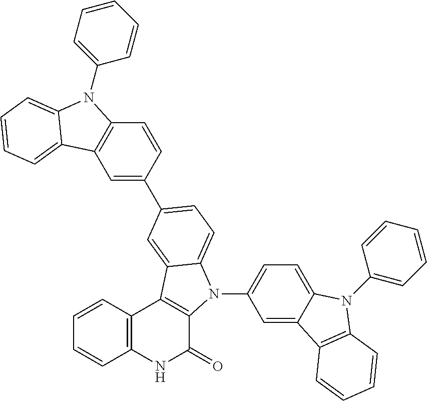 [SMILES: O=c1[nH]c2ccccc2c2c3cc(-c4ccc5c(c4)c4ccccc4n5-c4ccccc4)ccc3n(-c3ccc4c(c3)c3ccccc3n4-c3ccccc3)c12] | 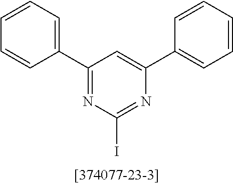 [SMILES: Ic1nc(-c2ccccc2)cc(-c2ccccc2)n1] |
| 15f | 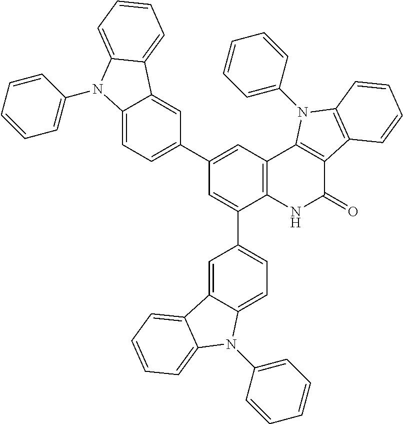 [SMILES: O=c1[nH]c2c(-c3ccc4c(c3)c3ccccc3n4-c3ccccc3)cc(-c3ccc4c(c3)c3ccccc3n4-c3ccccc3)cc2c2c1c1ccccc1n2-c1ccccc1] | 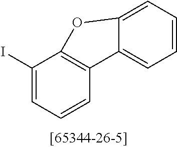 [SMILES: Ic1cccc2c1oc1ccccc12] |
| 16f | 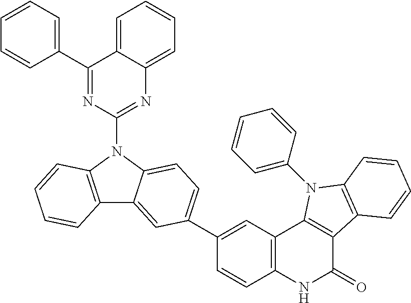 [SMILES: O=c1[nH]c2ccc(-c3ccc4c(c3)c3ccccc3n4-c3nc(-c4ccccc4)c4ccccc4n3)cc2c2c1c1ccccc1n2-c1ccccc1] | 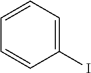 |
| 17f | 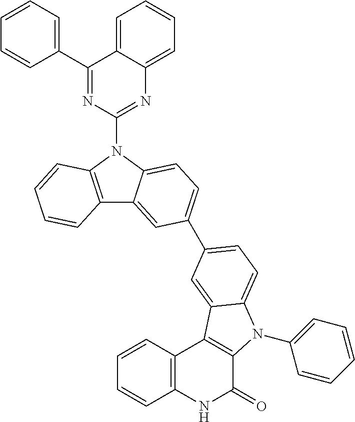 [SMILES: O=c1[nH]c2ccccc2c2c3cc(-c4ccc5c(c4)c4ccccc4n5-c4nc(-c5ccccc5)c5ccccc5n4)ccc3n(-c3ccccc3)c12] | 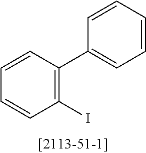 [SMILES: Ic1ccccc1-c1ccccc1] |
| 18f | 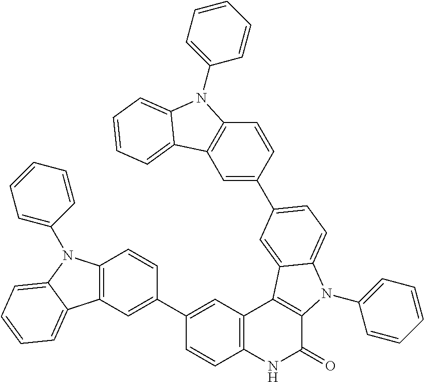 [SMILES: O=c1[nH]c2ccc(-c3ccc4c(c3)c3ccccc3n4-c3ccccc3)cc2c2c3cc(-c4ccc5c(c4)c4ccccc4n5-c4ccccc4)ccc3n(-c3ccccc3)c12] | 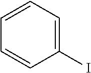 |
| 19f | 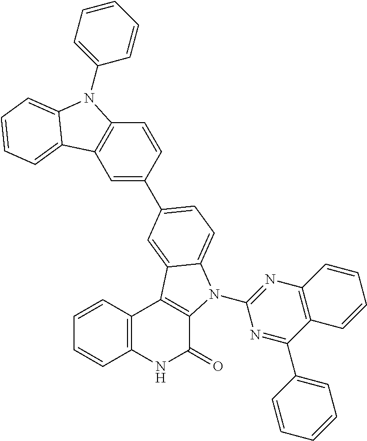 [SMILES: O=c1[nH]c2ccccc2c2c3cc(-c4ccc5c(c4)c4ccccc4n5-c4ccccc4)ccc3n(-c3nc(-c4ccccc4)c4ccccc4n3)c12] | 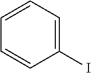 |
| 20f | 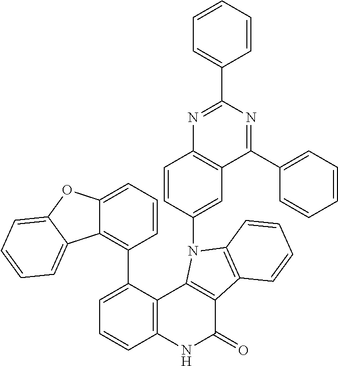 [SMILES: O=c1[nH]c2cccc(-c3cccc4oc5ccccc5c34)c2c2c1c1ccccc1n2-c1ccc2nc(-c3ccccc3)nc(-c3ccccc3)c2c1] | 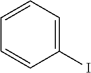 |
| 21f | 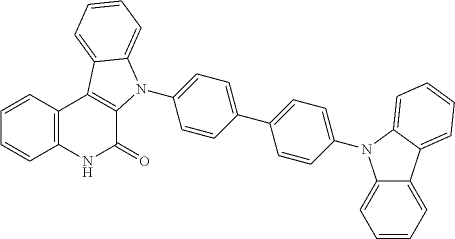 [SMILES: O=c1[nH]c2ccccc2c2c3ccccc3n(-c3ccc(-c4ccc(-n5c6ccccc6c6ccccc65)cc4)cc3)c12] | 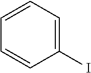 |
| 22f | 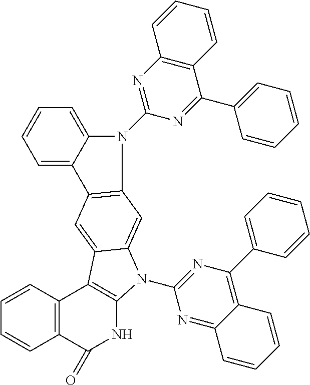 [SMILES: O=c1[nH]c2c(c3ccccc13)c1cc3c4ccccc4n(-c4nc(-c5ccccc5)c5ccccc5n4)c3cc1n2-c1nc(-c2ccccc2)c2ccccc2n1] | 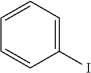 |
| 23f | 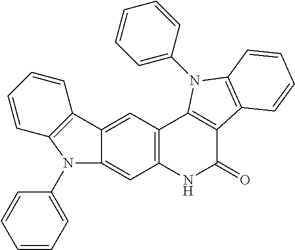 [SMILES: O=c1[nH]c2cc3c(cc2c2c1c1ccccc1n2-c1ccccc1)c1ccccc1n3-c1ccccc1] | 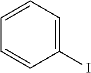 |
| 24f | 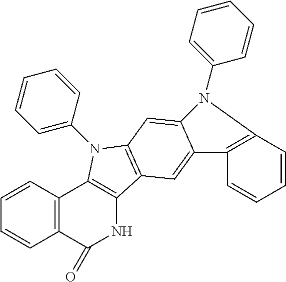 [SMILES: O=c1[nH]c2c3cc4c5ccccc5n(-c5ccccc5)c4cc3n(-c3ccccc3)c2c2ccccc12] | 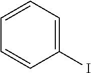 |
| 25f | 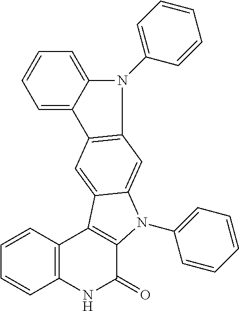 [SMILES: O=c1[nH]c2ccccc2c2c3cc4c5ccccc5n(-c5ccccc5)c4cc3n(-c3ccccc3)c12] | 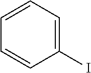 |
| 26f | 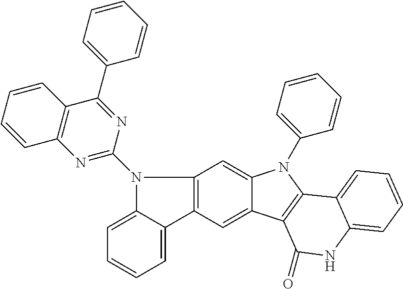 [SMILES: O=c1[nH]c2ccccc2c2c1c1cc3c4ccccc4n(-c4nc(-c5ccccc5)c5ccccc5n4)c3cc1n2-c1ccccc1] | 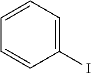 |
| 27f | 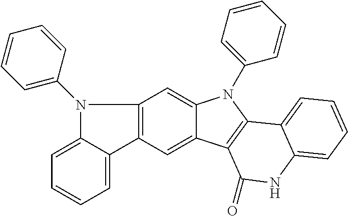 [SMILES: O=c1[nH]c2ccccc2c2c1c1cc3c4ccccc4n(-c4ccccc4)c3cc1n2-c1ccccc1] | 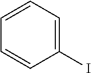 |
| | Product | Yield |
| | 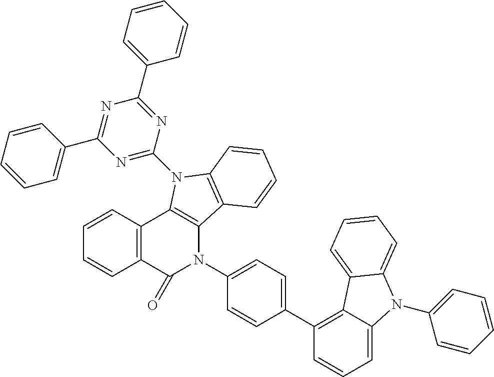 [SMILES: O=c1c2ccccc2c2c(c3ccccc3n2-c2nc(-c3ccccc3)nc(-c3ccccc3)n2)n1-c1ccc(-c2cccc3c2c2ccccc2n3-c2ccccc2)cc1] | 71% |
| 2f | 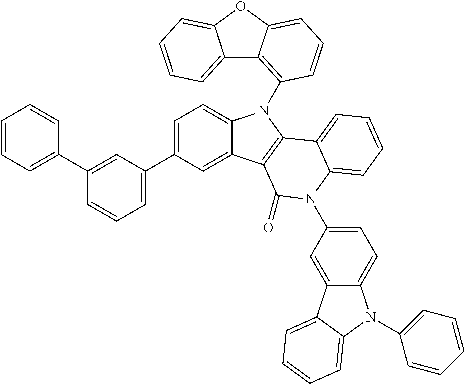 [SMILES: O=c1c2c3cc(-c4cccc(-c5ccccc5)c4)ccc3n(-c3cccc4oc5ccccc5c34)c2c2ccccc2n1-c1ccc2c(c1)c1ccccc1n2-c1ccccc1] | 74% |
| 3f | 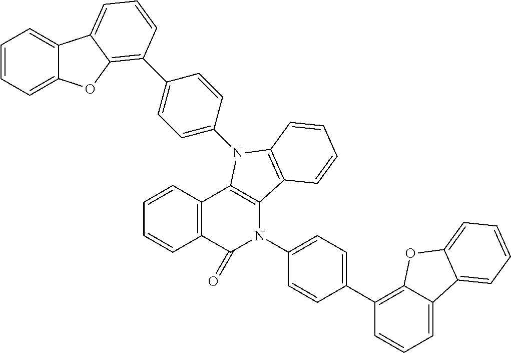 [SMILES: O=c1c2ccccc2c2c(c3ccccc3n2-c2ccc(-c3cccc4c3oc3ccccc34)cc2)n1-c1ccc(-c2cccc3c2oc2ccccc23)cc1] | 73% |
| 4f | 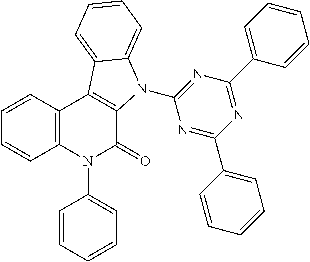 [SMILES: O=c1c2c(c3ccccc3n1-c1ccccc1)c1ccccc1n2-c1nc(-c2ccccc2)nc(-c2ccccc2)n1] | 75% |
| 5f | 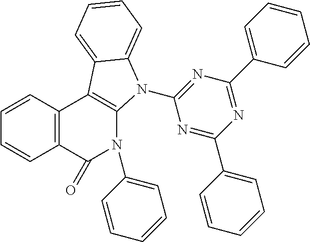 [SMILES: O=c1c2ccccc2c2c3ccccc3n(-c3nc(-c4ccccc4)nc(-c4ccccc4)n3)c2n1-c1ccccc1] | 79% |
| 6f | 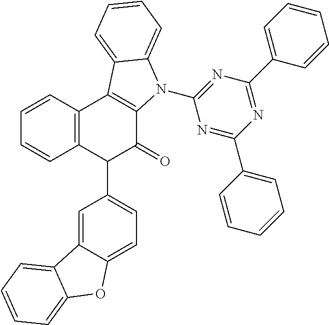 [SMILES: O=C1c2c(c3ccccc3n2-c2nc(-c3ccccc3)nc(-c3ccccc3)n2)-c2ccccc2C1c1ccc2oc3ccccc3c2c1] | 73% |
| 7f | 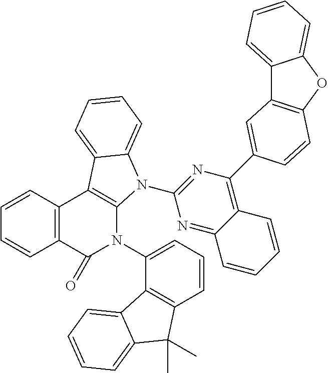 [SMILES: CC1(C)c2ccccc2-c2c(-n3c(=O)c4ccccc4c4c5ccccc5n(-c5nc(-c6ccc7oc8ccccc8c7c6)c6ccccc6n5)c43)cccc21] | 77% |
| 8f | 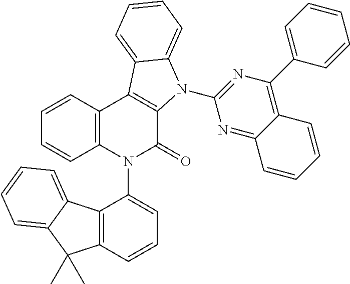 [SMILES: CC1(C)c2ccccc2-c2c(-n3c(=O)c4c(c5ccccc53)c3ccccc3n4-c3nc(-c4ccccc4)c4ccccc4n3)cccc21] | 79% |
| 9f | 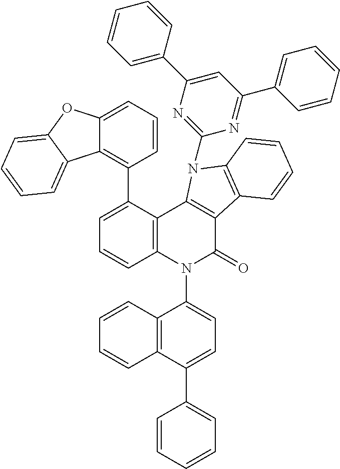 [SMILES: O=c1c2c3ccccc3n(-c3nc(-c4ccccc4)cc(-c4ccccc4)n3)c2c2c(-c3cccc4oc5ccccc5c34)cccc2n1-c1ccc(-c2ccccc2)c2ccccc12] | 68% |
| 10f | 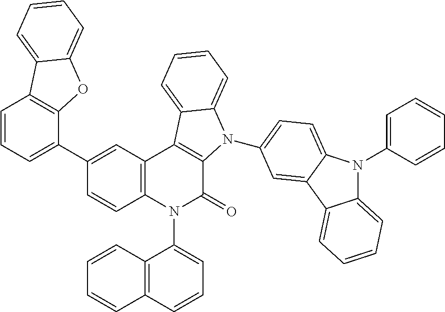 [SMILES: O=c1c2c(c3cc(-c4cccc5c4oc4ccccc45)ccc3n1-c1cccc3ccccc13)c1ccccc1n2-c1ccc2c(c1)c1ccccc1n2-c1ccccc1] | 71% |
| 11f | 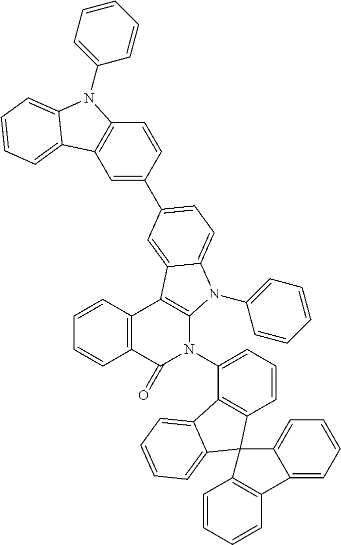 [SMILES: O=c1c2ccccc2c2c3cc(-c4ccc5c(c4)c4ccccc4n5-c4ccccc4)ccc3n(-c3ccccc3)c2n1-c1cccc2c1-c1ccccc1C21c2ccccc2-c2ccccc21] | 66% |
| 12f | 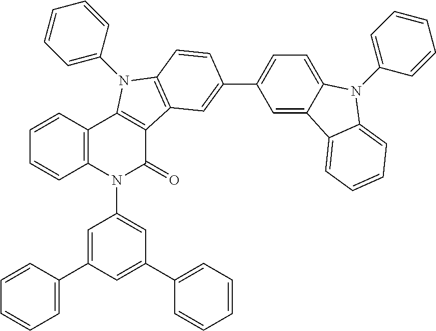 [SMILES: O=c1c2c3cc(-c4ccc5c(c4)c4ccccc4n5-c4ccccc4)ccc3n(-c3ccccc3)c2c2ccccc2n1-c1cc(-c2ccccc2)cc(-c2ccccc2)c1] | 65% |
| 13f | 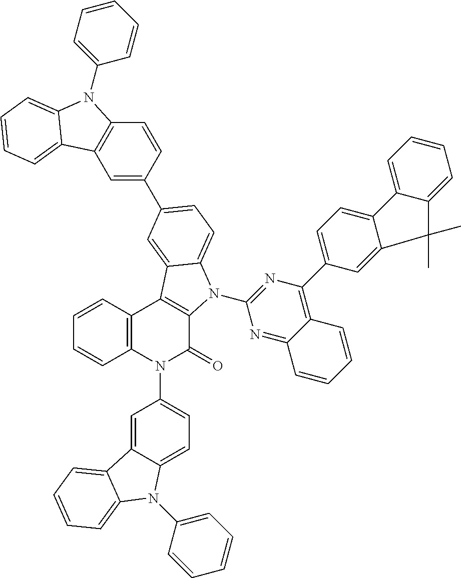 [SMILES: CC1(C)c2ccccc2-c2ccc(-c3nc(-n4c5ccc(-c6ccc7c(c6)c6ccccc6n7-c6ccccc6)cc5c5c6ccccc6n(-c6ccc7c(c6)c6ccccc6n7-c6ccccc6)c(=O)c54)nc4ccccc34)cc21] | 71% |
| 14f | 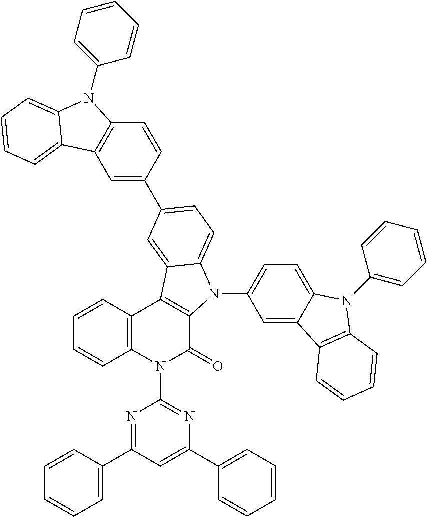 [SMILES: O=c1c2c(c3ccccc3n1-c1nc(-c3ccccc3)cc(-c3ccccc3)n1)c1cc(-c3ccc4c(c3)c3ccccc3n4-c3ccccc3)ccc1n2-c1ccc2c(c1)c1ccccc1n2-c1ccccc1] | 80% |
| 15f | 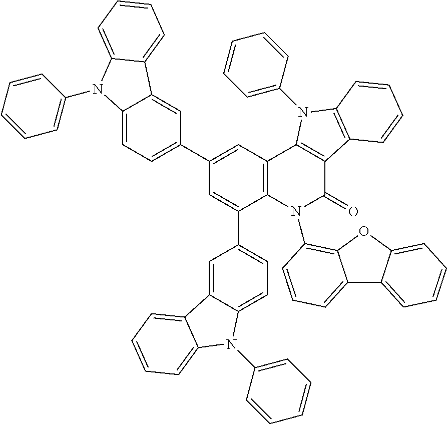 [SMILES: O=c1c2c3ccccc3n(-c3ccccc3)c2c2cc(-c3ccc4c(c3)c3ccccc3n4-c3ccccc3)cc(-c3ccc4c(c3)c3ccccc3n4-c3ccccc3)c2n1-c1cccc2c1oc1ccccc12] | 64% |
| 16f | 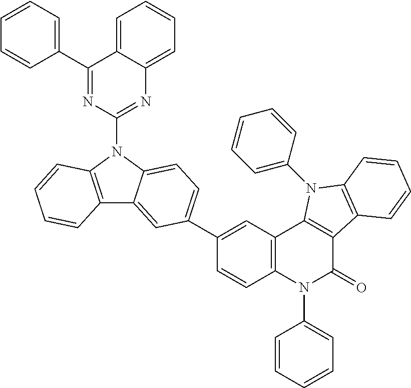 [SMILES: O=c1c2c3ccccc3n(-c3ccccc3)c2c2cc(-c3ccc4c(c3)c3ccccc3n4-c3nc(-c4ccccc4)c4ccccc4n3)ccc2n1-c1ccccc1] | 74% |
| 17f | 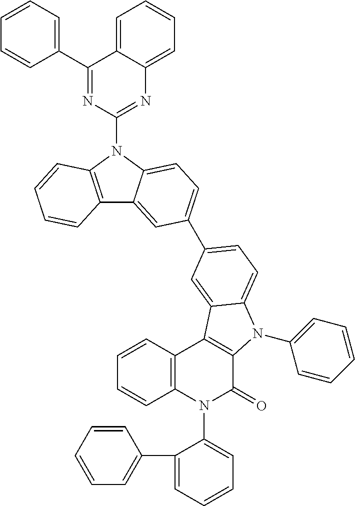 [SMILES: O=c1c2c(c3ccccc3n1-c1ccccc1-c1ccccc1)c1cc(-c3ccc4c(c3)c3ccccc3n4-c3nc(-c4ccccc4)c4ccccc4n3)ccc1n2-c1ccccc1] | 77% |
| 18f | 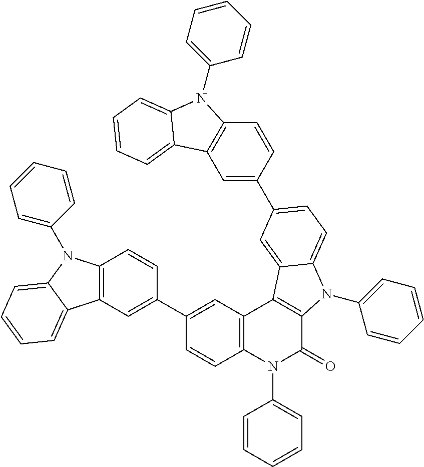 [SMILES: O=c1c2c(c3cc(-c4ccc5c(c4)c4ccccc4n5-c4ccccc4)ccc3n1-c1ccccc1)c1cc(-c3ccc4c(c3)c3ccccc3n4-c3ccccc3)ccc1n2-c1ccccc1] | 79% |
| 19f | 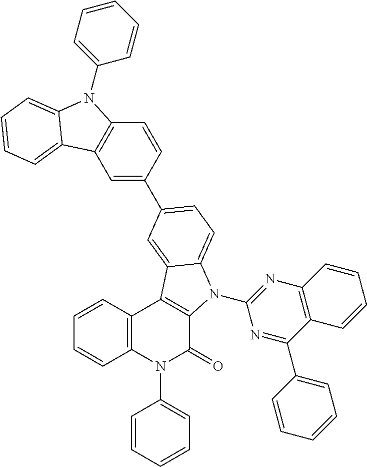 [SMILES: O=c1c2c(c3ccccc3n1-c1ccccc1)c1cc(-c3ccc4c(c3)c3ccccc3n4-c3ccccc3)ccc1n2-c1nc(-c2ccccc2)c2ccccc2n1] | 69% |
| 20f | 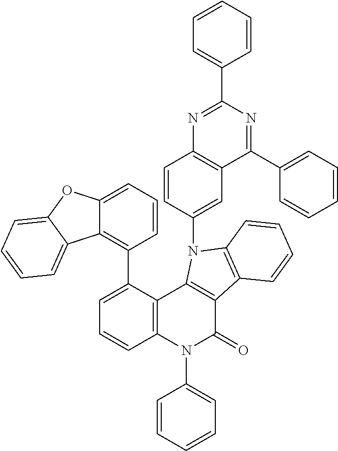 [SMILES: O=c1c2c3ccccc3n(-c3ccc4nc(-c5ccccc5)nc(-c5ccccc5)c4c3)c2c2c(-c3cccc4oc5ccccc5c34)cccc2n1-c1ccccc1] | 78% |
| 21f | 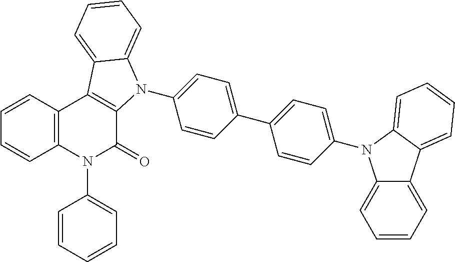 [SMILES: O=c1c2c(c3ccccc3n1-c1ccccc1)c1ccccc1n2-c1ccc(-c2ccc(-n3c4ccccc4c4ccccc43)cc2)cc1] | 62% |
| 22f | 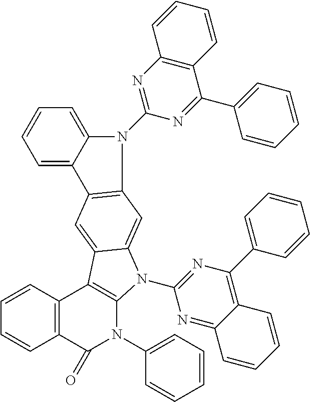 [SMILES: O=c1c2ccccc2c2c3cc4c5ccccc5n(-c5nc(-c6ccccc6)c6ccccc6n5)c4cc3n(-c3nc(-c4ccccc4)c4ccccc4n3)c2n1-c1ccccc1] | 80% |
| 23f | 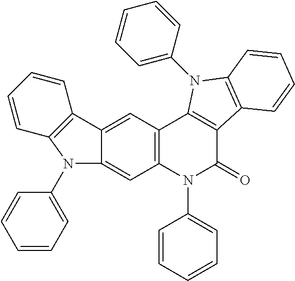 [SMILES: O=c1c2c3ccccc3n(-c3ccccc3)c2c2cc3c4ccccc4n(-c4ccccc4)c3cc2n1-c1ccccc1] | 76% |
| 24f | 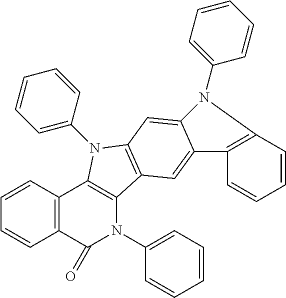 [SMILES: O=c1c2ccccc2c2c(c3cc4c5ccccc5n(-c5ccccc5)c4cc3n2-c2ccccc2)n1-c1ccccc1] | 83% |
| 25f | 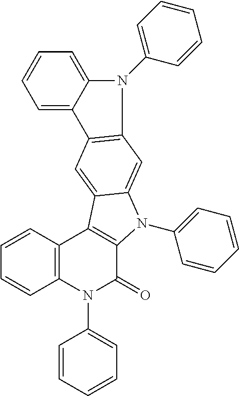 [SMILES: O=c1c2c(c3ccccc3n1-c1ccccc1)c1cc3c4ccccc4n(-c4ccccc4)c3cc1n2-c1ccccc1] | 77% |
| 26f | 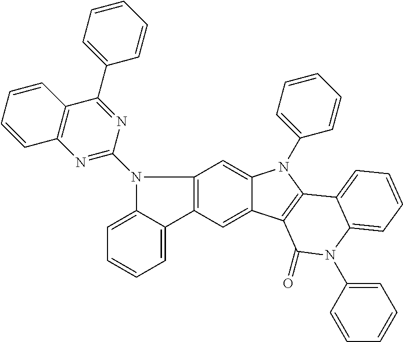 [SMILES: O=c1c2c3cc4c5ccccc5n(-c5nc(-c6ccccc6)c6ccccc6n5)c4cc3n(-c3ccccc3)c2c2ccccc2n1-c1ccccc1] | 81% |
| 27f | 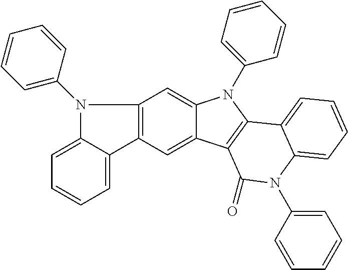 [SMILES: O=c1c2c3cc4c5ccccc5n(-c5ccccc5)c4cc3n(-c3ccccc3)c2c2ccccc2n1-c1ccccc1] | 86% |

Production of the OLEDs

Examples E1 to E9 which follow (see table 1) present the use of the materials of the invention in OLEDs.

Pretreatment for Examples C1, E1 to E9: Glass plates coated with structured ITO (indium tin oxide) of thickness 50 nm are treated prior to coating with an oxygen plasma, followed by an argon plasma. These plasma-treated glass plates form the substrates to which the OLEDs are applied.

The OLEDs basically have the following layer structure: substrate/hole injection layer (HIL)/hole transport layer (HTL)/electron blocker layer (EBL)/emission layer (EML)/optional hole blocker layer (HBL)/electron transport layer (ETL)/optional electron injection layer (EIL) and finally a cathode. The cathode is formed by an aluminum layer of thickness 100 nm. The exact structure of the OLEDs can be found in table 1. The materials required for production of the OLEDs are shown in table 2.

All materials are applied by thermal vapor deposition in a vacuum chamber. In this case, the emission layer always consists of at least one matrix material (host material) and an emitting dopant (emitter) which is added to the matrix material(s) in a particular proportion by volume by co-evaporation. Details given in such a form as IC1:SdT1:TEG1 (45%:45%:10%) mean here that the material IC1 is present in the layer in a proportion of 45%, SdT1 in a proportion of 45%, and TEG1 in a proportion of 10%. Analogously, the electron transport layer may also consist of a mixture of two materials.

The OLEDs are characterized in a standard manner. For this purpose, electroluminescence spectra, current efficiency (CE, measured in cd/A) and external quantum efficiency (EQE, measured in %) are determined as a function of luminance, calculated from current-voltage-luminance characteristics assuming Lambertian emission characteristics. Electroluminescence spectra are determined at a luminance of 1000 cd/m², and these are used to calculate the CIE 1931 x and y color coordinates. The results thus obtained can be found in table 3.

Use of the Materials of the Invention in OLEDs

The compounds EG1 to EG4 of the invention are used in examples E1 to E4 and E10 as matrix material in the emission layer of phosphorescent green OLEDs. For direct comparison, the compound according to the prior art SdT1 is characterized in the same device setup (V1). The compounds EG5 to EG9 of the invention are used in examples E5 to E9 as matrix material in the emission layer of phosphorescent red OLEDs.

TABLE 1
Structure of the OLEDs

| | HIL | HTL | EBL | EML | HBL | ETL | EIL |
|---|---|---|---|---|---|---|---|
| Ex. | Thickness | Thickness | Thickness | Thickness | Thickness | Thickness | Thickness |
| C1 | HATCN | SpMA1 | SpMA2 | IC1:SdT1:TEG | ST2 | ST2:LiQ (50%:50%) | LiQ |
| | | 230 nm | 20 nm | (49%:44%:7%) 40 nm | 5 nm | 30 nm | 1 nm |
| E1 | HATCN | SpMA1 | SpMA2 | IC1:EG1:TEG | ST2 | ST2:LiQ (50%:50%) | LiQ |
| | 5 nm | 230 nm | 20 nm | (49%:44%:7%) 40 nm | 5 nm | 30 nm | 1 nm |
| E2 | HATCN | SpMA1 | SpMA2 | IC1:EG2:TEG | ST2 | ST2:LiQ (50%:50%) | LiQ |
| | 5nm | 230 nm | 20 nm | (22%:71%:7%) 40 nm | 5 nm | 30 nm | 1 nm |
| E3 | HATCN | SpMA1 | SpMA2 | EG3:IC2:TEG | ST2 | ST2:LiQ (50%:50%) | LiQ |
| | 5 nm | 230 nm | 20 nm | (49%:44%:7%) 40 nm | 5 nm | 30 nm | 1 nm |
| E4 | HATCN | SpMA1 | SpMA2 | EG4:IC2:TEG | ST2 | ST2:LiQ (50%:50%) | LiQ |
| | 5 nm | 230 nm | 20 nm | (49%:44%:7%) 40 nm | 5 nm | 30 nm | 1 nm |
| E5 | HATCN | SpMA1 | SpMA2 | EG5:IC1:TER | ST2 | ST2:LiQ (50%:50%) | LiQ |
| | 5 nm | 125 nm | 10 nm | (50%:47%:3%) 35 nm | 10 nm | 30 nm | 1 nm |
| E6 | HATCN | SpMA1 | SpMA2 | EG6:TER | ST2 | ST2:LiQ (50%:50%) | LiQ |
| | 5 nm | 125 nm | 10 nm | (97%:3%) 35 nm | 10 nm | 30 nm | 1 nm |
| E7 | HATCN | SpMA1 | SpMA2 | EG7:IC1:TER | ST2 | ST2:LiQ (50%:50%) | LiQ |
| | 5 nm | 125 nm | 10 nm | (50%:47%:3%) 35 nm | 10 nm | 30 nm | 1 nm |
| E8 | HATCN | SpMA1 | SpMA2 | EG8:TER | ST2 | ST2:LiQ (50%:50%) | LiQ |
| | 5 nm | 125 nm | 10 nm | (97%:3%) 35 nm | 10 nm | 30 nm | 1 nm |
| E9 | HATCN | SpMA1 | SpMA2 | EG9:TER | ST2 | ST2:LiQ (50%:50%) | LiQ |
| | 5 nm | 125 nm | 10 nm | (97%:3%) 35 nm | 10 nm | 30 nm | 1 nm |
| E10 | HATCN | SpMA1 | SpMA2 | EG3:IC3:TEG | ST2 | ST2:LiQ (50%:50%) | LiQ |
| | 5 nm | 125 nm | 10 nm | (50%:47%:3%) 35 nm | 10 nm | 30 nm | 1 nm |

TABLE 2
Structural formulae of the materials for the OLEDs

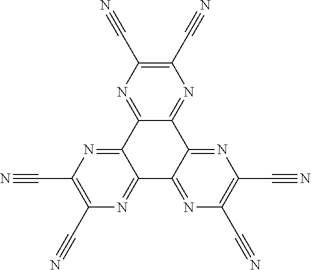 [SMILES: N#Cc1nc2c3nc(C#N)c(C#N)nc3c3nc(C#N)c(C#N)nc3c2nc1C#N]

HATCN

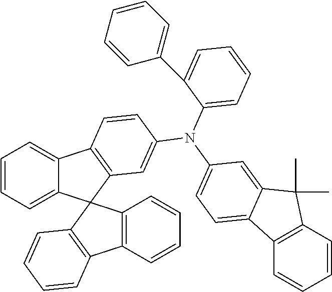 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

SpMA1

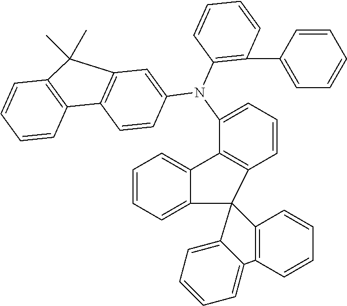 [SMILES: CC1(C)c2ccccc2-c2ccc(N(c3ccccc3-c3ccccc3)c3cccc4c3-c3ccccc3C43c4ccccc4-c4ccccc43)cc21]

SpMA3

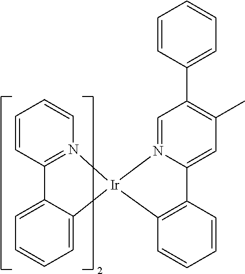

TEG

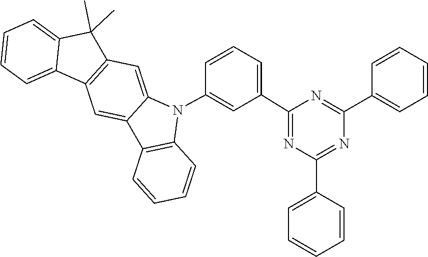 [SMILES: CC1(C)c2ccccc2-c2cc3c4ccccc4n(-c4cccc(-c5nc(-c6ccccc6)nc(-c6ccccc6)n5)c4)c3cc21]

IC1

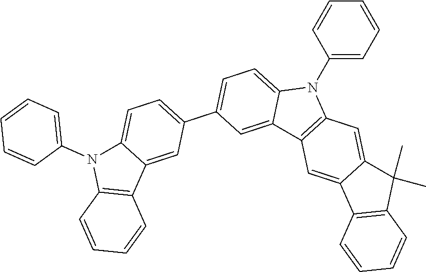 [SMILES: CC1(C)c2ccccc2-c2cc3c4cc(-c5ccc6c(c5)c5ccccc5n6-c5ccccc5)ccc4n(-c4ccccc4)c3cc21]

IC2

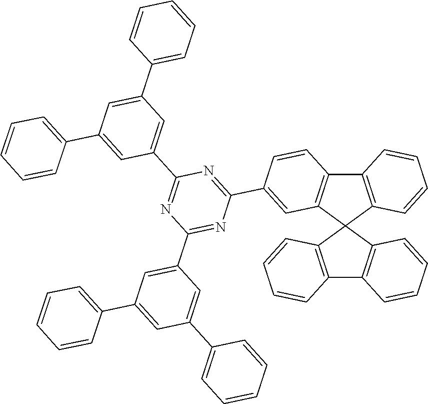 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1]

ST2

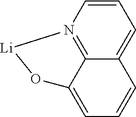

LiQ

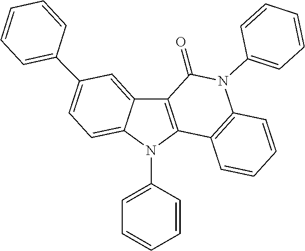 [SMILES: O=c1c2c3cc(-c4ccccc4)ccc3n(-c3ccccc3)c2c2ccccc2n1-c1ccccc1]

SdT1

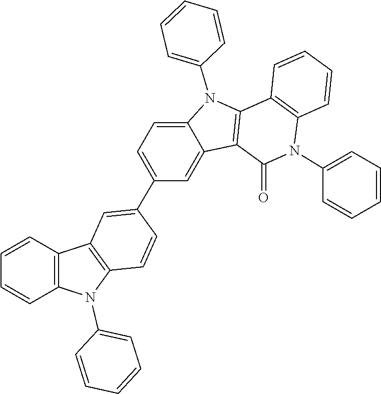 [SMILES: O=c1c2c3cc(-c4ccc5c(c4)c4ccccc4n5-c4ccccc4)ccc3n(-c3ccccc3)c2c2ccccc2n1-c1ccccc1]

EG1

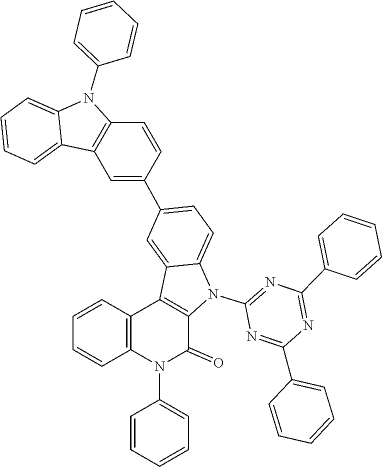 [SMILES: O=c1c2c(c3ccccc3n1-c1ccccc1)c1cc(-c3ccc4c(c3)c3ccccc3n4-c3ccccc3)ccc1n2-c1nc(-c2ccccc2)nc(-c2ccccc2)n1]

EG2

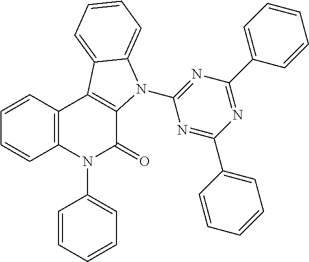 [SMILES: O=c1c2c(c3ccccc3n1-c1ccccc1)c1ccccc1n2-c1nc(-c2ccccc2)nc(-c2ccccc2)n1]

EG3

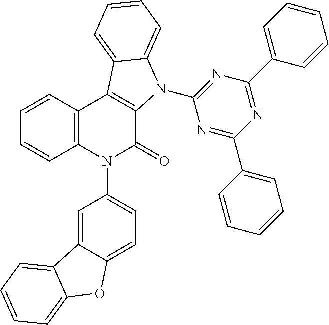 [SMILES: O=c1c2c(c3ccccc3n1-c1ccc3oc4ccccc4c3c1)c1ccccc1n2-c1nc(-c2ccccc2)nc(-c2ccccc2)n1]

EG4

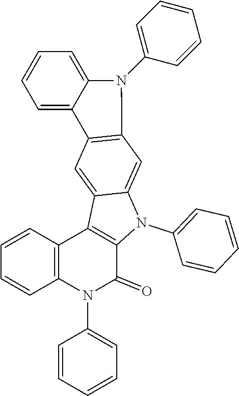 [SMILES: O=c1c2c(c3ccccc3n1-c1ccccc1)c1cc3c4ccccc4n(-c4ccccc4)c3cc1n2-c1ccccc1]

EG5

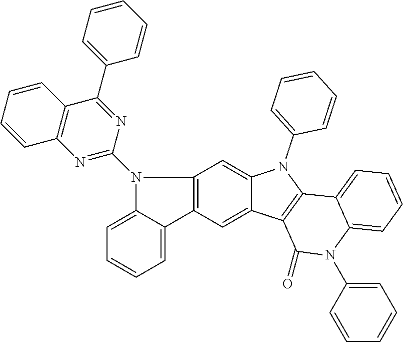 [SMILES: O=c1c2c3cc4c5ccccc5n(-c5nc(-c6ccccc6)c6ccccc6n5)c4cc3n(-c3ccccc3)c2c2ccccc2n1-c1ccccc1]

EG6

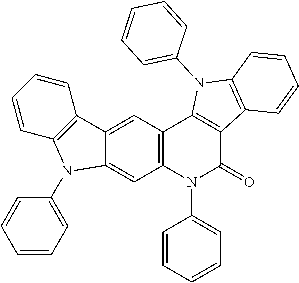 [SMILES: O=c1c2c3ccccc3n(-c3ccccc3)c2c2cc3c4ccccc4n(-c4ccccc4)c3cc2n1-c1ccccc1]

EG7

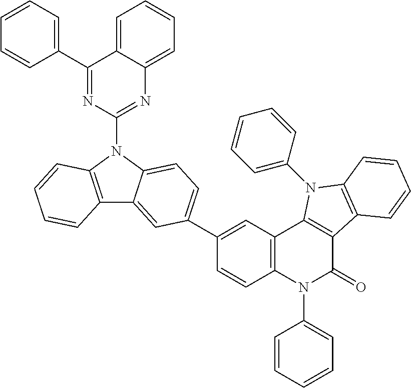 [SMILES: O=c1c2c3ccccc3n(-c3ccccc3)c2c2cc(-c3ccc4c(c3)c3ccccc3n4-c3nc(-c4ccccc4)c4ccccc4n3)ccc2n1-c1ccccc1]

EG8

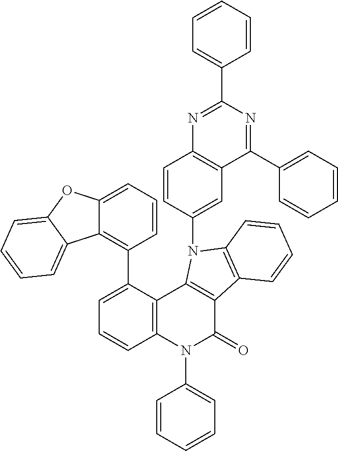 [SMILES: O=c1c2c3ccccc3n(-c3ccc4nc(-c5ccccc5)nc(-c5ccccc5)c4c3)c2c2c(-c3cccc4oc5ccccc5c34)cccc2n1-c1ccccc1]

EG9

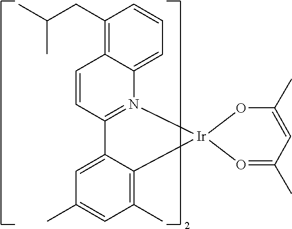

TER

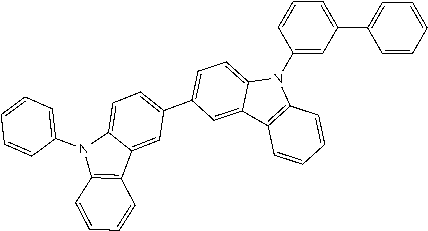 [SMILES: c1ccc(-c2cccc(-n3c4ccccc4c4cc(-c5ccc6c(c5)c5ccccc5n6-c5ccccc5)ccc43)c2)cc1]

IC3

TABLE 3
Data of the OLEDs

| | U1000 | SE1000 | EQE 1000 | CIE x/y at |
|---|---|---|---|---|
| Ex. | (V) | (cd/A) | (%) | 1000 cd/m² |
| V1 | 3.9 | 63 | 16 | 0.36/0.61 |
| E1 | 3.3 | 71 | 19 | 0.35/0.61 |
| E2 | 3.1 | 69 | 17 | 0.34/0.62 |
| E3 | 3.4 | 69 | 18 | 0.35/0.61 |
| E4 | 3.2 | 71 | 17 | 0.35/0.62 |
| E5 | 3.8 | 22 | 21 | 0.66/0.34 |
| E6 | 3.9 | 20 | 20 | 0.65/0.33 |
| E7 | 3.9 | 23 | 21 | 0.66/0.34 |
| E8 | 3.7 | 24 | 22 | 0.67/0.34 |
| E9 | 3.8 | 19 | 18 | 0.66/0.33 |
| E10 | 3.2 | 71 | 19 | 0.35/0.62 |

1.-14. (canceled)

15. A compound of formula (1) 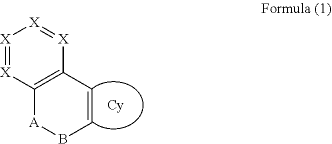
where the symbols used are as follows:
A, B are selected from the group consisting of NAr¹, C═O, C═S, C═NR, BR, PR, P(═O)R, SO and SO₂, with the proviso that one of the symbols A and B is NAr¹ and the other of the symbols A and B is C═O, C═S, C═NR, BR, PR, P(═O)R, SO or SO₂;
Cy together with the two carbon atoms shown explicitly is a group of the following formula (2): 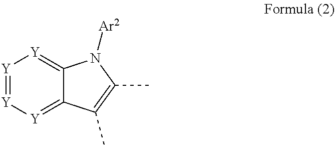
where the dotted bonds indicate the linkage of this group in the formula (1);
X is the same or different at each instance and is CR or N; or two adjacent X groups are a group of the formula (3), and the two other symbols X are the same or different at each instance and are CR or N, 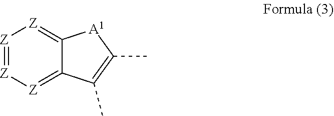
where the dotted bonds indicate the linkage of this group in the formula (1);
Y is the same or different at each instance and is CR or N; or two adjacent Y groups are a group of the formula (3), and the two other symbols Y are the same or different at each instance and are CR or N, 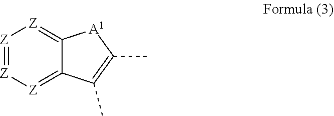
where the dotted bonds indicate the linkage of this group in the formula (1);
A¹ is the same or different at each instance and is NAr³, O, S or C(R)₂;
Z is the same or different at each instance and is CR or N;
Ar¹, Ar², Ar³ is the same or different at each instance and is an aromatic or heteroaromatic ring system which has 5 to 40 aromatic ring atoms and may be substituted by one or more R radicals;
R is the same or different at each instance and is H, D, F, Cl, Br, I, N(Ar′)₂, N(R¹)₂, OAr′, SAr′, CN, NO₂, OR¹, SR¹, COOR¹, C(═O)N(R¹)₂, Si(R¹)₃, B(OR¹)₂, C(═O)R¹, P(═O)(R¹)₂, S(═O)R¹, S(═O)₂R¹, OSO₂R¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R¹ radicals, where one or more nonadjacent CH₂ groups may be replaced by Si(R¹)₂, C═O, NR¹, O, S or CONR¹, or an aromatic or heteroaromatic ring system which has 5 to 60 aromatic ring atoms and may be substituted in each case by one or more R¹ radicals; at the same time, two R radicals together may also form a ring system;
Ar′ is the same or different at each instance and is an aromatic or heteroaromatic ring system which has 5 to 40 aromatic ring atoms and may be substituted by one or more R radicals;
R¹ is the same or different at each instance and is H, D, F, Cl, Br, I, N(R²)₂, CN, NO₂, OR², SR², Si(R²)₃, B(OR²)₂, C(═O)R², P(═O)(R²)₂, S(═O)R², S(═O)₂R², OSO₂R², a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may each be substituted by one or more R² radicals, where one or more nonadjacent CH₂ groups may be replaced by Si(R²)₂, C═O, NR², O, S or CONR² and where one or more hydrogen atoms in the alkyl, alkenyl or alkynyl group may be replaced by D, F, Cl, Br, I or CN, or an aromatic or heteroaromatic ring system which has 5 to 40 aromatic ring atoms and may be substituted in each case by one or more R² radicals; at the same time, two or more R¹ radicals together may form an aliphatic ring system;
R² is the same or different at each instance and is H, D, F, CN or an aliphatic, aromatic or heteroaromatic organic radical having 1 to 20 carbon atoms, in which one or more hydrogen atoms may also be replaced by F;
with the proviso that at least one R group is a heteroaromatic ring system and/or that at least one Ar¹ or Ar² group is a heteroaromatic ring system and/or that the compound has at least one group of formula (3).

16. The compound as claimed in claim 15, wherein the compound is of formula (4) or (5) 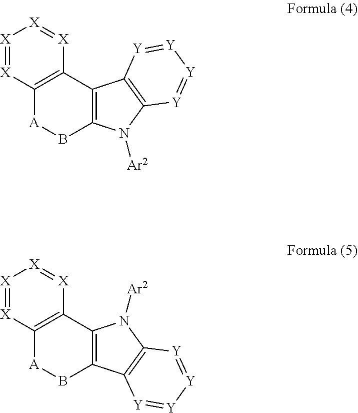
where the symbols used have the definitions given in claim 15.

17. The compound as claimed in claim 15, characterized in that one of the A and B groups is NAr¹ and the other of the A and B groups is C═O.

18. The compound as claimed in claim 15, wherein the compound is selected from the compounds of the formulae (4a), (4b), (5a) and (5b) 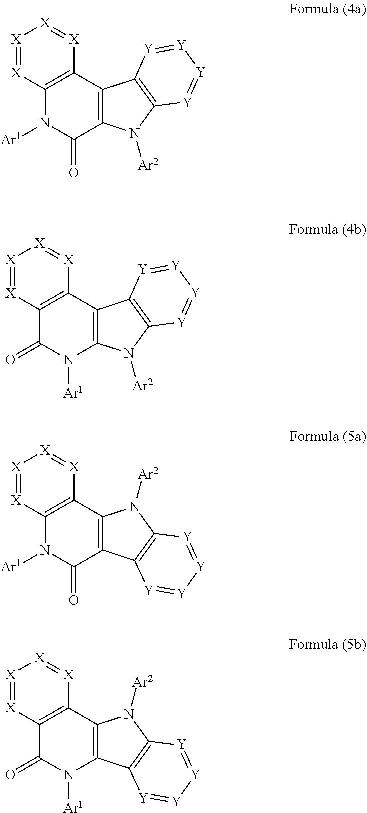
where the symbols used have the definitions given in claim 15.

19. The compound as claimed in claim 15, wherein the compound is selected from the compounds of the formulae (4a-3), (4b-3), (5a-3) and (5b-3) 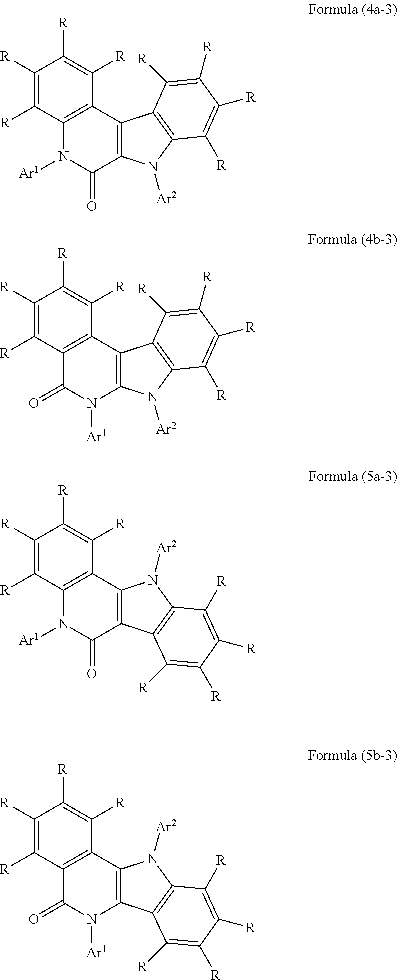
where the symbols used have the definitions given in claim 15.

20. The compound as claimed in claim 15, wherein the compound is selected from the compounds of the formulae (6) to (29) 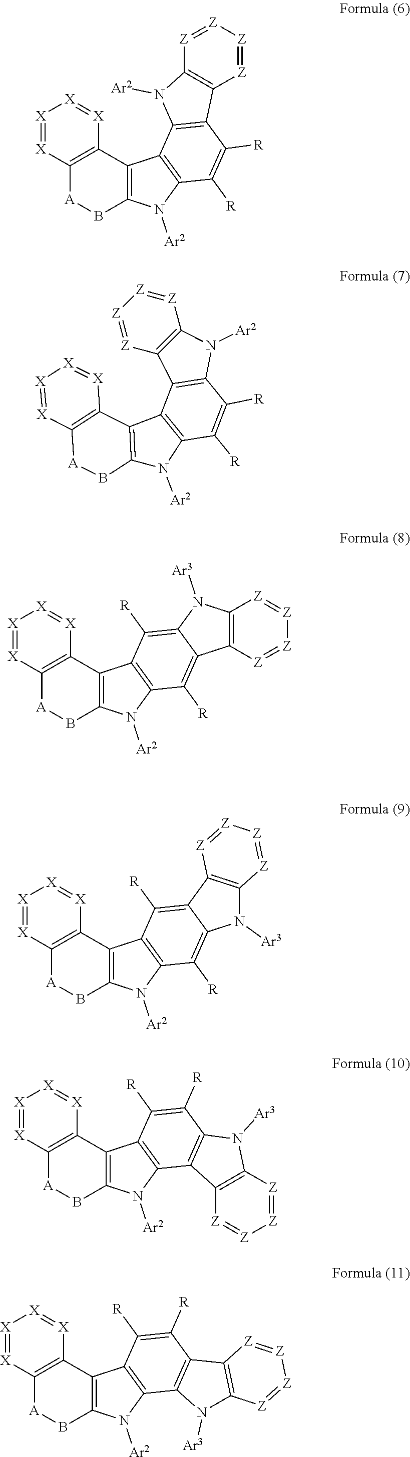 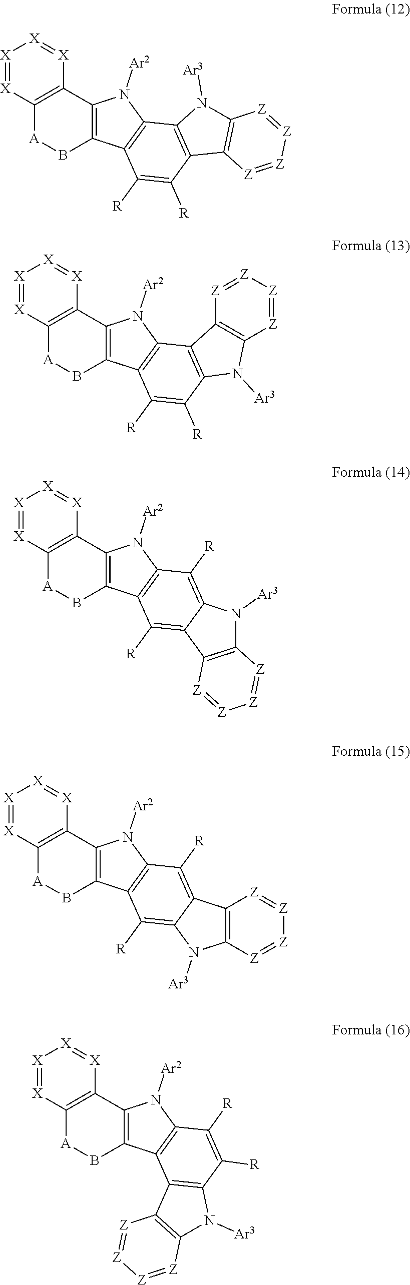 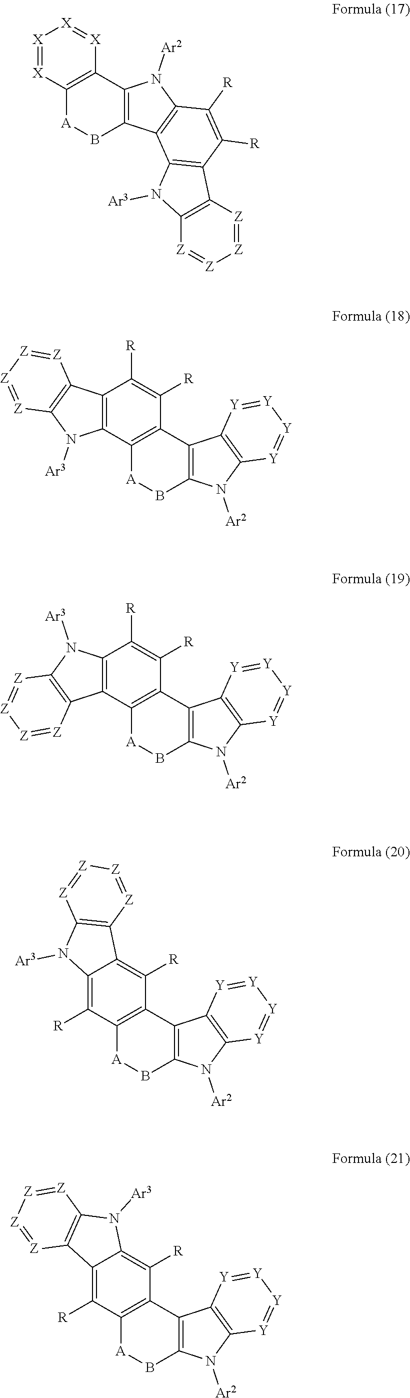 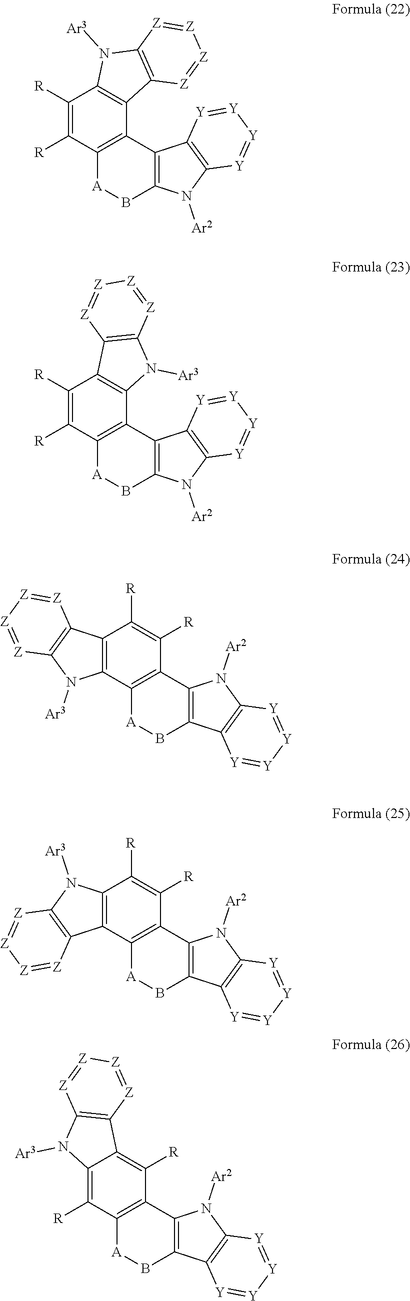 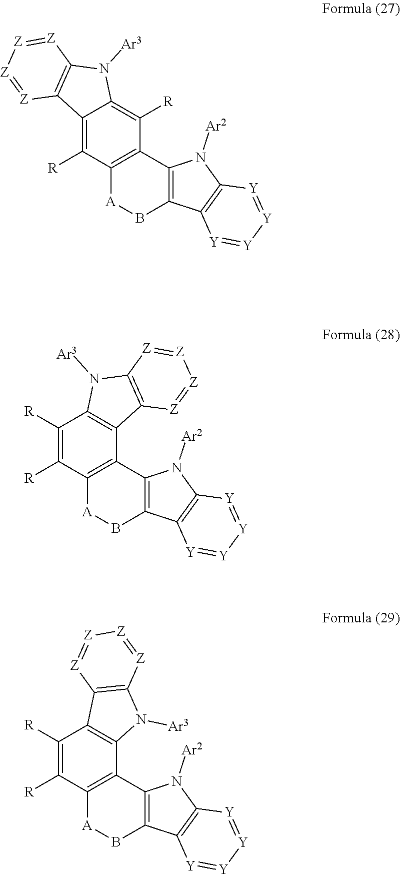
where the symbols used have the definitions given in claim 15.

21. The compound as claimed in claim 15, wherein the compound is selected from the compounds of the formulae (6-1) to (29-1) 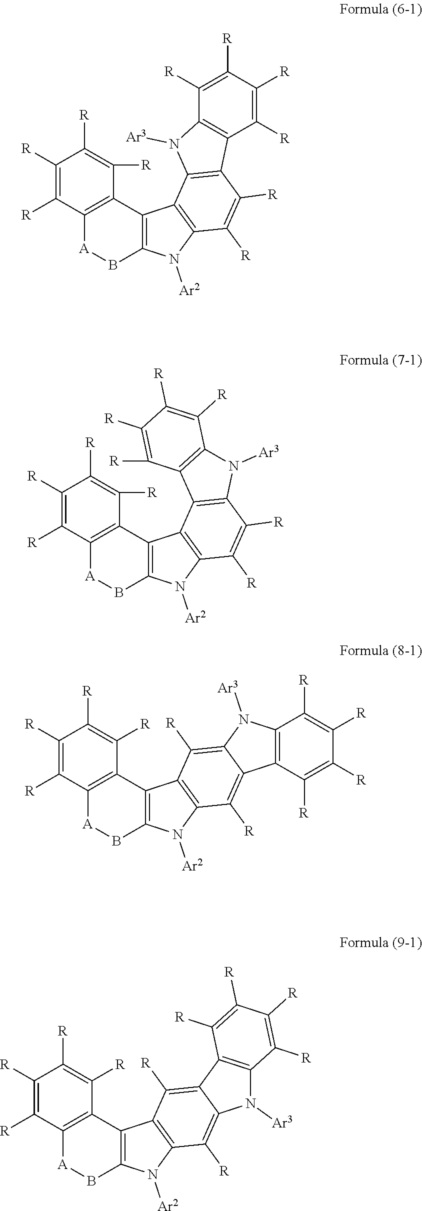 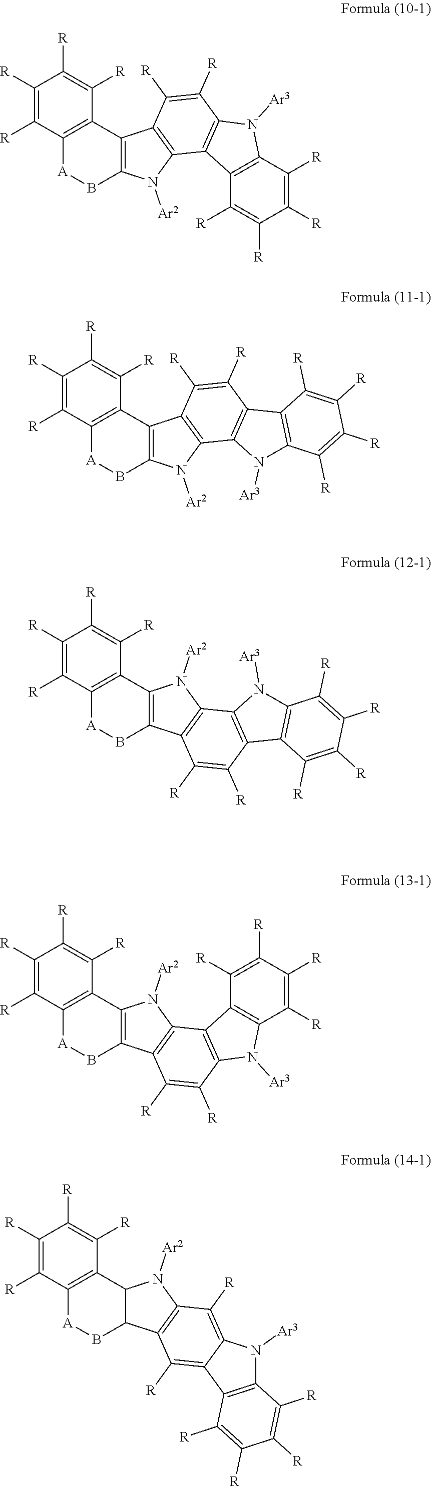 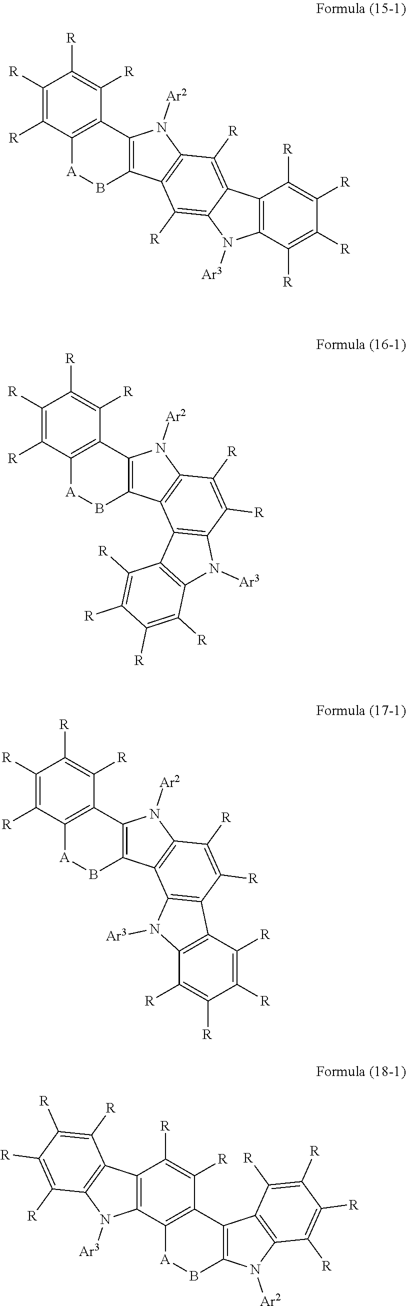 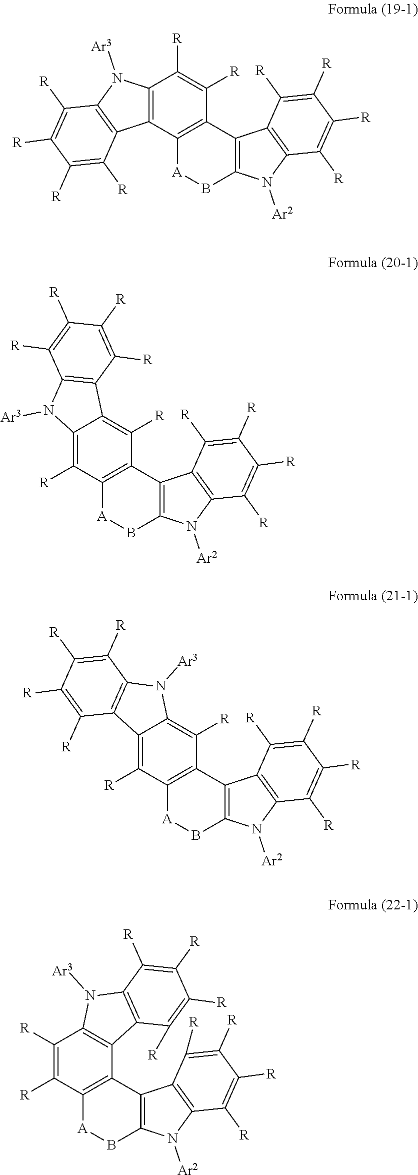 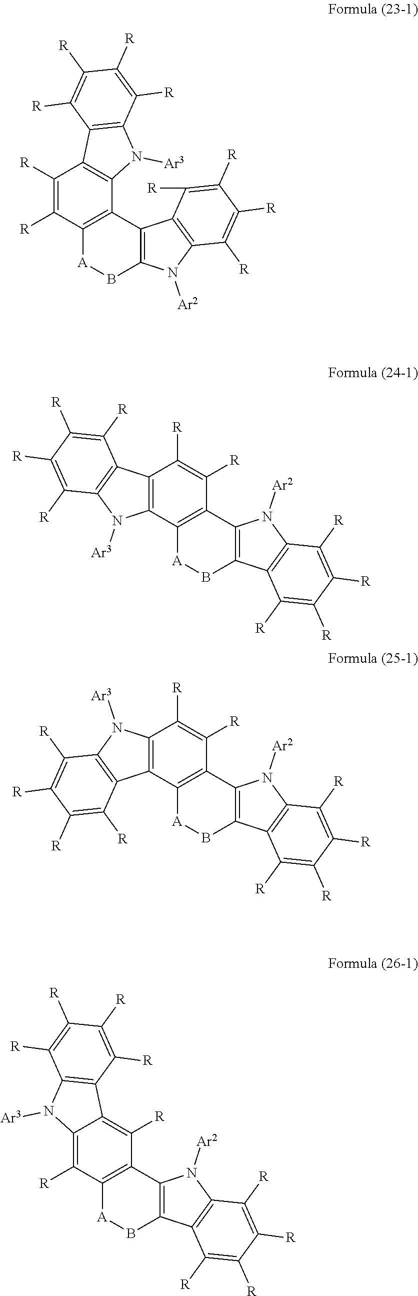 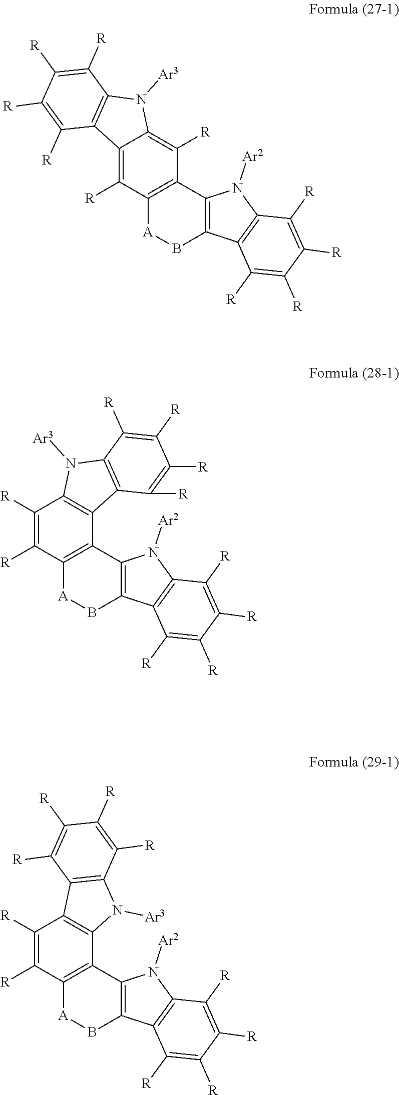
where the symbols used have the definitions given in claim 15.

22. The compound as claimed in claim 15, wherein the compound is selected from the compounds of the formulae (6a-1) to (29b-1) 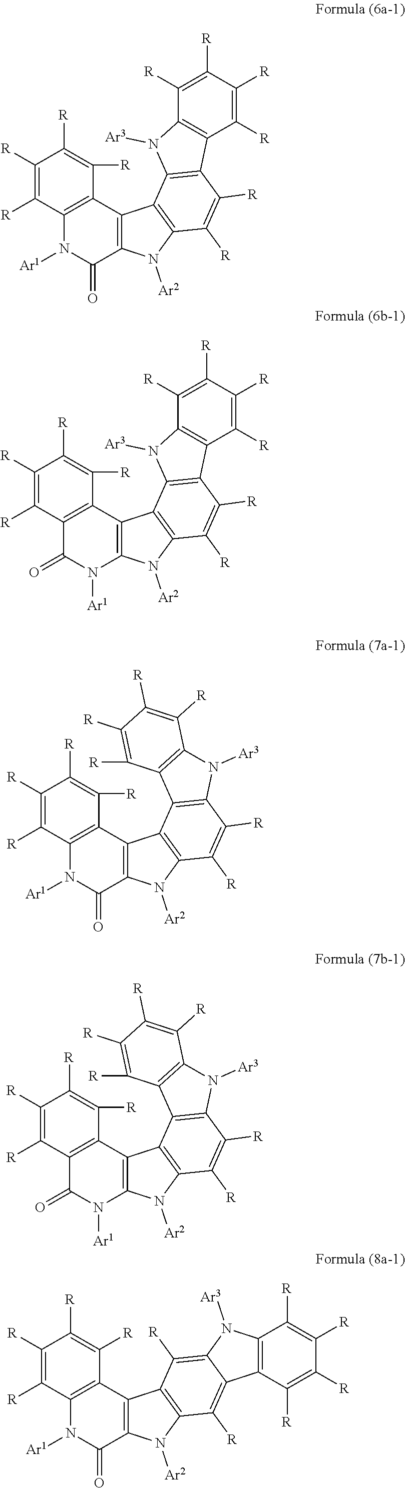 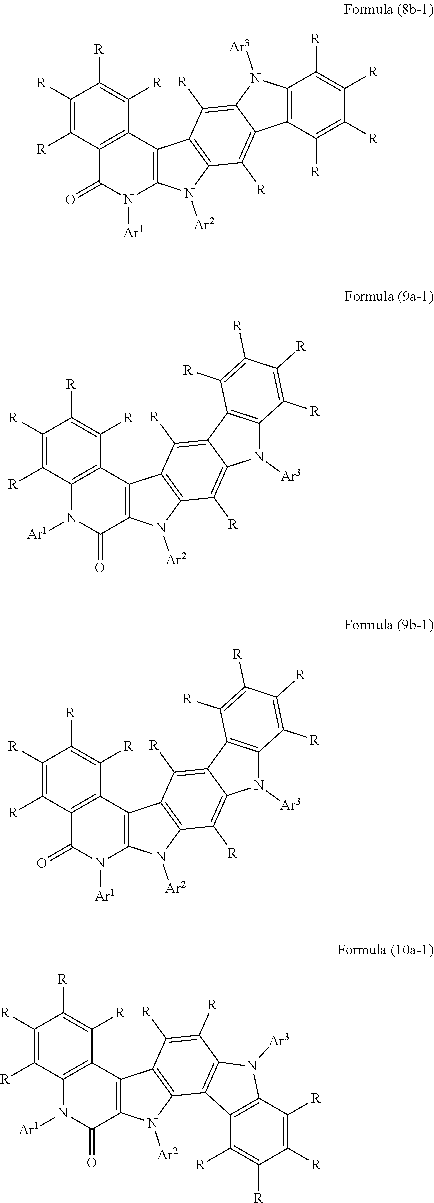 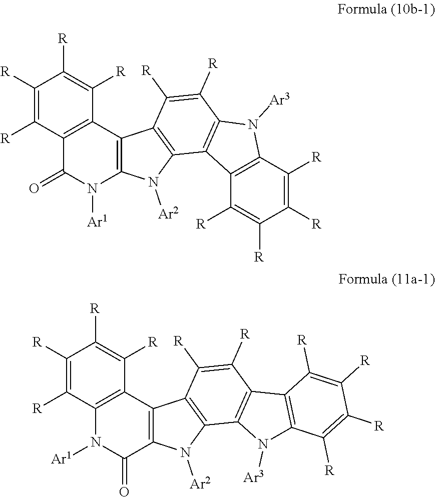 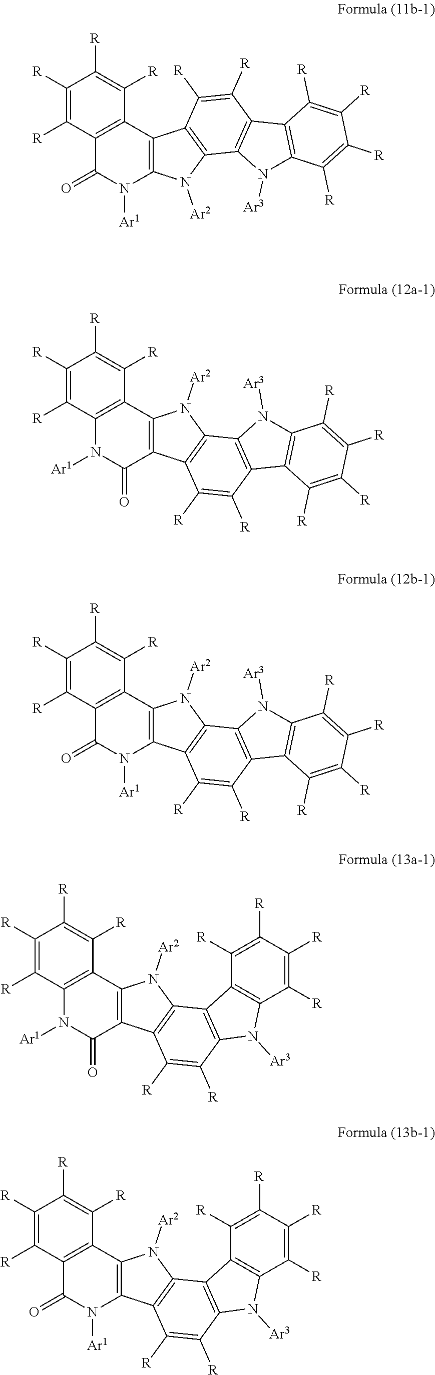 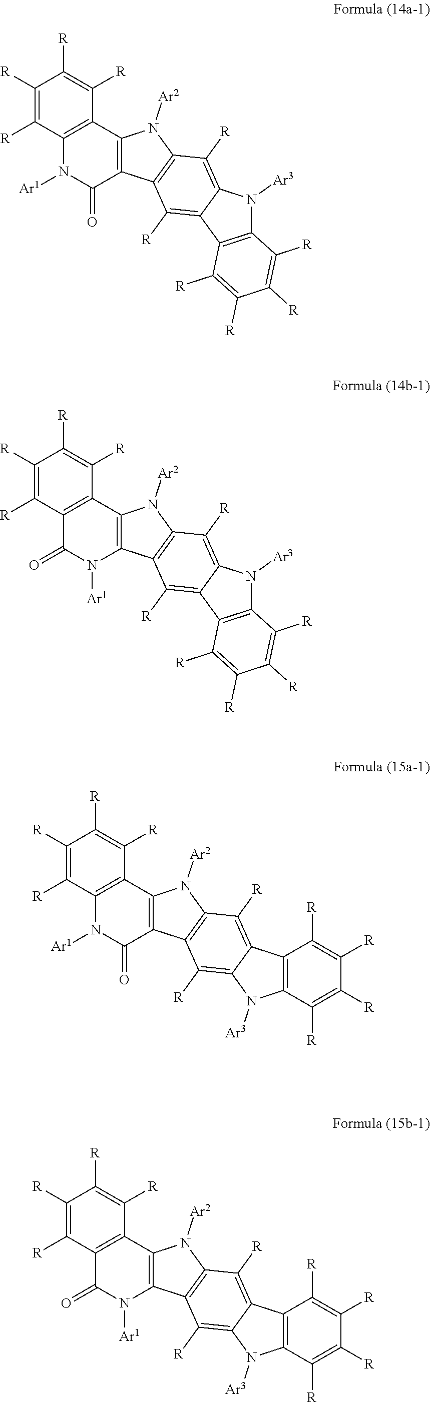 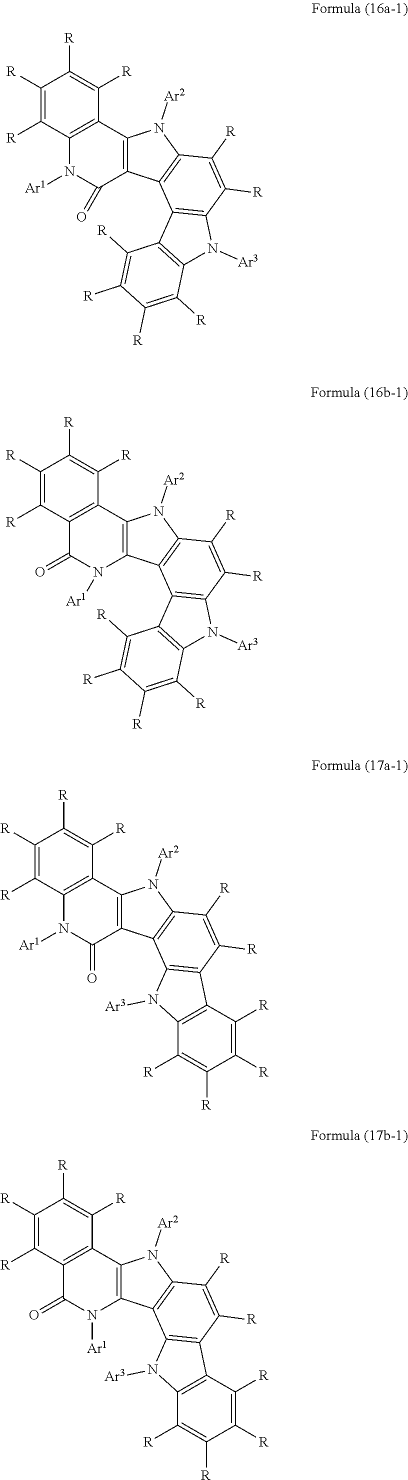 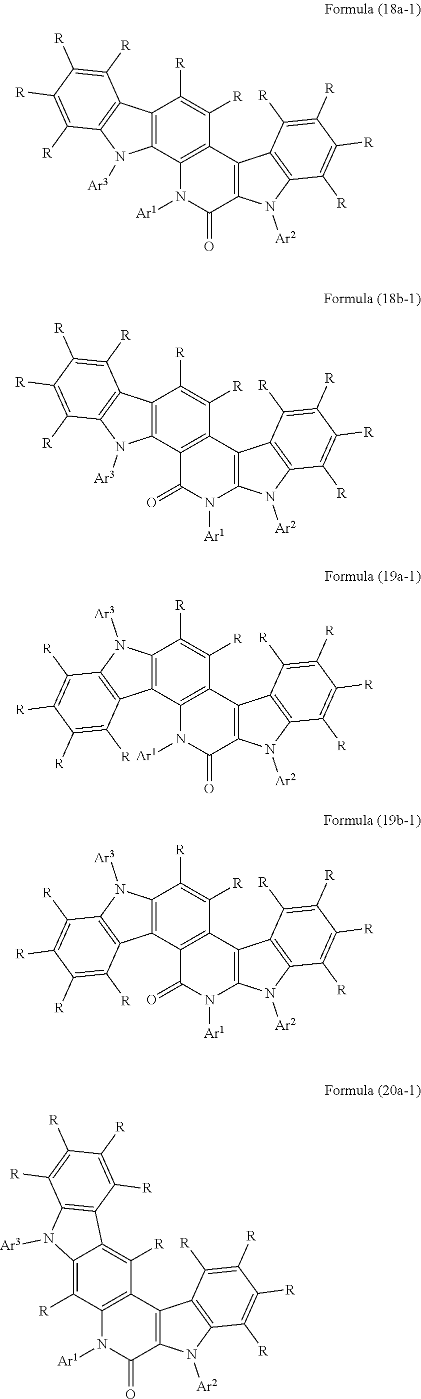 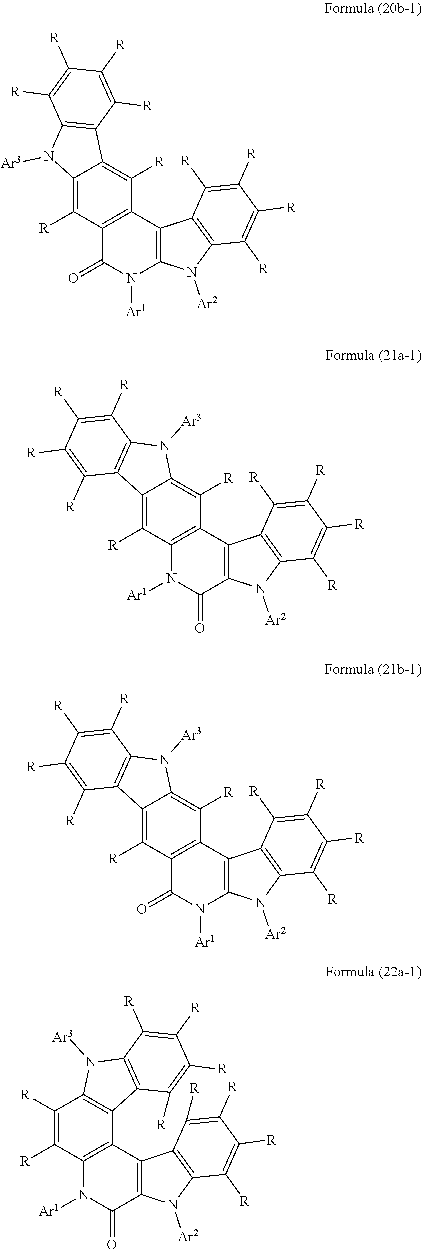 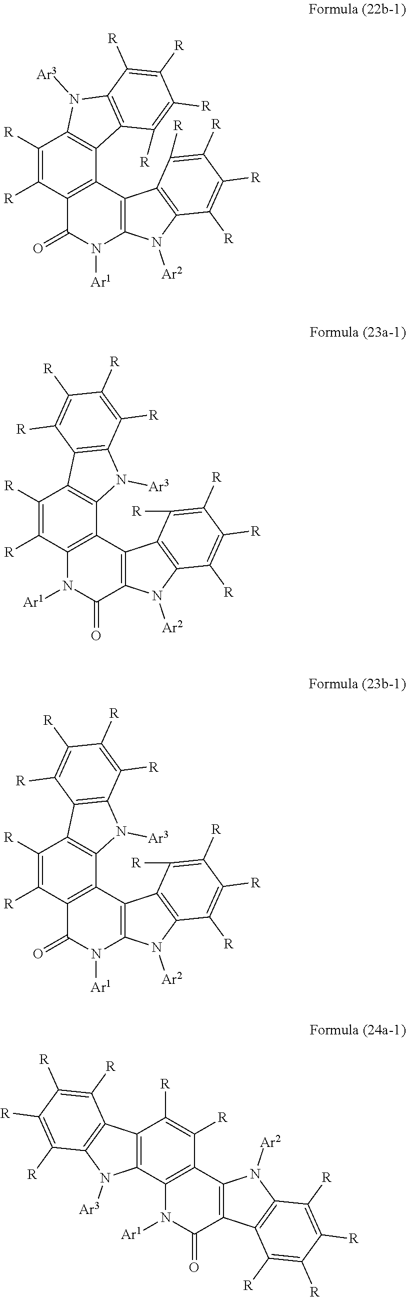 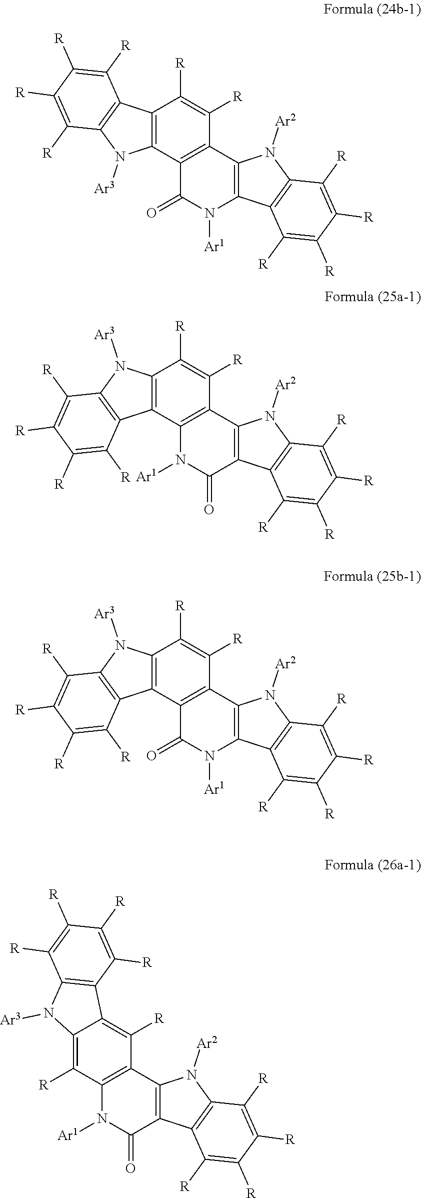 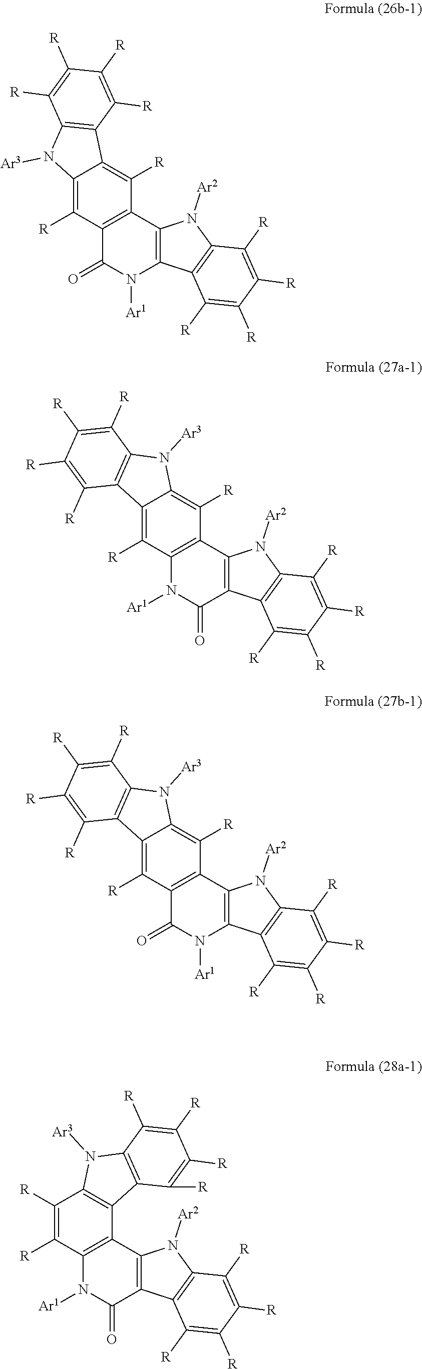 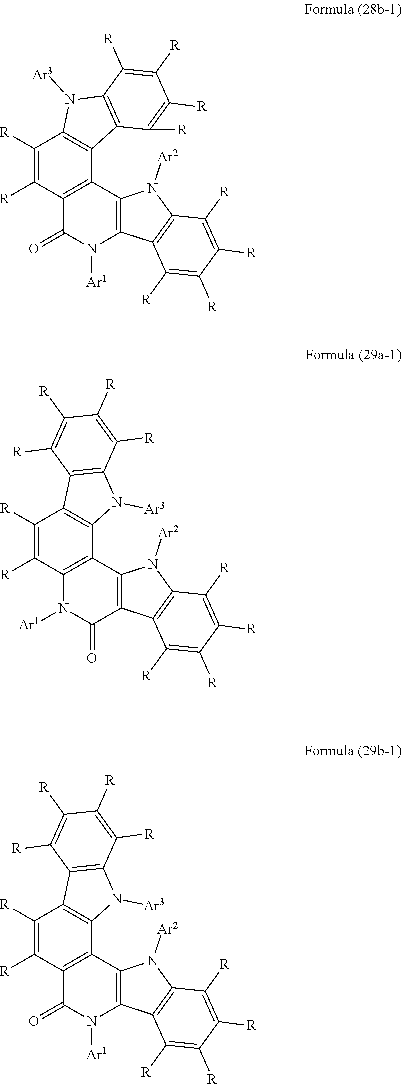
where the symbols used have the definitions given in claim 15.

23. The compound as claimed in claim 15, characterized in that Ar¹, Ar² and Ar³ are the same or different at each instance and are an aromatic or heteroaromatic ring system which has 6 to 24 aromatic ring atoms and may be substituted by one or more R radicals.

24. The compound as claimed in claim 15, characterized in that R is the same or different at each instance and is selected from the group consisting of H, D, F, N(Ar′)₂, CN, OR¹, a straight-chain alkyl group having 1 to 10 carbon atoms or an alkenyl group having 2 to 10 carbon atoms or a branched or cyclic alkyl group having 3 to 10 carbon atoms, where the alkyl or alkenyl group may in each case be substituted by one or more R¹ radicals, and where one or more nonadjacent CH₂ groups may be replaced by O, or an aromatic or heteroaromatic ring system which has 6 to 30 aromatic ring atoms and may be substituted in each case by one or more R¹ radicals; at the same time, two R radicals together may also form a ring system.

25. A formulation comprising at least one compound as claimed in claim 15 and at least one further compound and/or solvent.

26. An electronic device comprising at least one compound as claimed in claim 15.

27. An organic electroluminescent device, comprising the compound as claimed in claim 15 is used in an emitting layer as matrix material for phosphorescent emitters or for emitters that exhibit TADF (thermally activated delayed fluorescence), and/or in an electron transport layer and/or in a hole blocker layer and/or in a hole transport layer and/or in an exciton blocker layer.
